(19) United States
(12) Patent Application Publication
(10) Pub. No.: US 2023/0235358 A1
(43) Pub. Date: Jul. 27, 2023

(54) HOST DEFENSE SUPPRESSING METHODS AND COMPOSITIONS FOR MODULATING A GENOME

(71) Applicant: FLAGSHIP PIONEERING INNOVATIONS VI, LLC, Cambridge, MA (US)

(72) Inventors: Robert James CITORIK, Somerville, MA (US); Jacob Rosenblum RUBENS, Cambridge, MA (US); Cecilia Giovanna Silvia COTTA-RAMUSINO, Cambridge, MA (US); William Edward SALOMON, West Roxbury, MA (US); Zi Jun WANG, Arlington, MA (US)

(21) Appl. No.: 17/823,726

(22) Filed: Aug. 31, 2022

Related U.S. Application Data
(63) Continuation of application No. PCT/US2021/021213, filed on Mar. 5, 2021.
(60) Provisional application No. 63/147,529, filed on Feb. 9, 2021.
(60) Provisional application No. 63/035,653, filed on Jun. 5, 2020.
(60) Provisional application No. 62/985,750, filed on Mar. 5, 2020.

(51) Int. Cl. C12N 15/88; C12N 15/113; C12N 15/11; C12N 9/22; C12N 15/90

(52) U.S. Cl. CPC C12N 15/88; C12N 15/1135; C12N 15/1136; C12N 15/11; C12N 9/22; C12Y 301/13002; C12N 15/907; C12N 2310/14; C12N 2310/20; C12N 2800/80

(57) ABSTRACT

The disclosure provides, e.g., compositions and methods for modulating a host response to a Gene Writer system. In some embodiments, modulation of the host response results in increased integration of a heterologous nucleic acid sequence of interest into a target genome. In some embodiments, modulation of the host response results in an increased stability, e.g., maintenance of an insertion or expression thereof. In some embodiments, modulation of the host response results in decreased cytotoxicity.

### RELATED APPLICATIONS

This application is a continuation of International Application No. PCT/US2021/021213, filed Mar. 5, 2021, which claims priority to U.S. Ser. No. 62/985,750, filed Mar. 5, 2020, U.S. Ser. No. 63/035,653, filed Jun. 5, 2020, and U.S. Ser. No. 63/147,529, filed Feb. 9, 2021, the entire contents of each of which is incorporated herein by reference.

### SEQUENCE LISTING

The instant application contains a Sequence Listing which has been submitted electronically in ASCII format and is hereby incorporated by reference in its entirety. Said ASCII copy, created on Aug. 30, 2022, is named V2065-701120_SL.xml and is 554,773 bytes in size.

### BACKGROUND

Techniques for gene integration into the genome has advanced in recent years, yet the efficiency of gene integration still remains too low for certain applications. There is a need in the art for improved compositions and methods for increasing the efficiency of gene integration.

### SUMMARY OF THE INVENTION

The present disclosure provides, e.g., a method of modifying a target DNA molecule in a mammalian host cell, the method comprising:
a) contacting (e.g., directly or indirectly, e.g., by providing access to the cell, e.g., by systemic administration) the host cell with a gene modifying system; and
b) contacting (e.g., directly or indirectly, e.g., by providing access to the cell, e.g., by systemic administration) the host cell with an agent that promotes activity of the gene modifying system (e.g., a host response modulator or an epigenetic modifier),
wherein the gene modifying system comprises a Gene Writer polypeptide, or a nucleic acid encoding the Gene Writer polypeptide, and a template nucleic acid, the template nucleic acid comprising i) a sequence that binds the Gene Writer polypeptide and ii) a heterologous object sequence. The disclosure also provides, e.g., a method of modifying a target DNA molecule in a mammalian host cell, the method comprising, contacting (e.g., directly or indirectly, e.g., by providing access to the cell, e.g., by systemic administration) the host cell with:
I) a gene modifying system and optionally a delivery vehicle for the gene modifying system, wherein the gene modifying system comprises:
a) a Gene Writer polypeptide, or a nucleic acid encoding the Gene Writer polypeptide, and
b) a template nucleic acid, the template nucleic acid comprising i) a sequence that binds the Gene Writer polypeptide and ii) a heterologous object sequence; and
II) an agent that promotes activity of the gene modifying system, (e.g., a host response modulator or an epigenetic modifier), linked to a component of the gene modifying system or the delivery vehicle.

For example, the agent that promotes activity of the gene modifying system may be covalently linked to a component of the gene modifying system, e.g., is fused with a component of the gene modifying system, e.g., a Gene Writer polypeptide or nucleic acid encoding the Gene Writer polypeptide, e.g., a Gene Writer template nucleic acid (e.g., RNA or DNA template) or nucleic acid encoding a Gene Writer template (e.g., DNA encoding an RNA template), an additional nucleic acid of a Gene Writing system (e.g., a gRNA), or a delivery vehicle of a gene modifying system, e.g., an AAV or nanoparticle (e.g., LNP). In some embodiments, the agent that promotes activity of the gene modifying system is embedded in or co-formulated with the delivery vehicle.

The disclosure also provides a kit comprising:

a) a gene modifying system that comprises a Gene Writer polypeptide, or a nucleic acid encoding the Gene Writer polypeptide, and a template nucleic acid, the template nucleic acid comprising i) a sequence that binds the Gene Writer polypeptide and ii) a heterologous object sequence; and

b) an agent that promotes activity of the gene modifying system (e.g., a host response modulator or an epigenetic modifier).

The disclosure also provides a kit comprising,

a gene modifying system comprising a Gene Writer polypeptide, or a nucleic acid encoding the Gene Writer polypeptide, and a template nucleic acid, the template nucleic acid I) a gene modifying system and optionally a delivery vehicle for the gene modifying system, wherein the gene modifying system comprises:
a) a Gene Writer polypeptide, or a nucleic acid encoding the Gene Writer polypeptide, and
b) a template nucleic acid, the template nucleic acid comprising i) a sequence that binds the Gene Writer polypeptide and ii) a heterologous object sequence; and

II) an agent that promotes activity of the gene modifying system, (e.g., a host response modulator or an epigenetic modifier), linked to a component of the gene modifying system or the delivery vehicle.

For example, the agent that promotes activity of the gene modifying system may be covalently linked to a component of the gene modifying system, e.g., is fused with a component of the gene modifying system, e.g., a Gene Writer polypeptide or nucleic acid encoding the Gene Writer polypeptide, a Gene Writer template nucleic acid (e.g., RNA or DNA template) or nucleic acid encoding a Gene Writer template (e.g., DNA encoding an RNA template), an additional nucleic acid of a Gene Writing system (e.g., a gRNA), or a delivery vehicle of a gene modifying system, e.g., an AAV or nanoparticle (e.g., LNP). In some embodiments, the agent that promotes activity of the gene modifying system is embedded in or co-formulated with the delivery vehicle.

The disclosure also provides a composition comprising:

a) a gene modifying system that comprises a Gene Writer polypeptide, or a nucleic acid encoding the Gene Writer polypeptide, and a template nucleic acid, the template nucleic acid comprising i) a sequence that binds the Gene Writer polypeptide and ii) a heterologous object sequence; and
b) an agent that promotes activity of the gene modifying system (e.g., a host response modulator or an epigenetic modifier).

The disclosure also provides a composition comprising:

a gene modifying system comprising a Gene Writer polypeptide, or a nucleic acid encoding the Gene Writer polypeptide, and a template nucleic acid, the template nucleic acid I) a gene modifying system and optionally a delivery vehicle for the gene modifying system, wherein the gene modifying system comprises:
a) a Gene Writer polypeptide, or a nucleic acid encoding the Gene Writer polypeptide, and
b) a template nucleic acid, the template nucleic acid comprising i) a sequence that binds the Gene Writer polypeptide and ii) a heterologous object sequence; and

II) an agent that promotes activity of the gene modifying system, (e.g., a host response modulator or an epigenetic modifier), linked to a component of the gene modifying system or the delivery vehicle.

In some embodiments, the epigenetic modifier comprises an HDAC inhibitor or a histone methyltransferase inhibitor, e.g., as described herein.

In some embodiments, the agent that promotes activity of the gene modifying system comprises an antibody, a polypeptide (e.g., a dominant negative mutant of a polypeptide in a host response pathway), an enzyme (e.g., endopeptidase, e.g., Ig-cleaving endopeptidase, e.g., IdeS), a small molecule, or a nucleic acid (e.g., an RNAi molecule). In some embodiments, the enzyme is a wild-type enzyme or a functional fragment or variant thereof. In some embodiments, the agent that promotes activity of the gene modifying system comprises a nucleic acid that is covalently linked to the GeneWriter polypeptide or the template nucleic acid. For instance, the nucleic acid may encode a protein, e.g., a therapeutic protein, that promotes activity of the gene modifying system. In some embodiments, the agent that promotes activity of the gene modifying system is a small molecule. In some embodiments, the agent that promotes activity of the gene modifying system is a domain of a polypeptide.

In some embodiments, the agent that promotes activity of the gene modifying system (e.g., a host response inhibitor) comprises a protein or domain that inhibits a host process. In some embodiments, the agent inhibits or sequesters a host protein (e.g., host enzyme) or host complex. In some embodiments, the host protein (or the complex comprising the host protein) inhibits the gene modifying system. In some embodiments, the host enzyme (or the complex comprising a host enzyme) inhibits the gene modifying system. For example, the host protein could be a DNA repair enzyme that inhibits the gene modifying system. In some embodiments, the host protein is involved in Homology Directed Repair (HDR), e.g., a protein described herein.

In some embodiments, the host protein that is inhibited or sequestered is a protein that inhibits the desired editing outcome of the gene modifying system. In some embodiments, inhibiting the gene modifying system means inhibiting gene modification at one or more steps during a Gene Writing process, optionally including (i) target DNA binding, (ii) single-stranded target DNA cleavage, (iii) association of a Gene Writing template with the target DNA, e.g., template annealing, (iv) target-primed polymerization of DNA from the Gene Writing template, (v) second nick of opposite strand of target DNA, (vi) second-strand synthesis of DNA using newly polymerized DNA from (iv) as the polymerization template, or optionally second-strand synthesis using an additional Gene Writing template, (vii) flap exonuclease activity towards the target DNA, and/or (viii) ligation of newly synthesized DNA to a free 5′ end in the target genome. In some embodiments, the agent is fused to the Gene Writer polypeptide.

In some embodiments, the agent that promotes activity of the gene modifying system comprises a protein or domain that stimulates a host process. In some embodiments, the agent activates or recruits a host protein (e.g., host enzyme) or host complex. In some embodiments, the host enzyme is (or the complex comprises) a DNA repair enzyme that promotes activity of the gene modifying system, e.g., a DNA polymerase or a DNA ligase. In some embodiments, the agent is fused to the Gene Writer polypeptide.

In some embodiments, the agent that promotes activity of the gene modifying system comprises a protein or domain that binds a host cell protein. In some embodiments, the binding of the host cell protein to a component of the gene modifying system functions to recruit activity of that host protein (or complex containing the host protein) to the target site. In some embodiments, the host cell protein comprises a 5′ exonuclease, e.g., EXO1. In some embodiments, the host cell protein comprises a structure-specific endonuclease, e.g., FEN1. In some embodiments, the agent is fused to the Gene Writer polypeptide. In some embodiments, the Gene Writer polypeptide comprises a Cas domain, e.g., a Cas9 nickase domain or catalytically inactive Cas9 domain. In some embodiments, the template nucleic acid comprises, from 5′ to 3′ (1) a gRNA spacer; (2) a gRNA scaffold; (3) heterologous object sequence (4) 3′ homology domain.

In some embodiments, the agent that promotes activity of the gene modifying system comprises a protein or domain that replaces or supplements a host protein, complex, or pathway. In some embodiments, the agent comprises a 5′ exonuclease, e.g., EXO1 or an active fragment or variant thereof. In some embodiments, the agent (e.g., EXO1) comprises a sequence according to NCBI:NP_006018.4 or UniProt: Q9UQ84, each of which is herein incorporated by reference, or a sequence having at least 70%, 80%, 85%, 90%, 95%, 98%, or 99% identity thereto. In some embodiments, the agent comprises a structure-specific endonuclease, e.g., FEN1, or an active fragment or variant thereof. In some embodiments, the agent (e.g., FEN1) comprises a sequence according to NCBI:NP_004102.1 or UniProt: P39748, each of which is herein incorporated by reference, or a sequence having at least 70%, 80%, 85%, 90%, 95%, 98%, or 99% identity thereto. In some embodiments, the Gene Writer polypeptide comprises a Cas domain, e.g., a catalytically inactive Cas domain. In some embodiments, the template nucleic acid comprises, from 5′ to 3′ (1) a gRNA spacer; (2) a gRNA scaffold; (3) heterologous object sequence (4) 3′ homology domain.

In some embodiments, the agent is fused to delivery vehicle or a component of a delivery vehicle, e.g., an AAV, e.g., an AAV capsid. In some embodiments, the agent reduces a host immune response. In some embodiments, the agent comprises a protease, e.g., an exopeptidase or endopeptidase, that cleaves a component of the host immune response, e.g., an immunoglobulin or cytokine. In some embodiments, the agent comprises an endopeptidase that cleaves a host antibody, e.g., an antibody that binds the delivery vehicle, e.g., an antibody that neutralizes or inhibits the delivery vehicle, e.g., an antibody that neutralizes or inhibits AAV. In some embodiments, the endopeptidase is an Ig-cleaving endopeptidase, e.g., IdeS. In some embodiments, the IdeS cleaves IgG below the hinge region. Methods to prevent an immune response elicited by administration of a gene therapy or for treating a patient with pre-existing immunity to a viral capsid using IdeS and other immunoglobulin G-degrading enzyme polypeptides are described in Leborgne et al Nat Med 26:1096-1101 (2020) and in PCT/EP2019/069280.

In some embodiments, an IdeS protein used with the system is is a bacterial IgG endopeptidase or bacterial IdeS/Mac family cysteine endopeptidase. In some embodiments, an IdeS protein used with the system is the IgG endopeptidase or IdeS/Mac family cysteine endopeptidase from Streptococcus pyogenes or Streptococcus equi. In some embodiments, the Ig-cleaving endopeptidase (e.g., IdeS) comprises a sequence according to WP_012678049.1 or WP_002992557.1, or a sequence having at least 70%, 80%, 85%, 90%, 95%, 98%, or 99% identity thereto. In some embodiments, the IdeS may be a modified variant, e.g., an IdeS with the sequence corresponding to SEQ ID Nos:3-18, 23, or 48 from PCT/EP2019/069280 which is incorporated in here in its entirety including the sequences of IdeS corresponding to SEQ ID Nos: 18, 23, and 48. In some embodiments, the Ig-cleaving endopeptidase may be a IdeZ. In some embodiments, the Ig-cleaving endopeptidase (e.g., IdeZ) comprises a sequence according to WP_014622780.1, or a sequence having at least 70%, 80%, 85%, 90%, 95%, 98%, or 99% identity thereto. Other proteases that may be used in the current disclosure include, for example and without limitation, IgdE enzymes from S. suis, S. porcinus, and S. equi. In some embodiments the protease may be, an IdeMC or a homolog thereof. Other endopeptidases that may be used in the current disclosure include, for example and without limitation, IdeZ with and without the N-terminal methionine and signal peptide and IdeS/IdeZ hybrid proteins described in WO 2016/128559, which is incorporated herein by reference in its entirety. Other proteases that may be used in the current disclosure include, for example and without limitation, proteases described in Jordan et al. (N Engl. J Med. 377; 5, 2017), Lannergard and Guss (FEMS Microbiol Lett., 262(2006); 230-235) and Hulting et al., (FEMS Microbiol Lett., 298(2009), 44-50). In some embodiments, the agent promotes immunotolerance.

In some embodiments, the agent may be an immunosuppressive agent. In some embodiments, the agent may suppress macrophage engulfment, e.g., CD47 or a fragment or variant thereof, or an agent that promotes expression of CD47 in a target cell. In some embodiments the agent may be a soluble immunosuppressive cytokine, e.g., IL-10 or a fragment or variant thereof, or an agent that may promote expression of soluble immunosuppressive cytokine, e.g., IL-10 or a fragment or variant thereof in a target cell. In some embodiments the agent may be a soluble immunosuppressive protein or a fragment or variant thereof or an agent that may promote expression of a soluble immunosuppressive protein or a fragment or variant thereof in a target cell. In some embodiments the soluble immunosuppressive protein may be PD-1, PD-L1, CTLA4, or BTLA or a fragment or a variant thereof.

In some embodiments the agent may be a tolerogenic protein, e.g., an ILT-2 or ILT-4 agonist, e.g., HLA-E or HLA-G or any other endogenous ILT-2 or ILT-4 agonist or a functional fragment or variant thereof. In some embodiments, the agent may promote the expression of a tolerogenic protein, e.g., an ILT-2 or ILT-4 agonist, e.g., HLA-E or HLA-G or any other endogenous ILT-2 or ILT-4 agonist or a functional fragment or variant thereof. In some embodiments the agent may comprise a protein that suppresses complement activity, e.g., reduces activity of a complement regulatory protein, e.g., a protein that binds decay-accelerating factor (DAF, CD55), e.g., factor H (FH)-like protein-1 (FHL-1), e.g., C4b-binding protein (C4BP), e.g., complement receptor 1 (CD35), e.g., Membrane cofactor protein (MCP, CD46), e.g., Profectin (CD59). In some embodiments, the agent may promote expression of protein that suppresses complement activity, e.g., complement regulatory proteins, e.g., proteins that bind decay-accelerating factor (DAF, CD55), e.g., factor H (FH)-like protein-1 (FHL-1), e.g., C4b-binding protein (C4BP), e.g., complement receptor 1 (CD35), e.g., Membrane cofactor protein (MCP, CD46), e.g., Profectin (CD59).

In some embodiments, the agent may comprise a protein that inhibits a classical or alternative complement pathway CD/C5 convertase enzyme, e.g., a protein that regulates MAC assembly. In some embodiments, the agent may promote the expression of a protein that inhibits the classical or alternative complement pathway CD/C5 convertase enzymes, e.g., a protein that regulates MAC assembly. In some embodiments the agent may comprise a histocompatibility antigen, e.g., an HLA-E or an HLA-G. In some embodiments the agent may promote the expression of a histocompatibility antigen, e.g., an HLA-E or an HLA-G. In some embodiments, the agent comprises glycosylation, e.g., containing sialic acid, which acts to, e.g., suppress NK cell activation. In some embodiments the agent may promote surface glycosylation profile, e.g., containing sialic acid, which acts to, e.g., suppress NK cell activation.

In some embodiments, the agent may be a complement targeted therapeutic, e.g., a complement regulatory protein, e.g., complement inhibitor, e.g., a protein that binds to a complement component, e.g., C1-inhibitor, or a variant or fragment thereof. In some embodiments, the agent may be a soluble regulator. In some embodiments, the agent may be a membrane-bound regulator, e.g, DAF/CD55, MCP/CD46, or CD59. In some embodiments, the agent is a small molecule, a protein, a fusion protein, an antibody, or an antibody-drug conjugate. In some such instances a complement targeted therapeutic is described in Ricklin et al Nat Biotechnol 25(11): 1265-1275 (2007) and Schauber-Plewa et al Gene Ther 12(3): 238-45 (2005), both of which are incorporated by reference herein in their entirety.

In some embodiments, the agent may be an agent which reduces the level of an immune activating agent. In some embodiments, the agent suppresses expression of MHC class I or MHC class II. In some embodiments, the agent suppresses expression of one or more co-stimulatory proteins. In some embodiments, the co-stimulatory proteins include but are not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4. In some embodiments, the agent that reduces the level of an immune activating agent comprises a small molecule or an inhibitory RNA.

In some embodiments, the agent does not substantially elicit an immunogenic response by the immune system, e.g., innate immune system. In some embodiments, the immunogenic response by the innate immune system comprises a response by innate immune cells including, but not limited to NK cells, macrophages, neutrophils, basophils, eosinophils, dendritic cells, mast cells, or gamma/delta T cells.

In some embodiments, the agent does not substantially elicit an immunogenic response by the immune system, e.g., adaptive immune system. In some embodiments, the immunogenic response by the adaptive immune system comprises an immunogenic response by an adaptive immune cell including, but not limited to a change, e.g., increase, in number or activity of T lymphocytes (e.g., CD4 T cells, CD8 T cells, and or gamma-delta T cells), or B lymphocytes.

In some embodiments, the agent promotes immunotolerance to a delivery vehicle, e.g., a viral capsid, e.g., an AAV capsid. In some embodiments, the agent promotes immunotolerance to a component of the gene modifying system, e.g., a Gene Writer polypeptide or nucleic acid encoding the Gene Writer polypeptide, a Gene Writer template nucleic acid (e.g., RNA or DNA template) or nucleic acid encoding a Gene Writer template (e.g., DNA encoding an RNA template), an additional nucleic acid of a Gene Writing system (e.g., a gRNA), or a delivery vehicle of a gene modifying system, e.g., an AAV or nanoparticle. In some embodiments, the agent promotes immunotolerance to one or more products expressed from the genome after the activity of the gene modifying system, e.g., a therapeutic protein, e.g., a therapeutic protein expressed from a coding sequence integrated into the genome or a variant of a host protein created by the targeted modification of the endogenous coding sequence.

In some embodiments, the contacting of the host cell with the Gene Writer polypeptide and the agent that promotes activity of the gene modifying system results in increased levels of the heterologous object sequence in host cell genome compared to an otherwise similar cell not contacted with the agent that promotes activity of the gene modifying system, e.g., wherein the number of copies of heterologous object sequence in the genome of a population of host cells is at least 5%, 10%, 20%, 30%, 40%, 50%, 60%, 70%, 80%, 90%, or 100% higher, or at least 2-fold, 5-fold, or 10-fold higher, than the number of copies of heterologous object sequence in the genome of otherwise similar cells that were contacted with the gene modifying system but not with the agent that promotes activity of the gene modifying system.

### BRIEF DESCRIPTION OF THE DRAWINGS

FIGS. 1A and 1B describes luciferase activity assay for primary cells. LNPs formulated as according to Example 9 were analyzed for delivery of cargo to primary human (A) and mouse (B) hepatocytes, as according to Example 10. The luciferase assay revealed dose-responsive luciferase activity from cell lysates, indicating successful delivery of RNA to the cells and expression of Firefly luciferase from the mRNA cargo.

FIG. 2 shows LNP-mediated delivery of RNA cargo to the murine liver. Firefly luciferase mRNA-containing LNPs were formulated and delivered to mice by iv, and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours, post-administration.

### DETAILED DESCRIPTION

### Definitions

As used herein, the term “agent that promotes activity of the gene modifying system” refers to an agent (e.g., a compound, plurality of compounds, nucleic acid, polypeptide, or complex) that promotes a desired alteration to a target nucleic acid (e.g., insertion of a heterologous object sequence into a target site in the target nucleic acid) in the presence of the gene modifying system. In some embodiments, the agent that promotes activity of the gene modifying system is a host response modulator or an epigenetic modifier. In some embodiments, the agent that promotes activity of the gene modifying system acts on the target site, an endogenous protein, or an endogenous RNA.

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments, e.g., Fab, Fab′ and F(ab′)2, Fd, Fvs, single-chain Fvs (scFv), rlgG, single-chain antibodies, disulfide-linked Fvs (sdFv), fragments including either a VL or VH domain, fragments produced by an Fab expression library, and anti-idiotypic (anti-Id) antibodies. Antibody molecules can be of any type (e.g., IgG, IgE, IgM, IgD, IgA, and IgY), class (e.g., IgG1, IgG2, IgG3, IgG4, IgA1 and IgA2) or subclass of immunoglobulin molecule. Moreover, unless otherwise indicated, the term “monoclonal antibody” (mAb) is meant to include both intact molecules as well as antibody fragments (such as, for example, Fab and F(ab′)2 fragments) that are capable of specifically binding to a target protein. Fab and F(ab′)2 fragments lack the Fc fragment of an intact antibody. The term “inhibitory antibody” refers to antibodies that are capable of binding to a target antigen and inhibiting or reducing its function and/or attenuating one or more signal transduction pathways mediated by the antigen. For example, inhibitory antibodies may bind to and block a ligand-binding domain of a receptor, or to extracellular regions of a transmembrane protein. Inhibitory antibody molecules that enter a cell may block the function of an enzyme antigen or signaling molecule antigen. Inhibitory antibodies inhibit or reduce antigen function and/or attenuate one or more antigen-mediated signal transduction pathway by at least 10% (e.g., 10%, 20%, 30%, 40%, 50%, 60%, 70%, 80%, 90%, 95%, 98% or more). The term “agonist antibody” refers to antibodies that are capable of binding to a target antigen and increasing its activity or function, e.g., increasing or activating one or more signal transduction pathways mediated by the antigen. For example, an agonist antibody may bind to and agonize an extracellular region of a transmembrane protein. Agonist antibody molecules that enter a cell may increase the function of an enzyme antigen or signaling molecule antigen. Agonist antibodies activate or increase antigen function and/or one or more antigen-mediated signal transduction pathway by at least 10% (e.g., 10%, 20%, 30%, 40%, 50%, 60%, 70%, 80%, 90%, 95%, 98% or more).

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be a Fab, F(ab′)2, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (Ward et al., Nature 341:544-546, 1989) including VH and VL domains; (vi) a dAb fragment that consists of a VH domain; (vii) a dAb that consists of a VH or a VL domain; (viii) an isolated complementarity determining region (CDR); and (ix) a combination of two or more isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

A “Gene Writer” polypeptide, as used herein, refers to a polypeptide capable of integrating a nucleic acid sequence (e.g., a sequence provided on a template nucleic acid) into a target DNA molecule (e.g., in a mammalian host cell, such as a genomic DNA molecule in the host cell). In some embodiments, the Gene Writer polypeptide is capable of integrating the sequence substantially without relying on host machinery. In some embodiments, the Gene Writer polypeptide integrates a sequence into a random position in a genome, and in some embodiments, the Gene Writer polypeptide integrates a sequence into a specific target site. In some embodiments, a Gene Writer polypeptide includes one or more domains that, collectively, facilitate 1) binding the template nucleic acid, 2) binding the target DNA molecule, and 3) facilitate integration of the at least a portion of the template nucleic acid into the target DNA. Gene Writer polypeptides include both naturally occurring polypeptides, such as RNA retrotransposases, DNA recombinases (e.g., tyrosine recombinases, serine recombinases, etc.), and DNA transposases, as well as engineered variants of the foregoing, e.g., having one or more amino acid substitutions to the naturally occurring sequence. Gene Writer polypeptides also include heterologous constructs, e.g., where one or more of the domains recited above are heterologous to each other, whether through a heterologous fusion (or other conjugate) of otherwise wild-type domains, as well as fusions of modified domains, e.g., by way of replacement or fusion of a heterologous sub-domain or other substituted domain. Exemplary Gene Writer polypeptide, and systems comprising them and methods of using them, that can be used in the methods provided herein are described, e.g., in PCT/US19/48607, filed Aug. 28, 2019; 62/876,165, filed Jul. 19, 2019; 62/939,525, filed Nov. 22, 2019; and 62/967,934, filed Jan. 30, 2020, each of which are incorporated herein by reference.

A “host response modulator”, as used herein, refers to an agent that modifies systemic (e.g., adaptive, innate, or adaptive and innate immune responses), intracellular (DNA damage and repair response, cellular innate immunity), or systemic and intracellular responses to a Gene Writer polypeptide, a nucleic acid encoding a Gene Writer polypeptide, or the activity of a Gene Writer polypeptide. In some embodiments, the agent comprises a compound, a plurality of compounds, a nucleic acid, a polypeptide, or a complex. Exemplary agents include small molecules and large molecules, such as a biologic, e.g., a nucleic acid or polypeptide, as well as a combinations of large and small molecules, such as an antibody-drug conjugate. In certain embodiments, the host response modulator inhibits (reduces, represses, or blocks; e.g., by at least: 5, 10, 15, 20, 25, 30, 35, 40, 45, 50, 55, 60, 65, 70, 75, 80, 85, 90, 95, 96, 97, 98, 99%, or more, relative to control, e.g., by at least: 2, 4, 8, 10, 20, 50, 100, 200, 500, or 1000-fold) a host response, while in other embodiments the host response modulator increases (stimulates or promotes; e.g., by at least: 5, 10, 15, 20, 25, 30, 35, 40, 45, 50, 55, 60, 65, 70, 75, 80, 85, 90, 95, 96, 97, 98, 99%, or more, relative to control, e.g., by at least: 2, 4, 8, 10, 20, 50, 100, 200, 500, or 1000-fold) a host response. In some embodiments, host response modulator is a host response inhibitor. Thus, in some such embodiments, the response from the host inhibits Gene Writer activity, and the host response inhibitor reduces the host response, thereby promoting Gene Writer activity. In some embodiments, the host response modulator is a host response stimulator. Thus, in some such embodiments, the response from the host promotes the Gene Writer activity, and the host response stimulator increases the host response, thereby promoting Gene Writer activity. Exemplary classes of host response modulators include antibodies (including antibody conjugates), nucleic acid modulators (e.g., inhibitory RNAs, including conjugates comprising such molecules), CRISPR systems, other polypeptide-containing modulators (including dominant-negative polypeptides and conjugates comprising the same), small molecule modulators, and combinations of the foregoing. A host response modulator may be a component endogenous to the cell or a component foreign to the cell, e.g., a component that would not otherwise be found in the cell. In some embodiments, the host response modulator comprises a natural component of a host cell, e.g., a nucleic acid or protein, or nucleic acid encoding a protein, of the host cell. In some embodiments, the host response modulator does not comprise a natural component of a host cell, e.g., does not comprise a nucleic acid, protein, or nucleic acid encoding a protein, naturally occurring in the host cell. In some embodiments, the host response modulator comprises a component that is not naturally occurring in the cell, e.g., comprises a nucleic acid, protein, or small molecule not naturally occurring in the host cell.

As used herein, the term “epigenetic modifier” refers to an agent (e.g., a compound, plurality of compounds, nucleic acid, polypeptide, or complex) that changes the epigenetic state of a nucleic acid. In some embodiments, the epigenetic modifier increases or decreases DNA methylation. In some embodiments, the epigenetic modifier increases or decreases a covalent modification to a histone. In some embodiments, the epigenetic modifier increases or decreases the number of histones at nucleic acid region. In some embodiments, the epigenetic modifier alters the position of histones at a nucleic acid region.

Genome engineering promises tremendous therapeutic potential, including the ability to permanently address genetic diseases. Existing methods of genome engineering, however, are limited by, inter alia, the limited ability of existing systems to effectively integrate sequences, such as multi-base sequences, into DNA efficiently due, at least in part, to reliance on endogenous host machinery to effectuate the edits. Furthermore, even certain autonomous (i.e., without relying on endogenous host machinery) systems for genome engineering, for example, based on mobile genetic elements, may be inhibited by host response pathways, e.g., pathways that inhibit the activity of mobile genetic elements. Accordingly, a need exists for improved methods of genome engineering that account for both the need for improved systems for genome engineering while mitigating host response pathways that otherwise limit the effectiveness of these systems.

The invention provides, inter alia, methods of genome engineering that employ improved systems for genome engineering and inhibit host response pathways that inhibit these systems The invention is based, at least in part, on Applicant's observation that certain host defense pathways can inhibit methods of genome modification, e.g., by inhibiting systems that are otherwise capable of autonomously (i.e., without relying on endogenous host machinery) modifying a DNA molecule in a mammalian cell, such as the cell's genome. In some embodiments, modulation of the host response results in an increased stability, e.g., maintenance of an insertion or expression thereof. In some embodiments, modulation of the host response results in decreased cytotoxicity.

Host Responses Generally

In some embodiments, a gene modifying system described herein induces a host response. In some embodiments, the host response comprises increased level of an endogenous protein, decreased level of an endogenous protein, increased activity of an endogenous protein, decreased activity of an endogenous protein, increased level of an endogenous RNA, or decreased level of an endogenous RNA.

In some embodiments, the agent (e.g. a host response modulator or an epigenetic modifying agent) that promotes activity of the gene modifying system is not fused to a component of a gene modifying system. In some embodiment, the agent that promotes activity of the gene modifying system is fused to a component of a gene modifying system. In some embodiment, the agent that promotes activity of the gene modifying system is covalently linked to a component of a gene modifying system. In some embodiment, the agent is covalently linked or fused to the Gene Writer polypeptide or to a nucleic acid encoding the Gene Writer polypeptide. In some embodiment, the agent is covalently linked or fused to the template nucleic acid (e.g., RNA, DNA, or DNA encoding an RNA template). In some embodiment, the agent is covalently linked or fused to the gRNA. In some embodiments, the agent is a nucleic acid, e.g., an RNA, e.g., an inhibitory RNA, a small molecule, a large molecule, e.g., a biologic, e.g., a polypeptide, e.g., an antibody (including antibody-drug conjugates) or an enzyme, or a functional fragment thereof, e.g., a domain. In some embodiments, the agent modulates, e.g., inhibits or stimulates a host process.

In some embodiments, the host response (e.g., an undesired host response) by 10%, 20%, 30%, 40%, 50%, 60%, 70%, 80%, or 90% compared to an otherwise similar cell contacted with the gene modifying system but not the host response modulator or by the gene modifying system not fused with a host response modulator. In some embodiments, the host response modulator inhibits the host response to a level characteristic of an otherwise similar cell not contacted with the gene modifying system.

In some embodiments, the host response modulator inhibits a host process (e.g., inhibits or sequesters a host DNA repair enzyme that might interfere with Gene Writing) by 10%, 20%, 30%, 40%, 50%, 60%, 70%, 80%, or 90% compared to an otherwise similar cell contacted with the gene modifying system but not the host response modulator or by the gene modifying system not fused with a host response modulator.

In some embodiments, the host response modulator reduces host immune response (e.g., a modulator comprises an enzyme, e.g., an endopeptidase, e.g., Ig-cleaving endopeptidase, e.g., IdeS, that degrades host antibodies including anti-AAV neutralizing antibodies fused to a component of a delivery vehicle, e.g., an AAV, e.g., an AAV capsid or e.g., a molecule that promotes immunotolerance). In some embodiments, the host immune response is reduced by 10%, 20%, 30%, 40%, 50%, 60%, 70%, 80%, or 90% compared to an otherwise similar cell contacted with the gene modifying system not fused with a host response modulator.

In some embodiments, the host response modulator increases the host response (e.g., a desired host response) by 10%, 20%, 30%, 40%, 50%, 60%, 70%, 80%, or 90% compared to an otherwise similar cell contacted with the gene modifying system but not the host response modulator or by the gene modifying system not fused with a host response modulator.

In some embodiments, the host response modulator stimulates a host process, (e.g., activates or recruits a host protein or complex, e.g., a host DNA repair enzyme that stimulates Gene Writing) by 10%, 20%, 30%, 40%, 50%, 60%, 70%, 80%, or 90% compared to an otherwise similar cell contacted with the gene modifying system but not the host response modulator or by the gene modifying system not fused with a host response modulator.

In some embodiments, the host response modulator increases the level of a host molecule, e.g., a nucleic acid, protein, or nucleic acid encoding a protein, by providing additional copies of that molecule, e.g., more copies of a nucleic acid, protein, or nucleic acid encoding a protein. In some embodiments, the host response modulator is a protein endogenous to the cell and results in an increase in the levels of that protein in the cell. In some embodiments, the host response modulator is a nucleic acid endogenous to the cell and results in an increase in the levels of that nucleic acid in the cell. In some embodiments, the host response modulator is a nucleic acid encoding a protein that is endogenous to the cell. In some embodiments, the host response modulator is an RNA molecule, e.g., an mRNA, that encodes an endogenous protein of the cell and results in its overexpression, e.g., expression levels that are at least 10%, 20%, 30%, 40%, 50%, 60%, 70%, 80%, 90%, 100%, or higher, or by at least 2, 4, 8, 10, 20, 50, 100, 200, 500, or 1000-fold higher compared to an otherwise similar cell contacted with the gene modifying system but not the host response modulator or by the gene modifying system not fused with a host response modulator. In some embodiments, the host response modulator is a DNA molecule, e.g., an episomal DNA, that encodes an endogenous protein of the cell and results in its overexpression, e.g., expression levels that are at least 10%, 20%, 30%, 40%, 50%, 60%, 70%, 80%, 90%, 100%, or higher, or by at least 2, 4, 8, 10, 20, 50, 100, 200, 500, or 1000-fold higher compared to an otherwise similar cell contacted with the gene modifying system but not the host response modulator or by the gene modifying system not fused with a host response modulator.

In some embodiments, the host response modulator, e.g., host response enhancer or inhibitor is an enzyme. In some embodiments, the enzyme is fused to a component of a delivery vehicle, e.g., AAV. In some embodiments, the enzyme is fused to an AAV capsid. In some embodiments, the enzyme is an endopeptidase, e.g., Ig-cleaving endopeptidase. In some embodiments, the enzyme is an IdeS that degrades host antibodies including anti-AAV neutralizing antibodies.

In some embodiments, the host response modulator, e.g., host response enhancer is a protein or a functional fragment thereof, e.g., a domain. In some embodiments, the protein or the domain stimulates a host process, e.g., activates or recruits a host protein or complex. In some embodiments, the protein or the domain stimulates Gene Writing, e.g., by replacing or supplementing a host protein, complex, or pathway. In some embodiment, the protein is a host DNA repair enzyme that stimulates Gene Writing. In some embodiments, the protein or the domain stimulates trans writing. In some embodiments, the protein or the domain stimulates cis writing. In some embodiments, the domain is a domain that recruits a host 5′ exonuclease e.g., EXO1 for cis writing. In some embodiments, the domain is a domain that that recruits a structure-specific endonuclease, e.g., FEN1 for cis writing.

In some embodiments, the host response modulator, e.g., host response inhibitor is a protein or a functional fragment thereof, e.g., a domain. In some embodiments, the protein or the domain inhibits a host process, e.g., inhibits or sequesters a host DNA repair enzyme that might interfere with Gene Writing.

In some embodiments, the host response inhibitor inhibits or sequesters a host protein (e.g., host enzyme) or host complex. In some embodiments, the host protein is involved in Homology Directed Repair (HDR). In some embodiments, the host protein involved in HDR is chosen from PARP1, PARP2, MRE11, RAD50, NBS1, BARD1, BRCA2, BRCA1, RTS, RECQ5, RPA3, PP4, PALB2, DSS1, RAD51, BACH1, FANCJ, Topbp1, TOPO III, FEN1, MUS81, EME1, SLX1, SLX4, RECQ1, WRN, CtIP, EXO1, DNA2, MRN complex), Fanconi Anaemia complementation group (FANC) (e.g., FANCA, FANCB, FANCC, FANCD1, FANCD2, FANCE, FANCF, FANCG, FANCI, FANCJ, FANCL, FANCM, FANCN, FANCO, FANCP, FANCQ, FANCR, FANCS, FANCT), Anti-HDR (e.g., FBH1, RECQ5, BLM, FANCJ, PART, RECQ1, WRN, RTEL, RAP80, miR-155, miR-545, miR-107, miR-1255, miR-148, miR-193), Single Strand Annealing (SSA) (e.g., RPA, RPA1, RPA2, RPA3, RAD52, XPF, ERCC1), Canonical Non-Homologous End Joining (C-NHEJ) (e.g., DNA-PK, DNA-PKcs, 53BP1, XRCC4, LIG4, XLF, ARTEMIS, APLF, PNK, Rif1, PTIP, DNA polymerase, Ku70, Ku80), Alternative Non-Homologous End Joining (Alt-NHEJ) (PARP1, PARP2, CtIP, LIG3, MRE11, Rad50, Nbs1, XPF, ERCC1, LIG1, DNA Polymerase θ, MRN complex, XRCC1), Mismatch Repair (MMR) (e.g., EXO1, MSH2, MSH3, MSH6, MLH1, PMS2, MLH3, DNA polymerase delta, RPA, RFC, LIG1), Nucleotide Excision Repair (NER) (e.g., XPF, XPG, ERCC1, TTDA, UVSSA, USP7, CETN2, RAD23B, UV-DDB, CAK subcomplex, RPA, PCNA), Base Excision Repair (BER) (e.g., APE1, Pol β, Pol δ, Pol ε, XRCC1, LIG3, FEN-1, PCNA, RECQL4, WRN, MYH, PNKP, APTX), Single-Strand Break Repair (SSBR) (e.g., PARP1, PARP2, PARG, XRCC1, DNA pol β, DNA pol δ, DNA pol ε, PCNA, LIG1, PNK, PNKP, APE1, APTX, APLF, TDP1, LIG3, FEN1, CtIP, MRN, ERCC1), chromatin modification (e.g., Ezh2, HDAC-Class I, HDAC-Class IIKDM4A/JMJD2A, FACT), cell cycle (e.g., CDK1, CDC7, ATM, ATR), Translesion DNA Synthesis (TLS) (e.g., UBC13, or RAD18), cellular metabolism (e.g., mTOR), cell death (e.g., p53), or RNA:DNA resolution/R-Loop (e.g., SETX, RNH1, or RNH2), or Type I Interferon response (e.g., caspase-1, IFNα, IFNβ, NF-κB, TNF-α).

In some embodiments, the agent that promotes activity of the gene modifying system modulates a pathway listed in Table 0 in the column entitled “Pathway”. In some embodiments, the agent that promotes activity of the gene modifying system modulates the level or activity of a protein listed in Table 0 in the column entitled “Protein”. In some embodiments the agent stimulates or inhibits a Pathway or Protein listed in Table 0. In some embodiments, the agent that promotes activity of the gene modifying system is a Protein or fragment thereof listed in Table 0. In some embodiments, the agent that promotes activity of the gene modifying system comprises a composition listed in Table 0 in the column entitled “Molecule Name”, e.g., a composition as described in the column entitled “Citation”. In some embodiments, the agent is an inhibitor and the agent comprises a nucleic acid, e.g., an inhibitor RNA, e.g., a siRNA. In some embodiments, the agent comprises a small molecule, a protein, a fusion protein, an antibody, polypeptide (e.g., a dominant negative mutant of a polypeptide in a host response pathway), an enzyme (e.g., endopeptidase, e.g., Ig-cleaving endopeptidase, e.g., IdeS). In some embodiments, the agent that promotes activity of the gene modifying system comprises a nucleic acid that is covalently linked to the GeneWriter polypeptide or the template nucleic acid. In some embodiments, the agent that promotes activity of the gene modifying system is a small molecule. In some embodiments, the agent that promotes activity of the gene modifying system is a domain of a polypeptide.

TABLE 0
Host pathways and targets for modulation

| | | | Molecule | | Citation (incorporated herein by |
|---|---|---|---|---|---|
| Category | Pathway | Protein | Type | Molecule Name | reference in its entirety) |
| DNA sensing | cGAS- | cGAS | Inhibitor | PF-06928215 | Hall et al. (PLoS One. 12(9): |
| | STING | | | | e0184843, 2017) |
| DNA sensing | cGAS- | cGAS | Inhibitor | RU.365 | Vincent et al. (Nat Commun. |
| | STING | | | | 29; 8(1): 750, 2017) |
| DNA sensing | cGAS- | cGAS | Inhibitor | RU.521 | Vincent et al. (Nat Commun. 23; |
| | STING | | | | 8(1): 1827, 2017) |
| DNA sensing | cGAS- | cGAS | Inhibitor | Suramin | Wang et al. (Future Med Chem. |
| | STING | | | | 1; 10(11): 1301-1317, 2018) |
| DNA sensing | cGAS- | cGAS | Inhibitor | G150 | Lama et al. (Nat Commun. |
| | STING | | | | 21; 10(1): 2261, 2019) |
| DNA sensing | cGAS- | STING | Inhibitor | C-176 | Haag et al. (Nature. 2018 |
| | STING | | | | July; 559(7713): 269-273) |
| DNA sensing | cGAS- | STING | Inhibitor | C-178 | Haag et al. (Nature. 2018 |
| | STING | | | | July; 559(7713): 269-273) |
| DNA sensing | cGAS- | STING | Inhibitor | H151 | Haag et al. (Nature. 2018 |
| | STING | | | | July; 559(7713): 269-273) |
| DNA sensing | cGAS- | STING | Inhibitor | Compound 18 | Siu et al. (ACS Med Chem Lett. 2018 |
| | STING | | | | Dec. 6; 10(1): 92-97) |
| DNA sensing | cGAS- | STING | Inhibitor | Astin C | Li et al. (Cell Rep. 2018 Dec. |
| | STING | | | | 18; 25(12): 3405-3421.e7) |
| DNA sensing | cGAS- | STING | Inhibitor | Screening Hit 1 | Siu et al. (ACS Med Chem Lett. 2018 |
| | STING | | | | Dec. 6; 10(1): 92-97) |
| DNA sensing | cGAS- | STING | Inhibitor | Compound 13 | Siu et al. (ACS Med Chem Lett. 2018 |
| | STING | | | | Dec. 6; 10(1): 92-97) |
| DNA sensing | cGAS- | STING | Inhibitor | siRNA-1 | WO2018201144A1 |
| | STING | | | (SEQ ID NO: 6) | |
| DNA sensing | cGAS- | STING | Inhibitor | siRNA-2 | WO2018201144A1 |
| | STING | | | (SEQ ID NO: 7) | |
| DNA sensing | cGAS- | STING | Inhibitor | siRNA-3 | WO2018201144A1 |
| | STING | | | (SEQ ID NO: 8) | |
| DNA sensing | cGAS- | STING | Inhibitor | siRNA-4 | WO2018201144A1 |
| | STING | | | (SEQ ID NO: 9) | |
| DNA sensing | cGAS- | STING | Inhibitor | E1A (hAd5) | Lau L, (Science. 2015 Oct. |
| | STING | | | | 30; 350(6260): 568-71) |
| DNA sensing | cGAS- | STING | Inhibitor | E7 (HPV18) | Lau et al. (Science. 2015 Oct. |
| | STING | | | | 30; 350(6260): 568-71) |
| DNA sensing | cGAS- | TBK1 | Inhibitor | BX795 | Clark et al. (J Biol Chem. 2009 May |
| | STING | | | | 22; 284(21): 14136-46.) |
| DNA sensing | cGAS- | TBK1 | Inhibitor | Tozasertib | Richters et al. (ACS Chem Biol. 2015 |
| | STING | | | | Jan. 16; 10(1): 289-98) |
| DNA sensing | cGAS- | TBK1 | Inhibitor | Tozasertib-15a | Richters et al. (ACS Chem Biol. 2015 |
| | STING | | | | Jan. 16; 10(1): 289-98) |
| DNA sensing | cGAS- | TBK1 | Inhibitor | 20b | McIver et al. (Bioorg Med Chem Lett. |
| | STING | | | | 2012 Dec. 1; 22(23): 7169-73) |
| DNA sensing | cG AS- | TBK1 | Inhibitor | azabenzimidazole | Wang et al. (Bioorg Med Chem Lett. |
| | SLING | | | hit 1a | 2012 Mar. 1; 22(5): 2063-9) |
| DNA sensing | cGAS- | TBK1 | Inhibitor | CYT387 | Pardanani et al. (Leukemia. 2009 |
| | STING | | | | August; 23(8): 1441-5) |
| DNA sensing | cGAS- | TBK1 | Inhibitor | Domainex | Hasan et al. (Pharmacol Res. 2016 |
| | STING | | | | September; 11: 336-342) |
| DNA sensing | cGAS- | TBK1 | Inhibitor | Amgen Compound | Ou et al., (Mol Cell. 2011 Feb. |
| | STING | | | II | 18; 41(4): 458-70) |
| DNA sensing | cGAS- | TBK1 | Inhibitor | MRT67307 | Clark et al. (Biochem J. 2011 Feb. |
| | STING | | | | 15; 434(1): 93-104) |
| DNA sensing | cGAS- | TBK1 | Inhibitor | AZ13102909 | Vu et al. (Mol Cancer Res. 2014 |
| | STING | | | | October; 12(10): 1509-19) |
| DNA sensing | cGAS- | IRF3 | Inhibitor | siRNA-1 | WO2018201144A1 |
| | STING | | | (SEQ ID NO: 2) | |
| DNA sensing | cGAS- | IRF3 | Inhibitor | siRNA-2 | WO2018201144A1 |
| | STING | | | (SEQ ID NO: 3) | |
| DNA sensing | cGAS- | IRF3 | Inhibitor | siRNA-3 | WO2018201144A1 |
| | STING | | | (SEQ ID NO: 4) | |
| DNA sensing | cGAS- | IRF3 | Inhibitor | siRNA-4 | WO02018201144A1 |
| | STING | | | (SEQ ID NO: 5) | |
| DNA sensing | SIDSP | DNA-PK | Inhibitor | Nu-7441 | Leahy et al. (Bioorg Med Chem Lett. |
| | | | | | 2004 Dec. 20; 14(24): 6083-7) |
| DNA sensing | SIDSP | DNA-PK | Inhibitor | hAd5 E1A | Burleigh et al. (Sci Immunol. 2020 |
| | | | | | Jan. 24; 5(43): eaba4219) |
| DNA sensing | SIDSP | DNA-PK | Inhibitor | HSV-1 ICP0 | Burleigh et al. (Sci Immunol. 2020 |
| | | | | | Jan. 24; 5(43): eaba4219) |
| RNA sensing | IFN1 | RIG-I | Inhibitor | HIV-1 protease | Solis et al. (J Virol. 2011 |
| | | | | | February; 85(3): 1224-36) |
| RNA sensing | IFN1 | MDA5 | | | |
| RN A sensing | IFN1 | IKK | Inhibitor | BAY11 | Awe et al. (Stem Cell Res Ther. 2013 |
| | | complex | | | Feb. 6; 4(1): 15) |
| RNA sensing | IFN1 | TRIF | Inhibitor | Pepinh-TRIF | Toshchakov et al. (J Immunol. 2005 |
| | | | | | Jul. 1; 175(1): 494-500) |
| RNA sensing | IFN1 | MyD88 | Inhibitor | Pepinh-MYD | Loiarro et al. (J Biol Chem. 2005 Apr. |
| | | | | | 22; 280(16): 15809-14) |
| RNA sensing | IFN1 | IFN | Inhibitor | Vaccinia B18R | Kim et al. (PLoS One. 2017 Dec. |
| | | | | | 7; 12(12): e0189308) |
| | | Endosomal | Inhibitor | Chloroquine | |
| | | maturation | | | |
| | | | Inhibitor | TSA | |
| | | Endosomal | | Bafilomycin A1 | |
| | | maturation | | | |
| | | RNASEH2 | | | |
| | | ADAR1 | | | Orecchini et al. (RNA Biol. 2017 Nov. |
| | | | | | 2; 14(11): 1485-1491) |
| Antiviral | Type I | IFN-α | | | |
| | Interferon | | | | |
| Antiviral | Type I | IFN-β | | | |
| | Interferon | | | | |
| Antiviral | Type II | IFN-γ | | | |
| | Interferon | | | | |
| HDR | anti-HDR | FBH1 | Agonist | | |
| HDR | anti-HDR | RECQ5 | Agonist | | |
| HDR | anti-HDR | BLM | Agonist | | |
| HDR | anti-HDR | FANCJ | Agonist | | |
| HDR | anti-HDR | PARI | Agonist | | |
| HDR | anti-HDR | RECQ1 | Agonist | | |
| HDR | anti-HDR | WRN | Agonist | | |
| HDR | anti-HDR | RTEL | Agonist | | |
| HDR | anti-HDR | Rap80 | Agonist | | |
| HDR | anti-HDR | miR-155 | Agonist | | |
| HDR | anti-HDR | miR-545 | Agonist | | |
| HDR | anti-HDR | miR-107 | Agonist | | |
| HDR | anti-HDR | miR-1255 | Agonist | | |
| HDR | anti-HDR | miR-148 | Agonist | | |
| HDR | anti-HDR | miR-193 | Agonist | | |
| | | p53 | Inhibitor | | |
| | | p53 | Inhibitor | Dominant negative | Schiroli et al. (Cell Stem Cell. 2019 |
| | | | | mRNA | Apr. 4; 24(4): 551-565.e8) |
| | | BRCA1 | Inhibitor | Dominant negative | |
| | | | | mRNA | |
| DNA repair | | BRCA1 | Inhibitor | siBRCA1 | Mita et al. (Nat Struct Mol Biol. 2020 |
| | | | | | February; 27(2): 179-191) |
| DNA repair | | BRAC2 | Inhibitor | siBRCA2 | Mita et al. (Nat Struct Mol Biol. 2020 |
| | | | | | February; 27(2): 179-191) |
| DNA repair | | FANCD | Inhibitor | siFANCD2 | Mita et al. (Nat Struct Mol Biol. 2020 |
| | | | | | February; 27(2): 179-191) |
| | | MORC2 | Inhibitor | | Liu et al. (Nature. 2018 Jan. |
| | | | | | 11; 553(7687): 228-232) |
| | | TASOR | Inhibitor | | Liu et al. (Nature. 2018 Jan. |
| | | | | | 11; 553(7687): 228-232) |
| | | MPP8 | Inhibitor | | Liu et al. (Nature. 2018 Jan. |
| | | | | | 11; 553(7687): 228-232) |
| | | SETX | Inhibitor | | Liu et al. (Nature. 2018 Jan. |
| | | | | | 11; 553(7687): 228-232) |
| | | MOV10 | Inhibitor | | Liu et al. (Nature. 2018 Jan. |
| | | | | | 11; 553(7687): 228-232) |
| | | SAFB | Inhibitor | | Liu et al. (Nature. 2018 Jan. |
| | | | | | 11; 553(7687): 228-232) |
| | | RAD51 | Inhibitor | | Liu et al. (Nature. 2018 Jan. |
| | | | | | 11; 553(7687): 228-232) |
| Chromatin modifier | Chr | Ezh2 | | GSK343 | Zeidler M. et al. Neoplasia. |
| | | | | | November 2005; Chou D. et al |
| | | | | | PNAS 2010; Puppe et al, |
| | | | | | Breast cancer research 2009 |
| Chromatin modifier | Chr | Ezh2 | | EPZ-6438 | Zeidler M. et al. Neoplasia. |
| | | | | | November 2005; Chou D. et al |
| | | | | | PNAS 2010; Puppe et al, |
| | | | | | Breast cancer research 2009 |
| Chromatin modifier | Chr | Ezh2 | | GSK2816126 | Zeidler M. et al. Neoplasia. |
| | | | | | November 2005; Chou D. et al |
| | | | | | PNAS 2010; Puppe et al, |
| | | | | | Breast cancer research 2009 |
| Chromatin modifier | Chr | Ezh2 | | SureCN6120847 | Zeidler M. et al. Neoplasia. |
| | | | | | November 2005; Chou D. et al |
| | | | | | PNAS 2010; Puppe et al, |
| | | | | | Breast cancer research 2009 |
| Chromatin modifier | Chr | Ezh2 | | EPZ005687 | Zeidler M. et al. Neoplasia. |
| | | | | | November 2005; Chou D. et al |
| | | | | | PNAS 2010; Puppe et al, |
| | | | | | Breast cancer research 2009 |
| Chromatin modifier | Chr | HDAC-Class | | Trichostatin A | Tang et al, Nat Struct Mol |
| | | I/Class II | | (TSA) | Biol. 2013 |
| Chromatin modifier | Chr | HDAC-Class | | Sodium Butyrate | Tang et al, Nat Struct Mol |
| | | I/Class II | | (NaB) | Biol. 2013 |
| Chromatin modifier | Chr | KDM4A/ | | | Pfister et al, Cell |
| | | JMJD2A | | | Reports 2014 |
| Chromatin modifier | Chr | FACT | | CBL0137 | |
| Single-strand break | SSBR | PARP1/PAR | | Olaparib, | Caldecott Nature reviews |
| repair | | P2 | | AZD2281, | Genetic, 2008 |
| | | | | KU-0059436 | |
| Single-strand break | SSBR | PARP1/ | | Iniparib, BSI-201 | Caldecott Nature reviews |
| repair | | PARP2 | | | Genetic, 2008 |
| Single-strand break | SSBR | PARP1/ | | BMN 673 | Caldecott Nature reviews |
| repair | | PARP2 | | | Genetic, 2008 |
| Single-strand break | SSBR | PARP1/ | | Rucaparib, | Caldecott Nature reviews |
| repair | | PARP2 | | (AG014699, PF- | Genetic, 2008 |
| | | | | 01367338) | |
| Single-strand break | SSBR | PARP1/ | | Veliparib, ABT-888 | Caldecott Nature reviews |
| repair | | PARP2 | | | Genetic, 2008 |
| Single-strand break | SSBR | PARP1/ | | CEP 9722 | Caldecott Nature reviews |
| repair | | PARP2 | | | Genetic, 2008 |
| Single-strand break | SSBR | PARP1/ | | INO-1001 | Caldecott Nature reviews |
| repair | | PARP2 | | | Genetic, 2008 |
| Single-strand break | SSBR | PARP1/ | | MK 4827 | Caldecott Nature reviews |
| repair | | PARP2 | | | Genetic, 2008 |
| Single-strand break | SSBR | PARP1/ | | BGB-290 | Caldecott Nature reviews |
| repair | | PARP2 | | | Genetic, 2008 |
| Single-strand break | SSBR | PARP1/ | | E701, GPI21016 | Caldecott Nature reviews |
| repair | | PARP2 | | | Genetic, 2008 |
| Single-strand break | SSBR | PARP1/ | | MP-124 | Caldecott Nature reviews |
| repair | | PARP2 | | | Genetic, 2008 |
| Single-strand break | SSBR | PARP1/ | | LT-673 | Caldecott Nature reviews |
| repair | | PARP2 | | | Genetic, 2008 |
| Single-strand break | SSBR | PARP1/ | | NMS-P118 | Caldecott Nature reviews |
| repair | | PARP2 | | | Genetic, 2008 |
| Single-strand break | SSBR | PARP1/ | | XAV939 | Caldecott Nature reviews |
| repair | | PARP2 | | | Genetic, 2008 |
| Single-strand break | SSBR | PARP1/ | | 3-aminobenzamide | Caldecott Nature reviews Genetic, |
| repair | | PARP2 | | | 2008 |
| Mismatch repair | MMR | EXO1 | | | |
| Mismatch repair | MMR | MSH2 | | | Guo-Min Li 2008 Cell Research, |
| | | | | | 2013 Cancer Research |
| Mismatch repair | MMR | MSH3 | | | Guo-Min Li 2008 Cell Research, |
| | | | | | 2013 Cancer Research |
| Mismatch repair | MMR | MSH6 | | | Guo-Min Li 2008 Cell Research, |
| | | | | | 2013 Cancer Research |
| Mismatch repair | MMR | MLH1 | | | Guo-Min Li 2008 Cell Research, |
| | | | | | 2013 Cancer Research |
| Mismatch repair | MMR | PMS2 | | | Guo-Min Li 2008 Cell Research, |
| | | | | | 2013 Cancer Research |
| Mismatch repair | MMR | MLH3 | | | |
| Mismatch repair | MMR | DNA Pol | | | |
| | | delta | | | |
| Mismatch repair | MMR | RPA | | | |
| Mismatch repair | MMR | HMGB1 | | | |
| Mismatch repair | MMR | RFC | | | |
| Mismatch repair | MMR | DNA ligase I | | | |
| Nucleotide excision | NER | XPA-G | | | |
| repair | | | | | |
| Nucleotide excision | NER | POLH | | | |
| repair | | | | | |
| Nucleotide excision | NER | XPF | | | |
| repair | | | | | |
| Nucleotide excision | NER | ERCC1 | | | |
| repair | | | | | |
| Nucleotide excision | NER | XPA-G | | | |
| repair | | | | | |
| Nucleotide excision | NER | LIG1 | | | |
| repair | | | | | |
| Nucleotide excision | NER | CSA | | | |
| repair | | | | | |
| Nucleotide excision | NER | CCD | | | |
| repair | | | | | |
| Nucleotide excision | NER | XPA | | | |
| repair | | | | | |
| Nucleotide excision | NER | XPB | | | |
| repair | | | | | |
| Nucleotide excision | NER | XPC | | | |
| repair | | | | | |
| Nucleotide excision | NER | XPD | | | |
| repair | | | | | |
| Nucleotide excision | NER | XPF | | NSC 130813 | |
| repair | | | | | |
| Nucleotide excision | NER | XPG | | | |
| repair | | | | | |
| Nucleotide excision | NER | ERCC1 | | | |
| repair | | | | | |
| Nucleotide excision | NER | TTDA | | | |
| repair | | | | | |
| Nucleotide excision | NER | UVSSA | | | |
| repair | | | | | |
| Nucleotide excision | NER | USP7 | | | |
| repair | | | | | |
| Nucleotide excision | NER | CETN2 | | | |
| repair | | | | | |
| Nucleotide excision | NER | RAD23B | | | |
| repair | | | | | |
| Nucleotide excision | NER | UV-DDB | | | |
| repair | | | | | |
| Nucleotide excision | NER | CAK | | | |
| repair | | subcomplex | | | |
| Nucleotide excision | NER | RPA | | | |
| repair | | | | | |
| Nucleotide excision | NER | PCNA | | | |
| repair | | | | | |
| Base excision repair | BER | APE1 | | | |
| Base excision repair | BER | Pol beta | | | |
| Base excision repair | BER | Pol delta | | | |
| Base excision repair | BER | Pol epsilon | | | |
| Base excision repair | BER | XRCC1 | | | |
| Base excision repair | BER | Ligase III | | | |
| Base excision repair | BER | FEN-1 | | | |
| Base excision repair | BER | PCNA | | | |
| Base excision repair | BER | RECQL4 | | | |
| Base excision repair | BER | WRN | | | |
| Base excision repair | BER | MYH | | | |
| Base excision repair | BER | PNKP | | | |
| Base excision repair | BER | APTX | | | |
| Single-strand | SSA | RPA | | | |
| annealing | | | | | |
| Single-strand | SSA | RPA1 | | | |
| annealing | | | | | |
| Single-strand | SSA | RPA2 | | | |
| annealing | | | | | |
| Single-strand | CCA | RPA3 | | | |
| annealing | DDA | | | | |
| Single-strand | SSA | RAD52 | | AID 651668 | Ciccia et al, Mol Cell 2010 |
| annealing | | | | | |
| Single-strand | SSA | RAD52 | | AICAR | Ciccia et al, Mol Cell 2010 |
| annealing | | | | | |
| Single-strand | SSA | XPF | | NSC 130813 | Ciccia et al, Mol Cell 2010 |
| annealing | | | | | |
| Single-strand | SSA | ERCC1 | | NSC 130813 | Ciccia et al, Mol Cell 2010 |
| annealing | | | | | |
| Non-homologous | C-NHEJ | 53BP1 | | | Stephanie Panier and Simon J. |
| end-joining | | | | | Boulton, Nature Review 2014 |
| Non-homologous | C-NHEJ | XRCC4 | | | |
| end-joining | | | | | |
| Non-homologous | C-NHEJ | LIG4 | | SCR7 | |
| end-joining | | | | | |
| Translesion synthesis | TLS | Ubc13 | | | |
| Translesion synthesis | TLS | Rad18 | | | |
| Cellular metabolism | | mTOR | | rapamycin | |
| | | APOBEC | | | |
| Non-homologous | NHEJ | 53 BP1 | | | Stephanie Panier and Simon J. |
| end-joining | | | | | Boulton, Nature Review 2014 |
| Non-homologous | NHEJ | RIf1 | | | Di Virgilio M. et al Science 2013; |
| end-joining | | | | | Zimmermann M et al Science 2013 |
| Non-homologous | NHEJ | PTIP | | | Zimmermann M and De langhe, |
| end-joining | | | | | Trends in cell Biology, 2014 |
| Non-homologous | NHEJ | KU 70-80 | | | Betermier et al, PLoS Genetics 2014 |
| end-joining | | | | | |
| Non-homologous | NHEJ | DNApk | | | Betermier et al, PLoS Genetics 2014 |
| end-joining | | | | | |
| Non-homologous | NHEJ | Lig4 | | | |
| end-joining | | | | | |
| Non-homologous | NHEJ | XLF | | | |
| end-joining | | | | | |
| Non-homologous | NHEJ | Artemis | | | Betermier et al, PLoS Genetics 2014 |
| end-joining | | | | | |
| Alternative NHEJ | Alt- | Ligase I | | | |
| pathway | NHEJ | | | | |
| Alternative NHEJ | Alt- | Ligase III | | | |
| pathway | NHEJ | | | | |
| Alternative NHEJ | Alt- | Pol Theta | | | Koole et al, Nature com 2014; Chan et |
| pathway | NHEJ | | | | al Plos Genetics 2010 |
| anti- homology | Anti | Fbh1 | | | Chapman et al, Molecular Cell 2012 |
| directed repair | HDR | | | | |
| anti- homology | Anti | RTEL | | | Chapman et al, Molecular Cell 2012 |
| directed repair | HDR | | | | |
| anti- homology | Anti | PARI | | | Chapman et al, Molecular Cell 2012 |
| directed repair | HDR | | | | |
| anti- homology | Anti | Rap80 | | | Hu Y. et al, Genes and Dev 2011, |
| directed repair | HDR | | | | Adamson et al Nature Cell Bio 2012 |
| anti- homology | Anti | miRNA | | | Gasparini et all PNAS 2014; Dimitrov |
| directed repair | HDR | | | | et al, Genes&Dev 2014; Huang et al, |
| | | | | | Mol Cancer res 2013; Neijenhuis et al, |
| | | | | | DNA repair 2013, Choi et al, eLIFE |
| | | | | | 2014 |
| | SSR | PARP | | | Caldecott Nature reviews Genetic, |
| | | | | | 2008 |
| | SSR | XRCC1 | | | Caldecott Nature reviews Genetic, |
| | | | | | 2008 |
| Homology directed | HDR | BRCA1 | | | Lorraine Symington, Cold Spring Harb |
| repair | | | | | Perspect Biol 2014 |
| Homology directed | HDR | CtIP | | | Lorraine Symington, Cold Spring Harb |
| repair | | | | | Perspect Biol 2014 |
| Homology directed | HDR | EXo1 | | | Lorraine Symington, Cold Spring Harb |
| repair | | | | | Perspect Biol 2014 |
| Homology directed | HDR | DNA2 | | | Lorraine Symington, Cold Spring Harb |
| repair | | | | | Perspect Biol 2014 |
| Homology directed | HDR | MRN | | | Lorraine Symington, Cold Spring Harb |
| repair | | complex | | | Perspect Biol 2014 |
| Homology directed | HDR | BRCA2 | | | Wolf-Dietrich Heyer |
| repair | | | | | Annu. Rev. Genet. 2010 |
| Chormatin modifier | Chr | HDAC-Class | | | Tang et al, Nat Struct Mol Biol. 2013 |
| | | I | | | |
| Chormatin modifier | Chr | HDAC-Class | | | Tang et al, Nat Struct Mol Biol. 2013 |
| | | II | | | |
| Cell cycle | Cycle | CDK1 | | RO-3306 | |
| Cell cycle | Cycle | CDK1 | | AZD 5438 | |
| Cell cycle | Cycle | CDC7 | | XL413 | |
| Translesion synthesis | TLS | Ubc13 | | | |
| Translesion synthesis | TLS | Rad18 | | | |
| | R LOOP | SEtX | | | |
| | R LOOP | RNh1 and 2 | | | |
| Fanconi anemia | FANC | FANCM | | | |
| complementation | | | | | |
| group | | | | | |
| Fanconi anemia | FANC | FANCI | | | |
| complementation | | | | | |
| group | | | | | |
| Fanconi anemia | FANC | FANCD1 | | | |
| complementation | | | | | |
| group | | | | | |
| Fanconi anemia | FANC | FANCD2 | | | |
| complementation | | | | | |
| group | | | | | |
| Homology directed | HDR | PARP1/ | | Olaparib, | |
| repair | | PARP2 | | AZD2281, | |
| | | | | KU-0059436 | |
| Homology directed | HDR | PARP1/ | | Iniparib, BS1-201 | |
| repair | | PARP2 | | | |
| Homology directed | HDR | PARP1/ | | BMN 673 | |
| repair | | PARP2 | | | |
| Homology directed | HDR | PARP1/ | | Rucaparib, | |
| repair | | PARP2 | | (AG014699, | |
| | | | | PF-01367338) | |
| Homology directed | HDR | PARP1/ | | Veliparib, ABT-888 | |
| repair | | PARP2 | | | |
| Homology directed | HDR | PARP1/ | | CEP 9722 | |
| repair | | PARP2 | | | |
| Homology directed | HDR | PARP1/ | | INO-1001 | |
| repair | | PARP2 | | | |
| Homology directed | HDR | PARP1/ | | MK 4827 | |
| repair | | PARP2 | | | |
| Homology directed | HDR | PARP1/ | | BGB-290 | |
| repair | | PARP2 | | | |
| Homology directed | HDR | PARP1/ | | E701, GPI21016 | |
| repair | | PARP2 | | | |
| Homology directed | HDR | PARP1/ | | MP-124 | |
| repair | | PARP2 | | | |
| Homology directed | HDR | PARP1/ | | LT-673 | |
| repair | | PARP2 | | | |
| Homology directed | HDR | PARP1/ | | NMS-P118 | |
| repair | | PARP2 | | | |
| Homology directed | HDR | PARP1/ | | XAV939 | |
| repair | | PARP2 | | | |
| Homology directed | HDR | PARP1/ | | 3-aminobenzamide | |
| repair | | PARP2 | | | |
| Homology directed | HDR | MRE11 | | | |
| repair | | | | | |
| Homology directed | HDR | RAD50 | | | |
| repair | | | | | |
| Homology directed | HDR | NBS1 | | | |
| repair | | | | | |
| Homology directed | HDR | BARD1 | | | |
| repair | | | | | |
| Homology directed | HDR | BRCA2 | | siBRCA2 | Wolf-Dietrich Heyer |
| repair | | | | | Annu. Rev. Genet. 2010 |
| Homology directed | HDR | RTS | | | |
| repair | | | | | |
| Homology directed | HDR | RECQ5 | | | |
| repair | | | | | |
| Homology directed | HDR | RPA3 | | | |
| repair | | | | | |
| Homology directed | HDR | PP4 | | | |
| repair | | | | | |
| Homology directed | HDR | PALB2 | | | |
| repair | | | | | |
| Homology directed | HDR | DSS1 | | | |
| repair | | | | | |
| Homology directed | HDR | RAD51 | | B02 | Wolf-Dietrich Heyer |
| repair | | | | | Annu. Rev. Genet. 2010 |
| Homology directed | HDR | BACH1 | | | |
| repair | | | | | |
| Homology directed | HDR | FANCJ | | | |
| repair | | | | | |
| Homology directed | HDR | Topbp1 | | | |
| repair | | | | | |
| Homology directed | HDR | TOPO III | | | |
| repair | | | | | |
| Homology directed | HDR | FEN1 | | | |
| repair | | | | | |
| Homology directed | HDR | MUS81 | | | |
| repair | | | | | |
| Homology directed | HDR | EME1 | | | |
| repair | | | | | |
| Homology directed | HDR | SLX1 | | | |
| repair | | | | | |
| Homology directed | HDR | SLX4 | | | |
| repair | | | | | |
| Homology directed | HDR | RECQ1 | | | |
| repair | | | | | |
| Homology directed | HDR | WRN | | | |
| repair | | | | | |
| Fanconi anemia | FANC | FANC A | | | |
| complementation | | | | | |
| group | | | | | |
| Fanconi anemia | FANC | FANC B | | | |
| complementation | | | | | |
| group | | | | | |
| Fanconi anemia | FANC | FANC C | | | |
| complementation | | | | | |
| group | | | | | |
| Fanconi anemia | FANC | FANC D1 | | | |
| complementation | | | | | |
| group | | | | | |
| Fanconi anemia | FANC | FANC D2 | | siFANCD2 | |
| complementation | | | | | |
| group | | | | | |
| Fanconi anemia | FANC | FANC E | | | |
| complementation | | | | | |
| group | | | | | |
| Fanconi anemia | FANC | FANC F | | | |
| complementation | | | | | |
| group | | | | | |
| Fanconi anemia | FANC | FANC G | | | |
| complementation | | | | | |
| group | | | | | |
| Fanconi anemia | FANC | FANC I | | | |
| complementation | | | | | |
| group | | | | | |
| Fanconi anemia | FANC | FANC J | | | |
| complementation | | | | | |
| group | | | | | |
| Fanconi anemia | FANC | FANC L | | | |
| complementation | | | | | |
| group | | | | | |
| Fanconi anemia | FANC | FANC M | | | |
| complementation | | | | | |
| group | | | | | |
| Fanconi anemia | FANC | FANC N | | | |
| complementation | | | | | |
| group | | | | | |
| Fanconi anemia | FANC | FANC O | | | |
| complementation | | | | | |
| group | | | | | |
| Fanconi anemia | FANC | FANC P | | | |
| complementation | | | | | |
| group | | | | | |
| Fanconi anemia | FANC | FANC Q | | | |
| complementation | | | | | |
| group | | | | | |
| Fanconi anemia | FANC | FANC R | | | |
| complementation | | | | | |
| group | | | | | |
| Fanconi anemia | FANC | FANC S | | | |
| complementation | | | | | |
| group | | | | | |
| Fanconi anemia | FANC | FANC T | | | |
| complementation | | | | | |
| group | | | | | |
| anti- homology | anti-HDR | miR-155 | | miR-155-5p | |
| directed repair | | | | | |
| anti- homology | anti-HDR | miR-155 | | miR-155-3p | |
| directed repair | | | | | |
| anti- homology | anti-HDR | miR-545 | | miR-545-5p | |
| directed repair | | | | | |
| anti- homology | anti-HDR | miR-545 | | miR-545-3p | |
| directed repair | | | | | |
| anti- homology | anti-HDR | miR-107 | | miR-107 | |
| directed repair | | | | | |
| anti- homology | anti-HDR | miR-1255 | | miR-1255-A | |
| directed repair | | | | | |
| anti- homology | anti-HDR | miR-1255 | | miR-1255-B1 | |
| directed repair | | | | | |
| anti- homology | anti-HDR | miR-1255 | | miR-1255-B2 | |
| directed repair | | | | | |
| anti- homology | anti-HDR | miR-148 | | miR-148-5p | |
| directed repair | | | | | |
| anti- homology | anti-HDR | miR-148 | | miR-148-3p | |
| directed repair | | | | | |
| anti- homology | anti-HDR | miR-193 | | miR-193-5p | |
| directed repair | | | | | |
| anti- homology | anti-HDR | miR-193 | | miR-193-3p | |
| directed repair | | | | | |
| Non-homologous | C-NHEJ | DNA Pk | | NU7441 | Betermier et al, plos genetics 2014 |
| end-joining | | | | | |
| Non-homologous | C-NHEJ | DNA Pk | | CC115 | Betermier et al, plos genetics 2014 |
| end-joining | | | | | |
| Non-homologous | C-NHEJ | DNA Pk | | NK314 | Betermier et al, plos genetics 2014 |
| end-joining | | | | | |
| Non-homologous | C-NHEJ | DNA Pk | | Wortmannin | Betermier et al, plos genetics 2014 |
| end-joining | | | | | |
| Non-homologous | C-NHEJ | DNA Pk | | LY294002 | Betermier et al, plos genetics 2014 |
| end-joining | | | | | |
| Non-homologous | C-NHEJ | DNA Pk | | NU 7026 | Betermier et al, plos genetics 2014 |
| end-joining | | | | | |
| Non-homologous | C-NHEJ | DNA Pk | | IC86621 | Betermier et al, plos genetics 2014 |
| end-joining | | | | | |
| Non-homologous | C-NHEJ | DNA Pk | | IC87102 | Betermier et al, plos genetics 2014 |
| end-joining | | | | | |
| Non-homologous | C-NHEJ | DNA Pk | | IC87361 | Betermier et al, plos genetics 2014 |
| end-joining | | | | | |
| Non-homologous | C-NHEJ | DNA Pk | | OK1035 | Betermier et al, plos genetics 2014 |
| end-joining | | | | | |
| Non-homologous | C-NHEJ | DNA Pk | | SU11752 | Betermier et al, plos genetics 2014 |
| end-joining | | | | | |
| Non-homologous | C-NHEJ | DNA Pk | | IC486241 | Betermier et al, plos genetics 2014 |
| end-joining | | | | | |
| Non-homologous | C-NHEJ | DNA Pk | | Vaillin | Betermier et al, plos genetics 2014 |
| end-joining | | | | | |
| Non-homologous | C-NHEJ | DNA-PKcs | | | |
| end-joining | | | | | |
| Non-homologous | C-NHEJ | 53BP1 | | | Stephanie Panier and Simon J. |
| end-joining | | | | | Boulton, Nature Review 2014 |
| Non-homologous | C-NHEJ | XLF | | | |
| end-joining | | | | | |
| Non-homologous | C-NHEJ | ARTEMIS | | siRNA | Betermier et al, plos genetics 2014 |
| end-joining | | | | | |
| Non-homologous | C-NHEJ | APLF | | | |
| end-joining | | | | | |
| Non-homologous | C-NHEJ | PNK | | | |
| end-joining | | | | | |
| Non-homologous | C-NHEJ | Rif1 | | | Di Virgilio M. et al Science 2013; |
| end-joining | | | | | Zimmermann M et al Science 2013 |
| Non-homologous | C-NHEJ | PTIP | | | Zimmermann M and De langhe, |
| end-joining | | | | | Trends in cell Biology, 2014 |
| Non-homologous | C-NHEJ | DNA | | | |
| end-joining | | polymerase | | | |
| Non-homologous | C-NHEJ | Ku70 | | | Betermier et al, pl os genetics 2014 |
| end-joining | | | | | |
| Non-homologous | C-NHEJ | Ku80 | | | Betermier et al, plos genetics 2014 |
| end-joining | | | | | |
| Alternative NHEJ | Alt- | PARP1/ | | Olaparib, | |
| pathway | NHEJ | PARP2 | | AZD2281, | |
| | | | | KU-0059436 | |
| Alternative NHEJ | Alt- | PARP1/ | | Iniparib, BSI-201 | |
| pathway | NHEJ | PARP2 | | | |
| Alternative NHEJ | Alt- | PARP1/ | | BMN 673 | |
| pathway | NHEJ | PARP2 | | | |
| Alternative NHEJ | Alt- | PARP1/ | | Rucaparib, | |
| pathway | NHEJ | PARP2 | | (AG014699, | |
| | | | | PF-01367338) | |
| Alternative NHEJ | Alt- | PARP1/ | | Veliparib, ABT-888 | |
| pathway | NHEJ | PARP2 | | | |
| Alternative NHEJ | Alt- | PARP1/ | | CEP 9722 | |
| pathway | NHEJ | PARP2 | | | |
| Alternative NHEJ | Alt- | PARP1/ | | INO-1001 | |
| pathway | NHEJ | PARP2 | | | |
| Alternative NHEJ | Alt- | PARP1/ | | MK 4827 | |
| pathway | NHEJ | PARP2 | | | |
| Alternative NHEJ | Alt- | PARP1/ | | BGB-290 | |
| pathway | NHEJ | PARP2 | | | |
| Alternative NHEJ | Alt- | PARP1/ | | E701, GPI21016 | |
| pathway | NHEJ | PARP2 | | | |
| Alternative NHEJ | Alt- | PARP1/ | | MP-124 | |
| pathway | NHEJ | PARP2 | | | |
| Alternative NHEJ | Alt- | PARP1/ | | LT-673 | |
| pathway | NHEJ | PARP2 | | | |
| Alternative NHEJ | Alt- | PARP1/ | | NMS-P118 | |
| pathway | NHEJ | PARP2 | | | |
| Alternative NHEJ | Alt- | PARP1/ | | XAV939 | |
| pathway | NHEJ | PARP2 | | | |
| Alternative NHEJ | Alt- | PARP1/ | | 3-aminobenzamide | |
| pathway | NHEJ | PARP2 | | | |
| Alternative NHEJ | Alt- | CtIP | | | |
| pathway | NHEJ | | | | |
| Alternative NHEJ | Alt- | LIG-3 | | | |
| pathway | NHEJ | | | | |
| Alternative NHEJ | Alt- | MRE11 | | | |
| pathway | NHEJ | | | | |
| Alternative NHEJ | Alt- | Rad50 | | | |
| pathway | NHEJ | | | | |
| Alternative NHEJ | Alt- | Nbs1 | | | |
| pathway | NHEJ | | | | |
| Alternative NHEJ | Alt- | CtIP | | | |
| pathway | NHEJ | | | | |
| Alternative NHEJ | Alt- | XPF | | NSC 130813 | |
| pathway | NHEJ | | | | |
| Alternative NHEJ | Alt- | ERCC1 | | NSC 130813 | |
| pathway | NHEJ | | | | |
| Alternative NHEJ | Alt- | Ligase-1 | | | |
| pathway | NHEJ | | | | |
| Alternative NHEJ | Alt- | DNA Pol | | | Koole et al, Nature com 2014; Chan et |
| pathway | NHEJ | theta | | | al Plos Genetics 2010 |
| Alternative NHEJ | Alt- | MRN | | | |
| pathway | NHEJ | complex | | | |
| Alternative NHEJ | Alt- | XRCC1 | | | |
| pathway | NHEJ | | | | |
| Single-strand break | SSBR | PARG | | | |
| repair | | | | | |
| Single-strand break | SSBR | XRCC1 | | | Caldecott Nature reviews Genetic, |
| repair | | | | | 2008 |
| Single-strand break | SSBR | DNA | | | |
| repair | | polymerase | | | |
| | | beta | | | |
| Single-strand break | SSBR | DNA | | | |
| repair | | polymerase | | | |
| | | delta | | | |
| Single-strand break | SSBR | DNA | | | |
| repair | | polymerase | | | |
| | | epsilon | | | |
| Single-strand break | SSBR | PCNA | | | |
| repair | | | | | |
| Single-strand break | SSBR | LIG1 | | | |
| repair | | | | | |
| Single-strand break | SSBR | PNK | | | |
| repair | | | | | |
| Single-strand break | SSBR | PNKP | | | |
| repair | | | | | |
| Single-strand break | SSBR | APE1 | | | |
| repair | | | | | |
| Single-strand break | SSBR | APTX | | | |
| repair | | | | | |
| Single-strand break | SSBR | APLF | | | |
| repair | | | | | |
| Single-strand break | SSBR | TDP1 | | | |
| repair | | | | | |
| Single-strand break | SSBR | LIG3 | | | |
| repair | | | | | |
| Single-strand break | SSBR | FEN1 | | | |
| repair | | | | | |
| Single-strand break | SSBR | CtIP | | MLN4924 | |
| repair | | | | | |
| Single-strand break | SSBR | MRN | | | |
| repair | | | | | |
| Single-strand break | SSBR | ERCC1 | | NSC 130813 | |
| repair | | | | | |
| Cell cycle | Cycle | ATM | | | |
| Cellular RNA | Cell | MDA5 | | | |
| response | | | | | |
| | | LIG4 | | SCR7 | |
| | | ATM | | | |
| | | ATR | | | |
| p53 inhibitors | | | | | |
| ATR inhibitors | | | | | |
| DNA Repair | | p53, ATM, | | | |
| | | ATR | | | |
| Cell Cycle | | p53, ATM, | | | |
| | | ATR | | | |
| Cell Death | | p53 | | | |
| DNA sensing | TLR9 | TLR9 | Inhibitor | ODN A151 | Kath et al. (BioRXiV. |
| | | | | | 2021 Feb. 14 431017) |
| DNA sensing | cGAS | cGAS | Inhibitor | ODN A151 | Kath et al. (BioRXiV. |
| | | | | | 2021 Feb. 14 431017) |
| DNA sensing | AIM2 | AIM2 | Inhibitor | ODN A151 | Kath et al. (BioRXiV. |
| | | | | | 2021 Feb. 14 431017) |
| DNA sensing | cGAS | cGAS | Inhibitor | Ru.521 | Kath et al. (BioRXiV. |
| | | | | | 2021 Feb. 14 431017) |
| DNA sensing | STING | | Inhibitor | H151 | Kath et al. (BioRXiV. |
| | | | | | 2021 Feb. 14 431017) |
| DNA sensing | STING | Tbx1 | Inhibitor | BX795 | Kath et al. (BioRXiV. |
| | | | | | 2021 Feb. 14 431017) |

In some embodiments, the methods described herein involve modulating, e.g., upregulating or downregulating, one or more of the following: ADAR1, AICDA, AIM2, ALKBH1, APE, APOBEC1, APOBEC3, APOBEC3A, APOBEC3B, APOBEC3C, APOBEC3F, APOBEC3G, APOBEC3H, ASZ1, ATG5, ATM, BECN1, BRCA1, BRCA2, BST2, C3, C3ORF26, C3ORF37, CALCOCO2, CBX1, CBX3, CDK9, CHAF1, CMPK1, CORO1B, CSDA, DCLRE1C, DDX17, DDX21, DDX39A, DDX4, DDX5, DDX58, DDX6, DGCR8, DHX9, DICER1, DNMT1, DNMT3A, DNMT3B, DNMT3L, DROSHA, EHMT2, ELAVL1, ERAL1, ERCC1, ERCC2, ERCC4, EXD1, EZH2, FAM120A, FAM98A, FANCA, FANCB, FANCM, FASTKD2, FCGR1B, FKBP4, FKBP6, GTSF1, H1FX, HAX1, HECTD1, HENMT1, HEXIM1, HIST1H1C, HIST1H2BO, HNRNPA1, HNRNPA2B1, HNRNPAB, HNRNPC, HNRNPL, HNRNPU, HSP90AA1, HSP90AB1, HSPA1A, HSPA8, IGF2BP1, IGF2BP2, IGF2BP3, ILF2, ILF3, IPO7, ISG20, KDM1A, KIAA0430, KPNA2, KPTN, LARP1, LARP7, LIG4, Ligase IV, MAEL, MATR3, MAVS, MDA5, MECP2, MEPCE, MIR128-1, MORC1, MOV10, MOV10L1, MRE11A, MRPL28, MTNR1A, MX2, NAP1L1, NAP1L4, NCF4, NCL, NOP56, NPM1, NUSAP1, PABPC1, PABPC4, PABPC4L, PALB2, PARP1, PCBP2, PCNA, PIWIL1, PIWIL2, PIWIL4, PLD6, PRKDC, PURA, PURE, RAD50, RAD54L, RALY, RBMX, RCL1, RDH8, RIG-I, RIOK1, RNaseH1, RNaseH2, RNaseH2A, RNaseH2B, RNaseH2C, RNase L, RNASEL, RPRD2, RPS27A, SAMHD1, SERBP1, SETDB1, SF3B3, SIRT6, SNRNP70, SNUPN, SQSTM1, SRP14, SRSF1, SRSF10, SRSF6, SSB, STAU1, STAU2, STK17A, SUV39H, SYNCRIP, TBX1, TDRD1, TDRD12, TDRD5, TDRD9, TDRKH, TEX19, TIMM13, TIMM8B, TLR3, TLR9, TOMM40, TOP1, TRA2A, TRA2B, TREX1, TRIM28, TRIM5a, TROVE2, TUBB, TUBB2C, UBE2T, UHRF1, UNG, UQCRH, XRCC2, XRCC4, XRCC6, XRN1, YARS2, YBX1, YME1L1, ZAP, ZC3HAV1, ZCCHC3, and ZFR.

Inhibitory RNAs

In some embodiments, the host response modulator, e.g., host response inhibitor, comprises a nucleic acid molecule, e.g., RNA molecule. In some embodiments, the host response modulator, e.g., host response inhibitor is an inhibitory RNA molecule. In some embodiments, an inhibitory RNA molecule decreases the level (e.g., protein level or mRNA level) of a factor encoded by a gene described herein, i.e., that mediates host response.

Certain RNAs can inhibit gene expression through the biological process of RNA interference (RNAi). In some embodiments, RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-stranded RNAs (dsRNA), micro RNAs (miRNAs), short hairpin RNAs (shRNA), meroduplexes, and dicer substrates (U.S. Pat. Nos. 8,084,599 8,349,809 and 8,513,207). In some embodiments herein, the agent is an RNAi molecule that inhibits expression of a gene involved in host response.

In some embodiments, RNAi molecules comprise a sequence substantially complementary, or fully complementary, to all or a fragment of a target gene. RNAi molecules may complement sequences at the boundary between introns and exons to prevent the maturation of newly-generated nuclear RNA transcripts of specific genes into mRNA for transcription. RNAi molecules complementary to specific genes can hybridize with the mRNA for that gene, e.g., and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

RNAi molecules can be provided to the cell as “ready-to-use” RNA synthesized in vitro or as an antisense gene transfected into cells which will yield RNAi molecules upon transcription. Hybridization with mRNA, in some embodiments, results in degradation of the hybridized molecule by RNAse H and/or inhibition of the formation of translation complexes. Either may result in a failure to produce the product of the original gene.

The length of the RNAi molecule that hybridizes to the transcript of interest may be around 10 nucleotides, between about 15 or 30 nucleotides, or about 15, 16, 17, 18, 19, 20, 21, 22, 23, 24, 25, 26, 27, 28, 29, 30 or more nucleotides. The degree of identity of the antisense sequence to the targeted transcript may be at least 75%, at least 80%, at least 85%, at least 90%, or at least 95%.

RNAi molecules may also comprise an overhang (e.g., may comprise two overhangs), typically unpaired, overhanging nucleotides which are not directly involved in the double helical structure normally formed by the core sequences of the pair of sense strand and antisense strand. RNAi molecules may contain 3′ and/or 5′ overhangs that are each independently about 1-5 bases (e.g., 2 bases) on each of the sense strands and antisense strands. The sense and antisense strands of an RNAi molecule may contain the same number or a different number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another embodiment, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules typically comprise a nucleotide sequence that is identical to about 15 to about 25 contiguous nucleotides of the target mRNA. In some embodiments, the siRNA sequence commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. In some embodiments, exogenous siRNAs downregulate mRNAs with seed complementarity to the siRNA (Birmingham et al., Nat Methods 3:199-204, 2006. Multiple target sites within a 3′ UTR give stronger downregulation in some embodiments (Doench et al., Genes Dev 17:438-442, 2003).

MicroRNAs

miRNAs and other small interfering nucleic acids generally regulate gene expression via target RNA transcript cleavage/degradation or translational repression of the target messenger RNA (mRNA). miRNAs may, in some instances, be natively expressed, typically as final 19-25 non-translated RNA products. miRNAs generally exhibit their activity through sequence-specific interactions with the 3′ untranslated regions (UTR) of target mRNAs. These endogenously expressed miRNAs may form hairpin precursors that are subsequently processed into an miRNA duplex, and further into a mature single stranded miRNA molecule. This mature miRNA generally guides a multiprotein complex, miRISC, which identifies target 3′ UTR regions of target mRNAs based upon their complementarity to the mature miRNA. Useful transgene products may include, for example, miRNAs or miRNA binding sites that regulate the expression of a linked polypeptide. A non-limiting list of miRNA genes; the products of these genes and their homologues are useful as transgenes or as targets for small interfering nucleic acids (e.g., miRNA sponges, antisense oligonucleotides), e.g., in methods such as those listed in U.S. Ser. No. 10/300,146, 22:25-25:48, incorporated by reference. In some embodiments, one or more binding sites for one or more of the foregoing miRNAs are incorporated in a transgene, e.g., a transgene delivered by a rAAV vector, e.g., to inhibit the expression of the transgene in one or more tissues of an animal harboring the transgene. In some embodiments, a binding site may be selected to control the expression of a transgene in a tissue specific manner. For example, binding sites for the liver-specific miR-122 may be incorporated into a transgene to inhibit expression of that transgene in the liver. Additional exemplary miRNA sequences are described, for example, in U.S. patent Ser. No. 10/300,146 (incorporated herein by reference in its entirety). For liver-specific Gene Writing, however, overexpression of miR-122 may be utilized instead of using binding sites to effect miR-122-specific degradation. This miRNA is positively associated with hepatic differentiation and maturation, as well as enhanced expression of liver specific genes. Thus, in some embodiments, the coding sequence for miR-122 may be added to a component of a Gene Writing system to enhance a liver-directed therapy.

A miR inhibitor or miRNA inhibitor is generally an agent that blocks miRNA expression and/or processing. Examples of such agents include, but are not limited to, microRNA antagonists, microRNA specific antisense, microRNA sponges, and microRNA oligonucleotides (double-stranded, hairpin, short oligonucleotides) that inhibit miRNA interaction with a Drosha complex. MicroRNA inhibitors, e.g., miRNA sponges, can be expressed in cells from transgenes (e.g., as described in Ebert, M. S. Nature Methods, Epub Aug. 12, 2007; incorporated by reference herein in its entirety). In some embodiments, microRNA sponges, or other miR inhibitors, are used with the AAVs. microRNA sponges generally specifically inhibit miRNAs through a complementary heptameric seed sequence. In some embodiments, an entire family of miRNAs can be silenced using a single sponge sequence. Other methods for silencing miRNA function (derepression of miRNA targets) in cells will be apparent to one of ordinary skill in the art.

In some embodiments, a miRNA as described herein comprises a sequence listed in Table 4 of PCT Publication No. WO2020014209, incorporated herein by reference. Also incorporated herein by reference are the listing of exemplary miRNA sequences from WO2020014209.

In some embodiments, it is advantageous to silence one or more components of a Gene Writing system (e.g., mRNA encoding a Gene Writer polypeptide, a Gene Writer Template RNA, or a heterologous object sequence expressed from the genome after successful Gene Writing) in a portion of cells. In some embodiments, it is advantageous to restrict expression of a component of a Gene Writing system to select cell types within a tissue of interest.

For example, it is known that in a given tissue, e.g., liver, macrophages and immune cells, e.g., Kupffer cells in the liver, may engage in uptake of a delivery vehicle for one or more components of a Gene Writing system. In some embodiments, at least one binding site for at least one miRNA highly expressed in macrophages and immune cells, e.g., Kupffer cells, is included in at least one component of a Gene Writing system, e.g., nucleic acid encoding a Gene Writing polypeptide or a transgene. In some embodiments, a miRNA that targets the one or more binding sites is listed in a table referenced herein, e.g., miR-142, e.g., mature miRNA hsa-miR-142-5p or hsa-miR-142-3p.

In some embodiments, there may be a benefit to decreasing Gene Writer levels and/or Gene Writer activity in cells in which Gene Writer expression or overexpression of a transgene may have a toxic effect. For example, it has been shown that delivery of a transgene overexpression cassette to dorsal root ganglion neurons may result in toxicity of a gene therapy (see Hordeaux et al Sci Transl Med 12(569):eaba9188 (2020), incorporated herein by reference in its entirety). In some embodiments, at least one miRNA binding site may be incorporated into a nucleic acid component of a Gene Writing system to reduce expression of a system component in a neuron, e.g., a dorsal root ganglion neuron. In some embodiments, the at least one miRNA binding site incorporated into a nucleic acid component of a Gene Writing system to reduce expression of a system component in a neuron is a binding site of miR-182, e.g., mature miRNA hsa-miR-182-5p or hsa-miR-182-3p. In some embodiments, the at least one miRNA binding site incorporated into a nucleic acid component of a Gene Writing system to reduce expression of a system component in a neuron is a binding site of miR-183, e.g., mature miRNA hsa-miR-183-5p or hsa-miR-183-3p. In some embodiments, combinations of miRNA binding sites may be used to enhance the restriction of expression of one or more components of a Gene Writing system to a tissue or cell type of interest.

Table A5 below provides exemplary miRNAs and corresponding expressing cells, e.g., a miRNA for which one can, in some embodiments, incorporate binding sites (complementary sequences) in the transgene or polypeptide nucleic acid, e.g., to decrease expression in that off-target cell.

TABLE A5
Exemplary miRNA from off-target cells and tissues

| | miRNA | | | SEQ ID |
|---|---|---|---|---|
| Silenced cell type | name | Mature miRNA | miRNA sequence | NO |
| Kupffer cells | miR-142 | hsa-miR-142-5p | cauaaaguagaaagcacuacu | 459 |
| Kupffer cells | miR-142 | hsa-miR-142-3p | uguaguguuuccuacuuuaugga | 460 |
| Dorsal root ganglion | miR-182 | hsa-miR-182-5p | uuuggcaaugguagaacucacacu | 461 |
| neurons | | | | |
| Dorsal root ganglion | miR-182 | hsa-miR-182-3p | ugguucuagacuugccaacua | 462 |
| neurons | | | | |
| Dorsal root ganglion | miR-183 | hsa-miR-183-5p | uauggcacugguagaauucacu | 463 |
| neurons | | | | |
| Dorsal root ganglion | miR-183 | hsa-miR-183-3p | gugaauuaccgaagggccauaa | 464 |
| neurons | | | | |
| Hepatocytes | miR-122 | hsa-miR-122-5p | uggagugugacaaugguguuug | 465 |
| Hepatocytes | miR-122 | hsa-miR-122-3p | aacgccauuaucacacuaaaua | 466 |

RNAi molecules are readily designed and produced by technologies known in the art. In addition, there are computational tools that increase the chance of finding effective and specific sequence motifs (Pei et al. 2006, Reynolds et al. 2004, Khvorova et al. 2003, Schwarz et al. 2003, Ui-Tei et al. 2004, Heale et al. 2005, Chalk et al. 2004, Amarzguioui et al. 2004).

The RNAi molecule modulates expression of RNA encoded by a gene. Because multiple genes can share some degree of sequence homology with each other, in some embodiments, the RNAi molecule can be designed to target a class of genes with sufficient sequence homology. In some embodiments, the RNAi molecule can contain a sequence that has complementarity to sequences that are shared amongst different gene targets or are unique for a specific gene target. In some embodiments, the RNAi molecule can be designed to target conserved regions of an RNA sequence having homology between several genes thereby targeting several genes in a gene family (e.g., different gene isoforms, splice variants, mutant genes, etc.). In some embodiments, the RNAi molecule can be designed to target a sequence that is unique to a specific RNA sequence of a single gene.

In some embodiments, the RNAi molecule is linked to a delivery polymer, e.g., via a physiologically labile bond or linker. The physiologically labile linker is selected such that it undergoes a chemical transformation (e.g., cleavage) when present in certain physiological conditions, (e.g., disulfide bond cleaved in the reducing environment of the cell cytoplasm). Release of the molecule from the polymer, by cleavage of the physiologically labile linkage, facilitates interaction of the molecule with the appropriate cellular components for activity.

The RNAi molecule-polymer conjugate may be formed by covalently linking the molecule to the polymer. The polymer is polymerized or modified such that it contains a reactive group A. The RNAi molecule is also polymerized or modified such that it contains a reactive group B. Reactive groups A and B are chosen such that they can be linked via a reversible covalent linkage using methods known in the art.

Conjugation of the RNAi molecule to the polymer can be performed in the presence of an excess of polymer. Because the RNAi molecule and the polymer may be of opposite charge during conjugation, the presence of excess polymer can reduce or eliminate aggregation of the conjugate. Alternatively, an excess of a carrier polymer, such as a polycation, can be used. The excess polymer can be removed from the conjugated polymer prior to administration of the conjugate to the animal or cell culture. Alternatively, the excess polymer can be co-administered with the conjugate to the animal or cell culture.

For example, an inhibitory RNA molecule includes a short interfering RNA, short hairpin RNA, and/or a microRNA that targets gene expression of a gene involved in host response. A siRNA is a double-stranded RNA molecule that typically has a length of about 19-25 base pairs. A shRNA is a RNA molecule including a hairpin turn that decreases expression of a target gene, e.g., via RNAi. shRNAs can be delivered to cells in the form of plasmids, e.g., viral or bacterial vectors, e.g., by transfection, electroporation, or transduction). A microRNA is a non-coding RNA molecule that typically has a length of about 22 nucleotides. MiRNAs typically bind to target sites on mRNA molecules and silence the mRNA, e.g., by causing cleavage of the mRNA, destabilization of the mRNA, or inhibition of translation of the mRNA. In embodiments, the inhibitory RNA molecule decreases the level and/or activity of a negative regulator of function or a positive regulator of function. In other embodiments, the inhibitory RNA molecule decreases the level and/or activity of an inhibitor of a positive regulator of function.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity.

In some embodiments, the inhibitory RNA molecule decreases the level and/or activity or function of a factor encoded by a gene involved in host response. In embodiments, the inhibitory RNA molecule inhibits expression of a factor encoded by a gene involved in host response. In other embodiments, the inhibitory RNA molecule increases degradation of encoded by a gene involved in host response and/or decreases the half-life of a factor encoded by a gene involved in host response. The inhibitory RNA molecule can be chemically synthesized or transcribed in vitro.

The making and use of inhibitory therapeutic agents based on non-coding RNA such as ribozymes, RNAse P, siRNAs, and miRNAs are further described in Sioud, RNA Therapeutics: Function, Design, and Delivery (Methods in Molecular Biology). Humana Press 2010; and Kaczmarek et al. 2017. Advances in the delivery of RNA therapeutics: from concept to clinical reality. Genome Medicine 9:60.

CRISPR

A CRISPR system can be used to inhibit expression of a gene involved in host response, e.g., to inactivate a gene involved in host response as described herein, or to reduce or inhibit gene expression of a gene involved in host response (e.g., by genetic or epigenetic editing). In certain embodiments, an inhibitor CRISPR system comprises a negative effector and one or more guide RNA that targets a gene involved in host response.

CRISPR systems use RNA-guided nucleases termed CRISPR-associated or “Cas” endonucleases (e. g., Cas9 or Cpf1) to cleave DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically an about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be close to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPR1), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRNAs without the requirement of a tracrRNA; in other words a Cpf1 system may be used with only the Cpf1 nuclease and a crRNA to cleave the target DNA sequence. Cpf1 endonucleases are typically associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are typically used by Cas9 for DNA cleavage to occur; for Cpf1 at least about 16 nucleotides of gRNA sequence is typically used to achieve detectable DNA cleavage. In practice, guide RNA sequences are generally designed to have a length of between 17-24 nucleotides (e.g., 19, 20, or 21 nucleotides) and complementarity to the targeted gene or nucleic acid sequence. Custom gRNA generators and algorithms are available commercially for use in the design of effective guide RNAs. Gene editing has also been achieved using a chimeric “single guide RNA” (“sgRNA”), a single RNA molecule and contains both a tracrRNA reign (e.g., which binds the nuclease) and at least one crRNA region (e.g., which guides the nuclease to the sequence targeted for editing). sgRNAs are typically engineered molecules that mimic a naturally occurring crRNA-tracrRNA complex. Chemically modified sgRNAs have also been demonstrated to be effective in genome editing; see, for example, Hendel et al. (2015) Nature Biotechnol., 985-991.

Whereas wild-type Cas9 typically generates double-strand breaks (DSBs) at specific DNA sequences targeted by a gRNA, a number of CRISPR endonucleases having modified functionalities are available, for example: a “nickase” version of Cas9 generates a single-strand break; a catalytically inactive Cas9 (“dCas9”) interferes with transcription by steric hindrance, and generally does not cut the target DNA or does not cut it at detectable levels. dCas9 can further be fused with an effector to repress (CRISPRi) or activate (CRISPRa) expression of a target gene. For example, Cas9 can be fused to a transcriptional repressor (e.g., a KRAB domain) or a transcriptional activator (e.g., a dCas9-VP64 fusion). A catalytically inactive Cas9 (dCas9) fused to Fok1 nuclease (“dCas9-Fok1”) can be used to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, Mass. 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389. In one embodiment, an inhibitor disclosed herein comprises a CRISPRi system to reduce expression of a gene involved in host response.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. CRISPR technology for generating mtDNA dysfunction in the mitochondrial genome with the CRISPR/Cas9 system is disclosed in Jo, A., et al., BioMed Res. Int'l, vol 2015, article ID 305716, 10 pages, http://dx.doi.org/10.1155/2015/305716. Co-delivery of Cas9 and sgRNA with nanoparticles is disclosed in Mout, R., et al., ACS Nano, Jan. 31, 2017, article ID doi: 10.1021/acsnano.6b07600.

In some embodiments, the composition comprising a gRNA and a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease, are used to modulate gene expression. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (sgRNA) to modulate activity and/or expression of one or more target nucleic acids sequences (e.g., to methylate or demethylate a DNA sequence).

RNA Sensing

In some embodiments, the host response modulator inhibits one or more proteins involved in RNA sensing and response, e.g., TLR3, TLR4, TLR7, TLR8, MyD88, TRIF, IKK, NF-κB, IRF3, IRF7, IFN-α, TNFα, IL-6, IL-12, JAK-1, TYK-2, STAT1, STAT2, IRF-9, PKR, OAS, ADAR, RIG-I, MDA5, LGP2, MAVS, NLRP3, NOD2, or caspase 1, or any combination thereof.

Without wishing to be bound by theory, in some embodiments, activation of TLR4 blocks mRNA translation without reducing the cellular uptake of LNPs. The inhibition of TLR4 or its downstream effector protein kinase R can improve expression of mRNA delivered naked to cells or in LNPs (Lokugamage et al. Adv Materials 2019). In some embodiments, an inhibitor of TLR4 or a downstream effector, e.g., protein kinase R, is used to improve the efficiency of a Gene Writing system. In some embodiments, the host response modulator which is an inhibitor of one or more proteins involved in RNA sensing and response (e.g., TLR4) increases expression of a GeneWriter polypeptide from an mRNA, e.g., increases Gene Writer protein levels to at least 10%, 20%, 30%, 40%, 50%, 60%, 70%, 80%, 90%, 100%, 200%, or 300% higher than in an otherwise similar cell not contacted with the host response modulator.

Epigenetic Modifiers

In some embodiments, an agent that promotes activity of a Gene Writer polypeptide (e.g., promotes insertion of a heterologous object sequence by a Gene Writer polypeptide) is an epigenetic modifier. Without wishing to be bound by theory, in some embodiments the chromatin structure of the insertion site affects the efficiency of insertion, e.g., open chromatin may be more permissive than heterochromatin for insertion. Accordingly, Gene Writer activity may be increased by co-administration of an epigenetic modifier.

In some embodiments, the epigenetic modifier acts specifically at the target site. In some embodiments, the epigenetic modifier acts at a plurality of sites in the genome (e.g., globally), wherein one of the plurality of sites is the target site. An epigenetic modifier can comprise, e.g., a chromatin modifying enzyme (or a nucleic acid encoding the same), an inhibitor of an endogenous chromatin modifying enzyme (e.g., a nucleic acid inhibitor), or a small molecule (e.g., a small molecule inhibitor of an endogenous chromatin modifying enzyme).

In some embodiments, the epigenetic modifier that promotes transposition is an HDAC inhibitor or a histone methyltransferase inhibitor. These inhibitors act on histone deacetylases and histone methyltransferases, respectively, blocking their activities and allowing chromatin expansion, which may improve the accessibility of target DNA to Gene Writing systems. In some embodiments, HDAC inhibitors, histone methyltransferase inhibitors, or a combination of both may be provided along with a Gene Writing system in order to improve the efficiency of integration. HDAC inhibitors and histone methyltransferase inhibitor are described in WO2020077357A1, which is incorporated herein by reference in its entirety.

In some embodiments, the HDAC inhibitor is a pan-HDAC inhibitor, a class I HD AC inhibitor, a class II HDAC inhibitor or a class I and class II HDAC inhibitor. Non-limiting examples of pan-HDAC inhibitors include Trichostatin A (TSA), Vorinostat, CAY10433 (targets class I and II), or sodium phenylbutyrate (targets class I and IIa). Non-limiting examples of class I HDAC inhibitors (targeting HDAC 1, 2, 3, or 8) include MS-275, CAY10398, or Entinostat. Non limiting examples of class II HDAC inhibitors (targeting HDAC 4, 5, 6, 7, 9, or 10) include MC-1568, Scriptaid, or CAY10603. Valproic acid (VPA) can inhibits multiple histone deacetylases from both Class I and Class II.

The histone methyltransferase inhibitor can be a selective inhibitor of G9a/GLP histone methyltransferases, which methylate lysine 9 of histone 3 (H3K9). Non-limiting examples of G9a/GLP inhibitors include BIX01294, UNC0642, A-366, UNC0224, UNC0631, UNC0646, BRD4770, or UNC0631. Non-limiting examples of histone lysine methyltransferases include chaetocin, EPZ005687, EPZ6438, GSK126, GKS343, Ell, UNC199, EPZ004777, EPZ5676, LLY-507, AZ505, or A-893. The histone methyltransferase inhibitor can be 2-Cyclohexyl-N-(1-isopropylpiperidin-4-yl)-6-methoxy-7-(3-(pyrrolidin-1-yl)propoxy) quinazolin-4-amine (ETNC0638), BIX01294, ETNC0642, A-366, UNCO224, UNC0631, UNC0646, BRD4770, UNC0631, chaetocin, EPZ005687, EPZ6438, GSK126, GKS343, Ell, UNC199, EPZ004777, EPZ5676, LLY-507, AZ505 or A-893. In some embodiments, the histone methyltransferase inhibitor is UNC0638.

In some embodiments, the epigenetic modifier comprises a targeting moiety that directs it to the target site. In some embodiments, the targeting moiety comprises a DNA binding domain, e.g., a zinc finger domain, a TAL effector domain, or a catalytically inactive Cas protein.

Gene Writer Polypeptides

A Gene Writer polypeptide is typically a substantially autonomous protein machine capable of integrating a template nucleic acid into a target DNA molecule (e.g., in a mammalian host cell, such as a genomic DNA molecule in the host cell), substantially without relying on host machinery.

Gene Writers suitable for use in the compositions and methods described herein include, e.g., retrotransposases, DNA transposases, and recombinases (e.g., serine recombinases and tyrosine recombinases). Exemplary Gene Writer polypeptide, and systems comprising them and methods of using them are described, e.g., in PCT/US19/48607, filed Aug. 28, 2019; 62/876,165, filed Jul. 19, 2019; 62/939,525, filed Nov. 22, 2019; and 62/967,934, filed Jan. 30, 2020, each of which are incorporated herein by reference, including the amino acid and nucleic acid sequences therein.

For example, Table 3 of PCT/US19/48607 is herein incorporated by reference in its entirety. In some embodiments, a Gene Writer polypeptide comprises an amino acid sequence of column 8 of Table 3 of PCT/US19/48607, or any domain thereof (e.g., a DNA binding domain, RNA binding domain, endonuclease domain, or RT domain) or a sequence having at least 70%, 75%, 80%, 85%, 90%, 95%, 96%, 97%, 98%, or 99% identity thereto. In some embodiments, a template RNA comprises a sequence of Table 3 of PCT/US19/48607 (e.g., one or both of a 5′ untranslated region of column 6 and a 3′ untranslated region of column 7), or a sequence having at least 70%, 75%, 80%, 85%, 90%, 95%, 96%, 97%, 98%, or 99% identity thereto.

Exemplary GeneWriter polypeptides and RT domain sequences are also described, e.g., in U.S. Provisional Application No. 63/035,627 filed Jun. 5, 2020, e.g., at Table 1, Table 3, Table 30, and Table 31 therein; the entire application is incorporated by reference herein including said sequences and tables. Accordingly, a GeneWriter polypeptide described herein may comprise an amino acid sequence according to any of the Tables mentioned in this paragraph, or a domain thereof (e.g., an RT domain, a DNA binding domain, an RNA binding domain, or an endonuclease domain), or a functional fragment or variant of any of the foregoing, or an amino acid sequence having at least 70%, 80%, 85%, 90%, 95%, or 99% identity thereto.

In some embodiments, a Gene Writer polypeptide includes one or more domains that, collectively, facilitate 1) binding the template nucleic acid, 2) binding the target DNA molecule, and 3) facilitate integration of the at least a portion of the template nucleic acid into the target DNA. In some embodiments, the Gene Writer polypeptide is a naturally occurring polypeptide. In some embodiments, the Gene Writer polypeptide is an engineered polypeptide, e.g., having one or more amino acid substitutions to the naturally occurring sequence. In some embodiments, the Gene Writer polypeptides comprises two or more domains that are heterologous relative to each other, e.g., through a heterologous fusion (or other conjugate) of otherwise wild-type domains, or well as fusions of modified domains, e.g., by way of replacement or fusion of a heterologous sub-domain or other substituted domain. For instance, in some embodiments, one or more of: the RT domain is heterologous to the DBD; the DBD is heterologous to the endonuclease domain; or the RT domain is heterologous to the endonuclease domain.

In some embodiments, a Gene Writer system is capable of producing a substitution into the target site of at least 1, 2, 3, 4, 5, 6, 7, 8, 9, 10, 15, 20, 25, 30, 35, 40, 45, 50, 60, 70, 80, 90, or 100 or more nucleotides. In some embodiments, the substitution is a transition mutation. In some embodiments, the substitution is a transversion mutation. In some embodiments, the substitution converts an adenine to a thymine, an adenine to a guanine, an adenine to a cytosine, a guanine to a thymine, a guanine to a cytosine, a guanine to an adenine, a thymine to a cytosine, a thymine to an adenine, a thymine to a guanine, a cytosine to an adenine, a cytosine to a guanine, or a cytosine to a thymine.

In some embodiments, an insertion, deletion, substitution, or combination thereof, increases or decreases expression (e.g. transcription or translation) of a gene. In some embodiments, an insertion, deletion, substitution, or combination thereof, increases or decreases expression (e.g. transcription or translation) of a gene by altering, adding, or deleting sequences in a promoter or enhancer, e.g. sequences that bind transcription factors. In some embodiments, an insertion, deletion, substitution, or combination thereof alters translation of a gene (e.g. alters an amino acid sequence), inserts or deletes a start or stop codon, alters or fixes the translation frame of a gene. In some embodiments, an insertion, deletion, substitution, or combination thereof alters splicing of a gene, e.g. by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g. from the cytoplasm to a mitochondria, from the cytoplasm into the extracellular space (e.g. adds a secretion tag)). In some embodiments, an insertion, deletion, substitution, or combination thereof alters (e.g. improves) protein folding (e.g. to prevent accumulation of misfolded proteins). In some embodiments, an insertion, deletion, substitution, or combination thereof, alters, increases, decreases the activity of a gene, e.g. a protein encoded by the gene.

Retargeting (e.g., of a Gene Writer polypeptide or nucleic acid molecule, or of a system as described herein) generally comprises: (i) directing the polypeptide to bind and cleave at the target site; and/or (ii) designing the template RNA to have complementarity to the target sequence. In some embodiments, the template RNA has complementarity to the target sequence 5′ of the first-strand nick, e.g., such that the 3′ end of the template RNA anneals and the 5′ end of the target site serves as the primer, e.g., for TPRT. In some embodiments, the endonuclease domain of the polypeptide and the 5′ end of the RNA template are also modified as described.

In some embodiments, a Gene Writer polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, the functional domain comprises a zinc finger (e.g., a zinc finger that specifically binds to the target nucleic acid (e.g., DNA) sequence of interest. In some embodiments, the functional domain comprises a Cas domain (e.g., a Cas domain that specifically binds to the target nucleic acid (e.g., DNA) sequence of interest. In embodiments, the Cas domain comprises a Cas9 or a mutant or variant thereof (e.g., as described herein). In embodiments, the Cas domain is associated with a guide RNA (gRNA), e.g., as described herein. In embodiments, the Cas domain is directed to a target nucleic acid (e.g., DNA) sequence of interest by the gRNA. In embodiments, the Cas domain is encoded in the same nucleic acid (e.g., RNA) molecule as the gRNA. In embodiments, the Cas domain is encoded in a different nucleic acid (e.g., RNA) molecule from the gRNA.

In some embodiments, a Gene Writer polypeptide comprises a modification to an endonuclease domain, e.g., relative to the wild-type polypeptide. In some embodiments, the endonuclease domain comprises an addition, deletion, replacement, or modification to the amino acid sequence of the original endonuclease domain. In some embodiments, the endonuclease domain is modified to include a heterologous functional domain that binds specifically to and/or induces endonuclease cleavage of a target nucleic acid (e.g., DNA) sequence of interest. In some embodiments, the endonuclease domain comprises a zinc finger. In some embodiments, the endonuclease domain comprises a Cas domain (e.g., a Cas9 or a mutant or variant thereof). In embodiments, the endonuclease domain comprising the Cas domain is associated with a guide RNA (gRNA), e.g., as described herein. In some embodiments, the endonuclease domain is modified to include a functional domain that does not target a specific target nucleic acid (e.g., DNA) sequence. In embodiments, the endonuclease domain comprises a Fok1 domain.

In some embodiments, the reverse transcriptase (RT) domain exhibits enhanced stringency of target-primed reverse transcription (TPRT) initiation, e.g., relative to an endogenous RT domain. In some embodiments, the RT domain initiates TPRT when the 3 nt in the target site immediately upstream of the first strand nick, e.g., the genomic DNA priming the RNA template, have at least 66% or 100% complementarity to the 3 nt of homology in the RNA template. In some embodiments, the RT domain initiates TPRT when there are less than 5 nt mismatched (e.g., less than 1, 2, 3, 4, or 5 nt mismatched) between the template RNA homology and the target DNA priming reverse transcription. In some embodiments, the RT domain is modified such that the stringency for mismatches in priming the TPRT reaction is increased, e.g., wherein the RT domain does not tolerate any mismatches or tolerates fewer mismatches in the priming region relative to a wild-type (e.g., unmodified) RT domain. In some embodiments, the RT domain comprises a HIV-1 RT domain. In embodiments, the HIV-1 RT domain initiates lower levels of synthesis even with three nucleotide mismatches relative to an alternative RT domain (e.g., as described by Jamburuthugoda and Eickbush J Mol Biol 407(5):661-672 (2011); incorporated herein by reference in its entirety).

In some embodiments, a Gene Writing polypeptide has an endonuclease domain comprising a Cas9 nickase, e.g., Cas9 H840A. In embodiments, the Cas9 H840A has the following amino acid sequence:

Cas9 nickase (H840A):
(SEQ ID NO: 1)
DKKYSIGLDIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGD

In some embodiments, a Gene Writing polypeptide comprises the RT domain from a retroviral reverse transcriptase, e.g., a wild-type M-MLV RT, e.g., comprising the following sequence:

M-MLV (WT):
(SEQ ID NO: 2)
TLNIEDEYRLHETSKEPDVSLGSTWLSDFPQAWAETGGMGLAVRQAPLIIPLKATSTPVSI
KQYPMSQEARLGIKPHIQRLLDQGILVPCQSPWNTPLLPVKKPGTNDYRPVQDLREVNK
RVEDIHPTVPNPYNLLSGLPPSHQWYTVLDLKDAFFCLRLHPTSQPLFAFEWRDPEMGIS
GQLTWTRLPQGFKNSPTLFDEALHRDLADFRIQHPDLILLQYVDDLLLAATSELDCQQG
TRALLQTLGNLGYRASAKKAQICQKQVKYLGYLLKEGQRWLTEARKETVMGQPTPKT
PRQLREFLGTAGFCRLWIPGFAEMAAPLYPLTKTGTLFNWGPDQQKAYQEIKQALLTAP
ALGLPDLTKPFELFVDEKQGYAKGVLTQKLGPWRRPVAYLSKKLDPVAAGWPPCLRM
VAAIAVLTKDAGKLTMGQPLVILAPHAVEALVKQPPDRWLSNARMTHYQALLLDTDR
VQFGPVVALNPATLLPLPEEGLQHNCLDILAEAHGTRPDLTDQPLPDADHTWYTDGSSL
LQEGQRKAGAAVTTETEVIWAKALPAGTSAQRAELIALTQALKMAEGKKLNVYTDSRY
AFATAHIHGEIYRRRGLLTSEGKEIKNKDEILALLKALFLPKRLSIIHCPGHQKGHSAEAR
GNRMADQAARKAAITETPDTSTLLI

In some embodiments, a Gene Writing polypeptide comprises the RT domain from a retroviral reverse transcriptase, e.g., an M-MLV RT, e.g., comprising the following sequence:

(SEQ ID NO: 3)
TLNIEDEHRLHETSKEPDVSLGSTWLSDFPQAWAETGGMGLAVRQAPLIPLKATSTPVSI
KQYPMSQEARLGIKPHIQRLLDQGILVPCQSPWNTPLLPVKKPGTNDYRPVQDLREVNK
RVEDIHPTVPNPYNLLSGLPPSHQWYTVLDLKDAFFCLRLHPTSQPLFAFEWRDPEMGIS
GQLTWTRLPQGFKNSPTLFDEALHRDLADFRIQHPDLILLQYVDDLLLAATSELDCQQG
TRALLQTLGNLGYRASAKKAQICQKQVKYLGYLLKEGQRWLTEARKETVMGQPTPKT
PRQLREFLGTAGFCRLWIPGFAEMAAPLYPLTKTGTLFNWGPDQQKAYQEIKQALLTAP
ALGLPDLTKPFELFVDEKQGYAKGVLTQKLGPWRRPVAYLSKKLDPVAAGWPPCLRM
VAAIAVLTKDAGKLTMGQPLVILAPHAVEALVKQPPDRWLSNARMTHYQALLLDTDR
VQFGPVVALNPATLLPLPEEGLQHNCLDILAEAHGTRPDLTDQPLPDADHTWYTDGSSL
LQEGQRKAGAAVTTETEVIWAKALPAGTSAQRAELIALTQALKMAEGKKLNVYTDSRY
AFATAHIHGEIYRRRGLLTSEGKEIKNKDEILALLKALFLPKRLSIIHCPGHQKGHSAEAR
GNRMADQAARKAAITETPDTSTLL

In some embodiments, a Gene Writing polypeptide comprises the RT domain from a retroviral reverse transcriptase comprising the sequence of amino acids 659-1329 of NP_057933. In embodiments, the Gene Writing polypeptide further comprises one additional amino acid at the N-terminus of the sequence of amino acids 659-1329 of NP_057933, e.g., as shown below:

(SEQ ID NO: 4)
TLNIEDEHRLHETSKEPDVSLGSTWLSDFPQAWAETGGMGLAVRQAPLIPLKATSTPVSI
KQYPMSQEARLGIKPHIQRLLDQGILVPCQSPWNTPLLPVKKPGTNDYRPVQDLREVN
KRVEDIHPTVPNPYNLLSGLPPSHQWYTVLDLKDAFFCLRLHPTSQPLFAFEWRDP
EMGISGQLTWTRLPQGFKNSPTLFDEALHRDLADFRIQHPDLILLQYVDDLLLAAT
SELDCQQGTRALLQTLGNLGYRASAKKAQICQKQVKYLGYLLKEGQRWLTEARKE
TVMGQPTPKTPRQLREFLGTAGFCRLWIPGFAEMAAPLYPLTKTGTLFNWGPDQQKAY
QEIKQALLTAPALGLPDLTKPFELFVDEKQGYAKGVLTQKLGPWRRPVAYLSKKLDPV
AAGWPPCLRMVAAIAVLTKDAGKLTMGQPLVILAPHAVEALVKQPPDRWLSNARMTH
YQALLLDTDRVQFGPVVALNPATLLPLPEEGLQHNCLDILAEAHGTRPDLTDQPLPDAD
HTWYTDGSSLLQEGQRKAGAAVTTETEVIWAKALPAGTSAQRAELIALTQALKMAEGK
KLNVYTDSRYAFATAHIHGEIYRRRGLLTSEGKEIKNKDEILALLKALFLPKRLSIIHCPG
HQKGHSAEARGNRMADQAARKAA

Core RT (bold), annotated per above
RNAseH (underlined), annotated per above

In embodiments, the Gene Writing polypeptide further comprises one additional amino acid at the C-terminus of the sequence of amino acids 659-1329 of NP_057933. In embodiments, the Gene Writing polypeptide comprises an RNaseH1 domain (e.g., amino acids 1178-1318 of NP_057933).

In some embodiments, a retroviral reverse transcriptase domain, e.g., M-MLV RT, may comprise one or more mutations from a wild-type sequence that may improve features of the RT, e.g., thermostability, processivity, and/or template binding. In some embodiments, an M-MLV RT domain comprises, relative to the M-MLV (WT) sequence above, one or more mutations, e.g., selected from D200N, L603W, T330P, T306K, W313F, D524G, E562Q, D583N, P51L, S67R, E67K, T197A, H204R, E302K, F309N, L435G, N454K, H594Q, D653N, R110S, K103L, e.g., a combination of mutations, such as D200N, L603W, and T330P, optionally further including T306K and W313F. In some embodiments, an M-MLV RT used herein comprises the mutations D200N, L603W, T330P, T306K and W313F. In embodiments, the mutant M-MLV RT comprises the following amino acid sequence:

M-MLV (PE2):
(SEQ ID NO: 5)
TLNIEDEYRLHETSKEPDVSLGSTWLSDFPQAWAETGGMGLAVRQAPLIIPLKATSTPVSI
KQYPMSQEARLGIKPHIQRLLDQGILVPCQSPWNTPLLPVKKPGTNDYRPVQDLREVNK
RVEDIHPTVPNPYNLLSGLPPSHQWYTVLDLKDAFFCLRLHPTSQPLFAFEWRDPEMGIS
GQLTWTRLPQGFKNSPTLFNEALHRDLADFRIQHPDLILLQYVDDLLLAATSELDCQQG
TRALLQTLGNLGYRASAKKAQICQKQVKYLGYLLKEGQRWLTEARKETVMGQPTPKT
PRQLREFLGKAGFCRLFIPGFAEMAAPLYPLTKPGTLFNWGPDQQKAYQEIKQALLTAP
ALGLPDLTKPFELFVDEKQGYAKGVLTQKLGPWRRPVAYLSKKLDPVAAGWPPCLRM
VAAIAVLTKDAGKLTMGQPLVILAPHAVEALVKQPPDRWLSNARMTHYQALLLDTDR
VQFGPVVALNPATLLPLPEEGLQHNCLDILAEAHGTRPDLTDQPLPDADHTWYTDGSSL
LQEGQRKAGAAVTTETEVIWAKALPAGTSAQRAELIALTQALKMAEGKKLNVYTDSRY
AFATAHIHGEIYRRRGWLTSEGKEIKNKDEILALLKALFLPKRLSIIHCPGHQKGHSAEAR
GNRMADQAARKAAITETPDTSTLLI

In some embodiments, a Gene Writer polypeptide may comprise a linker, e.g., a peptide linker, e.g., a linker as described in Table 1. In some embodiments, a Gene Writer polypeptide comprises a flexible linker between the endonuclease and the RT domain, e.g., a linker comprising the amino acid sequence SGGSSGGSSGSETPGTSESATPESSGGSSGGSS (SEQ ID NO: 6). In some embodiments, an RT domain of a Gene Writer polypeptide may be located C-terminal to the endonuclease domain. In some embodiments, an RT domain of a Gene Writer polypeptide may be located N-terminal to the endonuclease domain.

TABLE 1
Exemplary linker sequences

| | SEQ |
|---|---|
| | ID |
| Amino Acid Sequence | NO |
| GGS | 101 |
| GGSGGS | 102 |
| GGSGGSGGS | 103 |
| GGSGGSGGSGGS | 104 |
| GGSGGSGGSGGSGGS | 105 |
| GGSGGSGGSGGSGGSGGS | 106 |
| GGGGS | 107 |
| GGGGSGGGGS | 108 |
| GGGGSGGGGSGGGGS | 109 |
| GGGGSGGGGSGGGGSGGGGS | 110 |
| GGGGSGGGGSGGGGSGGGGSGGGGS | 111 |
| GGGGSGGGGSGGGGSGGGGSGGGGSGGGGS | 112 |
| GGG | 113 |
| GGGG | 114 |
| GGGGG | 115 |
| GGGGGG | 116 |
| GGGGGGG | 117 |
| GGGGGGGG | 118 |
| GSS | 119 |
| GSSGSS | 120 |
| GSSGSSGSS | 121 |
| GSSGSSGSSGSS | 122 |
| GSSGSSGSSGSSGSS | 123 |
| GSSGSSGSSGSSGSSGSS | 124 |
| EAAAK | 125 |
| EAAAKEAAAK | 126 |
| EAAAKEAAAKEAAAK | 127 |
| EAAAKEAAAKEAAAKEAAAK | 128 |
| EAAAKEAAAKEAAAKEAAAKEAAAK | 129 |
| EAAAKEAAAKEAAAKEAAAKEAAAKEAAAK | 130 |
| PAP | 131 |
| PAPAP | 132 |
| PAPAPAP | 133 |
| PAPAPAPAP | 134 |
| PAPAPAPAPAP | 135 |
| PAPAPAP APAPAP | 136 |
| GGSGGG | 137 |
| GGGGGS | 138 |
| GGSGSS | 139 |
| GSSGGS | 140 |
| GGSEAAAK | 141 |
| EAAAKGGS | 142 |
| GGSPAP | 143 |
| PAPGGS | 144 |
| GGGGSS | 145 |
| GSSGGG | 146 |
| GGGEAAAK | 147 |
| EAAAKGGG | 148 |
| GGGPAP | 149 |
| PAPGGG | 150 |
| GSSEAAAK | 151 |
| EAAAKGSS | 152 |
| GSSPAP | 153 |
| PAPGSS | 154 |
| EAAAKPAP | 155 |
| PAPEAAAK | 156 |
| GGSGGGGSS | 157 |
| GGSGSSGGG | 158 |
| GGGGGSGSS | 159 |
| GGGGSSGGS | 160 |
| GSSGGSGGG | 161 |
| GSSGGGGGS | 162 |
| GGSGGGEAAAK | 163 |
| GGSEAAAKGGG | 164 |
| GGGGGSEAAAK | 165 |
| GGGEAAAKGGS | 166 |
| EAAAKGGSGGG | 167 |
| EAAAKGGGGGS | 168 |
| GGSGGGPAP | 169 |
| GGSPAPGGG | 170 |
| GGGGGSPAP | 171 |
| GGGPAPGGS | 172 |
| PAPGGSGGG | 173 |
| PAPGGGGGS | 174 |
| GGSGSSEAAAK | 175 |
| GGSEAAAKGSS | 176 |
| GSSGGSEAAAK | 177 |
| GSSEAAAKGGS | 178 |
| EAAAKGGSGSS | 179 |
| EAAAKGSSGGS | 180 |
| GGSGSSPAP | 181 |
| GGSPAPGSS | 182 |
| GSSGGSPAP | 183 |
| GSSPAPGGS | 184 |
| PAPGGSGSS | 185 |
| PAPGSSGGS | 186 |
| GGSEAAAKPAP | 187 |
| GGSPAPEAAAK | 188 |
| EAAAKGGSPAP | 189 |
| EAAAKPAPGGS | 190 |
| PAPGGSEAAAK | 191 |
| PAPEAAAKGGS | 192 |
| GGGGSSEAAAK | 193 |
| GGGEAAAKGSS | 194 |
| GSSGGGEAAAK | 195 |
| GSSEAAAKGGG | 196 |
| EAAAKGGGGSS | 197 |
| EAAAKGSSGGG | 198 |
| GGGGSSPAP | 199 |
| GGGPAPGSS | 200 |
| GSSGGGPAP | 201 |
| GSSPAPGGG | 202 |
| PAPGGGGSS | 203 |
| PAPGSSGGG | 204 |
| GGGEAAAKPAP | 205 |
| GGGPAPEAAAK | 206 |
| EAAAKGGGPAP | 207 |
| EAAAKPAPGGG | 208 |
| PAPGGGEAAAK | 209 |
| PAPEAAAKGGG | 210 |
| GSSEAAAKPAP | 211 |
| GSSPAPEAAAK | 212 |
| EAAAKGSSPAP | 213 |
| EAAAKPAPGSS | 214 |
| PAPGSSEAAAK | 215 |
| PAPEAAAKGSS | 216 |
| AEAAAKEAAAKEAAAKEAAAKALEAEAAAKEAAAKEAAAKEA | 217 |
| AAKA | |
| GGGGSEAAAKGGGGS | 218 |
| EAAAKGGGGSEAAAK | 219 |
| SGSETPGTSESATPES | 220 |
| GSAGSAAGSGEF | 221 |
| SGGSSGGSSGSETPGTSESATPESSGGSSGGSS | 222 |

In some embodiments, a Gene Writer polypeptide comprises a dCas9 sequence comprising a D10A and/or H840A mutation, e.g., the following sequence:

(SEQ ID NO: 7)
SMDKKYSIGLAIGTNSVGWAVITDDYKVPSKKFKVLGNTDRHSIKKNLIGALLFDSGET
AEATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRRYT
GWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQK
NSRERMKRIEEGIKELGSQILKEHPVENTQLQNEKLYLYYLQNGRDMYVDQELDINRLS
DYDVDAIVPQSFLKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIR
EVKVITLKSKLVSDFRKDFQFYKVREINNYHHAHDAYLNAVVGTALIKKYPKLESEFVY
GDYKVYDVRKMIAKSEQEIGKATAKYFFYSNIMNFFKTEITLANGEIRKRPLIETNGETG
ETVWDKGRDFATVRKVLSMPQVNTVKKTEVQTGGFSKESILPKRNSDKLIARKKDWDPK
KYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKG
SPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGD

In some embodiments, the Gene Writer polypeptide is covalently linked or fused with the agent that promotes activity of the gene modifying system, (e.g., a host response modulator or an epigenetic modifier). In some embodiments, the host response modulator, e.g., host response enhancer or inhibitor is a protein or a functional fragment thereof, e.g., a domain.

In some embodiments, the protein or the domain fused to the Gene Writing polypeptide stimulates a host process, e.g., activates or recruits a host protein or complex. In some embodiments, the protein or the domain stimulates Gene Writing, e.g., by replacing or supplementing a host protein, complex, or pathway. In some embodiment, the protein is a host DNA repair enzyme that stimulates Gene Writing. In some embodiments, the protein or the domain stimulates trans writing. In some embodiments, the protein or the domain stimulates cis writing. In some embodiments, the domain is a domain that recruits a host 5′ exonuclease, e.g., EXO1, for cis writing. In some embodiments, the domain is a domain that that recruits a structure-specific endonuclease, e.g., FEN1, for cis writing. In some embodiments, the protein or the domain fused to the Gene Writing polypeptide the protein or the domain inhibits a host process, e.g., inhibits or sequesters a host DNA repair enzyme that might interfere with Gene Writing.

In some embodiments a template nucleic acid described herein, e.g., a template RNA, is covalently linked or fused with the agent that promotes activity of the gene modifying system, (e.g., a host response modulator or an epigenetic modifier).

In some embodiments, a template RNA molecule for use in the system comprises, from 5′ to 3′ (1) a gRNA spacer; (2) a gRNA scaffold; (3) heterologous object sequence (4) 3′ homology domain. In some embodiments:
(1) Is a Cas9 spacer of ˜18-22 nt, e.g., is 20 nt
(2) Is a gRNA scaffold comprising one or more hairpin loops, e.g., 1, 2, of 3 loops for associating the template with a nickase Cas9 domain. In some embodiments, the gRNA scaffold carries the sequence, from 5′ to 3′,

(SEQ ID NO: 8)
GTTTTAGAGCTAGAAATAGCAAGTTAAAATAAGGCTAGTCCGTTATCAA
CTTGAAAAAGTGGGACCGAGTCGGTCC.

(3) In some embodiments, the heterologous object sequence is, e.g., 7-74, e.g., 10-20, 20-30, 30-40, 40-50, 50-60, 60-70, or 70-80 nt or, 80-90 nt in length. In some embodiments, the first (most 5′) base of the sequence is not C.
(4) In some embodiments, the 3′ homology domain that binds the target priming sequence after nicking occurs is e.g., 3-20 nt, e.g., 7-15 nt, e.g., 12-14 nt. In some embodiments, the 3′ homology domain has 40-60% GC content.

A second gRNA associated with the system may help drive complete integration. In some embodiments, the second gRNA may target a location that is 0-200 nt away from the first-strand nick, e.g., 0-50, 50-100, 100-200 nt away from the first-strand nick. In some embodiments, the second gRNA can only bind its target sequence after the edit is made, e.g., the gRNA binds a sequence present in the heterologous object sequence, but not in the initial target sequence.

In some embodiments, a Gene Writing system described herein is used to make an edit in HEK293, K562, U2OS, or HeLa cells. In some embodiment, a Gene Writing system is used to make an edit in primary cells, e.g., primary cortical neurons from E18.5 mice.

In some embodiments, a reverse transcriptase or RT domain (e.g., as described herein) comprises a MoMLV RT sequence or variant thereof. In embodiments, the MoMLV RT sequence comprises one or more mutations selected from D200N, L603W, T330P, T306K, W313F, D524G, E562Q, D583N, P51L, S67R, E67K, T197A, H204R, E302K, F309N, L435G, N454K, H594Q, D653N, R110S, and K103L. In embodiments, the MoMLV RT sequence comprises a combination of mutations, such as D200N, L603W, and T330P, optionally further including T306K and/or W313F.

In some embodiments, an endonuclease domain (e.g., as described herein) comprises nCAS9, e.g., comprising the H840A mutation.

In some embodiments, the heterologous object sequence (e.g., of a system as described herein) is about 1-50, 50-100, 100-200, 200-300, 300-400, 400-500, 500-600, 600-700, 700-800, 800-900, 900-1000, or more, nucleotides in length.

In some embodiments, the RT and endonuclease domains are joined by a flexible linker, e.g., comprising the amino acid sequence SGGSSGGSSGSETPGTSESATPESSGGSSGGSS (SEQ ID NO: 6).

In some embodiments, the endonuclease domain is N-terminal relative to the RT domain. In some embodiments, the endonuclease domain is C-terminal relative to the RT domain.

In some embodiments, the system incorporates a heterologous object sequence into a target site by TPRT, e.g., as described herein.

### Gene Writers Comprising Localization Sequences

In certain embodiments, a Gene Writer™ gene editor system RNA further comprises an intracellular localization sequence, e.g., a nuclear localization sequence. The nuclear localization sequence may be an RNA sequence that promotes the import of the RNA into the nucleus. In certain embodiments the nuclear localization signal is located on the template RNA. In certain embodiments, the retrotransposase polypeptide is encoded on a first RNA, and the template RNA is a second, separate, RNA, and the nuclear localization signal is located on the template RNA and not on an RNA encoding the retrotransposase polypeptide. While not wishing to be bound by theory, in some embodiments, the RNA encoding the retrotransposase is targeted primarily to the cytoplasm to promote its translation, while the template RNA is targeted primarily to the nucleus to promote its retrotransposition into the genome. In some embodiments the nuclear localization signal is at the 3′ end, 5′ end, or in an internal region of the template RNA. In some embodiments the nuclear localization signal is 3′ of the heterologous sequence (e.g., is directly 3′ of the heterologous sequence) or is 5′ of the heterologous sequence (e.g., is directly 5′ of the heterologous sequence). In some embodiments the nuclear localization signal is placed outside of the 5′ UTR or outside of the 3′ UTR of the template RNA. In some embodiments the nuclear localization signal is placed between the 5′ UTR and the 3′ UTR, wherein optionally the nuclear localization signal is not transcribed with the transgene (e.g., the nuclear localization signal is an anti-sense orientation or is downstream of a transcriptional termination signal or polyadenylation signal). In some embodiments the nuclear localization sequence is situated inside of an intron. In some embodiments a plurality of the same or different nuclear localization signals are in the RNA, e.g., in the template RNA. In some embodiments the nuclear localization signal is less than 5, 10, 25, 50, 75, 100, 150, 200, 250, 300, 350, 400, 450, 500, 600, 700, 800, 900 or 1000 bp in length. Various RNA nuclear localization sequences can be used. For example, Lubelsky and Ulitsky, Nature 555 (107-111), 2018 describe RNA sequences which drive RNA localization into the nucleus. In some embodiments, the nuclear localization signal is a SINE-derived nuclear RNA localization (SIRLOIN) signal. In some embodiments the nuclear localization signal binds a nuclear-enriched protein. In some embodiments the nuclear localization signal binds the HNRNPK protein. In some embodiments the nuclear localization signal is rich in pyrimidines, e.g., is a C/T rich, C/U rich, C rich, T rich, or U rich region. In some embodiments the nuclear localization signal is derived from a long non-coding RNA. In some embodiments the nuclear localization signal is derived from MALAT1 long non-coding RNA or is the 600 nucleotide M region of MALAT1 (described in Miyagawa et al., RNA 18, (738-751), 2012). In some embodiments the nuclear localization signal is derived from BORG long non-coding RNA or is a AGCCC motif (described in Zhang et al., Molecular and Cellular Biology 34, 2318-2329 (2014). In some embodiments the nuclear localization sequence is described in Shukla et al., The EMBO Journal e98452 (2018). In some embodiments the nuclear localization signal is derived from a non-LTR retrotransposon, an LTR retrotransposon, retrovirus, or an endogenous retrovirus.

In some embodiments, a polypeptide described herein comprises one or more (e.g., 2, 3, 4, 5) nuclear targeting sequences, for example a nuclear localization sequence (NLS). In some embodiments, the NLS is a bipartite NLS. In some embodiments, an NLS facilitates the import of a protein comprising an NLS into the cell nucleus. In some embodiments, the NLS is fused to the N-terminus of a Gene Writer described herein. In some embodiments, the NLS is fused to the C-terminus of the Gene Writer. In some embodiments, the NLS is fused to the N-terminus or the C-terminus of a Cas domain. In some embodiments, a linker sequence is disposed between the NLS and the neighboring domain of the Gene Writer.

In some embodiments, an NLS comprises the amino acid sequence MDSLLMNRRKFLYQFKNVRWAKGRRETYLC (SEQ ID NO: 9), PKKRKVEGADKRTADGSEFESPKKKRKV (SEQ ID NO: 10), RKSGKIAAIWKRPRKPKKKRKV (SEQ ID NO: 11) KRTADGSEFESPKKKRKV (SEQ ID NO: 12), KKTELQTTNAENKTKKL (SEQ ID NO: 13), or KRGINDRNFWRGENGRKTR (SEQ ID NO: 14), KRPAATKKAGQAKKKK (SEQ ID NO: 15), or a functional fragment or variant thereof. Exemplary NLS sequences are also described in PCT/EP2000/011690, the contents of which are incorporated herein by reference for their disclosure of exemplary nuclear localization sequences. In some embodiments, an NLS comprises an amino acid sequence as disclosed in Table 2. An NLS of this table may be utilized with one or more copies in a polypeptide in one or more locations in a polypeptide, e.g., 1, 2, 3 or more copies of an NLS in an N-terminal domain, between peptide domains, in a C-terminal domain, or in a combination of locations, in order to improve subcellular localization to the nucleus. Multiple unique sequences may be used within a single polypeptide. Sequences may be naturally monopartite or bipartite, e.g., having one or two stretches of basic amino acids, or may be used as chimeric bipartite sequences. Sequence references correspond to UniProt accession numbers, except where indicated as SeqNLS for sequences mined using a subcellular localization prediction algorithm (Lin et al BMC Bioinformat 13:157 (2012), incorporated herein by reference in its entirety).

TABLE 2
Exemplary nuclear localization signals for use in Gene Writing systems

| Sequence | Sequence References | SEQ ID No. |
|---|---|---|
| AHFKISGEKRPSTDPGKKAK | Q76IQ7 | 223 |
| NPKKKKKKDP | | |
| AHRAKKMSKTHA | P21827 | 224 |
| ASPEYVNLPINGNG | SeqNLS | 225 |
| CTKRPRW | O88622, Q86W56, Q9QYM2, O02776 | 226 |
| DKAKRVSRNKSEKKRR | O15516, Q5RAK8, Q91YB2, Q91YB0, | 227 |
| | Q8QGQ6, O08785, Q9WVS9, Q6YGZ4 | |
| EELRLKEELLKGIYA | Q9QY16, Q9UHL0, Q2TBP1, Q9QY15 | 228 |
| EEQLRRRKNSRLNNTG | G5EFF5 | 229 |
| EVLKVIRTGKRKKKAWKR | SeqNLS | 230 |
| MVTKVC | | |
| HHHHHHHHHHHHQPH | Q63934, G3V7L5, Q12837 | 231 |
| HKKKHPDASVNFSEFSK | P10103, Q4R844, P12682, B0CM99, | 232 |
| | A9RA84, Q6YKA4, P09429, P63159, | |
| | Q08IE6, P63158, Q9YH06, B1MTB0 | |
| HKRTKK | Q2R2D5 | 233 |
| IINGRKLKLKKSRRRSSQTS | SeqNLS | 234 |
| NNSFTSRRS | | |
| KAEQERRK | Q8LH59 | 235 |
| KEKRKRREELFIEQKKRK | SeqNLS | 236 |
| KKGKDEWFSRGKKP | P30999 | 237 |
| KKGPSVQKRKKT | Q6ZN17 | 238 |
| KKKTVINDLLHYKKEK | SeqNLS, P32354 | 239 |
| KKNGGKGKNKPSAKIKK | SeqNLS | 240 |
| KKPKWDDFKKKKK | Q15397, Q8BKS9, Q562C7 | 241 |
| KKRKKD | SeqNLS, Q91Z62, Q1A730, Q969P5, | 242 |
| | Q2KHT6, Q9CPU7 | |
| KKRRKRRRK | SeqNLS | 243 |
| KKRRRRARK | Q9UMS6, D4A702, Q91YE8 | 244 |
| KKSKRGR | Q9UBS0 | 245 |
| KKSRKRGS | B4FG96 | 246 |
| KKSTALSRELGKIMRRR | SeqNLS, P32354 | 247 |
| KKSYQDPEIIAHSRPRK | Q9U7C9 | 248 |
| KKTGKNRKLKSKRVKTR | Q9Z301, O54943, Q8K3T2 | 249 |
| KKVSIAGQSGKLWRWKR | Q6YUL8 | 250 |
| KKYENVVIKRSPRKRGRPR | SeqNLS | 251 |
| K | | |
| KNKKRK | SeqNLS | 252 |
| KPKKKR | SeqNLS | 253 |
| KRAMKDDSHGNSTSPKRRK | Q0E671 | 254 |
| KRANSNLVAAYEKAKKK | P23508 | 255 |
| KRASEDTTSGSPPKKSSAGP | Q9BZZ5, Q5R644 | 256 |
| KR | | |
| KRFKRRWMVRKMKTKK | SeqNLS | 257 |
| KRGLNSSFETSPKKVK | Q8IV63 | 258 |
| KRGNSSIGPNDLSKRKQRK | SeqNLS | 259 |
| K | | |
| KRIHSVSLSQSQIDPSKKVK | SeqNLS | 260 |
| RAK | | |
| KRKGKLKNKGSKRKK | O15381 | 261 |
| KRRRRRRREKRKR | Q96GM8 | 262 |
| KRSNDRTYSPEEEKQRRA | Q91ZF2 | 263 |
| KRTVATNGDASGAHRAKK | SeqNLS | 264 |
| MSK | | |
| KRVYNKGEDEQEHLPKGKK | SeqNLS | 265 |
| R | | |
| KSGKAPRRRAVSMDNSNK | Q9WVH4, O43524 | 266 |
| KVNFLDMSLDDIIIYKELE | Q9P127 | 267 |
| KVQHRIAKKTTRRRR | Q9DXE6 | 268 |
| LSPSLSPL | Q9Y261, P32182, P35583 | 269 |
| MDSLLMNRRKFLYQFKNVR | Q9GZX7 | 270 |
| WAKGRRETYLC | | |
| MPQNEYIELHRKRYGYRLD | SeqNLS | 271 |
| YHEKKRKKESREAHERSKK | | |
| AKKMIGLKAKLYHK | | |
| MVQLRPRASR | SeqNLS | 272 |
| NNKLLAKRRKGGASPKDDP | Q965G5 | 273 |
| MDDIK | | |
| NYKRPMDGTYGPPAKRHEG | O14497, A2BH40 | 274 |
| E | | |
| PDTKRAKLDSSETTMVKKK | SeqNLS | 275 |
| PEKRTKI | SeqNLS | 276 |
| PGGRGKKK | Q719N1, Q9UBP0, A2VDN5 | 277 |
| PGKMDKGEHRQERRDRPY | Q01844, Q61545 | 278 |
| PKKGDKYDKTD | Q45FA5 | 279 |
| PKKKSRK | O35914, Q01954 | 280 |
| PKKNKPE | Q22663 | 281 |
| PKKRAKV | P04295, P89438 | 282 |
| PKPKKLKVE | P55263, P55262, P55264, Q64640 | 283 |
| PKRGRGR | Q9FYS5, Q43386 | 284 |
| PKRRLVDDA | P0C797 | 285 |
| PKRRRTY | SeqNLS | 286 |
| PLFKRR | A8X6H4, Q9TXJ0 | 287 |
| PLRKAKR | Q86WB0, Q5R8V9 | 288 |
| PPAKRKCIF | Q6AZ28, O75928, Q8C5D8 | 289 |
| PPARRRRL | Q8NAG6 | 290 |
| PPKKKRKV | Q3L6L5, P03070, P14999, P03071 | 291 |
| PPNKRMKVKH | Q8BN78 | 292 |
| PPRIYPQLPSAPT | P0C799 | 293 |
| PQRSPFPKSSVKR | SeqNLS | 294 |
| PRPRKVPR | P0C799 | 295 |
| PRRRVQRKR | SeqNLS, Q5R448, Q5TAQ9 | 296 |
| PRRVRLK | Q58DJ0, P56477, Q13568 | 297 |
| PSRKRPR | Q62315, Q5F363, Q92833 | 298 |
| PSSKKRKV | SeqNLS | 299 |
| PTKKRVK | P07664 | 300 |
| QRPGPYDRP | SeqNLS | 301 |
| RGKGGKGLGKGGAKRHRK | SeqNLS | 302 |
| RKAGKGGGGHKTTKKRSA | B4FG96 | 303 |
| KDEKVP | | |
| RKIKLKRAK | A1L3G9 | 304 |
| RKIKRKRAK | B9X187 | 305 |
| RKKEAPGPREELRSRGR | O35126, P54258, Q5IS70, P54259 | 306 |
| RKKRKGK | SeqNLS, Q29243, Q62165, Q28685, | 307 |
| | O18738, Q9TSZ6, Q14118 | |
| RKKRRQRRR | P04326, P69697, P69698, P05907, | 308 |
| | P20879, P04613, P19553, P0C1J9, | |
| | P20893, P12506, P04612, Q73370, | |
| | P0C1K0, P05906, P35965, P04609, | |
| | P04610, P04614, P04608, P05905 | |
| RKKSIPLSIKNLKRKHKRKK | Q9C0C9 | 309 |
| NKITR | | |
| RKLVKPKNTKMKTKLRTNP | Q14190 | 310 |
| Y | | |
| RKRLILSDKGQLDWKK | SeqNLS, Q91Z62, Q1A730, Q2KHT6, | 311 |
| | Q9CPU7 | |
| RKRLKSK | Q13309 | 312 |
| RKRRVRDNM | Q8QPH4, Q809M7, A8C8X1, Q2VNC5, | 313 |
| | Q38SQ0, 089749, Q6DNQ9, Q809L9, | |
| | QOA429, Q20NV3, P16509, P16505, | |
| | Q6DNQ5, P16506, Q6XT06, P26118, | |
| | Q2ICQ2, Q2RCG8, Q0A2D0, Q0A2H9, | |
| | Q9IQ46, Q809M3, Q6J847, Q6J856, | |
| | B4URE4, A4GCM7, Q0A440, P26120, | |
| | P16511, | |
| RKRSPKDKKEKDLDGAGKR | Q7RTP6 | 314 |
| RKT | | |
| RKRTPRVDGQTGENDMNK | O94851 | 315 |
| RRRK | | |
| RLPVRRRRRR | P04499, P12541, P03269, P48313, | 316 |
| | P03270 | |
| RLRFRKPKSK | P69469 | 317 |
| RQQRKR | Q14980 | 318 |
| RRDLNSSFETSPKKVK | Q8K3G5 | 319 |
| RRDRAKLR | Q9SLB8 | 320 |
| RRGDGRRR | Q80WE1, Q5R9B4, Q06787, P35922 | 321 |
| RRGRKRKAEKQ | Q812D1, Q5XXA9, Q99JF8, Q8MJG1, | 322 |
| | Q66T72, 075475 | |
| RRKKRR | Q0VD86, Q58DS6, Q5R6G2, Q9ERI5, | 323 |
| | Q6AYK2, Q6NYC1 | |
| RRKRSKSEDMDSVESKRRR | Q7TT18 | 324 |
| RRKRSR | Q99PU7, D3ZHS6, Q92560, A2VDM8 | 325 |
| RRPKGKTLQKRKPK | Q6ZN17 | 326 |
| RRRGFERFGPDNMGRKRK | Q63014, Q9DBR0 | 327 |
| RRRGKNKVAAQNCRK | SeqNLS | 328 |
| RRRKRR | Q5FVH8, Q6MZT1, Q08DH5, Q8BQP9 | 329 |
| RRRQKQKGGASRRR | SeqNLS | 330 |
| RRRREGPRARRRR | P08313, P10231 | 331 |
| RRTIRLKLVYDKCDRSCKIQ | SeqNLS | 332 |
| KKNRNKCQYCRFHKCLSVG | | |
| MSHNAIRFGRMPRSEKAKL | | |
| KAE | | |
| RRVPQRKEVSRCRKCRK | Q5RJN4, Q32L09, Q8CAK3, Q9NUL5 | 333 |
| RVGGRRQAVECIEDLLNEP | P03255 | 334 |
| GQPLDLSCKRPRP | | |
| RVVKLRIAP | P52639, Q8JMN0 | 335 |
| RWRRR | P70278 | 336 |
| SKRKTKISRKTR | Q5RAY1, O00443 | 337 |
| SYVKTVPNRTRTYIKL | P21935 | 338 |
| TGKNEAKKRKIA | P52739, Q8K3J5, Q5RAU9 | 339 |
| TLSPASSPSSVSCPVIPASTD | SeqNLS | 340 |
| ESPGSALNI | | |
| VSKKQRTGKKIH | P52739, Q8K3J5, Q5RAU9 | 341 |
| SPKKKRKVE | | 342 |
| KRTAD GSEFE SPKKKRKVE | | 343 |
| PAAKRVKLD | | 344 |
| PKKKRKV | | 345 |
| MDSLLMNRRKFLYQFKNVR | | 346 |
| WAKGRRETYLC | | |
| SPKKKRKVEAS | | 347 |
| MAPKKKRKVGIHRGVP | | 348 |

In some embodiments, the NLS is a bipartite NLS. A bipartite NLS typically comprises two basic amino acid clusters separated by a spacer sequence (which may be, e.g., about 10 amino acids in length). A monopartite NLS typically lacks a spacer. An example of a bipartite NLS is the nucleoplasmin NLS, having the sequence KR[PAATKKAGQA]KKKK (SEQ ID NO: 15), wherein the spacer is bracketed. Another exemplary bipartite NLS has the sequence PKKKRKVEGADKRTADGSEFESPKKKRKV (SEQ ID NO: 16). Exemplary NLSs are described in International Application WO2020051561, which is herein incorporated by reference in its entirety, including for its disclosures regarding nuclear localization sequences.

In certain embodiments, a Gene Writer™ gene editor system polypeptide further comprises an intracellular localization sequence, e.g., a nuclear localization sequence and/or a nucleolar localization sequence. The nuclear localization sequence and/or nucleolar localization sequence may be amino acid sequences that promote the import of the protein into the nucleus and/or nucleolus, where it can promote integration of heterologous sequence into the genome. In certain embodiments, a Gene Writer™ gene editor system polypeptide (e.g., a retrotransposase) further comprises a nucleolar localization sequence. In certain embodiments, the retrotransposase polypeptide is encoded on a first RNA, and the template RNA is a second, separate, RNA, and the nucleolar localization signal is encoded on the RNA encoding the retrotransposase polypeptide and not on the template RNA. In some embodiments, the nucleolar localization signal is located at the N-terminus, C-terminus, or in an internal region of the polypeptide. In some embodiments, a plurality of the same or different nucleolar localization signals are used. In some embodiments, the nuclear localization signal is less than 5, 10, 25, 50, 75, or 100 amino acids in length. Various polypeptide nucleolar localization signals can be used. For example, Yang et al., Journal of Biomedical Science 22, 33 (2015), describe a nuclear localization signal that also functions as a nucleolar localization signal. In some embodiments, the nucleolar localization signal may also be a nuclear localization signal. In some embodiments, the nucleolar localization signal may overlap with a nuclear localization signal. In some embodiments, the nucleolar localization signal may comprise a stretch of basic residues. In some embodiments, the nucleolar localization signal may be rich in arginine and lysine residues. In some embodiments, the nucleolar localization signal may be derived from a protein that is enriched in the nucleolus. In some embodiments, the nucleolar localization signal may be derived from a protein enriched at ribosomal RNA loci. In some embodiments, the nucleolar localization signal may be derived from a protein that binds rRNA. In some embodiments, the nucleolar localization signal may be derived from MSP58. In some embodiments, the nucleolar localization signal may be a monopartite motif. In some embodiments, the nucleolar localization signal may be a bipartite motif. In some embodiments, the nucleolar localization signal may consist of a multiple monopartite or bipartite motifs. In some embodiments, the nucleolar localization signal may consist of a mix of monopartite and bipartite motifs. In some embodiments, the nucleolar localization signal may be a dual bipartite motif. In some embodiments, the nucleolar localization motif may be a KRASSQALGTIPKRRSSSRFIKRKK (SEQ ID NO: 17). In some embodiments, the nucleolar localization signal may be derived from nuclear factor-KB-inducing kinase. In some embodiments, the nucleolar localization signal may be an RKKRKKK motif (SEQ ID NO: 18) (described in Birbach et al., Journal of Cell Science, 117 (3615-3624), 2004).

### Gene Writers Comprising Cas Domains

In some embodiments, a GeneWriter described herein comprises a Cas domain. In some embodiments, the Cas domain can direct the GeneWriter to a target site specified by a gRNA, thereby writing in “cis”. In some embodiments, a transposase is fused to a Cas domain. In some embodiments, a Cas domain is used to replace an endogenous domain of a transposase, e.g., to replace an endonuclease domain or DNA-binding domain. In some embodiments, an endonuclease domain comprises a CRISPR/Cas domain (also referred to herein as a CRISPR-associated protein). In some embodiments, a DNA-binding domain comprises a CRISPR/Cas domain. In some embodiments, a CRISPR/Cas domain comprises a protein involved in the clustered regulatory interspaced short palindromic repeat (CRISPR) system, e.g., a Cas protein, and optionally binds a guide RNA, e.g., single guide RNA (sgRNA). Additional description of CRISPR systems can be found, e.g., in the section herein entitled “CRISPR”.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cash, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a DNA-binding domain or endonuclease domain includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a grampositive bacteria or a gram negative bacteria. In certain embodiments, a Cas protein may be from a Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter. In some embodiments, a Gene Writer may comprise a Cas protein as listed in Table 3 A or Table 4, or a functional fragment thereof, or a sequence having at least 70%, 75%, 80%, 85%, 90%, 95%, 96%, 97%, 98%, 99% identity thereto.

TABLE 3A
CRISPR/Cas Proteins, Species, and Mutations

| | | | | SEQ |
|---|---|---|---|---|
| | Parental | | Nickase | ID |
| Variant | Host | Protein Sequence | Mutation | No. |
| Nme2Cas9 | Neisseria | MAAFKPNPINYILGLDIGIASVGWAMVEIDEEENPIRLIDLGVRVFERAEVPKT | N611A | 349 |
| | meningitidis | GDSLAMARRLARSVRRLTRRRAHRLLRARRLLKREGVLQAADFDENGLIKSL | | |
| | | PNTPWQLRAAALDRKLTPLEWSAVLLHLIKHRGYLSQRKNEGETADKELGA | | |
| | | LLKGVANNAHALQTGDFRTPAELALNKFEKESGHIRNQRGDYSHTFSRKDLQ | | |
| | | AELILLFEKQKEFGNPHVSGGLKEGIETLLMTQRPALSGDAVQKMLGHCTFEP | | |
| | | AEPKAAKNTYTAERFIWLTKLNNLRILEQGSERPLTDTERATLMDEPYRKSKL | | |
| | | TYAQARKLLGLEDTAFFKGLRYGKDNAEASTLMEMKAYHAISRALEKEGLK | | |
| | | DKKSPLNLSSELQDEIGTAFSLFKTDEDITGRLKDRVQPEILEALLKHISFDKFV | | |
| | | QISLKALRRIVPLMEQGKRYDEACAEIYGDHYGKKNTEEKIYLPPIPADEIRNP | | |
| | | VVLRALSQARKVINGVVRRYGSPARIHIETAREVGKSFKDRKEIEKRQEENRK | | |
| | | DREKAAAKFREYFPNFVGEPKSKDILKLRLYEQQHGKCLYSGKEINLVRLNE | | |
| | | KGYVEIDHALPFSRTWDDSFNNKVLVLGSENQNKGNQTPYEYFNGKDNSRE | | |
| | | WQEFKARVETSRFPRSKKQRILLQKFDEDGFKECNLNDTRYVNRFLCQFVAD | | |
| | | HILLTGKGKRRVFASNGQITNLLRGFWGLRKVRAENDRHHALDAVVVACST | | |
| | | VAMQQKITRFVRYKEMNAFDGKTIDKETGKVLHQKTHFPQPWEFFAQEVMI | | |
| | | RVFGKPDGKPEFEEADTPEKLRTLLAEKLSSRPEAVHEYVTPLFVSRAPNRKM | | |
| | | SGAHKDTLRSAKRFVKHNEKISVKRVWLTEIKLADLENMVNYKNGREIELYE | | |
| | | ALKARLEAYGGNAKQAFDPKDNPFYKKGGQLVKAVRVEKTQESGVLLNKK | | |
| | | NAYTIADNGDMVRVDVFCKVDKKGKNQYFIVPIYAWQVAENILPDIDCKGY | | |
| | | RIDDSYTFCFSLHKYDLIAFQKDEKSKVEFAYYINCDSSNGRFYLAWHDKGSK | | |
| | | EQQFRISTQNLVLIQKYQVNELGKEIRPCRLKKRPPVR | | |
| PpnCas9 | Pasteurella | MQNNPLNYILGLDLGIASIGWAVVEIDEESSPIRLIDVGVRTFERAEVAKTGES | N605A | 350 |
| | pneumo- | LALSRRLARSSRRLIKRRAERLKKAKRLLKAEKILHSIDEKLPINVWQLRVKG | | |
| | tropica | LKEKLERQEWAAVLLHLSKHRGYLSQRKNEGKSDNKELGALLSGIASNHQM | | |
| | | LQSSEYRTPAEIAVKKFQVEEGHIRNQRGSYTHTFSRLDLLAEMELLFQRQAE | | |
| | | LGNSYTSTTLLENLTALLMWQKPALAGDAILKMLGKCTFEPSEYKAAKNSYS | | |
| | | AERFVWLTKLNNLRILENGTERALNDNERFALLEQPYEKSKLTYAQVRAMLA | | |
| | | LSDNAIFKGVRYLGEDKKTVESKTTLIEMKFYHQIRKTLGSAELKKEWNELK | | |
| | | GNSDLLDEIGTAFSLYKTDDDICRYLEGKLPERVLNALLENLNFDKFIQLSLKA | | |
| | | LHQILPLMLQGQRYDEAVSAIYGDHYGKKSTETTRLLPTIPADEIRNPVVLRTL | | |
| | | TQARKVINAVVRLYGSPARIHIETAREVGKSYQDRKKLEKQQEDNRKQRESA | | |
| | | VKKFKEMFPHFVGEPKGKDILKMRLYELQQAKCLYSGKSLELHRLLEKGYVE | | |
| | | VDHALPFSRTWDDSFNNKVLVLANENQNKGNLTPYEWLDGKNNSERWQHF | | |
| | | VVRVQTSGFSYAKKQRILNHKLDEKGFIERNLNDTRYVARFLCNFIADNMLL | | |
| | | VGKGKRNVFASNGQITALLRHRWGLQKVREQNDRHHALDAVVVACSTVAM | | |
| | | QQKITRFVRYNEGNVFSGERIDRETGEIIPLHFPSPWAFFKENVEIRIFSENPKLE | | |
| | | LENRLPDYPQYNHEWVQPLFVSRMPTRKMTGQGHMETVKSAKRLNEGLSVL | | |
| | | KVPLTQLKLSDLERMVNRDREIALYESLKARLEQFGNDPAKAFAEPFYKKGG | | |
| | | ALVKAVRLEQTQKSGVLVRDGNGVADNASMVRVDVFTKGGKYFLVPIYTW | | |
| | | QVAKGILPNRAATQGKDENDWDIMDEMATFQFSLCQNDLIKLVTKKKTIFGY | | |
| | | FNGLNRATSNINIKEHDLDKSKGKLGIYLEVGVKLAISLEKYQVDELGKNIRP | | |
| | | CRPTKRQHVR | | |
| SauCas9 | Staphy- | MKRNYILGLDIGITSVGYGIIDYETRDVIDAGVRLFKEANVENNEGRRSKRGA | N580A | 351 |
| | lococcus | RRLKRRRRHRIQRVKKLLFDYNLLTDHSELSGINPYEARVKGLSQKLSEEEFS | | |
| | aureus | AALLHLAKRRGVHNVNEVEEDTGNELSTKEQISRNSKALEEKYVAELQLERL | | |
| | | KKDGEVRGSINRFKTSDYVKEAKQLLKVQKAYHQLDQSFIDTYIDLLETRRT | | |
| | | YYEGPGEGSPFGWKDIKEWYEMLMGHCTYFPEELRSVKYAYNADLYNALN | | |
| | | DLNNLVITRDENEKLEYYEKFQIIENVFKQKKKPTLKQIAKEILVNEEDIKGYR | | |
| | | VTSTGKPEFTNLKVYHDIKDITARKEIIENAELLDQIAKILTIYQSSEDIQEELTN | | |
| | | LNSELTQEEIEQISNLKGYTGTHNLSLKAINLILDELWHTNDNQIAIFNRLKLVP | | |
| | | KKVDLSQQKEIPTTLVDDFILSPVVKRSFIQSIKVINAIIKKYGLPNDIIIELARE | | |
| | | KNSKDAQKMINEMQKRNRQTNERIEEIIRTTGKENAKYLIEKIKLHDMQEGKCL | | |
| | | YSLEAIPLEDLLNNPFNYEVDHIIPRSVSFDNSFNNKVLVKQEENSKKGNRTPF | | |
| | | QYLSSSDSKISYETFKKHILNLAKGKGRISKTKKEYLLEERDINRFSVQKDFIN | | |
| | | RNLVDTRYATRGLMNLLRSYFRVNNLDVKVKSINGGFTSFLRRKWKFKKER | | |
| | | NKGYKHHAEDALIIANADFIFKEWKKLDKAKKVMENQMFEEKQAESMPEIE | | |
| | | TEQEYKEIFITPHQIKHIKDFKDYKYSHRVDKKPNRELINDTLYSTRKDDKGN | | |
| | | TLIVNNLNGLYDKDNDKLKKLINKSPEKLLMYHHDPQTYQKLKLIMEQYGD | | |
| | | EKNPLYKYYEETGNYLTKYSKKDNGPVIKKIKYYGNKLNAHLDITDDYPNSR | | |
| | | NKVVKLSLKPYRFDVYLDNGVYKFVTVKNLDVIKKENYYEVNSKCYEEAKK | | |
| | | LKKISNQAEFIASFYNNDLIKINGELYRVIGVNNDLLNRIEVNMIDITYREYLEN | | |
| | | MNDKRPPRIIKTIASKTQSIKKYSTDILGNLYEVKSKKHPQIIKKG | | |
| SauCas9- | Staphy- | MKRNYILGLDIGITSVGYGIIDYETRDVIDAGVRLFKEANVENNEGRRSKRGA | N580A | 352 |
| KKH | lococcus | RRLKRRRRHRIQRVKKLLFDYNLLTDHSELSGINPYEARVKGLSQKLSEEEFS | | |
| | aureus | AALLHLAKRRGVHNVNEVEEDTGNELSTKEQISRNSKALEEKYVAELQLERL | | |
| | | KKDGEVRGSINRFKTSDYVKEAKQLLKVQKAYHQLDQSFIDTYIDLLETRRT | | |
| | | YYEGPGEGSPFGWKDIKEWYEMLMGHCTYFPEELRSVKYAYNADLYNALN | | |
| | | DLNNLVITRDENEKLEYYEKFQIIENVFKQKKKPTLKQIAKEILVNEEDIKGYR | | |
| | | VTSTGKPEFTNLKVYHDIKDITARKEIIENAELLDQIAKILTIYQSSEDIQEELTN | | |
| | | LNSELTQEEIEQISNLKGYTGTHNLSLKAINLILDELWHTNDNQIAIFNRLKLVP | | |
| | | KKVDLSQQKEIPTTLVDDFILSPVVKRSFIQSIKVINAIIKKYGLPNDIIIELARE | | |
| | | KNSKDAQKMINEMQKRNRQTNERIEEIIRTTGKENAKYLIEKIKLHDMQEGKCL | | |
| | | YSLEAIPLEDLLNNPFNYEVDHIIPRSVSFDNSFNNKVLVKQEENSKKGNRTPF | | |
| | | QYLSSSDSKISYETFKKHILNLAKGKGRISKTKKEYLLEERDINRFSVQKDFIN | | |
| | | RNLVDTRYATRGLMNLLRSYFRVNNLDVKVKSINGGFTSFLRRKWKFKKER | | |
| | | NKGYKHHAEDALIIANADFIFKEWKKLDKAKKVMENQMFEEKQAESMPEIE | | |
| | | TEQEYKEIFITPHQIKHIKDFKDYKYSHRVDKKPNRKLINDTLYSTRKDDKGN | | |
| | | TLIVNNLNGLYDKDNDKLKKLINKSPEKLLMYHHDPQTYQKLKLIMEQYGD | | |
| | | EKNPLYKYYEETGNYLTKYSKKDNGPVIKKIKYYGNKLNAHLDITDDYPNSR | | |
| | | NKVVKLSLKPYRFDVYLDNGVYKFVTVKNLDVIKKENYYEVNSKCYEEAKK | | |
| | | LKKISNQAEFIASFYKNDLIKINGELYRVIGVNNDLLNRIEVNMIDITYREYLEN | | |
| | | MNDKRPPHIIKTIASKTQSIKKYSTDILGNLYEVKSKKHPQIIKKG | | |
| SauriCas9 | Staphy- | MQENQQKQNYILGLDIGITSVGYGLIDSKTREVIDAGVRLFPEADSENNSNRR | N588A | 353 |
| | lococcus | SKRGARRLKRRRIHRLNRVKDLLADYQMIDLNNVPKSTDPYTIRVKGLREPL | | |
| | auricularis | TKEEFAIALLHIAKRRGLHNISVSMGDEEQDNELSTKQQLQKNAQQLQDKYV | | |
| | | CELQLERLTNINKVRGEKNRFKTEDFVKEVKQLCETQRQYHNIDDQFIQQYID | | |
| | | LVSTRREYFEGPGNGSPYGWDGDLLKWYEKLMGRCTYFPEELRSVKYAYSA | | |
| | | DLFNALNDLNNLVVTRDDNPKLEYYEKYHIIENVFKQKKNPTLKQIAKEIGV | | |
| | | QDYDIRGYRITKSGKPQFTSFKLYHDLKNIFEQAKYLEDVEMLDEIAKILTIYQ | | |
| | | DEISIKKALDQLPELLTESEKSQIAQLTGYTGTHRLSLKCIHIVIDELWESPENQ | | |
| | | MEIFTRLNLKPKKVEMSEIDSIPTTLVDEFILSPVVKRAFIQSIKVINAVINRFGL | | |
| | | PEDIIIELAREKNSKDRRKFINKLQKQNEATRKKIEQLLAKYGNTNAKYMIEKI | | |
| | | KLHDMQEGKCLYSLEAIPLEDLLSNPTHYEVDHIIPRSVSFDNSLNNKVLVKQ | | |
| | | SENSKKGNRTPYQYLSSNESKISYNQFKQHILNLSKAKDRISKKKRDMLLEER | | |
| | | DINKFEVQKEFINRNLVDTRYATRELSNLLKTYFSTHDYAVKVKTINGGFTNH | | |
| | | LRKVWDFKKHRNHGYKHHAEDALVIANADFLFKTHKALRRTDKILEQPGLE | | |
| | | VNDTTVKVDTEEKYQELFETPKQVKNIKQFRDFKYSHRVDKKPNRQLINDTL | | |
| | | YSTREIDGETYVVQTLKDLYAKDNEKVKKLFTERPQKILMYQHDPKTFEKLM | | |
| | | TILNQYAEAKNPLAAYYEDKGEYVTKYAKKGNGPAIHKIKYIDKKLGSYLDV | | |
| | | SNKYPETQNKLVKLSLKSFRFDIYKCEQGYKMVSIGYLDVLKKDNYYYIPKD | | |
| | | KYEAEKQKKKIKESDLFVGSFYYNDLIMYEDELFRVIGVNSDINNLVELNMV | | |
| | | DITYKDFCEVNNVTGEKRIKKTIGKRVVLIEKYTTDILGNLYKTPLPKKPQLIF | | |
| | | KRGEL | | |
| Sauri | Staphy- | MQENQQKQNYILGLDIGITSVGYGLIDSKTREVIDAGVRLFPEADSENNSNRR | N588A | 354 |
| Cas9- | lococcus | SKRGARRLKRRRIHRLNRVKDLLADYQMIDLNNVPKSTDPYTIRVKGLREPL | | |
| KKH | auricularis | TKEEFAIALLHIAKRRGLHNISVSMGDEEQDNELSTKQQLQKNAQQLQDKYV | | |
| | | CELQLERLTNINKVRGEKNRFKTEDFVKEVKQLCETQRQYHNIDDQFIQQYID | | |
| | | LVSTRREYFEGPGNGSPYGWDGDLLKWYEKLMGRCTYFPEELRSVKYAYSA | | |
| | | DLFNALNDLNNLVVTRDDNPKLEYYEKYHIIENVFKQKKNPTLKQIAKEIGV | | |
| | | QDYDIRGYRITKSGKPQFTSFKLYHDLKNIFEQAKYLEDVEMLDEIAKILTIYQ | | |
| | | DEISIKKALDQLPELLTESEKSQIAQLTGYTGTHRLSLKCIHIVIDELWESPENQ | | |
| | | MEIFTRLNLKPKKVEMSEIDSIPTTLVDEFILSPVVKRAFIQSIKVINAVINRFGL | | |
| | | PEDIIIELAREKNSKDRRKFINKLQKQNEATRKKIEQLLAKYGNTNAKYMIEKI | | |
| | | KLHDMQEGKCLYSLEAIPLEDLLSNPTHYEVDHIIPRSVSFDNSLNNKVLVKQ | | |
| | | SENSKKGNRTPYQYLSSNESKISYNQFKQHILNLSKAKDRISKKKRDMLLEER | | |
| | | DINKFEVQKEFINRNLVDTRYATRELSNLLKTYFSTHDYAVKVKTINGGFTNH | | |
| | | LRKVWDFKKHRNHGYKHHAEDALVIANADFLFKTHKALRRTDKILEQPGLE | | |
| | | VNDTTVKVDTEEKYQELFETPKQVKNIKQFRDFKYSHRVDKKPNRKLINDTL | | |
| | | YSTREIDGETYVVQTLKDLYAKDNEKVKKLFTERPQKILMYQHDPKTFEKLM | | |
| | | TILNQYAEAKNPLAAYYEDKGEYVTKYAKKGNGPAIHKIKYIDKKLGSYLDV | | |
| | | SNKYPETQNKLVKLSLKSFRFDIYKCEQGYKMVSIGYLDVLKKDNYYYIPKD | | |
| | | KYEAEKQKKKIKESDLFVGSFYKNDLIMYEDELFRVIGVNSDINNLVELNMV | | |
| | | DITYKDFCEVNNVTGEKHIKKTIGKRVVLIEKYTTDILGNLYKTPLPKKPQLIF | | |
| | | KRGEL | | |
| ScaCas9- | Strepto- | MEKKYSIGLDIGTNSVGWAVITDDYKVPSKKFKVLGNTNRKSIKKNLMGALL | N872A | 355 |
| Sc+ | coccus | FDSGETAEATRLKRTARRRYTRRKNRIRYLQEIFANEMAKLDDSFFQRLEESF | | |
| | canis | LVEEDKKNERHPIFGNLADEVAYHRNYPTIYHLRKKLADSPEKADLRLIYLAL | | |
| | | AHIIKFRGHFLIEGKLNAENSDVAKLFYQLIQTYNQLFEESPLDEIEVDAKGILS | | |
| | | ARLSKSKRLEKLIAVFPNEKKNGLFGNIIALALGLTPNFKSNFDLTEDAKLQLS | | |
| | | KDTYDDDLDELLGQIGDQYADLFSAAKNLSDAILLSDILRSNSEVTKAPLSAS | | |
| | | MVKRYDEHHQDLALLKTLVRQQFPEKYAEIFKDDTKNGYAGYVGADKKLR | | |
| | | KRSGKLATEEEFYKFIKPILEKMDGAEELLAKLNRDDLLRKQRTFDNGSIPHQI | | |
| | | HLKELHAILRRQEEFYPFLKENREKIEKILTFRIPYYVGPLARGNSRFAWLTRK | | |
| | | SEEAITPWNFEEVVDKGASAQSFIERMTNFDEQLPNKKVLPKHSLLYEYFTVY | | |
| | | NELTKVKYVTERMRKPEFLSGEQKKAIVDLLFKTNRKVTVKQLKEDYFKKIE | | |
| | | CFDSVEIIGVEDRFNASLGTYHDLLKIIKDKDFLDNEENEDILEDIVLTLTLFED | | |
| | | REMIEERLKTYAHLFDDKVMKQLKRRHYTGWGRLSRKMINGIRDKQSGKTIL | | |
| | | DFLKSDGFSNRNFMQLIHDDSLTFKEEIEKAQVSGQGDSLHEQIADLAGSPAIK | | |
| | | KGILQTVKIVDELVKVMGHKPENIVIEMARENQTTTKGLQQSRERKKRIEEGI | | |
| | | KELESQILKENPVENTQLQNEKLYLYYLQNGRDMYVDQELDINRLSDYDVD | | |
| | | HIVPQSFIKDDSIDNKVLTRSVENRGKSDNVPSEEVVKKMKNYWRQLLNAKL | | |
| | | ITQRKFDNLTKAERGGLSEADKAGFIKRQLVETRQITKHVARILDSRMNTKRD | | |
| | | KNDKPIREVKVITLKSKLVSDFRKDFQLYKVRDINNYHHAHDAYLNAVVGTA | | |
| | | LIKKYPKLESEFVYGDYKVYDVRKMIAKSEQEIGKATAKRFFYSNIMNFFKTE | | |
| | | VKLANGEIRKRPLIETNGETGEVVWNKEKDFATVRKVLAMPQVNIVKKTEV | | |
| | | QTGGFSKESILSKRESAKLIPRKKGWDTRKYGGFGSPTVAYSILVVAKVEKGK | | |
| | | AKKLKSVKVLVGITIMEKGSYEKDPIGFLEAKGYKDIKKELIFKLPKYSLFELE | | |
| | | NGRRRMLASAKELQKANELVLPQHLVRLLYYTQNISATTGSNNLGYIEQHRE | | |
| | | EFKEIFEKIIDFSEKYILKNKVNSNLKSSFDEQFAVSDSILLSNSFVSLLKYTSFG | | |
| | | ASGGFTFLDLDVKQGRLRYQTVTEVLDATLIYQSITGLYETRTDLSQLGGD | | |
| SpyCas9 | Strepto- | MDKKYSIGLDIGTNSVGWAVITDEYKVPSKKFKVLGNTDRHSIKKNLIGALLF | N863A | 356 |
| | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | LPKRNSDKLIARKKDWDPKKYGGFDSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SAGELQKGNELALPSKYVNFLYLASHYEKLKGSPEDNEQKQLFVEQHKHYL | | |
| | | DEIIEQISEFSKRVILADANLDKVLSAYNKHRDKPIREQAENIIHLFTLTNLGAP | | |
| | | AAFKYFDTTIDRKRYTSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 357 |
| NG | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | RPKRNSDKLIARKKDWDPKKYGGFVSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SARFLQKGNELALPSKYVNFLYLASHYEKLKGSPEDNEQKQLFVEQHKHYLD | | |
| | | EIIEQISEFSKRVILADANLDKVLSAYNKHRDKPIREQAENIIHLFTLTNLGAPR | | |
| | | AFKYFDTTIDRKVYRSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 358 |
| SpRY | coccus | DSGETAERTRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | RPKRNSDKLIARKKDWDPKKYGGFLWPTVAYSVLVVAKVEKGKSKKLKSV | | |
| | | KELLGITIMERSSFEKNPIDFLEAKGYKEVKKDLIIKLPKYSLFELENGRKRML | | |
| | | ASAKQLQKGNELALPSKYVNFLYLASHYEKLKGSPEDNEQKQLFVEQHKHY | | |
| | | LDEIIEQISEFSKRVILADANLDKVLSAYNKHRDKPIREQAENIIHLFTLTRLGA | | |
| | | PRAFKYFDTTIDPKQYRSTKEVLDATLIHQSITGLYETRIDLSQLGGD | | |
| St1Cas9 | Strepto- | MSDLVLGLDIGIGSVGVGILNKVTGEIIHKNSRIFPAAQAENNLVRRTNRQGR | N622A | 359 |
| | coccus | RLARRKKHRRVRLNRLFEESGLITDFTKISINLNPYQLRVKGLTDELSNEELFI | | |
| | thermophilus | ALKNMVKHRGISYLDDASDDGNSSVGDYAQIVKENSKQLETKTPGQIQLERY | | |
| | | QTYGQLRGDFTVEKDGKKHRLINVFPTSAYRSEALRILQTQQEFNPQITDEFIN | | |
| | | RYLEILTGKRKYYHGPGNEKSRTDYGRYRTSGETLDNIFGILIGKCTFYPDEFR | | |
| | | AAKASYTAQEFNLLNDLNNLTVPTETKKLSKEQKNQIINYVKNEKAMGPAKL | | |
| | | FKYIAKLLSCDVADIKGYRIDKSGKAEIHTFEAYRKMKTLETLDIEQMDRETL | | |
| | | DKLAYVLTLNTEREGIQEALEHEFADGSFSQKQVDELVQFRKANSSIFGKGW | | |
| | | HNFSVKLMMELIPELYETSEEQMTILTRLGKQKTTSSSNKTKYIDEKLLTEEIY | | |
| | | NPVVAKSVRQAIKIVNAAIKEYGDFDNIVIEMARETNEDDEKKAIQKIQKANK | | |
| | | DEKDAAMLKAANQYNGKAELPHSVFHGHKQLATKIRLWHQQGERCLYTGK | | |
| | | TISIHDLINNSNQFEVDHILPLSITFDDSLANKVLVYATANQEKGQRTPYQALD | | |
| | | SMDDAWSFRELKAFVRESKTLSNKKKEYLLTEEDISKFDVRKKFIERNLVDTR | | |
| | | YASRVVLNALQEHFRAHKIDTKVSVVRGQFTSQLRRHWGIEKTRDTYHHHA | | |
| | | VDALIIAASSQLNLWKKQKNTLVSYSEDQLLDIETGELISDDEYKESVFKAPY | | |
| | | QHFVDTLKSKEFEDSILFSYQVDSKFNRKISDATIYATRQAKVGKDKADETYV | | |
| | | LGKIKDIYTQDGYDAFMKIYKKDKSKFLMYRHDPQTFEKVIEPILENYPNKQI | | |
| | | NEKGKEVPCNPFLKYKEEHGYIRKYSKKGNGPEIKSLKYYDSKLGNHIDITPK | | |
| | | DSNNKVVLQSVSPWRADVYFNKTTGKYEILGLKYADLQFEKGTGTYKISQEK | | |
| | | YNDIKKKEGVDSDSEFKFTLYKNDLLLVKDTETKEQQLFRFLSRTMPKQKHY | | |
| | | VELKPYDKQKFEGGEALIKVLGNVANSGQCKKGLGKSNISIYKVRTDVLGNQ | | |
| | | HIIKNEGDKPKLDF | | |
| BlatCas9 | Brevi- | MAYTMGIDVGIASCGWAIVDLERQRIIDIGVRTFEKAENPKNGEALAVPRREA | N607A | 360 |
| | bacillus | RSSRRRLRRKKHRIERLKHMFVRNGLAVDIQHLEQTLRSQNEIDVWQLRVDG | | |
| | laterosporus | LDRMLTQKEWLRVLIHLAQRRGFQSNRKTDGSSEDGQVLVNVTENDRLMEE | | |
| | | KDYRTVAEMMVKDEKFSDHKRNKNGNYHGVVSRSSLLVEIHTLFETQRQHH | | |
| | | NSLASKDFELEYVNIWSAQRPVATKDQIEKMIGTCTFLPKEKRAPKASWHFQ | | |
| | | YFMLLQTINHIRITNVQGTRSLNKEEIEQVVNMALTKSKVSYHDTRKILDLSEE | | |
| | | YQFVGLDYGKEDEKKKVESKETIIKLDDYHKLNKIFNEVELAKGETWEADDY | | |
| | | DTVAYALTFFKDDEDIRDYLQNKYKDSKNRLVKNLANKEYTNELIGKVSTLS | | |
| | | FRKVGHLSLKALRKIIPFLEQGMTYDKACQAAGFDFQGISKKKRSVVLPVIDQ | | |
| | | ISNPVVNRALTQTRKVINALIKKYGSPETIHIETARELSKTFDERKNITKDYKEN | | |
| | | RDKNEHAKKHLSELGIINPTGLDIVKYKLWCEQQGRCMYSNQPISFERLKESG | | |
| | | YTEVDHIIPYSRSMNDSYNNRVLVMTRENREKGNQTPFEYMGNDTQRWYEF | | |
| | | EQRVTTNPQIKKEKRQNLLLKGFTNRRELEMLERNLNDTRYITKYLSHFISTN | | |
| | | LEFSPSDKKKKVVNTSGRITSHLRSRWGLEKNRGQNDLHHAMDAIVIAVTSD | | |
| | | SFIQQVTNYYKRKERRELNGDDKFPLPWKFFREEVIARLSPNPKEQIEALPNHF | | |
| | | YSEDELADLQPIFVSRMPKRSITGEAHQAQFRRVVGKTKEGKNITAKKTALV | | |
| | | DISYDKNGDFNMYGRETDPATYEAIKERYLEFGGNVKKAFSTDLHKPKKDGT | | |
| | | KGPLIKSVRIMENKTLVHPVNKGKGVVYNSSIVRTDVFQRKEKYYLLPVYVT | | |
| | | DVTKGKLPNKVIVAKKGYHDWIEVDDSFTFLFSLYPNDLIFIRQNPKKKISLK | | |
| | | KRIESHSISDSKEVQEIHAYYKGVDSSTAAIEFIIHDGSYYAKGVGVQNLDCFE | | |
| | | KYQVDILGNYFKVKGEKRLELETSDSNHKGKDVNSIKSTSR | | |
| cCas9- | Staphy- | MKRNYILGLDIGITSVGYGIIDYETRDVIDAGVRLFKEANVENNEGRRSKRGA | N580A | 361 |
| v16 | lococcus | RRLKRRRRHRIQRVKKLLFDYNLLTDHSELSGINPYEARVKGLSQKLSEEEFS | | |
| | aureus | AALLHLAKRRGVHNVNEVEEDTGNELSTKEQISRNSKALEEKYVAELQLERL | | |
| | | KKDGEVRGSINRFKTSDYVKEAKQLLKVQKAYHQLDQSFIDTYIDLLETRRT | | |
| | | YYEGPGEGSPFGWKDIKEWYEMLMGHCTYFPEELRSVKYAYNADLYNALN | | |
| | | DLNNLVITRDENEKLEYYEKFQIIENVFKQKKKPTLKQIAKEILVNEEDIKGYR | | |
| | | VTSTGKPEFTNLKVYHDIKDITARKEIIENAELLDQIAKILTIYQSSEDIQEELTN | | |
| | | LNSELTQEEIEQISNLKGYTGTHNLSLKAINLILDELWHTNDNQIAIFNRLKLVP | | |
| | | KKVDLSQQKEIPTTLVDDFILSPVVKRSFIQSIKVINAIIKKYGLPNDIIIELARE | | |
| | | KNSKDAQKMINEMQKRNRQTNERIEEIIRTTGKENAKYLIEKIKLHDMQEGKCL | | |
| | | YSLEAIPLEDLLNNPFNYEVDHIIPRSVSFDNSFNNKVLVKQEENSKKGNRTPF | | |
| | | QYLSSSDSKISYETFKKHILNLAKGKGRISKTKKEYLLEERDINRFSVQKDFIN | | |
| | | RNLVDTRYATRGLMNLLRSYFRVNNLDVKVKSINGGFTSFLRRKWKFKKER | | |
| | | NKGYKHHAEDALIIANADFIFKEWKKLDKAKKVMENQMFEEKQAESMPEIE | | |
| | | TEQEYKEIFITPHQIKHIKDFKDYKYSHRVDKKPNRKLINDTLYSTRKDDKGN | | |
| | | TLIVNNLNGLYDKDNDKLKKLINKSPEKLLMYHHDPQTYQKLKLIMEQYGD | | |
| | | EKNPLYKYYEETGNYLTKYSKKDNGPVIKKIKYYGNKLNAHLDITDDYPNSR | | |
| | | NKVVKLSLKPYRFDVYLDNGVYKFVTVKNLDVIKKENYYEVNSKCYEEAKK | | |
| | | LKKISNQAEFIASFYKNDLIKINGELYRVIGVNSDKNNLIEVNMIDITYREYLEN | | |
| | | MNDKRPPHIIKTIASKTQSIKKYSTDILGNLYEVKSKKHPQIIKKG | | |
| cCas9- | Staphy- | MKRNYILGLDIGITSVGYGIIDYETRDVIDAGVRLFKEANVENNEGRRSKRGA | N580A | 362 |
| V17 | lococcus | RRLKRRRRHRIQRVKKLLFDYNLLTDHSELSGINPYEARVKGLSQKLSEEEFS | | |
| | aureus | AALLHLAKRRGVHNVNEVEEDTGNELSTKEQISRNSKALEEKYVAELQLERL | | |
| | | KKDGEVRGSINRFKTSDYVKEAKQLLKVQKAYHQLDQSFIDTYIDLLETRRT | | |
| | | YYEGPGEGSPFGWKDIKEWYEMLMGHCTYFPEELRSVKYAYNADLYNALN | | |
| | | DLNNLVITRDENEKLEYYEKFQIIENVFKQKKKPTLKQIAKEILVNEEDIKGYR | | |
| | | VTSTGKPEFTNLKVYHDIKDITARKEIIENAELLDQIAKILTIYQSSEDIQEELTN | | |
| | | LNSELTQEEIEQISNLKGYTGTHNLSLKAINLILDELWHTNDNQIAIFNRLKLVP | | |
| | | KKVDLSQQKEIPTTLVDDFILSPVVKRSFIQSIKVINAIIKKYGLPNDIIIELARE | | |
| | | KNSKDAQKMINEMQKRNRQTNERIEEIIRTTGKENAKYLIEKIKLHDMQEGKCL | | |
| | | YSLEAIPLEDLLNNPFNYEVDHIIPRSVSFDNSFNNKVLVKQEENSKKGNRTPF | | |
| | | QYLSSSDSKISYETFKKHILNLAKGKGRISKTKKEYLLEERDINRFSVQKDFIN | | |
| | | RNLVDTRYATRGLMNLLRSYFRVNNLDVKVKSINGGFTSFLRRKWKFKKER | | |
| | | NKGYKHHAEDALIIANADFIFKEWKKLDKAKKVMENQMFEEKQAESMPEIE | | |
| | | TEQEYKEIFITPHQIKHIKDFKDYKYSHRVDKKPNRKLINDTLYSTRKDDKGN | | |
| | | TLIVNNLNGLYDKDNDKLKKLINKSPEKLLMYHHDPQTYQKLKLIMEQYGD | | |
| | | EKNPLYKYYEETGNYLTKYSKKDNGPVIKKIKYYGNKLNAHLDITDDYPNSR | | |
| | | NKVVKLSLKPYRFDVYLDNGVYKFVTVKNLDVIKKENYYEVNSKCYEEAKK | | |
| | | LKKISNQAEFIASFYKNDLIKINGELYRVIGVNNSTRNIVELNMIDITYREYLEN | | |
| | | MNDKRPPHIIKTIASKTQSIKKYSTDILGNLYEVKSKKHPQIIKKG | | |
| cCas9- | Staphy- | MKRNYILGLDIGITSVGYGIIDYETRDVIDAGVRLFKEANVENNEGRRSKRGA | N580A | 363 |
| v21 | lococcus | RRLKRRRRHRIQRVKKLLFDYNLLTDHSELSGINPYEARVKGLSQKLSEEEFS | | |
| | aureus | AALLHLAKRRGVHNVNEVEEDTGNELSTKEQISRNSKALEEKYVAELQLERL | | |
| | | KKDGEVRGSINRFKTSDYVKEAKQLLKVQKAYHQLDQSFIDTYIDLLETRRT | | |
| | | YYEGPGEGSPFGWKDIKEWYEMLMGHCTYFPEELRSVKYAYNADLYNALN | | |
| | | DLNNLVITRDENEKLEYYEKFQIIENVFKQKKKPTLKQIAKEILVNEEDIKGYR | | |
| | | VTSTGKPEFTNLKVYHDIKDITARKEIIENAELLDQIAKILTIYQSSEDIQEELTN | | |
| | | LNSELTQEEIEQISNLKGYTGTHNLSLKAINLILDELWHTNDNQIAIFNRLKLVP | | |
| | | KKVDLSQQKEIPTTLVDDFILSPVVKRSFIQSIKVINAIIKKYGLPNDIIIELARE | | |
| | | KNSKDAQKMINEMQKRNRQTNERIEEIIRTTGKENAKYLIEKIKLHDMQEGKCL | | |
| | | YSLEAIPLEDLLNNPFNYEVDHIIPRSVSFDNSFNNKVLVKQEENSKKGNRTPF | | |
| | | QYLSSSDSKISYETFKKHILNLAKGKGRISKTKKEYLLEERDINRFSVQKDFIN | | |
| | | RNLVDTRYATRGLMNLLRSYFRVNNLDVKVKSINGGFTSFLRRKWKFKKER | | |
| | | NKGYKHHAEDALIIANADFIFKEWKKLDKAKKVMENQMFEEKQAESMPEIE | | |
| | | TEQEYKEIFITPHQIKHIKDFKDYKYSHRVDKKPNRKLINDTLYSTRKDDKGN | | |
| | | TLIVNNLNGLYDKDNDKLKKLINKSPEKLLMYHHDPQTYQKLKLIMEQYGD | | |
| | | EKNPLYKYYEETGNYLTKYSKKDNGPVIKKIKYYGNKLNAHLDITDDYPNSR | | |
| | | NKVVKLSLKPYRFDVYLDNGVYKFVTVKNLDVIKKENYYEVNSKCYEEAKK | | |
| | | LKKISNQAEFIASFYKNDLIKINGELYRVIGVNSDDRNIIELNMIDITYREYLEN | | |
| | | MNDKRPPHIIKTIASKTQSIKKYSTDILGNLYEVKSKKHPQIIKKG | | |
| cCas9- | Staphy- | MKRNYILGLDIGITSVGYGIIDYETRDVIDAGVRLFKEANVENNEGRRSKRGA | N580A | 364 |
| v42 | lococcus | RRLKRRRRHRIQRVKKLLFDYNLLTDHSELSGINPYEARVKGLSQKLSEEEFS | | |
| | aureus | AALLHLAKRRGVHNVNEVEEDTGNELSTKEQISRNSKALEEKYVAELQLERL | | |
| | | KKDGEVRGSINRFKTSDYVKEAKQLLKVQKAYHQLDQSFIDTYIDLLETRRT | | |
| | | YYEGPGEGSPFGWKDIKEWYEMLMGHCTYFPEELRSVKYAYNADLYNALN | | |
| | | DLNNLVITRDENEKLEYYEKFQIIENVFKQKKKPTLKQIAKEILVNEEDIKGYR | | |
| | | VTSTGKPEFTNLKVYHDIKDITARKEIIENAELLDQIAKILTIYQSSEDIQEELTN | | |
| | | LNSELTQEEIEQISNLKGYTGTHNLSLKAINLILDELWHTNDNQIAIFNRLKLVP | | |
| | | KKVDLSQQKEIPTTLVDDFILSPVVKRSFIQSIKVINAIIKKYGLPNDIIIELARE | | |
| | | KNSKDAQKMINEMQKRNRQTNERIEEIIRTTGKENAKYLIEKIKLHDMQEGKCL | | |
| | | YSLEAIPLEDLLNNPFNYEVDHIIPRSVSFDNSFNNKVLVKQEENSKKGNRTPF | | |
| | | QYLSSSDSKISYETFKKHILNLAKGKGRISKTKKEYLLEERDINRFSVQKDFIN | | |
| | | RNLVDTRYATRGLMNLLRSYFRVNNLDVKVKSINGGFTSFLRRKWKFKKER | | |
| | | NKGYKHHAEDALIIANADFIFKEWKKLDKAKKVMENQMFEEKQAESMPEIE | | |
| | | TEQEYKEIFITPHQIKHIKDFKDYKYSHRVDKKPNRKLINDTLYSTRKDDKGN | | |
| | | TLIVNNLNGLYDKDNDKLKKLINKSPEKLLMYHHDPQTYQKLKLIMEQYGD | | |
| | | EKNPLYKYYEETGNYLTKYSKKDNGPVIKKIKYYGNKLNAHLDITDDYPNSR | | |
| | | NKVVKLSLKPYRFDVYLDNGVYKFVTVKNLDVIKKENYYEVNSKCYEEAKK | | |
| | | LKKISNQAEFIASFYKNDLIKINGELYRVIGVNNNRLNKIELNMIDITYREYLEN | | |
| | | MNDKRPPHIIKTIASKTQSIKKYSTDILGNLYEVKSKKHPQIIKKG | | |
| CdiCas9 | Coryne- | MKYHVGIDVGTFSVGLAAIEVDDAGMPIKTLSLVSHIHDSGLDPDEIKSAVTR | H573A | 365 |
| | bacterium | LASSGIARRTRRLYRRKRRRLQQLDKFIQRQGWPVIELEDYSDPLYPWKVRA | (Alter- | |
| | diphtheriae | ELAASYIADEKERGEKLSVALRHIARHRGWRNPYAKVSSLYLPDGPSDAFKAI | nate) | |
| | | REEIKRASGQPVPETATVGQMVTLCELGTLKLRGEGGVLSARLQQSDYAREI | | |
| | | QEICRMQEIGQELYRKIIDVVFAAESPKGSASSRVGKDPLQPGKNRALKASDA | | |
| | | FQRYRIAALIGNLRVRVDGEKRILSVEEKNLVFDHLVNLTPKKEPEWVTIAEIL | | |
| | | GIDRGQLIGTATMTDDGERAGARPPTHDTNRSIVNSRIAPLVDWWKTASALE | | |
| | | QHAMVKALSNAEVDDFDSPEGAKVQAFFADLDDDVHAKLDSLHLPVGRAA | | |
| | | YSEDTLVRLTRRMLSDGVDLYTARLQEFGIEPSWTPPTPRIGEPVGNPAVDRV | | |
| | | LKTVSRWLESATKTWGAPERVIIEHVREGFVTEKRAREMDGDMRRRAARNA | | |
| | | KLFQEMQEKLNVQGKPSRADLWRYQSVQRQNCQCAYCGSPITFSNSEMDHI | | |
| | | VPRAGQGSTNTRENLVAVCHRCNQSKGNTPFAIWAKNTSIEGVSVKEAVERT | | |
| | | RHWVTDTGMRSTDFKKFTKAVVERFQRATMDEEIDARSMESVAWMANELR | | |
| | | SRVAQHFASHGTTVRVYRGSLTAEARRASGISGKLKFFDGVGKSRLDRRHHA | | |
| | | IDAAVIAFTSDYVAETLAVRSNLKQSQAHRQEAPQWREFTGKDAEHRAAWR | | |
| | | VWCQKMEKLSALLTEDLRDDRVVVMSNVRLRLGNGSAHKETIGKLSKVKLS | | |
| | | SQLSVSDIDKASSEALWCALTREPGFDPKEGLPANPERHIRVNGTHVYAGDNI | | |
| | | GLFPVSAGSIALRGGYAELGSSFHHARVYKITSGKKPAFAMLRVYTIDLLPYR | | |
| | | NQDLFSVELKPQTMSMRQAEKKLRDALATGNAEYLGWLVVDDELVVDTSKI | | |
| | | ATDQVKAVEAELGTIRRWRVDGFFSPSKLRLRPLQMSKEGIKKESAPELSKIID | | |
| | | RPGWLPAVNKLFSDGNVTVVRRDSLGRVRLESTAHLPVTWKVQ | | |
| CjeCas9 | Campy- | MARILAFDIGISSIGWAFSENDELKDCGVRIFTKVENPKTGESLALPRRLARSA | N582A | 366 |
| | lobacter | RKRLARRKARLNHLKHLIANEFKLNYEDYQSFDESLAKAYKGSLISPYELRFR | | |
| | jejuni | ALNELLSKQDFARVILHIAKRRGYDDIKNSDDKEKGAILKAIKQNEEKLANYQ | | |
| | | SVGEYLYKEYFQKFKENSKEFTNVRNKKESYERCIAQSFLKDELKLIFKKQRE | | |
| | | FGFSFSKKFEEEVLSVAFYKRALKDFSHLVGNCSFFTDEKRAPKNSPLAFMFV | | |
| | | ALTRIINLLNNLKNTEGILYTKDDLNALLNEVLKNGTLTYKQTKKLLGLSDDY | | |
| | | EFKGEKGTYFIEFKKYKEFIKALGEHNLSQDDLNEIAKDITLIKDEIKLKKALA | | |
| | | KYDLNQNQIDSLSKLEFKDHLNISFKALKLVTPLMLEGKKYDEACNELNLKV | | |
| | | AINEDKKDFLPAFNETYYKDEVTNPVVLRAIKEYRKVLNALLKKYGKVHKIN | | |
| | | IELAREVGKNHSQRAKIEKEQNENYKAKKDAELECEKLGLKINSKNILKLRLF | | |
| | | KEQKEFCAYSGEKIKISDLQDEKMLEIDHIYPYSRSFDDSYMNKVLVFTKQNQ | | |
| | | EKLNQTPFEAFGNDSAKWQKIEVLAKNLPTKKQKRILDKNYKDKEQKNFKD | | |
| | | RNLNDTRYIARLVLNYTKDYLDFLPLSDDENTKLNDTQKGSKVHVEAKSGM | | |
| | | LTSALRHTWGFSAKDRNNHLHHAIDAVIIAYANNSIVKAFSDFKKEQESNSAE | | |
| | | LYAKKISELDYKNKRKFFEPFSGFRQKVLDKIDEIFVSKPERKKPSGALHEETF | | |
| | | RKEEEFYQSYGGKEGVLKALELGKIRKVNGKIVKNGDMFRVDIFKHKKTNKF | | |
| | | YAVPIYTMDFALKVLPNKAVARSKKGEIKDWILMDENYEFCFSLYKDSLILIQ | | |
| | | TKDMQEPEFVYYNAFTSSTVSLIVSKHDNKFETLSKNQKILFKNANEKEVIAK | | |
| | | SIGIQNLKVFEKYIVSALGEVTKAEFRQREDFKK | | |
| GeoCas9 | Geobacillus | MRYKIGLDIGITSVGWAVMNLDIPRIEDLGVRIFDRAENPQTGESLALPRRLAR | N605A | 367 |
| | stearo- | SARRRLRRRKHRLERIRRLVIREGILTKEELDKLFEEKHEIDVWQLRVEALDR | | |
| | thermophilus | KLNNDELARVLLHLAKRRGFKSNRKSERSNKENSTMLKHIEENRAILSSYRTV | | |
| | | GEMIVKDPKFALHKRNKGENYTNTIARDDLEREIRLIFSKQREFGNMSCTEEF | | |
| | | ENEYITIWASQRPVASKDDIEKKVGFCTFEPKEKRAPKATYTFQSFIAWEHINK | | |
| | | LRLISPSGARGLTDEERRLLYEQAFQKNKITYHDIRTLLHLPDDTYFKGIVYDR | | |
| | | GESRKQNENIRFLELDAYHQIRKAVDKVYGKGKSSSFLPIDFDTFGYALTLFK | | |
| | | DDADIHSYLRNEYEQNGKRMPNLANKVYDNELIEELLNLSFTKFGHLSLKAL | | |
| | | RSILPYMEQGEVYSSACERAGYTFTGPKKKQKTMLLPNIPPIANPVVMRALTQ | | |
| | | ARKVVNAIIKKYGSPVSIHIELARDLSQTFDERRKTKKEQDENRKKNETAIRQ | | |
| | | LMEYGLTLNPTGHDIVKFKLWSEQNGRCAYSLQPIEIERLLEPGYVEVDHVIP | | |
| | | YSRSLDDSYTNKVLVLTRENREKGNRIPAEYLGVGTERWQQFETFVLTNKQF | | |
| | | SKKKRDRLLRLHYDENEETEFKNRNLNDTRYISRFFANFIREHLKFAESDDKQ | | |
| | | KVYTVNGRVTAHLRSRWEFNKNREESDLHHAVDAVIVACTTPSDIAKVTAFY | | |
| | | QRREQNKELAKKTEPHFPQPWPHFADELRARLSKHPKESIKALNLGNYDDQK | | |
| | | LESLQPVFVSRMPKRSVTGAAHQETLRRYVGIDERSGKIQTVVKTKLSEIKLD | | |
| | | ASGHFPMYGKESDPRTYEAIRQRLLEHNNDPKKAFQEPLYKPKKNGEPGPVIR | | |
| | | TVKIIDTKNQVIPLNDGKTVAYNSNIVRVDVFEKDGKYYCVPVYTMDIMKGI | | |
| | | LPNKAIEPNKPYSEWKEMTEDYTFRFSLYPNDLIRIELPREKTVKTAAGEEINV | | |
| | | KDVFVYYKTIDSANGGLELISHDHRFSLRGVGSRTLKRFEKYQVDVLGNIYK | | |
| | | VRGEKRVGLASSAHSKPGKTIRPLQSTRD | | |
| iSpyMac | Streptoccus | MDKKYSIGLDIGTNSVGWAVITDEYKVPSKKFKVLGNTDRHSIKKNLIGALLF | N863A | 368 |
| Cas9 | spp. | DSGETAEATRLKRTARRRYTRRKNRICYLQEIFSNEMAKVDDSFFHRLEESFL | | |
| | | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRKLENLIAQLPGEKKNGLFGNLIALSLGLTPNFKSNFDLAEDAKLQL | | |
| | | SKDTYDDDLDNLLAQIGDQYADLFLAAKNLSDAILLSDILRVNTEITKAPLSA | | |
| | | SMIKRYDEHHQDLTLLKALVRQQLPEKYKEIFFDQSKNGYAGYIDGGASQEE | | |
| | | FYKFIKPILEKMDGTEELLVKLKREDLLRKQRTFDNGSIPHQIHLGELHAILRR | | |
| | | QEDFYPFLKDNREKIEKILTFRIPYYVGPLARGNSRFAWMTRKSEETITPWNFE | | |
| | | EVVDKGASAQSFIERMTNFDKNLPNEKVLPKHSLLYEYFTVYNELTKVKYVT | | |
| | | EGMRKPAFLSGEQKKAIVDLLFKTNRKVTVKQLKEDYFKKIECFDSVEISGVE | | |
| | | DRFNASLGTYHDLLKIIKDKDFLDNEENEDILEDIVLTLTLFEDREMIEERLKT | | |
| | | YAHLFDDKVMKQLKRRRYTGWGRLSRKLINGIRDKQSGKTILDFLKSDGFAN | | |
| | | RNFMQLIHDDSLTFKEDIQKAQVSGQGDSLHEHIANLAGSPAIKKGILQTVKV | | |
| | | VDELVKVMGRHKPENIVIEMARENQTTQKGQKNSRERMKRIEEGIKELGSQIL | | |
| | | KEHPVENTQLQNEKLYLYYLQNGRDMYVDQELDINRLSDYDVDHIVPQSFL | | |
| | | KDDSIDNKVLTRSDKNRGKSDNVPSEEVVKKMKNYWRQLLNAKLITQRKFD | | |
| | | NLTKAERGGLSELDKAGFIKRQLVETRQITKHVAQILDSRMNTKYDENDKLIR | | |
| | | EVKVITLKSKLVSDFRKDFQFYKVREINNYHHAHDAYLNAVVGTALIKKYPK | | |
| | | LESEFVYGDYKVYDVRKMIAKSEQEIGKATAKYFFYSNIMNFFKTEITLANGE | | |
| | | IRKRPLIETNGETGEIVWDKGRDFATVRKVLSMPQVNIVKKTEIQTVGQNGGL | | |
| | | FDDNPKSPLEVTPSKLVPLKKELNPKKYGGYQKPTTAYPVLLITDTKQLIPISV | | |
| | | MNKKQFEQNPVKFLRDRGYQQVGKNDFIKLPKYTLVDIGDGIKRLWASSKEI | | |
| | | HKGNQLVVSKKSQILLYHAHHLDSDLSNDYLQNHNQQFDVLFNEIISFSKKC | | |
| | | KLGKEHIQKIENVYSNKKNSASIEELAESFIKLLGFTQLGATSPFNFLGVKLNQ | | |
| | | KQYKGKKDYILPCTEGTLIRQSITGLYETRVDLSKIGEDSGGSGGSKRTADGSE | | |
| | | FES | | |
| NmeCas9 | Neisseria | MAAFKPNSINYILGLDIGIASVGWAMVEIDEEENPIRLIDLGVRVFERAEVPKT | N611A | 369 |
| | meningitidis | GDSLAMARRLARSVRRLTRRRAHRLLRTRRLLKREGVLQAANFDENGLIKSL | | |
| | | PNTPWQLRAAALDRKLTPLEWSAVLLHLIKHRGYLSQRKNEGETADKELGA | | |
| | | LLKGVAGNAHALQTGDFRTPAELALNKFEKESGHIRNQRSDYSHTFSRKDLQ | | |
| | | AELILLFEKQKEFGNPHVSGGLKEGIETLLMTQRPALSGDAVQKMLGHCTFEP | | |
| | | AEPKAAKNTYTAERFIWLTKLNNLRILEQGSERPLTDTERATLMDEPYRKSKL | | |
| | | TYAQARKLLGLEDTAFFKGLRYGKDNAEASTLMEMKAYHAISRALEKEGLK | | |
| | | DKKSPLNLSPELQDEIGTAFSLFKTDEDITGRLKDRIQPEILEALLKHISFDKFV | | |
| | | QISLKALRRIVPLMEQGKRYDEACAEIYGDHYGKKNTEEKIYLPPIPADEIRNP | | |
| | | VVLRALSQARKVINGVVRRYGSPARIHIETAREVGKSFKDRKEIEKRQEENRK | | |
| | | DREKAAAKFREYFPNFVGEPKSKDILKLRLYEQQHGKCLYSGKEINLGRLNE | | |
| | | KGYVEIDHALPFSRTWDDSFNNKVLVLGSENQNKGNQTPYEYFNGKDNSRE | | |
| | | WQEFKARVETSRFPRSKKQRILLQKFDEDGFKERNLNDTRYVNRFLCQFVAD | | |
| | | RMRLTGKGKKRVFASNGQITNLLRGFWGLRKVRAENDRHHALDAVVVACS | | |
| | | TVAMQQKITRFVRYKEMNAFDGKTIDKETGEVLHQKTHFPQPWEFFAQEVMI | | |
| | | RVFGKPDGKPEFEEADTLEKLRTLLAEKLSSRPEAVHEYVTPLFVSRAPNRKM | | |
| | | SGQGHMETVKSAKRLDEGVSVLRVPLTQLKLKDLEKMVNREREPKLYEALK | | |
| | | ARLEAHKDDPAKAFAEPFYKYDKAGNRTQQVKAVRVEQVQKTGVWVRNH | | |
| | | NGIADNATMVRVDVFEKGDKYYLVPIYSWQVAKGILPDRAVVQGKDEEDW | | |
| | | QLIDDSFNFKFSLHPNDLVEVITKKARMFGYFASCHRGTGNINIRIHDLDHKIG | | |
| | | KNGILEGIGVKTALSFQKYQIDELGKEIRPCRLKKRPPVR | | |
| ScaCas9 | Strepto- | MEKKYSIGLDIGTNSVGWAVITDDYKVPSKKFKVLGNTNRKSIKKNLMGALL | N872A | 370 |
| | coccus | FDSGETAEATRLKRTARRRYTRRKNRIRYLQEIFANEMAKLDDSFFQRLEESF | | |
| | canis | LVEEDKKNERHPIFGNLADEVAYHRNYPTIYHLRKKLADSPEKADLRLIYLAL | | |
| | | AHIIKFRGHFLIEGKLNAENSDVAKLFYQLIQTYNQLFEESPLDEIEVDAKGILS | | |
| | | ARLSKSKRLEKLIAVFPNEKKNGLFGNIIALALGLTPNFKSNFDLTEDAKLQLS | | |
| | | KDTYDDDLDELLGQIGDQYADLFSAAKNLSDAILLSDILRSNSEVTKAPLSAS | | |
| | | MVKRYDEHHQDLALLKTLVRQQFPEKYAEIFKDDTKNGYAGYVGIGIKHRK | | |
| | | RTTKLATQEEFYKFIKPILEKMDGAEELLAKLNRDDLLRKQRTFDNGSIPHQIH | | |
| | | LKELHAILRRQEEFYPFLKENREKIEKILTFRIPYYVGPLARGNSRFAWLTRKS | | |
| | | EEAITPWNFEEVVDKGASAQSFIERMTNFDEQLPNKKVLPKHSLLYEYFTVYN | | |
| | | ELTKVKYVTERMRKPEFLSGEQKKAIVDLLFKTNRKVTVKQLKEDYFKKIEC | | |
| | | FDSVEIIGVEDRFNASLGTYHDLLKIIKDKDFLDNEENEDILEDIVLTLTLFEDR | | |
| | | EMIEERLKTYAHLFDDKVMKQLKRRHYTGWGRLSRKMINGIRDKQSGKTIL | | |
| | | DFLKSDGFSNRNFMQLIHDDSLTFKEEIEKAQVSGQGDSLHEQIADLAGSPAIK | | |
| | | KGILQTVKIVDELVKVMGHKPENIVIEMARENQTTTKGLQQSRERKKRIEEGI | | |
| | | KELESQILKENPVENTQLQNEKLYLYYLQNGRDMYVDQELDINRLSDYDVD | | |
| | | HIVPQSFIKDDSIDNKVLTRSVENRGKSDNVPSEEVVKKMKNYWRQLLNAKL | | |
| | | ITQRKFDNLTKAERGGLSEADKAGFIKRQLVETRQITKHVARILDSRMNTKRD | | |
| | | KNDKPIREVKVITLKSKLVSDFRKDFQLYKVRDINNYHHAHDAYLNAVVGTA | | |
| | | LIKKYPKLESEFVYGDYKVYDVRKMIAKSEQEIGKATAKRFFYSNIMNFFKTE | | |
| | | VKLANGEIRKRPLIETNGETGEVVWNKEKDFATVRKVLAMPQVNIVKKTEV | | |
| | | QTGGFSKESILSKRESAKLIPRKKGWDTRKYGGFGSPTVAYSILVVAKVEKGK | | |
| | | AKKLKSVKVLVGITIMEKGSYEKDPIGFLEAKGYKDIKKELIFKLPKYSLFELE | | |
| | | NGRRRMLASATELQKANELVLPQHLVRLLYYTQNISATTGSNNLGYIEQHRE | | |
| | | EFKEIFEKIIDFSEKYILKNKVNSNLKSSFDEQFAVSDSILLSNSFVSLLKYTSF | | |
| | | GASGGFTFLDLDVKQGRLRYQTVTEVLDATLIYQSITGLYETRTDLSQLGGD | | |
| ScaCas9- | Strepto- | MEKKYSIGLDIGTNSVGWAVITDDYKVPSKKFKVLGNTNRKSIKKNLMGALL | N872A | 371 |
| HiFi- | coccus | FDSGETAEATRLKRTARRRYTRRKNRIRYLQEIFANEMAKLDDSFFQRLEESF | | |
| Sc+ | canis | LVEEDKKNERHPIFGNLADEVAYHRNYPTIYHLRKKLADSPEKADLRLIYLAL | | |
| | | AHIIKFRGHFLIEGKLNAENSDVAKLFYQLIQTYNQLFEESPLDEIEVDAKGILS | | |
| | | ARLSKSKRLEKLIAVFPNEKKNGLFGNIIALALGLTPNFKSNFDLTEDAKLQLS | | |
| | | KDTYDDDLDELLGQIGDQYADLFSAAKNLSDAILLSDILRSNSEVTKAPLSAS | | |
| | | MVKRYDEHHQDLALLKTLVRQQFPEKYAEIFKDDTKNGYAGYVGADKKLR | | |
| | | KRSGKLATEEEFYKFIKPILEKMDGAEELLAKLNRDDLLRKQRTFDNGSIPHQI | | |
| | | HLKELHAILRRQEEFYPFLKENREKIEKILTFRIPYYVGPLARGNSRFAWLTRK | | |
| | | SEEAITPWNFEEVVDKGASAQSFIERMTNFDEQLPNKKVLPKHSLLYEYFTVY | | |
| | | NELTKVKYVTERMRKPEFLSGEQKKAIVDLLFKTNRKVTVKQLKEDYFKKIE | | |
| | | CFDSVEIIGVEDRFNASLGTYHDLLKIIKDKDFLDNEENEDILEDIVLTLTLFED | | |
| | | REMIEERLKTYAHLFDDKVMKQLKRRHYTGWGRLSRKMINGIRDKQSGKTIL | | |
| | | DFLKSDGFSNANFMQLIHDDSLTFKEEIEKAQVSGQGDSLHEQIADLAGSPAI | | |
| | | KKGILQTVKIVDELVKVMGHKPENIVIEMARENQTTTKGLQQSRERKKRIEEG | | |
| | | IKELESQILKENPVENTQLQNEKLYLYYLQNGRDMYVDQELDINRLSDYDVD | | |
| | | HIVPQSFIKDDSIDNKVLTRSVENRGKSDNVPSEEVVKKMKNYWRQLLNAKL | | |
| | | ITQRKFDNLTKAERGGLSEADKAGFIKRQLVETRQITKHVARILDSRMNTKRD | | |
| | | KNDKPIREVKVITLKSKLVSDFRKDFQLYKVRDINNYHHAHDAYLNAVVGTA | | |
| | | LIKKYPKLESEFVYGDYKVYDVRKMIAKSEQEIGKATAKRFFYSNIMNFFKTE | | |
| | | VKLANGEIRKRPLIETNGETGEVVWNKEKDFATVRKVLAMPQVNIVKKTEV | | |
| | | QTGGFSKESILSKRESAKLIPRKKGWDTRKYGGFGSPTVAYSILVVAKVEKGK | | |
| | | AKKLKSVKVLVGITIMEKGSYEKDPIGFLEAKGYKDIKKELIFKLPKYSLFELE | | |
| | | NGRRRMLASAKELQKANELVLPQHLVRLLYYTQNISATTGSNNLGYIEQHRE | | |
| | | EFKEIFEKIIDFSEKYILKNKVNSNLKSSFDEQFAVSDSILLSNSFVSLLKYTSF | | |
| | | GASGGFTFLDLDVKQGRLRYQTVTEVLDATLIYQSITGLYETRTDLSQLGGD | | |
| SpyCas9- | Strepto- | MDKKYSIGLDIGTNSVGWAVITDEYKVPSKKFKVLGNTDRHSIKKNLIGALLF | N863A | 372 |
| 3var- | coccus | DSGETAEATRLKRTARRRYTRRKNRICYLQEIFSNEMAKVDDSFFHRLEESFL | | |
| NRRH | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MVKRYDEHHQDLTLLKALVRQQLPEKYKEIFFDQSKNGYAGYIDGGASQEEF | | |
| | | YKFIKPILEKMDGTEELLVKLNREDLLRKQRTFDNGIIPHQIHLGELHAILRRQ | | |
| | | GDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRLRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGGHKPENIVIEMARENQTTQKGQKNSRERMKRIEEGIKELGSQIL | | |
| | | KEHPVENTQLQNEKLYLYYLQNGRDMYVDQELDINRLSDYDVDHIVPQSFL | | |
| | | KDDSIDNKVLTRSDKNRGKSDNVPSEEVVKKMKNYWRQLLNAKLITQRKFD | | |
| | | NLTKAERGGLSELDKAGFIKRQLVETRQITKHVAQILDSRMNTKYDENDKLIR | | |
| | | EVKVITLKSKLVSDFRKDFQFYKVREINNYHHAHDAYLNAVVGTALIKKYPK | | |
| | | LESEFVYGDYKVYDVRKMIAKSEQEIGKATAKYFFYSNIMNFFKTEITLANGE | | |
| | | IRKRPLIETNGETGEIVWDKGRDFATVRKVLSMPQVNIVKKTEVQTGGFSKES | | |
| | | ILPKGNSDKLIARKKDWDPKKYGGFNSPTAAYSVLVVAKVEKGKSKKLKSV | | |
| | | KELLGITIMERSSFEKNPIGFLEAKGYKEVKKDLIIKLPKYSLFELENGRKRML | | |
| | | ASAGVLHKGNELALPSKYVNFLYLASHYEKLKGSPEDNEQKQLFVEQHKHY | | |
| | | LDEIIEQISEFSKRVILADANLDKVLSAYNKHRDKPIREQAENIIHLFTLTNLGV | | |
| | | PAAFKYFDTTIDKKRYTSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 373 |
| 3var- | coccus | DSGETAEATRLKRTARRRYTRRKNRICYLQEIFSNEMAKVDDSFFHRLEESFL | | |
| NRTH | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MVKRYDEHHQDLTLLKALVRQQLPEKYKEIFFDQSKNGYAGYIDGGASQEEF | | |
| | | YKFIKPILEKMDGTEELLVKLNREDLLRKQRTFDNGIIPHQIHLGELHAILRRQ | | |
| | | GDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRLRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGGHKPENIVIEMARENQTTQKGQKNSRERMKRIEEGIKELGSQIL | | |
| | | KEHPVENTQLQNEKLYLYYLQNGRDMYVDQELDINRLSDYDVDHIVPQSFL | | |
| | | KDDSIDNKVLTRSDKNRGKSDNVPSEEVVKKMKNYWRQLLNAKLITQRKFD | | |
| | | NLTKAERGGLSELDKAGFIKRQLVETRQITKHVAQILDSRMNTKYDENDKLIR | | |
| | | EVKVITLKSKLVSDFRKDFQFYKVREINNYHHAHDAYLNAVVGTALIKKYPK | | |
| | | LESEFVYGDYKVYDVRKMIAKSEQEIGKATAKYFFYSNIMNFFKTEITLANGE | | |
| | | IRKRPLIETNGETGEIVWDKGRDFATVRKVLSMPQVNIVKKTEVQTGGFSKES | | |
| | | ILPKGNSDKLIARKKDWDPKKYGGFNSPTVAYSVLVVAKVEKGKSKKLKSV | | |
| | | KELLGITIMERSSFEKNPIGFLEAKGYKEVKKDLIIKLPKYSLFELENGRKRML | | |
| | | ASASVLHKGNELALPSKYVNFLYLASHYEKLKGSSEDNKQKQLFVEQHKHY | | |
| | | LDEIIEQISEFSKRVILADANLDKVLSAYNKHRDKPIREQAENIIHLFTLTNLGA | | |
| | | SAAFKYFDTTIGRKLYTSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 374 |
| 3var- | coccus | DSGETAEATRLKRTARRRYTRRKNRICYLQEIFSNEMAKVDDSFFHRLEESFL | | |
| NRCH | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MVKRYDEHHQDLTLLKALVRQQLPEKYKEIFFDQSKNGYAGYIDGGASQEEF | | |
| | | YKFIKPILEKMDGTEELLVKLNREDLLRKQRTFDNGIIPHQIHLGELHAILRRQ | | |
| | | GDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRLRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGGHKPENIVIEMARENQTTQKGQKNSRERMKRIEEGIKELGSQIL | | |
| | | KEHPVENTQLQNEKLYLYYLQNGRDMYVDQELDINRLSDYDVDHIVPQSFL | | |
| | | KDDSIDNKVLTRSDKNRGKSDNVPSEEVVKKMKNYWRQLLNAKLITQRKFD | | |
| | | NLTKAERGGLSELDKAGFIKRQLVETRQITKHVAQILDSRMNTKYDENDKLIR | | |
| | | EVKVITLKSKLVSDFRKDFQFYKVREINNYHHAHDAYLNAVVGTALIKKYPK | | |
| | | LESEFVYGDYKVYDVRKMIAKSEQEIGKATAKYFFYSNIMNFFKTEITLANGE | | |
| | | IRKRPLIETNGETGEIVWDKGRDFATVRKVLSMPQVNIVKKTEVQTGGFSKES | | |
| | | ILPKGNSDKLIARKKDWDPKKYGGFNSPTVAYSVLVVAKVEKGKSKKLKSV | | |
| | | KELLGITIMERSSFEKNPIDFLEAKGYKEVKKDLIIKLPKYSLFELENGRKRML | | |
| | | ASAGVLQKGNELALPSKYVNFLYLASHYEKLKGSPEDNEQKQLFVEQHKHY | | |
| | | LDEIIEQISEFSKRVILADANLDKVLSAYNKHRDKPIREQAENIIHLFTLTNLGA | | |
| | | PAAFKYFDTTINRKQYNTTKEVLDATLIRQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 375 |
| HF1 | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | LPKRNSDKLIARKKDWDPKKYGGFDSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SAGELQKGNELALPSKYVNFLYLASHYEKLKGSPEDNEQKQLFVEQHKHYL | | |
| | | DEIIEQISEFSKRVILADANLDKVLSAYNKHRDKPIREQAENIIHLFTLTNLGAP | | |
| | | AAFKYFDTTIDRKRYTSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 376 |
| QQR1 | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | LPKRNSDKLIARKKDWDPKKYGGFDSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SARELQKGNELALPSKYVNFLYLASHYEKLKGSPEDNEQKQLFVEQHKHYLD | | |
| | | EIIEQISEFSKRVILADAQLDKVLSAYNKHRDKPIREQAENIIHLFTLTNLGAPA | | |
| | | AFKYFDTTFKQKQYRSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 377 |
| SpG | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | LPKRNSDKLIARKKDWDPKKYGGFLWPTVAYSVLVVAKVEKGKSKKLKSV | | |
| | | KELLGITIMERSSFEKNPIDFLEAKGYKEVKKDLIIKLPKYSLFELENGRKRML | | |
| | | ASAKQLQKGNELALPSKYVNFLYLASHYEKLKGSPEDNEQKQLFVEQHKHY | | |
| | | LDEIIEQISEFSKRVILADANLDKVLSAYNKHRDKPIREQAENIIHLFTLTNLGA | | |
| | | PAAFKYFDTTIDRKQYRSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 378 |
| VQR | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | LPKRNSDKLIARKKDWDPKKYGGFVSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SAGELQKGNELALPSKYVNFLYLASHYEKLKGSPEDNEQKQLFVEQHKHYL | | |
| | | DEIIEQISEFSKRVILADANLDKVLSAYNKHRDKPIREQAENIIHLFTLTNLGAP | | |
| | | AAFKYFDTTIDRKQYRSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 379 |
| VRER | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDAKLQLS | | |
| | | KDTYDDDLDNLLAQIGDQYADLFLAAKNLSDAILLSDILRVNTEITKAPLSAS | | |
| | | MIKRYDEHHQDLTLLKALVRQQLPEKYKEIFFDQSKNGYAGYIDGGASQEEF | | |
| | | YKFIKPILEKMDGTEELLVKLNREDLLRKQRTFDNGSIPHQIHLGELHAILRRQ | | |
| | | EDFYPFLKDNREKIEKILTFRIPYYVGPLARGNSRFAWMTRKSEETITPWNFEE | | |
| | | VVDKGASAQSFIERMTNFDKNLPNEKVLPKHSLLYEYFTVYNELTKVKYVTE | | |
| | | GMRKPAFLSGEQKKAIVDLLFKTNRKVTVKQLKEDYFKKIECFDSVEISGVED | | |
| | | RFNASLGTYHDLLKIIKDKDFLDNEENEDILEDIVLTLTLFEDREMIEERLKTY | | |
| | | AHLFDDKVMKQLKRRRYTGWGRLSRKLINGIRDKQSGKTILDFLKSDGFANR | | |
| | | NFMQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | LPKRNSDKLIARKKDWDPKKYGGFVSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SARELQKGNELALPSKYVNFLYLASHYEKLKGSPEDNEQKQLFVEQHKHYLD | | |
| | | EIIEQISEFSKRVILADANLDKVLSAYNKHRDKPIREQAENIIHLFTLTNLGAPA | | |
| | | AFKYFDTTIDRKEYRSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 380 |
| xCas | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDTKLQLS | | |
| | | KDTYDDDLDNLLAQIGDQYADLFLAAKNLSDAILLSDILRVNTEITKAPLSAS | | |
| | | MIKLYDEHHQDLTLLKALVRQQLPEKYKEIFFDQSKNGYAGYIDGGASQEEF | | |
| | | YKFIKPILEKMDGTEELLVKLNREDLLRKQRTFDNGIIPHQIHLGELHAILRRQ | | |
| | | EDFYPFLKDNREKIEKILTFRIPYYVGPLARGNSRFAWMTRKSEETITPWNFEK | | |
| | | VVDKGASAQSFIERMTNFDKNLPNEKVLPKHSLLYEYFTVYNELTKVKYVTE | | |
| | | GMRKPAFLSGDQKKAIVDLLFKTNRKVTVKQLKEDYFKKIECFDSVEISGVE | | |
| | | DRFNASLGTYHDLLKIIKDKDFLDNEENEDILEDIVLTLTLFEDREMIEERLKT | | |
| | | YAHLFDDKVMKQLKRRRYTGWGRLSRKLINGIRDKQSGKTILDFLKSDGFAN | | |
| | | RNFIQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | LPKRNSDKLIARKKDWDPKKYGGFDSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SAGVLQKGNELALPSKYVNFLYLASHYEKLKGSPEDNEQKQLFVEQHKHYL | | |
| | | DEIIEQISEFSKRVILADANLDKVLSAYNKHRDKPIREQAENIIHLFTLTNLGAP | | |
| | | AAFKYFDTTIDRKRYTSTKEVLDATLIHQSITGLYETRIDLSQLGGD | | |
| SpyCas9- | Strepto- | MDKKYSIGLDIGTNSVGWAVITDEYKVPSKKFKVLGNTDRHSIKKNLIGALLF | N863A | 381 |
| xCas-NG | coccus | DSGETAEATRLKRTARRRYTRRKNRICYLQEIFSNEMAKVDDSFFHRLEESFL | | |
| | pyogenes | VEEDKKHERHPIFGNIVDEVAYHEKYPTIYHLRKKLVDSTDKADLRLIYLALA | | |
| | | HMIKFRGHFLIEGDLNPDNSDVDKLFIQLVQTYNQLFEENPINASGVDAKAILS | | |
| | | ARLSKSRRLENLIAQLPGEKKNGLFGNLIALSLGLTPNFKSNFDLAEDTKLQLS | | |
| | | KDTYDDDLDNLLAQIGDQYADLFLAAKNLSDAILLSDILRVNTEITKAPLSAS | | |
| | | MIKLYDEHHQDLTLLKALVRQQLPEKYKEIFFDQSKNGYAGYIDGGASQEEF | | |
| | | YKFIKPILEKMDGTEELLVKLNREDLLRKQRTFDNGIIPHQIHLGELHAILRRQ | | |
| | | EDFYPFLKDNREKIEKILTFRIPYYVGPLARGNSRFAWMTRKSEETITPWNFEK | | |
| | | VVDKGASAQSFIERMTNFDKNLPNEKVLPKHSLLYEYFTVYNELTKVKYVTE | | |
| | | GMRKPAFLSGDQKKAIVDLLFKTNRKVTVKQLKEDYFKKIECFDSVEISGVE | | |
| | | DRFNASLGTYHDLLKIIKDKDFLDNEENEDILEDIVLTLTLFEDREMIEERLKT | | |
| | | YAHLFDDKVMKQLKRRRYTGWGRLSRKLINGIRDKQSGKTILDFLKSDGFAN | | |
| | | RNFIQLIHDDSLTFKEDIQKAQVSGQGDSLHEHIANLAGSPAIKKGILQTVKVV | | |
| | | DELVKVMGRHKPENIVIEMARENQTTQKGQKNSRERMKRIEEGIKELGSQILK | | |
| | | EHPVENTQLQNEKLYLYYLQNGRDMYVDQELDINRLSDYDVDHIVPQSFLK | | |
| | | DDSIDNKVLTRSDKNRGKSDNVPSEEVVKKMKNYWRQLLNAKLITQRKFDN | | |
| | | LTKAERGGLSELDKAGFIKRQLVETRQITKHVAQILDSRMNTKYDENDKLIRE | | |
| | | VKVITLKSKLVSDFRKDFQFYKVREINNYHHAHDAYLNAVVGTALIKKYPKL | | |
| | | ESEFVYGDYKVYDVRKMIAKSEQEIGKATAKYFFYSNIMNFFKTEITLANGEI | | |
| | | RKRPLIETNGETGEIVWDKGRDFATVRKVLSMPQVNIVKKTEVQTGGFSKESI | | |
| | | RPKRNSDKLIARKKDWDPKKYGGFVSPTVAYSVLVVAKVEKGKSKKLKSVK | | |
| | | ELLGITIMERSSFEKNPIDFLEAKGYKEVKKDLIIKLPKYSLFELENGRKRMLA | | |
| | | SARFLQKGNELALPSKYVNFLYLASHYEKLKGSPEDNEQKQLFVEQHKHYLD | | |
| | | EIIEQISEFSKRVILADANLDKVLSAYNKHRDKPIREQAENIIHLFTLTNLGAPR | | |
| | | AFKYFDTTIDRKVYRSTKEVLDATLIHQSITGLYETRIDLSQLGGD | | |
| St1Cas9- | Strepto- | MSDLVLGLDIGIGSVGVGILNKVTGEIIHKNSRIFPAAQAENNLVRRTNRQGR | N622A | 382 |
| CNRZ1066 | coccus | RLARRKKHRRVRLNRLFEESGLITDFTKISINLNPYQLRVKGLTDELSNEELFI | | |
| | thermophilus | ALKNMVKHRGISYLDDASDDGNSSVGDYAQIVKENSKQLETKTPGQIQLERY | | |
| | | QTYGQLRGDFTVEKDGKKHRLINVFPTSAYRSEALRILQTQQEFNPQITDEFIN | | |
| | | RYLEILTGKRKYYHGPGNEKSRTDYGRYRTSGETLDNIFGILIGKCTFYPDEFR | | |
| | | AAKASYTAQEFNLLNDLNNLTVPTETKKLSKEQKNQIINYVKNEKAMGPAKL | | |
| | | FKYIAKLLSCDVADIKGYRIDKSGKAEIHTFEAYRKMKTLETLDIEQMDRETL | | |
| | | DKLAYVLTLNTEREGIQEALEHEFADGSFSQKQVDELVQFRKANSSIFGKGW | | |
| | | HNFSVKLMMELIPELYETSEEQMTILTRLGKQKTTSSSNKTKYIDEKLLTEEIY | | |
| | | NPVVAKSVRQAIKIVNAAIKEYGDFDNIVIEMARETNEDDEKKAIQKIQKANK | | |
| | | DEKDAAMLKAANQYNGKAELPHSVFHGHKQLATKIRLWHQQGERCLYTGK | | |
| | | TISIHDLINNSNQFEVDHILPLSITFDDSLANKVLVYATANQEKGQRTPYQALD | | |
| | | SMDDAWSFRELKAFVRESKTLSNKKKEYLLTEEDISKFDVRKKFIERNLVDTR | | |
| | | YASRVVLNALQEHFRAHKIDTKVSVVRGQFTSQLRRHWGIEKTRDTYHHHA | | |
| | | VDALIIAASSQLNLWKKQKNTLVSYSEEQLLDIETGELISDDEYKESVFKAPY | | |
| | | QHFVDTLKSKEFEDSILFSYQVDSKFNRKISDATIYATRQAKVGKDKKDETYV | | |
| | | LGKIKDIYTQDGYDAFMKIYKKDKSKFLMYRHDPQTFEKVIEPILENYPNKQ | | |
| | | MNEKGKEVPCNPFLKYKEEHGYIRKYSKKGNGPEIKSLKYYDSKLLGNPIDIT | | |
| | | PENSKNKVVLQSLKPWRTDVYFNKATGKYEILGLKYADLQFEKGTGTYKISQ | | |
| | | EKYNDIKKKEGVDSDSEFKFTLYKNDLLLVKDTETKEQQLFRFLSRTLPKQK | | |
| | | HYVELKPYDKQKFEGGEALIKVLGNVANGGQCIKGLAKSNISIYKVRTDVLG | | |
| | | NQHIIKNEGDKPKLDF | | |
| St1Cas9- | Strepto- | MSDLVLGLDIGIGSVGVGILNKVTGEIIHKNSRIFPAAQAENNLVRRTNRQGR | N622A | 383 |
| LMG1831 | ccocus | RLARRKKHRRVRLNRLFEESGLITDFTKISINLNPYQLRVKGLTDELSNEELFI | | |
| | thermophilus | ALKNMVKHRGISYLDDASDDGNSSVGDYAQIVKENSKQLETKTPGQIQLERY | | |
| | | QTYGQLRGDFTVEKDGKKHRLINVFPTSAYRSEALRILQTQQEFNPQITDEFIN | | |
| | | RYLEILTGKRKYYHGPGNEKSRTDYGRYRTSGETLDNIFGILIGKCTFYPDEFR | | |
| | | AAKASYTAQEFNLLNDLNNLTVPTETKKLSKEQKNQIINYVKNEKAMGPAKL | | |
| | | FKYIAKLLSCDVADIKGYRIDKSGKAEIHTFEAYRKMKTLETLDIEQMDRETL | | |
| | | DKLAYVLTLNTEREGIQEALEHEFADGSFSQKQVDELVQFRKANSSIFGKGW | | |
| | | HNFSVKLMMELIPELYETSEEQMTILTRLGKQKTTSSSNKTKYIDEKLLTEEIY | | |
| | | NPVVAKSVRQAIKIVNAAIKEYGDFDNIVIEMARETNEDDEKKAIQKIQKANK | | |
| | | DEKDAAMLKAANQYNGKAELPHSVFHGHKQLATKIRLWHQQGERCLYTGK | | |
| | | TISIHDLINNSNQFEVDHILPLSITFDDSLANKVLVYATANQEKGQRTPYQALD | | |
| | | SMDDAWSFRELKAFVRESKTLSNKKKEYLLTEEDISKFDVRKKFIERNLVDTR | | |
| | | YASRVVLNALQEHFRAHKIDTKVSVVRGQFTSQLRRHWGIEKTRDTYHHHA | | |
| | | VDALIIAASSQLNLWKKQKNTLVSYSEEQLLDIETGELISDDEYKESVFKAPY | | |
| | | QHFVDTLKSKEFEDSILFSYQVDSKFNRKISDATIYATRQAKVGKDKKDETYV | | |
| | | LGKIKDIYTQDGYDAFMKIYKKDKSKFLMYRHDPQTFEKVIEPILENYPNKQ | | |
| | | MNEKGKEVPCNPFLKYKEEHGYIRKYSKKGNGPEIKSLKYYDSKLLGNPIDIT | | |
| | | PENSKNKVVLQSLKPWRTDVYFNKNTGKYEILGLKYADLQFEKKTGTYKISQ | | |
| | | EKYNGIMKEEGVDSDSEFKFTLYKNDLLLVKDTETKEQQLFRFLSRTMPNVK | | |
| | | YYVELKPYSKDKFEKNESLIEILGSADKSGRCIKGLGKSNISIYKVRTDVLGNQ | | |
| | | HIIKNEGDKPKLDF | | |
| St1Cas9- | Strepto- | MSDLVLGLDIGIGSVGVGILNKVTGEIIHKNSRIFPAAQAENNLVRRTNRQGR | N622A | 384 |
| MTH17C | coccus | RLARRKKHRRVRLNRLFEESGLITDFTKISINLNPYQLRVKGLTDELSNEELFI | | |
| L396 | thermophilus | ALKNMVKHRGISYLDDASDDGNSSVGDYAQIVKENSKQLETKTPGQIQLERY | | |
| | | QTYGQLRGDFTVEKDGKKHRLINVFPTSAYRSEALRILQTQQEFNPQITDEFIN | | |
| | | RYLEILTGKRKYYHGPGNEKSRTDYGRYRTSGETLDNIFGILIGKCTFYPDEFR | | |
| | | AAKASYTAQEFNLLNDLNNLTVPTETKKLSKEQKNQIINYVKNEKAMGPAKL | | |
| | | FKYIAKLLSCDVADIKGYRIDKSGKAEIHTFEAYRKMKTLETLDIEQMDRETL | | |
| | | DKLAYVLTLNTEREGIQEALEHEFADGSFSQKQVDELVQFRKANSSIFGKGW | | |
| | | HNFSVKLMMELIPELYETSEEQMTILTRLGKQKTTSSSNKTKYIDEKLLTEEIY | | |
| | | NPVVAKSVRQAIKIVNAAIKEYGDFDNIVIEMARETNEDDEKKAIQKIQKANK | | |
| | | DEKDAAMLKAANQYNGKAELPHSVFHGHKQLATKIRLWHQQGERCLYTGK | | |
| | | TISIHDLINNSNQFEVDHILPLSITFDDSLANKVLVYATANQEKGQRTPYQALD | | |
| | | SMDDAWSFRELKAFVRESKTLSNKKKEYLLTEEDISKFDVRKKFIERNLVDTR | | |
| | | YASRVVLNALQEHFRAHKIDTKVSVVRGQFTSQLRRHWGIEKTRDTYHHHA | | |
| | | VDALIIAASSQLNLWKKQKNTLVSYSEDQLLDIETGELISDDEYKESVFKAPY | | |
| | | QHFVDTLKSKEFEDSILFSYQVDSKFNRKISDATIYATRQAKVGKDKADETYV | | |
| | | LGKIKDIYTQDGYDAFMKIYKKDKSKFLMYRHDPQTFEKVIEPILENYPNKQI | | |
| | | NEKGKEVPCNPFLKYKEEHGYIRKYSKKGNGPEIKSLKYYDSKLGNHIDITPK | | |
| | | DSNNKVVLQSLKPWRTDVYFNKNTGKYEILGLKYSDMQFEKGTGKYSISKE | | |
| | | QYENIKVREGVDENSEFKFTLYKNDLLLLKDSENGEQILLRFTSRNDTSKHYV | | |
| | | ELKPYNRQKFEGSEYLIKSLGTVAKGGQCIKGLGKSNISIYKVRTDVLGNQHII | | |
| | | KNEGDKPKLDF | | |
| St1Cas9- | Strepto | MSDLVLGLDIGIGSVGVGILNKVTGEIIHKNSRIFPAAQAENNLVRRTNRQGR | N622A | 385 |
| TH1477 | coccus | RLARRKKHRRVRLNRLFEESGLITDFTKISINLNPYQLRVKGLTDELSNEELFI | | |
| | thermophilus | ALKNMVKHRGISYLDDASDDGNSSVGDYAQIVKENSKQLETKTPGQIQLERY | | |
| | | QTYGQLRGDFTVEKDGKKHRLINVFPTSAYRSEALRILQTQQEFNPQITDEFIN | | |
| | | RYLEILTGKRKYYHGPGNEKSRTDYGRYRTSGETLDNIFGILIGKCTFYPDEFR | | |
| | | AAKASYTAQEFNLLNDLNNLTVPTETKKLSKEQKNQIINYVKNEKAMGPAKL | | |
| | | FKYIAKLLSCDVADIKGYRIDKSGKAEIHTFEAYRKMKTLETLDIEQMDRETL | | |
| | | DKLAYVLTLNTEREGIQEALEHEFADGSFSQKQVDELVQFRKANSSIFGKGW | | |
| | | HNFSVKLMMELIPELYETSEEQMTILTRLGKQKTTSSSNKTKYIDEKLLTEEIY | | |
| | | NPVVAKSVRQAIKIVNAAIKEYGDFDNIVIEMARETNEDDEKKAIQKIQKANK | | |
| | | DEKDAAMLKAANQYNGKAELPHSVFHGHKQLATKIRLWHQQGERCLYTGK | | |
| | | TISIHDLINNSNQFEVDHILPLSITFDDSLANKVLVYATANQEKGQRTPYQALD | | |
| | | SMDDAWSFRELKAFVRESKTLSNKKKEYLLTEEDISKFDVRKKFIERNLVDTR | | |
| | | YASRVVLNALQEHFRAHKIDTKVSVVRGQFTSQLRRHWGIEKTRDTYHHHA | | |
| | | VDALIIAASSQLNLWKKQKNTLVSYSEDQLLDIETGELISDDEYKESVFKAPY | | |
| | | QHFVDTLKSKEFEDSILFSYQVDSKFNRKISDATIYATRQAKVGKDKADETYV | | |
| | | LGKIKDIYTQDGYDAFMKIYKKDKSKFLMYRHDPQTFEKVIEPILENYPNKQI | | |
| | | NEKGKEVPCNPFLKYKEEHGYIRKYSKKGNGPEIKSLKYYDSKLGNHIDITPK | | |
| | | DSNNKVVLQSLKPWRTDVYFNKNTGKYEILGLKYSDMQFEKGTGKYSISKE | | |
| | | QYENIKVREGVDENSEFKFTLYKNDLLLLKDSENGEQILLRFTSRNDTSKHYV | | |
| | | ELKPYNRQKFEGSEYLIKSLGTVVKGGRCIKGLGKSNISIYKVRTDVLGNQHII | | |
| | | KNEGDKPKLDF | | |

Table 3B provides parameters to define the necessary components for designing gRNA and/or Template RNAs to apply Cas variants listed in Table 3A for Gene Writing. Tier indicates preferred Cas variants if they are available for use at a given locus. The cut site indicates the validated or predicted protospacer adjacent motif (PAM) requirements, validated or predicted location of cut site (relative to the most upstream base of the PAM site). The gRNA for a given enzyme can be assembled by concatenating the crRNA, Tetraloop, and tracrRNA sequences, and further adding a 5′ spacer of a length within Spacer (min) and Spacer (max) that matches a protospacer at a target site. Further, the predicted location of the ssDNA nick at the target is important for designing the 3′ region of a Template RNA that needs to anneal to the sequence immediately 5′ of the nick in order to initiate target primed reverse transcription.

TABLE 3B
parameters to define the necessary components for designing gRNA and/or Temple
RNAs to apply Cas variants listed in Table 3A for Gene Writing

| | | | | Spacer | Spacer | | | |
|---|---|---|---|---|---|---|---|---|
| Variant | PAM(s) | Cut | Tier | (min) | (max) | crRNA | Tetraloop | tracrRNA |
| Nme2Cas9 | NNNNC | −3 | 1 | 22 | 24 | GTTGT | GAAA | CGAAATGAG |
| | C | | | | | AGCTC | | AACCGTTGC |
| | | | | | | CCTTTC | | TACAATAAG |
| | | | | | | TCATTT | | GCCGTCTGA |
| | | | | | | CG (SEQ | | AAAGATGTG |
| | | | | | | ID NO: | | CCGCAACGC |
| | | | | | | 386) | | TCTGCCCCTT |
| | | | | | | | | AAAGCTTCT |
| | | | | | | | | GCTTTAAGG |
| | | | | | | | | GGCATCGTT |
| | | | | | | | | TA (SEQ ID |
| | | | | | | | | NO: 387) |
| PpnCas9 | NNNNR | | 1 | 21 | 24 | GTTGT | GAAA | GCGAAATGA |
| | TT | | | | | AGCTC | | AAAACGTTG |
| | | | | | | CCTTTT | | TTACAATAA |
| | | | | | | TCATTT | | GAGATGAAT |
| | | | | | | CGC | | TTCTCGCAA |
| | | | | | | (SEQ ID | | AGCTCTGCC |
| | | | | | | NO: 388) | | TCTTGAAAT |
| | | | | | | | | TTCGGTTTCA |
| | | | | | | | | AGAGGCATC |
| | | | | | | | | TTTTT (SEQ |
| | | | | | | | | ID NO: 389) |
| SauCas9 | NNGRR; | −3 | 1 | 21 | 23 | GTTTT | GAAA | CAGAATCTA |
| | NNGRR | | | | | AGTAC | | CTAAAACAA |
| | T | | | | | TCTG | | GGCAAAATG |
| | | | | | | (SEQ ID | | CCGTGTTTAT |
| | | | | | | NO: 390) | | CTCGTCAAC |
| | | | | | | | | TTGTTGGCG |
| | | | | | | | | AGA (SEQ ID |
| | | | | | | | | NO: 391) |
| SauCas9- | NNNRR; | −3 | 1 | 21 | 21 | GTTTT | GAAA | ATTACAGAA |
| KKH | NNNRR | | | | | AGTAC | | TCTACTAAA |
| | T | | | | | TCTGT | | ACAAGGCAA |
| | | | | | | AAT | | AATGCCGTG |
| | | | | | | (SEQ ID | | TTTATCTCGT |
| | | | | | | NO: 392) | | CAACTTGTT |
| | | | | | | | | GGCGAGA |
| | | | | | | | | (SEQ ID NO: |
| | | | | | | | | 393) |
| SauriCas9 | NNGG | −3 | 1 | 21 | 21 | GTTTT | GAAA | CAGAATCTA |
| | | | | | | AGTAC | | CTAAAACAA |
| | | | | | | TCTG | | GGCAAAATG |
| | | | | | | (SEQ ID | | CCGTGTTTAT |
| | | | | | | NO: 394) | | CTCGTCAAC |
| | | | | | | | | TTGTTGGCG |
| | | | | | | | | AGATTTTT |
| | | | | | | | | (SEQ ID NO: |
| | | | | | | | | 395) |
| SauriCas9 | NNRG | −3 | 1 | 21 | 21 | GTTTT | GAAA | CAGAATCTA |
| -KKH | | | | | | AGTAC | | CTAAAACAA |
| (SEQ ID | | | | | | TCTG | | GGCAAAATG |
| NO: 401) | | | | | | (SEQ ID | | CCGTGTTTAT |
| | | | | | | NO: 396) | | CTCGTCAAC |
| | | | | | | | | TTGTTGGCG |
| | | | | | | | | AGATTTTT |
| | | | | | | | | (SEQ ID NO: |
| | | | | | | | | 397) |
| ScaCas9- | NNG | −3 | 1 | 20 | 20 | GTTTT | GAAA | TAGCAAGTT |
| Sc++ | | | | | | AGAGC | | AAAATAAGG |
| | | | | | | TA (SEQ | | CTAGTCCGT |
| | | | | | | ID NO: | | TATCAACTT |
| | | | | | | 398) | | GAAAAAGTG |
| | | | | | | | | GCACCGAGT |
| | | | | | | | | CGGTGC |
| | | | | | | | | (SEQ ID NO: |
| | | | | | | | | 399) |
| SpyCas9 | NGG | −3 | 1 | 20 | 20 | GTTTT | GAAA | TAGCAAGTT |
| | | | | | | AGAGC | | AAAATAAGG |
| | | | | | | TA (SEQ | | CTAGTCCGT |
| | | | | | | | | TATCAACTT |
| | | | | | | ID NO: | | GAAAAAGTG |
| | | | | | | 400) | | GCACCGAGT |
| | | | | | | | | CGGTGC |
| | | | | | | | | (SEQ ID NO: |
| | | | | | | | | 401) |
| SpyCas9- | NG | −3 | 1 | 20 | 20 | GTTTA | GAAA | CAGCATAGC |
| NG | (NGG = N | | | | | AGAGC | | AAGTTTAAA |
| | GA = NG | | | | | TATGC | | TAAGGCTAG |
| | T > NGC) | | | | | TG (SEQ | | TCCGTTATC |
| | | | | | | ID NO: | | AACTTGAAA |
| | | | | | | 402) | | AAGTGGCAC |
| | | | | | | | | CGAGTCGGT |
| | | | | | | | | GC (SEQ ID |
| | | | | | | | | NO: 403) |
| SpyCas9- | NRN > N | −3 | 1 | 20 | 20 | GTTTT | GAAA | TAGCAAGTT |
| SpRY | YN | | | | | AGAGC | | AAAATAAGG |
| | | | | | | TA (SEQ | | CTAGTCCGT |
| | | | | | | ID NO: | | TATCAACTT |
| | | | | | | 404) | | GAAAAAGTG |
| | | | | | | | | GCACCGAGT |
| | | | | | | | | CGGTGC |
| | | | | | | | | (SEQ ID NO: |
| | | | | | | | | 405) |
| St1Cas9 | NNAGA | −3 | 1 | 20 | 20 | GTCTTT | GTAC | CAGAAGCTA |
| | AW > NN | | | | | GTACT | | CAAAGATAA |
| | AGGAW | | | | | CTG | | GGCTTCATG |
| | = NNGG | | | | | (SEQ ID | | CCGAAATCA |
| | AAW | | | | | NO: 407) | | ACACCCTGT |
| | | | | | | | | CATTTTATG |
| | | | | | | | | GCAGGGTGT |
| | | | | | | | | TTT (SEQ ID |
| | | | | | | | | NO: 406) |
| BlatCas9 | NNNNC | −3 | 1 | 19 | 23 | GCTAT | GAAA | GGTAAGTTG |
| | NAA > N | | | | | AGTTC | | CTATAGTAA |
| | NNNCN | | | | | CTTAC | | GGGCAACAG |
| | DD > NN | | | | | T(SEQ | | ACCCGAGGC |
| | NNC | | | | | ID NO: | | GTTGGGGAT |
| | | | | | | 408) | | CGCCTAGCC |
| | | | | | | | | CGTGTTTAC |
| | | | | | | | | GGGCTCTCC |
| | | | | | | | | CCATATTCA |
| | | | | | | | | AAATAATGA |
| | | | | | | | | CAGACGAGC |
| | | | | | | | | ACCTTGGAG |
| | | | | | | | | CATTTATCTC |
| | | | | | | | | CGAGGTGCT |
| | | | | | | | | (SEQ ID NO: |
| | | | | | | | | 409) |
| cCas9-v16 | NNVAC | −3 | 2 | 21 | 21 | GUCUU | GAAA | CAGAAUCUA |
| | T; NNVA | | | | | AGUAC | | CUAAGACAA |
| | TGM; N | | | | | UCUG | | GGCAAAAUG |
| | NVATT; | | | | | (SEQ ID | | CCGUGUUUA |
| | NNVGC | | | | | NO: 410) | | UCUCGUCAA |
| | T; NNVG | | | | | | | CUUGUUGGC |
| | TG; NNV | | | | | | | GAGAUUUUU |
| | GTT | | | | | | | UU (SEQ ID |
| | | | | | | | | NO: 411) |
| cCas9-v17 | NNVRR | −3 | 2 | 21 | 21 | GUCUU | GAAA | CAGAAUCUA |
| | N | | | | | AGUAC | | CUAAGACAA |
| | | | | | | UCUG | | GGCAAAAUG |
| | | | | | | (SEQ ID | | CCGUGUUUA |
| | | | | | | NO: 412) | | UCUCGUCAA |
| | | | | | | | | CUUGUUGGC |
| | | | | | | | | GAGAUUUUU |
| | | | | | | | | UU (SEQ ID |
| | | | | | | | | NO: 413) |
| cCas9-v21 | NNVAC | −3 | 2 | 21 | 21 | GUCUU | GAAA | CAGAAUCUA |
| | T; NNVA | | | | | AGUAC | | CUAAGACAA |
| | TGM; N | | | | | UCUG | | GGCAAAAUG |
| | NVATT; | | | | | (SEQ ID | | CCGUGUUUA |
| | NNVGC | | | | | NO: 414) | | UCUCGUCAA |
| | T; NNVG | | | | | | | CUUGUUGGC |
| | TG; NNV | | | | | | | GAGAUUUUU |
| | GTT | | | | | | | UU (SEQ ID |
| | | | | | | | | NO: 415) |
| cCas9-v42 | NNVRR | −3 | 2 | 21 | 21 | GUCUU | GAAA | CAGAAUCUA |
| | N | | | | | AGUAC | | CUAAGACAA |
| | | | | | | UCUG | | GGCAAAAUG |
| | | | | | | (SEQ ID | | CCGUGUUUA |
| | | | | | | NO: 416) | | UCUCGUCAA |
| | | | | | | | | CUUGUUGGC |
| | | | | | | | | GAGAUUUUU |
| | | | | | | | | UU (SEQ ID |
| | | | | | | | | NO: 417) |
| CdiCas9 | NNRHH | | 2 | 22 | 22 | ACUGG | GAAA | CUGAACCUC |
| | HY; NNR | | | | | GGUUC | | AGUAAGCAU |
| | AAAY | | | | | AG | | UGGCUCGUU |
| | | | | | | (SEQ ID | | UCCAAUGUU |
| | | | | | | NO: 418) | | GAUUGCUCC |
| | | | | | | | | GCCGGUGCU |
| | | | | | | | | CCUUAUUUU |
| | | | | | | | | UAAGGGCGC |
| | | | | | | | | CGGC (SEQ ID |
| | | | | | | | | NO: 419) |
| CjeCas9 | NNNNR | −3 | 2 | 21 | 23 | GTTTT | GAAA | AGGGACTAA |
| | YAC | | | | | AGTCC | | AATAAAGAG |
| | | | | | | CT (SEQ | | TTTGCGGGA |
| | | | | | | ID NO: | | CTCTGCGGG |
| | | | | | | 420) | | GTTACAATC |
| | | | | | | | | CCCTAAAAC |
| | | | | | | | | CGCTTTTTT |
| | | | | | | | | (SEQ ID NO: |
| | | | | | | | | 424) |
| GeoCas9 | NNNNC | | 2 | 21 | 23 | GUCAU | GAAA | UCAGGGUUA |
| | RAA | | | | | AGUUC | | CUAUGAUAA |
| | | | | | | CCCUG | | GGGCUUUCU |
| | | | | | | A (SEQ | | GCCUAAGGC |
| | | | | | | ID NO: | | AGACUGACC |
| | | | | | | 421) | | CGCGGCGUU |
| | | | | | | | | GGGGAUCGC |
| | | | | | | | | CUGUCGCCC |
| | | | | | | | | GCUUUUGGC |
| | | | | | | | | GGGCAUUCC |
| | | | | | | | | CCAUCCUU |
| | | | | | | | | (SEQ ID NO: |
| | | | | | | | | 422) |
| iSpyMac | NAAN | −3 | 2 | 19 | 21 | GTTTT | GAAA | TAGCAAGTT |
| Cas9 | | | | | | AGAGC | | AAAATAAGG |
| | | | | | | TA (SEQ | | CTAGTCCGT |
| | | | | | | ID NO: | | TATCAACTT |
| | | | | | | 423) | | GAAAAAGTG |
| | | | | | | | | GCACCGAGT |
| | | | | | | | | CGGTGC |
| | | | | | | | | (SEQ ID NO: |
| | | | | | | | | 44) |
| NmeCas9 | NNNNG | −3 | 2 | 20 | 24 | GTTGT | GAAA | CGAAATGAG |
| | AYT; NN | | | | | AGCTC | | AACCGTTGC |
| | NNGYT | | | | | CCTTTC | | TACAATAAG |
| | T; NNNN | | | | | TCATTT | | GCCGTCTGA |
| | GAYA; | | | | | CG (SEQ | | AAAGATGTG |
| | NNNNG | | | | | ID NO: | | CCGCAACGC |
| | TCT | | | | | 425) | | TCTGCCCCTT |
| | | | | | | | | AAAGCTTCT |
| | | | | | | | | GCTTTAAGG |
| | | | | | | | | GGCATCGTT |
| | | | | | | | | TA (SEQ ID |
| | | | | | | | | NO: 426) |
| ScaCas9 | NNG | −3 | 2 | 20 | 20 | GTTTT | GAAA | TAGCAAGTT |
| | | | | | | AGAGC | | AAAATAAGG |
| | | | | | | TA (SEQ | | CTAGTCCGT |
| | | | | | | ID NO: | | TATCAACTT |
| | | | | | | 427) | | GAAAAAGTG |
| | | | | | | | | GCACCGAGT |
| | | | | | | | | CGGTGC |
| | | | | | | | | (SEQ ID NO: |
| | | | | | | | | 428) |
| ScaCas9- | NNG | −3 | 2 | 20 | 20 | GTTTT | GAAA | TAGCAAGTT |
| HiFi-Sc++ | | | | | | AGAGC | | AAAATAAGG |
| | | | | | | TA (SEQ | | CTAGTCCGT |
| | | | | | | ID NO: | | TATCAACTT |
| | | | | | | 429) | | GAAAAAGTG |
| | | | | | | | | GCACCGAGT |
| | | | | | | | | CGGTGC |
| | | | | | | | | (SEQ ID NO: |
| | | | | | | | | 430) |
| SpyCas9- | NRRH | −3 | 2 | 20 | 20 | GTTTA | GAAA | CAGCATAGC |
| 3var- | | | | | | AGAGC | | AAGTTTAAA |
| NRRH | | | | | | TATGC | | TAAGGCTAG |
| | | | | | | TG (SEQ | | TCCGTTATC |
| | | | | | | ID NO: | | AACTTGAAA |
| | | | | | | 431) | | AAGTGGCAC |
| | | | | | | | | CGAGTCGGT |
| | | | | | | | | GC (SEQ ID |
| | | | | | | | | NO: 432) |
| SpyCas9- | NRTH | −3 | 2 | 20 | 20 | GTTTA | GAAA | CAGCATAGC |
| 3var- | | | | | | AGAGC | | AAGTTTAAA |
| NRTH | | | | | | TATGC | | TAAGGCTAG |
| | | | | | | TG (SEQ | | TCCGTTATC |
| | | | | | | ID NO: | | AACTTGAAA |
| | | | | | | 433) | | AAGTGGCAC |
| | | | | | | | | CGAGTCGGT |
| | | | | | | | | GC (SEQ ID |
| | | | | | | | | NO: 434) |
| SpyCas9- | NRCH | −3 | 2 | 20 | 20 | GTTTA | GAAA | CAGCATAGC |
| 3var- | | | | | | AGAGC | | AAGTTTAAA |
| NRCH | | | | | | TATGC | | TAAGGCTAG |
| | | | | | | TG (SEQ | | TCCGTTATC |
| | | | | | | ID NO: | | AACTTGAAA |
| | | | | | | 435) | | AAGTGGCAC |
| | | | | | | | | CGAGTCGGT |
| | | | | | | | | GC (SEQ ID |
| | | | | | | | | NO: 436) |
| SpyCas9- | NGG | −3 | 2 | 20 | 20 | GTTTT | GAAA | TAGCAAGTT |
| HF1 | | | | | | AGAGC | | AAAATAAGG |
| | | | | | | TA (SEQ | | CTAGTCCGT |
| | | | | | | ID NO: | | TATCAACTT |
| | | | | | | 437) | | GAAAAAGTG |
| | | | | | | | | GCACCGAGT |
| | | | | | | | | CGGTGC |
| | | | | | | | | (SEQ ID NO: |
| | | | | | | | | 438) |
| SpyCas9- | NAAG | −3 | 2 | 20 | 20 | GTTTT | GAAA | TAGCAAGTT |
| QQRi | | | | | | AGAGC | | AAAATAAGG |
| | | | | | | TA (SEQ | | CTAGTCCGT |
| | | | | | | ID NO: | | TATCAACTT |
| | | | | | | 439) | | GAAAAAGTG |
| | | | | | | | | GCACCGAGT |
| | | | | | | | | CGGTGC |
| | | | | | | | | (SEQ ID NO: |
| | | | | | | | | 440) |
| SpyCas9- | NGN | −3 | 2 | 20 | 20 | GTTTT | GAAA | TAGCAAGTT |
| SpG | | | | | | AGAGC | | AAAATAAGG |
| | | | | | | TA (SEQ | | CTAGTCCGT |
| | | | | | | ID NO: | | TATCAACTT |
| | | | | | | 441) | | GAAAAAGTG |
| | | | | | | | | GCACCGAGT |
| | | | | | | | | CGGTGC |
| | | | | | | | | (SEQ ID NO: |
| | | | | | | | | 442) |
| SpyCas9- | NGAN | −3 | 2 | 20 | 20 | GTTTT | GAAA | TAGCAAGTT |
| VQR | | | | | | AGAGC | | AAAATAAGG |
| | | | | | | TA (SEQ | | CTAGTCCGT |
| | | | | | | ID NO: | | TATCAACTT |
| | | | | | | 443) | | GAAAAAGTG |
| | | | | | | | | GCACCGAGT |
| | | | | | | | | CGGTGC |
| | | | | | | | | (SEQ ID NO: |
| | | | | | | | | 444) |
| SpyCas9- | NGCG | −3 | 2 | 20 | 20 | GTTTT | GAAA | TAGCAAGTT |
| VRER | | | | | | AGAGC | | AAAATAAGG |
| | | | | | | TA (SEQ | | CTAGTCCGT |
| | | | | | | ID NO: | | TATCAACTT |
| | | | | | | 445) | | GAAAAAGTG |
| | | | | | | | | GCACCGAGT |
| | | | | | | | | CGGTGC |
| | | | | | | | | (SEQ ID NO: |
| | | | | | | | | 446) |
| SpyCas9- | NG; GA | −3 | 2 | 20 | 20 | GTTTA | GAAA | CAGCATAGC |
| xCas | A; GAT | | | | | AGAGC | | AAGTTTAAA |
| | | | | | | TATGC | | TAAGGCTAG |
| | | | | | | TG (SEQ | | TCCGTTATC |
| | | | | | | ID NO: | | AACTTGAAA |
| | | | | | | 447) | | AAGTGGCAC |
| | | | | | | | | CGAGTCGGT |
| | | | | | | | | GC (SEQ ID |
| | | | | | | | | NO: 448) |
| SpyCas9- | NG | −3 | 2 | 20 | 20 | GTTTA | GAAA | CAGCATAGC |
| xCas-NG | | | | | | AGAGC | | AAGTTTAAA |
| | | | | | | TATGC | | TAAGGCTAG |
| | | | | | | TG (SEQ | | TCCGTTATC |
| | | | | | | ID NO: | | AACTTGAAA |
| | | | | | | 449) | | AAGTGGCAC |
| | | | | | | | | CGAGTCGGT |
| | | | | | | | | GC (SEQ ID |
| | | | | | | | | NO: 450) |
| St1Cas9- | NNACA | −3 | 2 | 20 | 20 | GTCTTT | GTAC | CAGAAGCTA |
| CNRZ1066 | A | | | | | GTACT | | CAAAGATAA |
| | | | | | | CTG | | GGCTTCATG |
| | | | | | | (SEQ ID | | CCGAAATCA |
| | | | | | | NO: 451) | | ACACCCTGT |
| | | | | | | | | CATTTTATG |
| | | | | | | | | GCAGGGTGT |
| | | | | | | | | TTT (SEQ ID |
| | | | | | | | | NO: 452) |
| St1Cas9- | NNGCA | −3 | 2 | 20 | 20 | GTCTTT | GTAC | CAGAAGCTA |
| LMG1831 | A | | | | | GTACT | | CAAAGATAA |
| | | | | | | CTG | | GGCTTCATG |
| | | | | | | (SEQ ID | | CCGAAATCA |
| | | | | | | NO: 453) | | ACACCCTGT |
| | | | | | | | | CATTTTATG |
| | | | | | | | | GCAGGGTGT |
| | | | | | | | | TTT (SEQ ID |
| | | | | | | | | NO: 454) |
| St1Cas9- | NNAAA | −3 | 2 | 20 | 20 | GTCTTT | GTAC | CAGAAGCTA |
| MTH17C | A | | | | | GTACT | | CAAAGATAA |
| L396 | | | | | | CTG | | GGCTTCATG |
| | | | | | | (SEQ ID | | CCGAAATCA |
| | | | | | | NO: 455) | | ACACCCTGT |
| | | | | | | | | CATTTTATG |
| | | | | | | | | GCAGGGTGT |
| | | | | | | | | TTT (SEQ ID |
| | | | | | | | | NO: 456) |
| St1Cas9- | NNGAA | −3 | 2 | 20 | 20 | GTCTTT | GTAC | CAGAAGCTA |
| TH1477 | A | | | | | GTACT | | CAAAGATAA |
| | | | | | | CTG | | GGCTTCATG |
| | | | | | | (SEQ ID | | CCGAAATCA |
| | | | | | | NO: 457) | | ACACCCTGT |
| | | | | | | | | CATTTTATG |
| | | | | | | | | GCAGGGTGT |
| | | | | | | | | TTT (SEQ ID |
| | | | | | | | | NO: 458) |

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNGRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 4. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions.

TABLE 4
CRISPR/Cas Proteins, Species, and Mutations

| | | | | | Mutations to | |
|---|---|---|---|---|---|---|
| | | | # of | | alter PAM | Mutations to make |
| Name | Enzyme | Species | AAs | PAM | recognition | catalytically dead |
| FnCas9 | Cas9 | Francisella | 1629 | 5′-NGG-3′ | Wt | D11A/H969A/N995A |
| | | novicida | | | | |
| FnCas9 | Cas9 | Francisella | 1629 | 5′-YG-3′ | E1369R/E1449H/ | D11A/H969A/N995A |
| RHA | | novicida | | | R1556A | |
| SaCas9 | Cas9 | Staphylococcus | 1053 | 5′- | Wt | D10A/H557A |
| | | aureus | | NNGRRT-3′ | | |
| SaCas9 | Cas9 | Staphylococcus | 1053 | 5′- | E782K/N968K/ | D10A/H557A |
| KKH | | aureus | | NNNRRT-3′ | R1015H | |
| SpCas9 | Cas9 | Streptococcus | 1368 | 5′-NGG-3′ | Wt | D10A/D839A/H840A/ |
| | | pyogenes | | | | N863A |
| SpCas9 | Cas9 | Streptococcus | 1368 | 5′-NGA-3′ | D1135V/R1335Q/ | D10A/D839A/H840A/ |
| VQR | | pyogenes | | | T1337R | N863A |
| AsCpf1 | Cpf1 | Acidamino- | 1307 | 5′-TYCV-3′ | S542R/K607R | E993A |
| RR | | coccus sp. | | | | |
| | | BV3L6 | | | | |
| AsCpf1 | Cpf1 | Acidamino- | 1307 | 5′-TATV-3′ | S542R/K548V/ | E993A |
| RVR | | coccus sp. | | | N552R | |
| | | BV3L6 | | | | |
| FnCpf1 | Cpf1 | Francisella | 1300 | 5′-NTTN-3′ | Wt | D917A/E1006A/ |
| | | novicida | | | | D1255A |
| NmCas9 | Cas9 | Neisseria | 1082 | 5′- | Wt | D16A/D587A/H588A/ |
| | | meningitidis | | NNNGATT-3′ | | N611A |

In some embodiments, the Cas protein is catalytically active and cuts one or both strands of the target DNA site. In some embodiments, cutting the target DNA site is followed by formation of an alteration, e.g., an insertion or deletion, e.g., by the cellular repair machinery.

In some embodiments, the Cas protein is modified to deactivate or partially deactivate the nuclease, e.g., nuclease-deficient Cas9. Whereas wild-type Cas9 generates double-strand breaks (DSBs) at specific DNA sequences targeted by a gRNA, a number of CRISPR endonucleases having modified functionalities are available, for example: a “nickase” version of Cas9 that has been partially deactivated generates only a single-strand break; a catalytically inactive Cas9 (“dCas9”) does not cut target DNA. In some embodiments, dCas9 binding to a DNA sequence may interfere with transcription at that site by steric hindrance. In some embodiments, dCas9 binding to an anchor sequence may interfere with (e.g., decrease or prevent) genomic complex (e.g., ASMC) formation and/or maintenance. In some embodiments, a DNA-binding domain comprises a catalytically inactive Cas9, e.g., dCas9. Many catalytically inactive Cas9 proteins are known in the art. In some embodiments, dCas9 comprises mutations in each endonuclease domain of the Cas protein, e.g., D10A and H840A or N863A mutations. In some embodiments, a catalytically inactive or partially inactive CRISPR/Cas domain comprises a Cas protein comprising one or more mutations, e.g., one or more of the mutations listed in Table 4. In some embodiments, a Cas protein described on a given row of Table 4 comprises one, two, three, or all of the mutations listed in the same row of Table 4. In some embodiments, a Cas protein, e.g., not described in Table 4, comprises one, two, three, or all of the mutations listed in a row of Table 4 or a corresponding mutation at a corresponding site in that Cas protein.

In some embodiments, a catalytically inactive, e.g., dCas9, or partially deactivated Cas9 protein comprises a D11 mutation (e.g., D11A mutation) or an analogous substitution to the amino acid corresponding to said position. In some embodiments, a catalytically inactive Cas9 protein, e.g., dCas9, or partially deactivated Cas9 protein comprises a H969 mutation (e.g., H969A mutation) or an analogous substitution to the amino acid corresponding to said position. In some embodiments, a catalytically inactive Cas9 protein, e.g., dCas9, or partially deactivated Cas9 protein comprises a N995 mutation (e.g., N995A mutation) or an analogous substitution to the amino acid corresponding to said position. In some embodiments, a catalytically inactive Cas9 protein, e.g., dCas9, comprises mutations at one, two, or three of positions D11, H969, and N995 (e.g., D11A, H969A, and N995A mutations) or analogous substitutions to the amino acids corresponding to said positions.

In some embodiments, a catalytically inactive Cas9 protein, e.g., dCas9, or partially deactivated Cas9 protein comprises a D10 mutation (e.g., a D10A mutation) or an analogous substitution to the amino acid corresponding to said position. In some embodiments, a catalytically inactive Cas9 protein, e.g., dCas9, or partially deactivated Cas9 protein comprises a H557 mutation (e.g., a H557A mutation) or an analogous substitution to the amino acid corresponding to said position. In some embodiments, a catalytically inactive Cas9 protein, e.g., dCas9, comprises a D10 mutation (e.g., a D10A mutation) and a H557 mutation (e.g., a H557A mutation) or analogous substitutions to the amino acids corresponding to said positions.

In some embodiments, a catalytically inactive Cas9 protein, e.g., dCas9, or partially deactivated Cas9 protein comprises a D839 mutation (e.g., a D839A mutation) or an analogous substitution to the amino acid corresponding to said position. In some embodiments, a catalytically inactive Cas9 protein, e.g., dCas9, or partially deactivated Cas9 protein comprises a H840 mutation (e.g., a H840A mutation) or an analogous substitution to the amino acid corresponding to said position. In some embodiments, a catalytically inactive Cas9 protein, e.g., dCas9, or partially deactivated Cas9 protein comprises a N863 mutation (e.g., a N863A mutation) or an analogous substitution to the amino acid corresponding to said position. In some embodiments, a catalytically inactive Cas9 protein, e.g., dCas9, comprises a D10 mutation (e.g., D10A), a D839 mutation (e.g., D839A), a H840 mutation (e.g., H840A), and a N863 mutation (e.g., N863A) or analogous substitutions to the amino acids corresponding to said positions.

In some embodiments, a catalytically inactive Cas9 protein, e.g., dCas9, or partially deactivated Cas9 protein comprises a E993 mutation (e.g., a E993A mutation) or an analogous substitution to the amino acid corresponding to said position.

In some embodiments, a catalytically inactive Cas9 protein, e.g., dCas9, or partially deactivated Cas9 protein comprises a D917 mutation (e.g., a D917A mutation) or an analogous substitution to the amino acid corresponding to said position. In some embodiments, a catalytically inactive Cas9 protein, e.g., dCas9, or partially deactivated Cas9 protein comprises a a E1006 mutation (e.g., a E1006A mutation) or an analogous substitution to the amino acid corresponding to said position. In some embodiments, a catalytically inactive Cas9 protein, e.g., dCas9, or partially deactivated Cas9 protein comprises a D1255 mutation (e.g., a D1255A mutation) or an analogous substitution to the amino acid corresponding to said position. In some embodiments, a catalytically inactive Cas9 protein, e.g., dCas9, comprises a D917 mutation (e.g., D917A), a E1006 mutation (e.g., E1006A), and a D1255 mutation (e.g., D1255A) or analogous substitutions to the amino acids corresponding to said positions.

In some embodiments, a catalytically inactive Cas9 protein, e.g., dCas9, or partially deactivated Cas9 protein comprises a D16 mutation (e.g., a D16A mutation) or an analogous substitution to the amino acid corresponding to said position. In some embodiments, a catalytically inactive Cas9 protein, e.g., dCas9, or partially deactivated Cas9 protein comprises a D587 mutation (e.g., a D587A mutation) or an analogous substitution to the amino acid corresponding to said position. In some embodiments, a catalytically inactive Cas9 protein, e.g., dCas9, or partially deactivated Cas9 protein comprises a H588 mutation (e.g., a H588A mutation) or an analogous substitution to the amino acid corresponding to said position. In some embodiments, a catalytically inactive Cas9 protein, e.g., dCas9, or partially deactivated Cas9 protein comprises a N611 mutation (e.g., a N611A mutation) or an analogous substitution to the amino acid corresponding to said position. In some embodiments, a catalytically inactive Cas9 protein, e.g., dCas9, comprises a D16 mutation (e.g., D16A), a D587 mutation (e.g., D587A), a H588 mutation (e.g., H588A), and a N611 mutation (e.g., N611A) or analogous substitutions to the amino acids corresponding to said positions.

In some embodiments, a DNA-binding domain or endonuclease domain may comprise a Cas molecule comprising or linked (e.g., covalently) to a gRNA (e.g., a template nucleic acid, e.g., template RNA, comprising a gRNA).

In some embodiments, an endonuclease domain or DNA binding domain comprises a Streptococcus pyogenes Cas9 (SpCas9) or a functional fragment or variant thereof. In some embodiments, the endonuclease domain or DNA binding domain comprises a modified SpCas9. In embodiments, the modified SpCas9 comprises a modification that alters protospacer-adjacent motif (PAM) specificity. In embodiments, the PAM has specificity for the nucleic acid sequence 5′-NGT-3′. In embodiments, the modified SpCas9 comprises one or more amino acid substitutions, e.g., at one or more of positions L1111, D1135, G1218, E1219, A1322, of R1335, e.g., selected from L1111R, D1135V, G1218R, E1219F, A1322R, R1335V. In embodiments, the modified SpCas9 comprises the amino acid substitution T1337R and one or more additional amino acid substitutions, e.g., selected from L1111, D1135L, S1136R, G1218S, E1219V, D1332A, D1332S, D1332T, D1332V, D1332L, D1332K, D1332R, R1335Q, T1337, T1337L, T1337Q, T1337I, T1337V, T1337F, T1337S, T1337N, T1337K, T1337H, T1337Q, and T1337M, or corresponding amino acid substitutions thereto. In embodiments, the modified SpCas9 comprises: (i) one or more amino acid substitutions selected from D1135L, S1136R, G1218S, E1219V, A1322R, R1335Q, and T1337; and (ii) one or more amino acid substitutions selected from L1111R, G1218R, E1219F, D1332A, D1332S, D1332T, D1332V, D1332L, D1332K, D1332R, T1337L, T1337I, T1337V, T1337F, T1337S, T1337N, T1337K, T1337R, T1337H, T1337Q, and T1337M, or corresponding amino acid substitutions thereto.

In some embodiments, the endonuclease domain or DNA binding domain comprises a Cas domain, e.g., a Cas9 domain. In embodiments, the endonuclease domain or DNA binding domain comprises a nuclease-active Cas domain, a Cas nickase (nCas) domain, or a nuclease-inactive Cas (dCas) domain. In embodiments, the endonuclease domain or DNA binding domain comprises a nuclease-active Cas9 domain, a Cas9 nickase (nCas9) domain, or a nuclease-inactive Cas9 (dCas9) domain. In some embodiments, the endonuclease domain or DNA binding domain comprises a Cas9 domain of Cas9 (e.g., dCas9 and nCas9), Cas12a/Cpf1, Cas12b/C2c1, Cas12c/C2c3, Cas12d/CasY, Cas12e/CasX, Cas12 g, Cas12h, or Cas12i. In some embodiments, the endonuclease domain or DNA binding domain comprises a Cas9 (e.g., dCas9 and nCas9), Cas12a/Cpf1, Cas12b/C2c1, Cas12c/C2c3, Cas12d/CasY, Cas12e/CasX, Cas12 g, Cas12h, or Cas12i. In some embodiments, the endonuclease domain or DNA binding domain comprises an S. pyogenes or an S. thermophilus Cas9, or a functional fragment thereof. In some embodiments, the endonuclease domain or DNA binding domain comprises a Cas9 sequence, e.g., as described in Chylinski, Rhun, and Charpentier (2013) RNA Biology 10:5, 726-737; incorporated herein by reference. In some embodiments, the endonuclease domain or DNA binding domain comprises the HNH nuclease subdomain and/or the RuvC1 subdomain of a Cas, e.g., Cas9, e.g., as described herein, or a variant thereof. In some embodiments, the endonuclease domain or DNA binding domain comprises Cas12a/Cpf1, Cas12b/C2c1, Cas12c/C2c3, Cas12d/CasY, Cas12e/CasX, Cas12 g, Cas12h, or Cas12i. In some embodiments, the endonuclease domain or DNA binding domain comprises a Cas polypeptide (e.g., enzyme), or a functional fragment thereof. In embodiments, the Cas polypeptide (e.g., enzyme) is selected from Cas1, Cas1B, Cas2, Cas3, Cas4, Cas5, Cas5d, Cas5t, Cas5h, Cas5a, Cash, Cas7, Cas8, Cas8a, Cas8b, Cas8c, Cas9 (e.g., Csn1 or Csx12), Cas10, Cas10d, Cas12a/Cpf1, Cas12b/C2c1, Cas12c/C2c3, Cas12d/CasY, Cas12e/CasX, Cas12 g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2c1, Cas12c/C2c3, Cas12b/C2c1, Cas12c/C2c3, SpCas9(K855A), eSpCas9(1.1), SpCas9-HF1, hyper accurate Cas9 variant (HypaCas9), homologues thereof, modified or engineered versions thereof, and/or functional fragments thereof. In embodiments, the Cas9 comprises one or more substitutions, e.g., selected from H840A, D10A, P475A, W476A, N477A, D1125A, W1126A, and D1127A. In embodiments, the Cas9 comprises one or more mutations at positions selected from: D10, G12, G17, E762, H840, N854, N863, H982, H983, A984, D986, and/or A987, e.g., one or more substitutions selected from D10A, G12A, G17A, E762A, H840A, N854A, N863A, H982A, H983A, A984A, and/or D986A. In some embodiments, the endonuclease domain or DNA binding domain comprises a Cas (e.g., Cas9) sequence from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Campylobacter jejuni, Neisseria meningitidis, Streptococcus pyogenes, or Staphylococcus aureus, or a fragment or variant thereof.

In some embodiments, the endonuclease domain or DNA binding domain comprises a Cpf1 domain, e.g., comprising one or more substitutions, e.g., at position D917, E1006A, D1255 or any combination thereof, e.g., selected from D917A, E1006A, D1255A, D917A/E1006A, D917A/D1255A, E1006A/D1255A, and D917A/E1006A/D1255A.

In some embodiments, the endonuclease domain or DNA binding domain comprises spCas9, spCas9-VRQR (SEQ ID NO: 19), spCas9-VRER (SEQ ID NO: 20), xCas9 (sp), saCas9, saCas9-KKH, spCas9-MQKSER (SEQ ID NO: 21), spCas9-LRKIQK (SEQ ID NO: 22), or spCas9-LRVSQL (SEQ ID NO: 23).

In some embodiments, the endonuclease domain or DNA-binding domain comprises an amino acid sequence as listed in Table 37 below, or an amino acid sequence having at least 80%, 85%, 90%, 95%, 96%, 97%, 98%, or 99% sequence identity thereto. In some embodiments, the endonuclease domain or DNA-binding domain comprises an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 differences (e.g., mutations) relative to any of the amino acid sequences described herein.

TABLE 37
Each of the Reference Sequences are incorporated by reference in their entirety.

| Name | Amino Acid Sequence or Reference Sequence |
|---|---|
| Streptococcus pyogenes | |
| Cas9 | |
| Exemplary Linker | SGSETPGTSESATPES (SEQ ID NO: 24) |
| Exemplary Linker Motif | (SGGS)ₙ (SEQ ID NO: 25) |
| Exemplary Linker Motif | (GGGS)ₙ (SEQ ID NO: 26) |
| Exemplary Linker Motif | (GGGGS)ₙ (SEQ ID NO: 27) |
| Exemplary Linker Motif | (G)ₙ |
| Exemplary Linker Motif | (EAAAK)ₙ (SEQ ID NO: 28) |
| Exemplary Linker Motif | (GGS)ₙ |
| Exemplary Linker Motif | (XP)ₙ |
| Cas9 from Streptococcus | NCBI Reference Sequence: NC_002737.2 and Uniprot |
| pyogenes | Reference Sequence: Q99ZW2 |
| Cas9 from Corynebacterium | NCBI Refs: NC_015683.1, NC_017317.1 |
| ulcerans | |
| Cas9 from Corynebacterium | NCBI Refs: NC_016782.1, NC_016786.1 |
| diphtheria | |
| Cas9 from Spiroplasma | NCBI Ref: NC_021284.1 |
| syrphidicola | |
| Cas9 from Prevotella | NCBI Ref: NC_017861.1 |
| intermedia | |
| Cas9 from Spiroplasma | NCBI Ref: NC_021846.1 |
| taiwanense | |
| Cas9 from Streptococcus | NCBI Ref: NC_021314.1 |
| iniae | |
| Cas9 from Belliella baltica | NCBI Ref: NC_018010.1 |
| Cas9 from Psychroflexus | NCBI Ref: NC_018721.1 |
| torquisi | |
| Cas9 from Streptococcus | NCBI Ref: YP_820832.1 |
| thermophilus | |
| Cas9 from Listeria innocua | NCBI Ref: NP_472073.1 |
| Cas9 from Campylobacter | NCBI Ref: YP_002344900.1 |
| jejuni | |
| Cas9 from Neisseria | NCBI Ref: YP_002342100.1 |
| meningitidis | |
| dCas9 (D10A and H840A) | |
| Catalytically inactive Cas9 | |
| (dCas9) | |
| Cas9 nickase (nCas9) | |
| Catalytically active Cas9 | |
| CasY | ((ncbi.nlm.nih.gov/protein/APG80656.1) |
| | >APG80656.1 CRISPR-associated protein CasY [uncultured |
| | Parcubacteria group bacterium]) |
| CasX | uniprot.org/uniprot/F0NN87; uniprot.org/uniprot/F0NH53 |
| CasX | >tr\|F0NH53\|F0NH53_SULIR CRISPR associated protein, Casx |
| | OS = Sulfolobus islandicus (strain REY15A) GN = SiRe_0771 |
| | PE = 4 SV = 1 |
| Deltaproteobacteria CasX | |
| Cas12b/C2c1 | ((uniprot.org/uniprot/T0D7A2#2) sp\|TOD7A2\|C2C1_ALIAG |
| | CRISPR-associated endonuclease C2c1 OS = Alicyclobacillus |
| | acido-terrestris (strain ATCC 49025/DSM 3922/CIP 106132/ |
| | NCIMB 13137/GD3B) GN = c2c1 PE = 1 SV = 1) |
| BhCas12b (Bacillus | NCBI Reference Sequence: WP_095142515 |
| hisashii) | |
| BvCas12b (Bacillus sp. V3- | NCBI Reference Sequence: WP_101661451.1 |
| 13) | |
| Wild-type Francisella | |
| novicida Cpf1 | |
| Francisella novicida Cpf1 | |
| D917A | |
| Francisella novicida Cpf1 | |
| E1006A | |
| Francisella novicida Cpf1 | |
| D1255A | |
| Francisella novicida Cpf1 | |
| D917A/E1006A | |
| Francisella novicida Cpf1 | |
| D917A/D1255A | |
| Francisella novicida Cpf1 | |
| E1006A/D1255A | |
| Francisella novicida Cpf1 | |
| D917A/E1006A | |
| SaCas9 | |
| SaCas9n | |
| PAM-binding SpCas9 | |
| PAM-binding SpCas9n | |
| PAM-binding SpEQR Cas9 | |
| PAM-binding SpVQR Cas9 | |
| PAM-binding SpVRER | |
| Cas9 | |
| PAM-binding SpVRQR | |
| Cas9 | |
| SpyMacCas9 | |

In some embodiments, a portion or fragment of the agent that promotes activity of the gene modifying system, (e.g., a host response modulator or an epigenetic modifier) is fused to an AAV capsid protein. In some embodiments, the agent is a molecule that promotes immunotolerance. In some embodiments, the agent is an enzyme that reduces host immune response by degrading host antibodies including anti-AAV neutralizing antibodies. In some embodiments, the enzyme is an endopeptidase, e.g., Ig-cleaving endopeptidase, e.g., IdeS or a variant thereof.

### Evolved Variants of Gene Writers

In some embodiments, the invention provides evolved variants of Gene Writers. Evolved variants can, in some embodiments, be produced by mutagenizing a reference Gene Writer, or one of the fragments or domains comprised therein. In some embodiments, one or more of the domains (e g., the reverse transcriptase, DNA binding (including, for example, sequence-guided DNA binding elements), RNA-binding, or endonuclease domain) is evolved One or more of such evolved variant domains can, in some embodiments, be evolved alone or together with other domains. An evolved variant domain or domains may, in some embodiments, be combined with unevolved cognate component(s) or evolved variants of the cognate component(s), e.g., which may have been evolved in either a parallel or serial manner.

In some embodiments, the process of mutagenizing a reference Gene Writer, or fragment or domain thereof, comprises mutagenizing the reference Gene Writer or fragment or domain thereof. In embodiments, the mutagenesis comprises a continuous evolution method (e.g., PACE) or non-continuous evolution method (e.g., PANCE), e.g, as described herein. In some embodiments, the evolved Gene Writer, or a fragment or domain thereof, comprises one or more amino acid variations introduced into its amino acid sequence relative to the amino acid sequence of the reference Gene Writer, or fragment or domain thereof. In embodiments, amino acid sequence variations may include one or more mutated residues (e.g., conservative substitutions, non-conservative substitutions, or a combination thereof) within the amino acid sequence of a reference Gene Writer, e.g., as a result of a change in the nucleotide sequence encoding the gene writer that results in, e g., a change in the codon at any particular position in the coding sequence, the deletion of one or more amino acids (e.g., a truncated protein), the insertion of one or more amino acids, or any combination of the foregoing. The evolved variant Gene Writer may include variants in one or more components or domains of the Gene Writer (e.g., variants introduced into a reverse transcriptase domain, endonuclease domain. DNA binding domain, RNA binding domain, or combinations thereof).

In some aspects, the disclosure provides Gene Writers, systems, kits, and methods using or comprising an evolved variant of a Gene Writer, e.g., employs an evolved variant of a Gene Writer or a Gene Writer produced or produceable by PACE or PANCE. In embodiments, the unevolved reference Gene Writer is a Gene Writer as disclosed herein.

The term “phage-assisted continuous evolution (PACE),” as used herein, generally refers to continuous evolution that employs phage as viral vectors Examples of PACE technology have been described, for example, in International PCT Application No. PCT/US 2009/056194, filed Sep. 8, 2009, published as WO 2010/028347 on Mar. 11, 2010: International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017, U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; and International PCT Application, PCT/US2016/027795, filed Apr. 15, 2016, published as WO 2016/168631 on Oct. 20, 2016, the entire contents of each of which are incorporated herein by reference.

The term “phage-assisted non-continuous evolution (PANCE),” as used herein, generally refers to non-continuous evolution that employs phage as viral vectors. Examples of PANCE technology have been described, for example, in Suzuki T. et al, Crystal structures reveal an elusive functional domain of pyrrolysyl-tRNA synthetase, Nat Chem Biol 13(12): 1261-1266 (2017), incorporated herein by reference in its entirety. Briefly, PANCE is a technique for rapid in vivo directed evolution using serial flask transfers of evolving selection phage (SP), which contain a gene of interest to be evolved, across fresh host cells (e.g., E. coli cells) Genes inside the host cell may be held constant while genes contained in the SP continuously evolve. Following phage growth, an aliquot of infected cells may be used to transfect a subsequent flask containing host E. coli. This process can be repeated and/or continued until the desired phenotype is evolved, e g., for as many transfers as desired.

Methods of applying PACE and PANCE to Gene Writers may be readily appreciated by the skilled artisan by reference to, inter aha, the foregoing references. Additional exemplary methods for directing continuous evolution of genome-modifying proteins or systems, e.g., in a population of host cells, e.g., using phage particles, can be applied to generate evolved variants of Gene Writers, or fragments or subdomains thereof. Non-limiting examples of such methods are described in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012, U.S. Pat. No. 9,023,594, issued May 5, 2015, U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, filed Jun. 14, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Patent Publication No. PCT/US2019/47996, filed Aug. 23, 2019, each of which is incorporated herein by reference in its entirety.

In some non-limiting illustrative embodiments, a method of evolution of a evolved variant Gene Writer, of a fragment or domain thereof, comprises: (a) contacting a population of host cells with a population of viral vectors comprising the gene of interest (the starting Gene Writer or fragment or domain thereof), wherein: (1) the host cell is amenable to infection by the viral vector, (2) the host cell expresses viral genes required for the generation of viral particles; (3) the expression of at least one viral gene required for the production of an infectious viral particle is dependent on a function of the gene of interest: and/or (4) the viral vector allows for expression of the protein in the host cell, and can be replicated and packaged into a viral particle by the host cell. In some embodiments, the method comprises (b) contacting the host cells with a mutagen, using host cells with mutations that elevate mutation rate (e.g, either by carrying a mutation plasmid or some genome modification—e.g., proofing-impaired DNA polymerase, SOS genes, such as UmuC, UmuD′, and/or RecA, which mutations, if plasmid-bound, may be under control of an inducible promoter). or a combination thereof. In some embodiments, the method comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. In some embodiments, the cells are incubated under conditions allowing for the gene of interest to acquire a mutation. In some embodiments, the method further comprises (d) isolating a mutated version of the viral vector, encoding an evolved gene product (e.g., an evolved variant Gene Writer, or fragment or domain thereof), from the population of host cells.

The skilled artisan will appreciate a variety of features employable within the above-described framework. For example, in some embodiments, the viral vector or the phage is a filamentous phage, for example, an M13 phage, e.g., an M13 selection phage. In certain embodiments, the gene required for the production of infectious viral particles is the M13 gene III (gIII). In embodiments, the phage may lack a functional gIII, but otherwise comprise gI, gII, gIV, gV, gVI, gVII, gVIII, gIX, and a gX. In some embodiments, the generation of infectious VSV particles involves the envelope protein VSV-G. Various embodiments can use different retroviral vectors, for example, Murine Leukemia Virus vectors, or Lentiviral vectors. In embodiments, the retroviral vectors can efficiently be packaged with VSV-G envelope protein, e.g., as a substitute for the native envelope protein of the virus.

In some embodiments, host cells are incubated according to a suitable number of viral life cycles, e.g., at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at 5 least 4000, at least 5000, at least 7500, at least 10000, or more consecutive viral life cycles, which in on illustrative and non-limiting examples of M13 phage is 10-20 minutes per virus life cycle. Similarly, conditions can be modulated to adjust the time a host cell remains in a population of host cells, e.g., about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes. Host cell populations can be controlled in part by density of the host cells, or, in some embodiments, the host cell density in an inflow, e.g., 10³ cells/ml, about 10⁴ cells/ml, about 10⁵ cells/ml, about 5-10⁵ cells/ml, about 10⁶ cells/nil, about 5-10⁶ cells/nil, about 10⁷ cells/ml, about 5-10⁷ cells/ml, about 10⁸ cells/ml, about 5-10⁸ cells/ml, about 10⁹ cells/ml, about 5-10⁹ cells/ml, about 10¹⁰ cells/ml, or about 5-10¹¹ cells/m1.

Inteins

In some embodiments, as described in more detail below, Intein-N may be fused to the N-terminal portion of a first domain described herein, and intein-C may be fused to the C-terminal portion of a second domain described herein for the joining of the N-terminal portion to the C-terminal portion, thereby joining the first and second domains. In some embodiments, the first and second domains are each independent chosen from a DNA binding domain, an RNA binding domain, an RT domain, and an endonuclease domain.

As used herein, “intein” refers to a self-splicing protein intron (e.g., peptide), e.g., which ligates flanking N-terminal and C-terminal exteins (e.g., fragments to be joined). An intein may, in some instances, comprise a fragment of a protein that is able to excise itself and join the remaining fragments (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Use of inteins for joining heterologous protein fragments is described, for example, in Wood et al., J. Biol. Chem. 289(21); 14512-9 (2014) (incorporated herein by reference in its entirety). For example, when fused to separate protein fragments, the inteins IntN and IntC may recognize each other, splice themselves out, and/or simultaneously ligate the flanking N- and C-terminal exteins of the protein fragments to which they were fused, thereby reconstituting a full-length protein from the two protein fragments.

In some embodiments, a synthetic intein based on the dnaE intein, the Cfa-N (e.g., split intein-N) and Cfa-C (e.g., split intein-C) intein pair, is used. Examples of such inteins have been described, e.g., in Stevens et al., J Am Chem Soc. 2016 Feb. 24; 138(7):2162-5 (incorporated herein by reference in its entirety). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments, Intein-N and intein-C may be fused to the N-terminal portion of the split Cas9 and the C-terminal portion of a split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]˜C. In some embodiments, an intein-C is fused to the N-terminus of the C-terminal portion of the split Cas9, i.e., to form a structure of N-[intein-C]˜[C-terminal portion of the split Cas9]-C. The mechanism of intein-mediated protein splicing for joining the proteins the inteins are fused to (e.g., split Cas9) is described in Shah et al., Chem Sci. 2014; 5(0:446-461, incorporated herein by reference. Methods for designing and using inteins are known in the art and described, for example by WO2020051561, WO2014004336, WO2017132580, US20150344549, and US20180127780, each of which is incorporated herein by reference in their entirety.

In some embodiments, a split refers to a division into two or more fragments. In some embodiments, a split Cas9 protein or split Cas9 comprises a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a reconstituted Cas9 protein. In embodiments, the Cas9 protein is divided into two fragments within a disordered region of the protein, e.g., as described in Nishimasu et al., Cell, Volume 156, Issue 5, pp. 935-949, 2014, or as described in Jiang et al. (2016) Science 351: 867-871 and PDB file: 5F9R (each of which is incorporated herein by reference in its entirety). A disordered region may be determined by one or more protein structure determination techniques known in the art, including, without limitation, X-ray crystallography, NMR spectroscopy, electron microscopy (e.g., cryoEM), and/or in silico protein modeling. In some embodiments, the protein is divided into two fragments at any C, T, A, or S, e.g., within a region of SpCas9 between amino acids A292-G364, F445-K483, or E565-T637, or at corresponding positions in any other Cas9, Cas9 variant (e.g., nCas9, dCas9), or other napDNAbp. In some embodiments, protein is divided into two fragments at SpCas9 T310, T313, A456, S469, or C574. In some embodiments, the process of dividing the protein into two fragments is referred to as splitting the protein.

In some embodiments, a protein fragment ranges from about 2-1000 amino acids (e.g., between 2-10, 10-50, 50-100, 100-200, 200-300, 300-400, 400-500, 500-600, 600-700, 700-800, 800-900, or 900-1000 amino acids) in length. In some embodiments, a protein fragment ranges from about 5-500 amino acids (e.g., between 5-10, 10-50, 50-100, 100-200, 200-300, 300-400, or 400-500 amino acids) in length. In some embodiments, a protein fragment ranges from about 20-200 amino acids (e.g., between 20-30, 30-40, 40-50, 50-100, or 100-200 amino acids) in length.

In some embodiments, a portion or fragment of a Gene Writer (e.g., Cas9-R2Tg) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some embodiments, an endonuclease domain (e.g., a nickase Cas9 domain) is fused to intein-N and a polypeptide comprising an RT domain is fused to an intein-C.

Exemplary nucleotide and amino acid sequences of interns are provided below:

DnaE Intein-N DNA:
(SEQ ID NO: 29)
TGCCTGTCATACGAAACCGAGATACTGACAGTAGA
ATATGGCCTTCTGCCAATCGGGAAGATTGTGGAGA
AACGGATAGAATGCACAGTTTACTCTGTCGATAAC
AATGGTAACATTTATACTCAGCCAGTTGCCCAGTG
GCACGACCGGGGAGAGCAGGAAGTATTCGAATACT
GTCTGGAGGATGGAAGTCTCATTAGGGCCACTAAG
GACCACAAATTTATGACAGTCGATGGCCAGATGCT
GCCTATAGACGAAATCTTTGAGCGAGAGTTGGACC
TCATGCGAGTTGACAACCTTCCTAAT
DnaE Intein-N Protein:
(SEQ ID NO: 30)
CLSYETEILTVEYGLLPIGKIVEKRIECTVYSVDN
NGNIYTQPVAQWHDRGEQEVFEYCLEDGSLIRATK
DHKFMTVDGQMLPIDEIFERELDLMRVDNLPN
DnaE Intein-C DNA:
(SEQ ID NO: 31)
ATGATCAAGATAGCTACAAGGAAGTATCTTGGCAA
ACAAAACGTTTATGATATTGGAGTCGAAAGAGATC
ACAACTTTGCTCTGAAGAACGGATTCATAGCTTCT
AAT
Intein-C:
(SEQ ID NO: 32)
MIKIATRKYLGKQNVYDIGVERDHNFALKNGFIASN
Cfa-N DNA:
(SEQ ID NO: 33)
TGCCTGTCTTATGATACCGAGATACTTACCGTTGA
ATATGGCTTCTTGCCTATTGGAAAGATTGTCGAAG
AGAGAATTGAATGCACAGTATATACTGTAGACAAG
AATGGTTTCGTTTACACACAGCCCATTGCTCAATG
GCACAATCGCGGCGAACAAGAAGTATTTGAGTACT
GTCTCGAGGATGGAAGCATCATACGAGCAACTAAA
GATCATAAATTCATGACCACTGACGGGCAGATGTT
GCCAATAGATGAGATATTCGAGCGGGGCTTGGATC
TCAAACAAGTGGATGGATTGCCA
Cfa-N Protein:
(SEQ ID NO: 34)
CLSYDTEILTVEYGFLPIGKIVEERIECTVYTVDK
NGFVYTQPIAQWHNRGEQEVFEYCLEDGSIIRATK
DHKFMTTDGQMLPIDEIFERGLDLKQVDGLP
Cfa-C DNA:
(SEQ ID NO: 35)
ATGAAGAGGACTGCCGATGGATCAGAGTTTGAATC
TCCCAAGAAGAAGAGGAAAGTAAAGATAATATCTC
GAAAAAGTCTTGGTACCCAAAATGTCTATGATATT
GGAGTGGAGAAAGATCACAACTTCCTTCTCAAGAA
CGGTCTCGTAGCCAGCAAC
Cfa-C Protein:
(SEQ ID NO: 36)
MKRTADGSEFESPKKKRKVKIISRKSLGTQNVYDI
GVEKDHNFLLKNGLVASN

Template Nucleic Acids

In some embodiments, the template nucleic acid comprises one or more sequence (e.g., 2 sequences) that binds the Gene Writer polypeptide. In some embodiments the template nucleic acid, e.g., template RNA, is covalently linked or fused with the agent that promotes activity of the gene modifying system, (e.g., a host response modulator or an epigenetic modifier). In some embodiments, the template nucleic acid comprises a 5′ UTR that binds the Gene Writer polypeptide and/or a 3′ UTR that binds the Gene Writer polypeptide. In some embodiments, the template nucleic acid comprises a first inverted repeat sequence and a second inverted repeat sequence that each binds the Gene Writer polypeptide.

In some embodiments, the template nucleic acid comprises RNA. In some embodiments, the template nucleic acid comprises DNA (e.g., single stranded or double stranded DNA).

In some embodiments, the template nucleic acid comprises one or more (e.g., 2) homology domains that have homology to the target sequence. In some embodiments, the homology domains are about 10-20, 20-50, or 50-100 nucleotides in length.

The Gene Writer™ systems described herein can modify a host target DNA site using a template nucleic acid sequence. In some embodiments, the Gene Writer™ systems described herein transcribe an RNA sequence template into host target DNA sites by target-primed reverse transcription (TPRT). By writing DNA sequence(s) via reverse transcription of the RNA sequence template directly into the host genome, the Gene Writer™ system can insert an object sequence into a target genome without the need for exogenous DNA sequences to be introduced into the host cell (unlike, for example, CRISPR systems), as well as eliminate an exogenous DNA insertion step. The Gene Writer™ system can also delete a sequence from the target genome or introduce a substitution using an object sequence. Therefore, the Gene Writer™ system provides a platform for the use of customized RNA sequence templates containing object sequences, e.g., sequences comprising heterologous gene coding and/or function information.

In some embodiments, a template RNA can comprise a gRNA sequence, e.g., to direct the GeneWriter to a target site of interest. In some embodiments, a template RNA comprises (e.g., from 5′ to 3′) (i) optionally a sequence (e.g., a CRISPR spacer) that binds a target site (e.g., a second strand of a site in a target genome), (ii) optionally a sequence that binds a polypeptide described herein (e.g., a GeneWriter or a Cas polypeptide), (iii) a heterologous object sequence, and (iv) a 3′ target homology domain.

In some embodiments, a template RNA can comprise a gRNA sequence, e.g., to direct the GeneWriter to a target site of interest. In some embodiments, a template RNA comprises (e.g., from 5′ to 3′) (i) optionally a sequence (e.g., a CRISPR spacer) that binds a target site (e.g., a second strand of a site in a target genome), (ii) optionally a sequence that binds a polypeptide described herein (e.g., a GeneWriter or a Cas polypeptide), (iii) a heterologous object sequence, and (iv) 5′ homology domain and/or a 3′ target homology domain.

In some embodiments, the template nucleic acid molecule comprises a 5′ homology domain and/or a 3′ homology domain. In some embodiments, the 5′ homology domain comprises a nucleic acid sequence having at least 85%, 90%, 95%, 96%, 97%, 98%, 99%, or 100% sequence identity with a nucleic acid sequence comprised in a target nucleic acid molecule. In embodiments, the nucleic acid sequence in the target nucleic acid molecule is within about 1, 2, 3, 4, 5, 6, 7, 8, 9, 10, 15, 20, 25, 30, 35, 40, 45, 50, 60, 70, 80, 90, or 100 nucleotides of (e.g., 5′ relative to) a target insertion site, e.g., for a heterologous object sequence, e.g., comprised in the template nucleic acid molecule.

In some embodiments, the 3′ homology domain comprises a nucleic acid sequence having at least 85%, 90%, 95%, 96%, 97%, 98%, 99%, or 100% sequence identity with a nucleic acid sequence comprised in a target nucleic acid molecule. In embodiments, the nucleic acid sequence in the target nucleic acid molecule is within about 1, 2, 3, 4, 5, 6, 7, 8, 9, 10, 15, 20, 25, 30, 35, 40, 45, 50, 60, 70, 80, 90, or 100 nucleotides of (e.g., 3′ relative to) a target insertion site, e.g., for a heterologous object sequence, e.g., comprised in the template nucleic acid molecule. In some embodiments, the 5′ homology domain is heterologous to the remainder of the template nucleic acid molecule. In some embodiments, the 3′ homology domain is heterologous to the remainder of the template nucleic acid molecule.

In some embodiments, a template nucleic acid (e.g., template RNA) comprises a 3′ target homology domain. In some embodiments, a 3′ target homology domain is disposed 3′ of the heterologous object sequence and is complementary to a sequence adjacent to a site to be modified by a system described herein, or comprises no more than 1, 2, 3, 4, or 5 mismatches to a sequence complementary to the sequence adjacent to a site to be modified by the system/Gene Writer™. In some embodiments, the 3′ homology domain binds within 1, 2, 3, 4, 5, 6, 7, 8, 9, or 10 nucleotides of a nick site in the target nucleic acid molecule. In some embodiments, binding of the 3′ homology domain to the target nucleic acid molecule permits initiation of target-primed reverse transcription (TPRT), e.g., with the 3′ homology domain acting as a primer for TPRT.

In some embodiments, a template nucleic acid (e.g., template RNA) comprises a heterologous object sequence. In some embodiments, the heterologous object sequence may be transcribed by the RT domain of a Gene Writer™ polypeptide, e.g., thereby introducing an alteration into a target site in genomic DNA. In some embodiments, the heterologous object sequence is at least 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, or 1,000 nucleotides (nts) in length, or at least 1, 1.5, 2, 2.5, 3, 3.5, 4, 4.5, 5, 5.5, 6, 6.5, 7, 7.5, 8, 8.5, 9, 9.5, or 10 kilobases in length. In some embodiments, the heterologous object sequence is no more than 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, 1,000, or 2000 nucleotides (nts) in length, or no more than 20, 15, 10, 9, 8, 7, 6, 5, 4, or 3 kilobases in length. In some embodiments, the heterologous object sequence is 30-1000, 40-1000, 50-1000, 60-1000, 70-1000, 74-1000, 75-1000, 76-1000, 77-1000, 78-1000, 79-1000, 80-1000, 85-1000, 90-1000, 100-1000, 120-1000, 140-1000, 160-1000, 180-1000, 200-1000, 500-1000, 30-500, 40-500, 50-500, 60-500, 70-500, 74-500, 75-500, 76-500, 77-500, 78-500, 79-500, 80-500, 85-500, 90-500, 100-500, 120-500, 140-500, 160-500, 180-500, 200-500, 30-200, 40-200, 50-200, 60-200, 70-200, 74-200, 75-200, 76-200, 77-200, 78-200, 79-200, 80-200, 85-200, 90-200, 100-200, 120-200, 140-200, 160-200, 180-200, 30-100, 40-100, 50-100, 60-100, 70-100, 74-100, 75-100, 76-100, 77-100, 78-100, 79-100, 80-100, 85-100, or 90-100 nucleotides (nts) in length, or 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 5-20, 5-15, 5-10, 5-9, 5-8, 5-7, 5-6, 6-20, 6-15, 6-10, 6-9, 6-8, 6-7, 7-20, 7-15, 7-10, 7-9, 7-8, 8-20, 8-15, 8-10, 8-9, 9-20, 9-15, 9-10, 10-15, 10-20, or 15-20 kilobases in length. In some embodiments, the heterologous object sequence is 10-100, 10-90, 10-80, 10-70, 10-60, 10-50, 10-40, 10-30, or 10-20 nt in length, e.g., 10-80, 10-50, or 10-20 nt in length, e.g., about 10-20 nt in length.

The template nucleic acid (e.g., template RNA) may have some homology to the target DNA. In some embodiments, the template nucleic acid (e.g., template RNA) 3′ target homology domain may serve as an annealing region to the target DNA, such that the target DNA is positioned to prime the reverse transcription of the template nucleic acid (e.g., template RNA). In some embodiments the template nucleic acid (e.g., template RNA) has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the RNA. In some embodiments the template nucleic acid (e.g., template RNA) has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 175, 180, or 200 or more bases of at least 50%, 60%, 70%, 80%, 85%, 90%, 95%, 97%, 98%, 99% or 100% homology to the target DNA, e.g., at the 5′ end of the template nucleic acid (e.g., template RNA).

The template nucleic acid (e.g., template RNA) component of a Gene Writer™ genome editing system described herein typically is able to bind the Gene Writer™ genome editing protein of the system. In some embodiments the template nucleic acid (e.g., template RNA) has a 3′ region that is capable of binding a Gene Writer™ genome editing protein. The binding region, e.g., 3′ region, may be a structured RNA region, e.g., having at least 1, 2 or 3 hairpin loops, capable of binding the Gene Writer™ genome editing protein of the system. The binding region may associate the template nucleic acid (e.g., template RNA) with any of the polypeptide modules. In some embodiments, the binding region of the template nucleic acid (e.g., template RNA) may associate with an RNA-binding domain in the polypeptide. In some embodiments, the binding region of the template nucleic acid (e.g., template RNA) may associate with the reverse transcription domain of the polypeptide (e.g., specifically bind to the RT domain). For example, where the reverse transcription domain is derived from a non-LTR retrotransposon, the template nucleic acid (e.g., template RNA) may contain a binding region derived from a non-LTR retrotransposon, e.g., a 3′ UTR from a non-LTR retrotransposon. In some embodiments, the template nucleic acid (e.g., template RNA) may associate with the DNA binding domain of the polypeptide, e.g., a gRNA associating with a Cas9-derived DNA binding domain. In some embodiments, the binding region may also provide DNA target recognition, e.g., a gRNA hybridizing to the target DNA sequence and binding the polypeptide, e.g., a Cas9 domain. In some embodiments, the template nucleic acid (e.g., template RNA) may associate with multiple components of the polypeptide, e.g., DNA binding domain and reverse transcription domain. For example, the template nucleic acid (e.g., template RNA) may comprise a gRNA region that associates with a Cas9-derived DNA binding domain and a 3′ UTR from a non-LTR retrotransposon that associated with a non-LTR retrotransposon-derived reverse transcription domain.

The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous sequence, wherein the reverse transcription will result in insertion of the heterologous sequence into the target DNA. In other embodiments, the RNA template may be designed to write a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to write an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

It is contemplated that it may be useful to employ circular and/or linear RNA states during the formulation, delivery, or Gene Writing reaction within the target cell. Thus, in some embodiments of any of the aspects described herein, a Gene Writing system comprises one or more circular RNAs (circRNAs). In some embodiments of any of the aspects described herein, a Gene Writing system comprises one or more linear RNAs. In some embodiments, a nucleic acid as described herein (e.g., a template nucleic acid, a nucleic acid molecule encoding a Gene Writer polypeptide, or both) is a circRNA. In some embodiments, a circular RNA molecule encodes the Gene Writer polypeptide. In some embodiments, the circRNA molecule encoding the Gene Writer polypeptide is delivered to a host cell. In some embodiments, a circular RNA molecule encodes a recombinase, e.g., as described herein. In some embodiments, the circRNA molecule encoding the recombinase is delivered to a host cell. In some embodiments, the circRNA molecule encoding the Gene Writer polypeptide is linearized (e.g., in the host cell, e.g., in the nucleus of the host cell) prior to translation.

Circular RNAs (circRNAs) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018). In some embodiments, the Gene Writer™ polypeptide is encoded as circRNA. In certain embodiments, the template nucleic acid is a DNA, such as a dsDNA or ssDNA. In certain embodiments, the circDNA comprises a template RNA.

In some embodiments, the circRNA comprises one or more ribozyme sequences. In some embodiments, the ribozyme sequence is activated for autocleavage, e.g., in a host cell, e.g., thereby resulting in linearization of the circRNA. In some embodiments, the ribozyme is activated when the concentration of magnesium reaches a sufficient level for cleavage, e.g., in a host cell. In some embodiments the circRNA is maintained in a low magnesium environment prior to delivery to the host cell. In some embodiments, the ribozyme is a protein-responsive ribozyme. In some embodiments, the ribozyme is a nucleic acid-responsive ribozyme. In some embodiments, the circRNA comprises a cleavage site. In some embodiments, the circRNA comprises a second cleavage site.

In some embodiments, the circRNA is linearized in the nucleus of a target cell. In some embodiments, linearization of a circRNA in the nucleus of a cell involves components present in the nucleus of the cell, e.g., to activate a cleavage event. For example, the B2 and ALU retrotransposons contain self-cleaving ribozymes whose activity is enhanced by interaction with the Polycomb protein, EZH2 (Hernandez et al. PNAS 117(1):415-425 (2020)). Thus, in some embodiments, a ribozyme, e.g., a ribozyme from a B2 or ALU element, that is responsive to a nuclear element, e.g., a nuclear protein, e.g., a genome-interacting protein, e.g., an epigenetic modifier, e.g., EZH2, is incorporated into a circRNA, e.g., of a Gene Writing system. In some embodiments, nuclear localization of the circRNA results in an increase in autocatalytic activity of the ribozyme and linearization of the circRNA.

In some embodiments, the ribozyme is heterologous to one or more of the other components of the Gene Writing system. In some embodiments, an inducible ribozyme (e.g., in a circRNA as described herein) is created synthetically, for example, by utilizing a protein ligand-responsive aptamer design. A system for utilizing the satellite RNA of tobacco ringspot virus hammerhead ribozyme with an MS2 coat protein aptamer has been described (Kennedy et al. Nucleic Acids Res 42(19):12306-12321 (2014), incorporated herein by reference in its entirety) that results in activation of the ribozyme activity in the presence of the MS2 coat protein. In embodiments, such a system responds to protein ligand localized to the cytoplasm or the nucleus. In some embodiments the protein ligand is not MS2. Methods for generating RNA aptamers to target ligands have been described, for example, based on the systematic evolution of ligands by exponential enrichment (SELEX) (Tuerk and Gold, Science 249(4968):505-510 (1990); Ellington and Szostak, Nature 346(6287):818-822 (1990); the methods of each of which are incorporated herein by reference) and have, in some instances, been aided by in silico design (Bell et al. PNAS 117(15):8486-8493, the methods of which are incorporated herein by reference). Thus, in some embodiments, an aptamer for a target ligand is generated and incorporated into a synthetic ribozyme system, e.g., to trigger ribozyme-mediated cleavage and circRNA linearization, e.g., in the presence of the protein ligand. In some embodiments, circRNA linearization is triggered in the cytoplasm, e.g., using an aptamer that associates with a ligand in the cytoplasm. In some embodiments, circRNA linearization is triggered in the nucleus, e.g., using an aptamer that associates with a ligand in the nucleus. In embodiments, the ligand in the nucleus comprises an epigenetic modifier or a transcription factor. In some embodiments the ligand that triggers linearization is present at higher levels in on-target cells than off-target cells.

It is further contemplated that a nucleic acid-responsive ribozyme system can be employed for circRNA linearization. For example, biosensors that sense defined target nucleic acid molecules to trigger ribozyme activation are described, e.g., in Penchovsky (Biotechnology Advances 32(5):1015-1027 (2014), incorporated herein by reference). By these methods, a ribozyme naturally folds into an inactive state and is only activated in the presence of a defined target nucleic acid molecule (e.g., an RNA molecule). In some embodiments, a circRNA of a Gene Writing system comprises a nucleic acid-responsive ribozyme that is activated in the presence of a defined target nucleic acid, e.g., an RNA, e.g., an mRNA, miRNA, guide RNA, gRNA, sgRNA, ncRNA, lncRNA, tRNA, snRNA, or mtRNA. In some embodiments the nucleic acid that triggers linearization is present at higher levels in on-target cells than off-target cells.

In some embodiments of any of the aspects herein, a Gene Writing system incorporates one or more ribozymes with inducible specificity to a target tissue or target cell of interest, e.g., a ribozyme that is activated by a ligand or nucleic acid present at higher levels in a target tissue or target cell of interest. In some embodiments, the Gene Writing system incorporates a ribozyme with inducible specificity to a subcellular compartment, e.g., the nucleus, nucleolus, cytoplasm, or mitochondria. In some embodiments, the ribozyme that is activated by a ligand or nucleic acid present at higher levels in the target subcellular compartment. In some embodiments, an RNA component of a Gene Writing system is provided as circRNA, e.g., that is activated by linearization. In some embodiments, linearization of a circRNA encoding a Gene Writing polypeptide activates the molecule for translation. In some embodiments, a signal that activates a circRNA component of a Gene Writing system is present at higher levels in on-target cells or tissues, e.g., such that the system is specifically activated in these cells.

In some embodiments, an RNA component of a Gene Writing system is provided as a circRNA that is inactivated by linearization. In some embodiments, a circRNA encoding the Gene Writer polypeptide is inactivated by cleavage and degradation. In some embodiments, a circRNA encoding the Gene Writing polypeptide is inactivated by cleavage that separates a translation signal from the coding sequence of the polypeptide. In some embodiments, a signal that inactivates a circRNA component of a Gene Writing system is present at higher levels in off-target cells or tissues, such that the system is specifically inactivated in these cells.

### Production of Compositions and Systems

As will be appreciated by one of skill, methods of designing and constructing nucleic acid constructs and proteins or polypeptides (such as the systems, constructs and polypeptides described herein) are routine in the art. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

The disclosure provides, in part, a nucleic acid, e.g., vector, encoding a Gene Writer polypeptide described herein, a template nucleic acid described herein, or both. In some embodiments, a vector comprises a selective marker, e.g., an antibiotic resistance marker. In some embodiments, the antibiotic resistance marker is a kanamycin resistance marker. In some embodiments, the antibiotic resistance marker does not confer resistance to beta-lactam antibiotics. In some embodiments, the vector does not comprise an ampicillin resistance marker. In some embodiments, the vector comprises a kanamycin resistance marker and does not comprise an ampicillin resistance marker. In some embodiments, a vector encoding a Gene Writer polypeptide is integrated into a target cell genome (e.g., upon administration to a target cell, tissue, organ, or subject). In some embodiments, a vector encoding a Gene Writer polypeptide is not integrated into a target cell genome (e.g., upon administration to a target cell, tissue, organ, or subject). In some embodiments, a vector encoding a template nucleic acid (e.g., template RNA) is not integrated into a target cell genome (e.g., upon administration to a target cell, tissue, organ, or subject). In some embodiments, if a vector is integrated into a target site in a target cell genome, the selective marker is not integrated into the genome. In some embodiments, if a vector is integrated into a target site in a target cell genome, genes or sequences involved in vector maintenance (e.g., plasmid maintenance genes) are not integrated into the genome. In some embodiments, if a vector is integrated into a target site in a target cell genome, transfer regulating sequences (e.g., inverted terminal repeats, e.g., from an AAV) are not integrated into the genome. In some embodiments, administration of a vector (e.g., encoding a Gene Writer polypeptide described herein, a template nucleic acid described herein, or both) to a target cell, tissue, organ, or subject results in integration of a portion of the vector into one or more target sites in the genome(s) of said target cell, tissue, organ, or subject. In some embodiments, less than 99, 95, 90, 80, 70, 60, 50, 40, 30, 20, 10, 5, 4, 3, 2, or 1% of target sites (e.g., no target sites) comprising integrated material comprise a selective marker (e.g., an antibiotic resistance gene), a transfer regulating sequence (e.g., an inverted terminal repeat, e.g., from an AAV), or both from the vector.

Exemplary methods for producing a therapeutic pharmaceutical protein or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture recombinant protein. Examples of mammalian expression systems include CHO, COS, HEK293, HeLA, and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

Applications

In some embodiments, a Gene Writer™ system as described herein can be used to modify an animal cell, plant cell, or fungal cell. In some embodiments, a Gene Writer™ system as described herein can be used to modify a mammalian cell (e.g., a human cell). In some embodiments, a Gene Writer™ system as described herein can be used to modify a cell from a livestock animal (e.g., a cow, horse, sheep, goat, pig, llama, alpaca, camel, yak, chicken, duck, goose, or ostrich). In some embodiments, a Gene Writer™ system as described herein can be used as a laboratory tool or a research tool, or used in a laboratory method or research method, e.g., to modify an animal cell, e.g., a mammalian cell (e.g., a human cell), a plant cell, or a fungal cell.

### Plant-Modification Methods

Gene Writer systems described herein may be used to modify a plant or a plant part (e.g., leaves, roots, flowers, fruits, or seeds), e.g., to increase the fitness of a plant.

A. Delivery to a Plant

Provided herein are methods of delivering a Gene Writer system described herein to a plant. Included are methods for delivering a Gene Writer system to a plant by contacting the plant, or part thereof, with a Gene Writer system. The methods are useful for modifying the plant to, e.g., increase the fitness of a plant.

More specifically, in some embodiments, a nucleic acid described herein (e.g., a nucleic acid encoding a GeneWriter) may be encoded in a vector, e.g., inserted adjacent to a plant promoter, e.g., a maize ubiquitin promoter (ZmUBI) in a plant vector (e.g., pHUC411). In some embodiments, the nucleic acids described herein are introduced into a plant (e.g., japonica rice) or part of a plant (e.g., a callus of a plant) via agrobacteria. In some embodiments, the systems and methods described herein can be used in plants by replacing a plant gene (e.g., hygromycin phosphotransferase (HPT)) with a null allele (e.g., containing a base substitution at the start codon). Systems and methods for modifying a plant genome are described in Xu et. al. Development of plant prime-editing systems for precise genome editing, 2020, Plant Communications.

In one aspect, provided herein is a method of increasing the fitness of a plant, the method including delivering to the plant the Gene Writer system described herein (e.g., in an effective amount and duration) to increase the fitness of the plant relative to an untreated plant (e.g., a plant that has not been delivered the Gene Writer system).

An increase in the fitness of the plant as a consequence of delivery of a Gene Writer system can manifest in a number of ways, e.g., thereby resulting in a better production of the plant, for example, an improved yield, improved vigor of the plant or quality of the harvested product from the plant, an improvement in pre- or post-harvest traits deemed desirable for agriculture or horticulture (e.g., taste, appearance, shelf life), or for an improvement of traits that otherwise benefit humans (e.g., decreased allergen production). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional plant-modifying agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. In some instances, the method is effective to increase yield by about 2×-fold, 5×-fold, 10×-fold, 25×-fold, 50×-fold, 75×-fold, 100×-fold, or more than 100×-fold relative to an untreated plant. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a Gene Writer system can also be measured by other means, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional plant-modifying agents (e.g., plant-modifying agents delivered without PMPs).

Accordingly, provided herein is a method of modifying a plant, the method including delivering to the plant an effective amount of any of the Gene Writer systems provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%, 10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield under water-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g. nematode stress, insect herbivory stress, fungal pathogen stress, bacterial pathogen stress, or viral pathogen stress. The stress may be temporary, e.g. several hours, several days, several months, or permanent, e.g. for the life of the plant.

In some instances, the increase in plant fitness is an increase (e.g., by about 1%, 2%, 5%, 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of any of the Gene Writer systems disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount of any of the Gene Writer systems described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of any of the Gene Writer system described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

B. Application Methods

A plant described herein can be exposed to any of the Gene Writer system compositions described herein in any suitable manner that permits delivering or administering the composition to the plant. The Gene Writer system may be delivered either alone or in combination with other active (e.g., fertilizing agents) or inactive substances and may be applied by, for example, spraying, injection (e.g., microinjection), through plants, pouring, dipping, in the form of concentrated liquids, gels, solutions, suspensions, sprays, powders, pellets, briquettes, bricks and the like, formulated to deliver an effective concentration of the plant-modifying composition.

Amounts and locations for application of the compositions described herein are generally determined by the habitat of the plant, the lifecycle stage at which the plant can be targeted by the plant-modifying composition, the site where the application is to be made, and the physical and functional characteristics of the plant-modifying composition.

In some instances, the composition is sprayed directly onto a plant, e.g., crops, by e.g., backpack spraying, aerial spraying, crop spraying/dusting etc. In instances where the Gene Writer system is delivered to a plant, the plant receiving the Gene Writer system may be at any stage of plant growth. For example, formulated plant-modifying compositions can be applied as a seed-coating or root treatment in early stages of plant growth or as a total plant treatment at later stages of the crop cycle. In some instances, the plant-modifying composition may be applied as a topical agent to a plant.

Further, the Gene Writer system may be applied (e.g., in the soil in which a plant grows, or in the water that is used to water the plant) as a systemic agent that is absorbed and distributed through the tissues of a plant. In some instances, plants or food organisms may be genetically transformed to express the Gene Writer system.

Delayed or continuous release can also be accomplished by coating the Gene Writer system or a composition with the plant-modifying composition(s) with a dissolvable or bioerodable coating layer, such as gelatin, which coating dissolves or erodes in the environment of use, to then make the plant-modifying com Gene Writer system position available, or by dispersing the agent in a dissolvable or erodable matrix. Such continuous release and/or dispensing means devices may be advantageously employed to consistently maintain an effective concentration of one or more of the plant-modifying compositions described herein.

In some instances, the Gene Writer system is delivered to a part of the plant, e.g., a leaf, seed, pollen, root, fruit, shoot, or flower, or a tissue, cell, or protoplast thereof. In some instances, the Gene Writer system is delivered to a cell of the plant. In some instances, the Gene Writer system is delivered to a protoplast of the plant. In some instances, the Gene Writer system is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the Gene Writer system is delivered to a plant embryo.

C. Plants

A variety of plants can be delivered to or treated with a Gene Writer system described herein. Plants that can be delivered a Gene Writer system (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes (e.g., a vineyard), kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. In some instances, the composition is delivered to pollen of the plant. In some instances, the composition is delivered to a seed of the plant. In some instances, the composition is delivered to a protoplast of the plant. In some instances, the composition is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the composition is delivered to a plant embryo. In some instances, the composition is delivered to a plant cell. The stages of vegetative and reproductive growth are also referred to herein as “adult” or “mature” plants.

In instances where the Gene Writer system is delivered to a plant part, the plant part may be modified by the plant-modifying agent. Alternatively, the Gene Writer system may be distributed to other parts of the plant (e.g., by the plant's circulatory system) that are subsequently modified by the plant-modifying agent.

### AAV Administration

In some embodiments, an adeno-associated virus (AAV) is used in conjunction with the system, template nucleic acid, and/or polypeptide described herein. In some embodiments, an AAV is used to deliver, administer, or package the system, template nucleic acid, and/or polypeptide described herein. In some embodiments, the AAV is a recombinant AAV (rAAV).

In some embodiments, a system comprises (a) a polypeptide described herein or a nucleic acid encoding the same, (b) a template nucleic acid (e.g., template RNA) described herein, and (c) one or more first tissue-specific expression-control sequences specific to the target tissue, wherein the one or more first tissue-specific expression-control sequences specific to the target tissue are in operative association with (a), (b), or (a) and (b), wherein, when associated with (a), (a) comprises a nucleic acid encoding the polypeptide.

In some embodiments, a system described herein further comprises a first recombinant adeno-associated virus (rAAV) capsid protein; wherein the at least one of (a) or (b) is associated with the first rAAV capsid protein, wherein at least one of (a) or (b) is flanked by AAV inverted terminal repeats (ITRs).

In some embodiments, (a) and (b) are associated with the first rAAV capsid protein.

In some embodiments, (a) and (b) are on a single nucleic acid.

In some embodiments, the system further comprises a second rAAV capsid protein, wherein at least one of (a) or (b) is associated with the second rAAV capsid protein, and wherein the at least one of (a) or (b) associated with the second rAAV capsid protein is different from the at least one of (a) or (b) is associated with the first rAAV capsid protein.

In some embodiments, the at least one of (a) or (b) is associated with the first or second rAAV capsid protein is dispersed in the interior of the first or second rAAV capsid protein, which first or second rAAV capsid protein is in the form of an AAV capsid particle.

In some embodiments, the system further comprises a nanoparticle, wherein the nanoparticle is associated with at least one of (a) or (b).

In some embodiments, (a) and (b), respectively are associated with: a) a first rAAV capsid protein and a second rAAV capsid protein; b) a nanoparticle and a first rAAV capsid protein; c) a first rAAV capsid protein; d) a first adenovirus capsid protein; e) a first nanoparticle and a second nanoparticle; or f) a first nanoparticle.

Viral vectors are useful for delivering all or part of a system provided by the invention, e.g., for use in methods provided by the invention. Systems derived from different viruses have been employed for the delivery of polypeptides, nucleic acids, or transposons; for example: integrase-deficient lentivirus, adenovirus, adeno-associated virus (AAV), herpes simplex virus, and baculovirus (reviewed in Hodge et al. Hum Gene Ther 2017; Narayanavari et al. Crit Rev Biochem Mol Biol 2017; Boehme et al. Curr Gene Ther 2015).

Adenoviruses are common viruses that have been used as gene delivery vehicles given well-defined biology, genetic stability, high transduction efficiency, and ease of large-scale production (see, for example, review by Lee et al. Genes & Diseases 2017). They possess linear dsDNA genomes and come in a variety of serotypes that differ in tissue and cell tropisms. In order to prevent replication of infectious virus in recipient cells, adenovirus genomes used for packaging are deleted of some or all endogenous viral proteins, which are provided in trans in viral production cells. This renders the genomes helper-dependent, meaning they can only be replicated and packaged into viral particles in the presence of the missing components provided by so-called helper functions. A helper-dependent adenovirus system with all viral ORFs removed may be compatible with packaging foreign DNA of up to −37 kb (Parks et al. J Virol 1997). In some embodiments, an adenoviral vector is used to deliver DNA corresponding to the polypeptide or template component of the Gene Writing™ system, or both are contained on separate or the same adenoviral vector. In some embodiments, the adenovirus is a helper-dependent adenovirus (HD-AdV) that is incapable of self-packaging. In some embodiments, the adenovirus is a high-capacity adenovirus (HC-AdV) that has had all or a substantial portion of endogenous viral ORFs deleted, while retaining the necessary sequence components for packaging into adenoviral particles. For this type of vector, the only adenoviral sequences required for genome packaging are noncoding sequences: the inverted terminal repeats (ITRs) at both ends and the packaging signal at the 5′-end (Jager et al. Nat Protoc 2009). In some embodiments, the adenoviral genome also comprises stuffer DNA to meet a minimal genome size for optimal production and stability (see, for example, Hausl et al. Mol Ther 2010). Adenoviruses have been used in the art for the delivery of transposons to various tissues. In some embodiments, an adenovirus is used to deliver a Gene Writing™ system to the liver.

In some embodiments, an adenovirus is used to deliver a Gene Writing™ system to HSCs, e.g., HDAd5/35++. HDAd5/35++ is an adenovirus with modified serotype 35 fibers that de-target the vector from the liver (Wang et al. Blood Adv 2019). In some embodiments, the adenovirus that delivers a Gene Writing™ system to HSCs utilizes a receptor that is expressed specifically on primitive HSCs, e.g., CD46.

Adeno-associated viruses (AAV) belong to the parvoviridae family and more specifically constitute the dependoparvovirus genus. The AAV genome is composed of a linear single-stranded DNA molecule which contains approximately 4.7 kilobases (kb) and consists of two major open reading frames (ORFs) encoding the non-structural Rep (replication) and structural Cap (capsid) proteins. A second ORF within the cap gene was identified that encodes the assembly-activating protein (AAP). The DNAs flanking the AAV coding regions are two cis-acting inverted terminal repeat (ITR) sequences, approximately 145 nucleotides in length, with interrupted palindromic sequences that can be folded into energetically stable hairpin structures that function as primers of DNA replication. In addition to their role in DNA replication, the ITR sequences have been shown to be involved in viral DNA integration into the cellular genome, rescue from the host genome or plasmid, and encapsidation of viral nucleic acid into mature virions (Muzyczka, (1992) Curr. Top. Micro. Immunol. 158:97-129). In some embodiments, one or more Gene Writing™ nucleic acid components is flanked by ITRs derived from AAV for viral packaging. See, e.g., WO2019113310.

In some embodiments, one or more components of the Gene Writing™ system are carried via at least one AAV vector. In some embodiments, the at least one AAV vector is selected for tropism to a particular cell, tissue, organism. In some embodiments, the AAV vector is pseudotyped, e.g., AAV2/8, wherein AAV2 describes the design of the construct but the capsid protein is replaced by that from AAV8. It is understood that any of the described vectors could be pseudotype derivatives, wherein the capsid protein used to package the AAV genome is derived from that of a different AAV serotype. Without wishing to be limited in vector choice, a list of exemplary AAV serotypes can be found in Table 36. In some embodiments, an AAV to be employed for Gene Writing™ may be evolved for novel cell or tissue tropism as has been demonstrated in the literature (e.g., Davidsson et al. Proc Natl Acad Sci USA 2019).

In some embodiments, the AAV delivery vector is a vector which has two AAV inverted terminal repeats (ITRs) and a nucleotide sequence of interest (for example, a sequence coding for a Gene Writer™ polypeptide or a DNA template, or both), each of said ITRs having an interrupted (or noncontiguous) palindromic sequence, i.e., a sequence composed of three segments: a first segment and a last segment that are identical when read 5′—>3′ but hybridize when placed against each other, and a segment that is different that separates the identical segments. Such sequences, notably the ITRs, form hairpin structures. See, for example, WO2012123430.

Conventionally, AAV virions with capsids are produced by introducing a plasmid or plasmids encoding the rAAV or scAAV genome, Rep proteins, and Cap proteins (Grimm et al, 1998). Upon introduction of these helper plasmids in trans, the AAV genome is “rescued” (i.e., released and subsequently recovered) from the host genome, and is further encapsidated to produce infectious AAV. In some embodiments, one or more Gene Writing™ nucleic acids are packaged into AAV particles by introducing the ITR-flanked nucleic acids into a packaging cell in conjunction with the helper functions.

In some embodiments, the AAV genome is a so called self-complementary genome (referred to as scAAV), such that the sequence located between the ITRs contains both the desired nucleic acid sequence (e.g., DNA encoding the Gene Writer™ polypeptide or template, or both) in addition to the reverse complement of the desired nucleic acid sequence, such that these two components can fold over and self-hybridize. In some embodiments, the self-complementary modules are separated by an intervening sequence that permits the DNA to fold back on itself, e.g., forms a stem-loop. An scAAV has the advantage of being poised for transcription upon entering the nucleus, rather than being first dependent on ITR priming and second-strand synthesis to form dsDNA. In some embodiments, one or more Gene Writing™ components is designed as an scAAV, wherein the sequence between the AAV ITRs contains two reverse complementing modules that can self-hybridize to create dsDNA.

In some embodiments, nucleic acid (e.g., encoding a polypeptide, or a template, or both) delivered to cells is closed-ended, linear duplex DNA (CELiD DNA or ceDNA). In some embodiments, ceDNA is derived from the replicative form of the AAV genome (Li et al. PLoS One 2013). In some embodiments, the nucleic acid (e.g., encoding a polypeptide, or a template DNA, or both) is flanked by ITRs, e.g., AAV ITRs, wherein at least one of the ITRs comprises a terminal resolution site and a replication protein binding site (sometimes referred to as a replicative protein binding site). In some embodiments, the ITRs are derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, the ITRs are symmetric. In some embodiments, the ITRs are asymmetric. In some embodiments, at least one Rep protein is provided to enable replication of the construct. In some embodiments, the at least one Rep protein is derived from an adeno-associated virus, e.g., AAV1, AAV2, AAV3, AAV4, AAV5, AAV6, AAV7, AAV8, AAV9, AAV10, AAV11, AAV12, or a combination thereof. In some embodiments, ceDNA is generated by providing a production cell with (i) DNA flanked by ITRs, e.g., AAV ITRs, and (ii) components required for ITR-dependent replication, e.g., AAV proteins Rep78 and Rep52 (or nucleic acid encoding the proteins). In some embodiments, ceDNA is free of any capsid protein, e.g., is not packaged into an infectious AAV particle. In some embodiments, ceDNA is formulated into LNPs (see, for example, WO2019051289A1).

In some embodiments, the ceDNA vector consists of two self complementary sequences, e.g., asymmetrical or symmetrical or substantially symmetrical ITRs as defined herein, flanking said expression cassette, wherein the ceDNA vector is not associated with a capsid protein. In some embodiments, the ceDNA vector comprises two self-complementary sequences found in an AAV genome, where at least one ITR comprises an operative Rep-binding element (RBE) (also sometimes referred to herein as “RBS”) and a terminal resolution site (trs) of AAV or a functional variant of the RBE. See, for example, WO2019113310.

In some embodiments, the AAV genome comprises two genes that encode four replication proteins and three capsid proteins, respectively. In some embodiments, the genes are flanked on either side by 145-bp inverted terminal repeats (ITRs). In some embodiments, the virion comprises up to three capsid proteins (Vp1, Vp2, and/or Vp3), e.g., produced in a 1:1:10 ratio. In some embodiments, the capsid proteins are produced from the same open reading frame and/or from differential splicing (Vp1) and alternative translational start sites (Vp2 and Vp3, respectively). Generally, Vp3 is the most abundant subunit in the virion and participates in receptor recognition at the cell surface defining the tropism of the virus. In some embodiments, Vp1 comprises a phospholipase domain, e.g., which functions in viral infectivity, in the N-terminus of Vp1.

In some embodiments, packaging capacity of the viral vectors limits the size of the base editor that can be packaged into the vector. For example, the packaging capacity of the AAVs can be about 4.5 kb (e.g., about 3.0, 3.5, 4.0, 4.5, 5.0, 5.5, or 6.0 kb), e.g., including one or two inverted terminal repeats (ITRs), e.g., 145 base ITRs.

In some embodiments, recombinant AAV (rAAV) comprises cis-acting 145-bp ITRs flanking vector transgene cassettes, e.g., providing up to 4.5 kb for packaging of foreign DNA. Subsequent to infection, rAAV can, in some instances, express a fusion protein of the invention and persist without integration into the host genome by existing episomally in circular head-to-tail concatemers. rAAV can be used, for example, in vitro and in vivo. In some embodiments, AAV-mediated gene delivery requires that the length of the coding sequence of the gene is equal or greater in size than the wild-type AAV genome.

AAV delivery of genes that exceed this size and/or the use of large physiological regulatory elements can be accomplished, for example, by dividing the protein(s) to be delivered into two or more fragments. In some embodiments, the N-terminal fragment is fused to a split intein-N. In some embodiments, the C-terminal fragment is fused to a split intein-C. In embodiments, the fragments are packaged into two or more AAV vectors.

In some embodiments, dual AAV vectors are generated by splitting a large transgene expression cassette in two separate halves (5 and 3 ends, or head and tail), e.g., wherein each half of the cassette is packaged in a single AAV vector (of <5 kb). The re-assembly of the full-length transgene expression cassette can, in some embodiments, then be achieved upon co-infection of the same cell by both dual AAV vectors. In some embodiments, co-infection is followed by one or more of: (1) homologous recombination (HR) between 5 and 3 genomes (dual AAV overlapping vectors); (2) ITR-mediated tail-to-head concatemerization of 5 and 3 genomes (dual AAV trans-splicing vectors); and/or (3) a combination of these two mechanisms (dual AAV hybrid vectors). In some embodiments, the use of dual AAV vectors in vivo results in the expression of full-length proteins. In some embodiments, the use of the dual AAV vector platform represents an efficient and viable gene transfer strategy for transgenes of greater than about 4.0, 4.1, 4.2, 4.3, 4.4, 4.5, 4.6, 4.7, 4.8, 4.9, or 5.0 kb in size. In some embodiments, AAV vectors can also be used to transduce cells with target nucleic acids, e.g., in the in vitro production of nucleic acids and peptides. In some embodiments, AAV vectors can be used for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994); each of which is incorporated herein by reference in their entirety). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989) (incorporated by reference herein in their entirety).

In some embodiments, a Gene Writer described herein (e.g., with or without one or more guide nucleic acids) can be delivered using AAV, lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as described in U.S. Pat. No. 8,454,972 and as in clinical trials involving AAV. For Adenovirus, the route of administration, formulation and dose can be as described in U.S. Pat. No. 8,404,658 and as in clinical trials involving adenovirus. For plasmid delivery, the route of administration, formulation and dose can be as described in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. In some embodiments, the viral vectors can be injected into the tissue of interest. For cell-type specific Gene Writing, the expression of the Gene Writer and optional guide nucleic acid can, in some embodiments, be driven by a cell-type specific promoter.

In some embodiments, AAV allows for low toxicity, for example, due to the purification method not requiring ultracentrifugation of cell particles that can activate the immune response. In some embodiments, AAV allows low probability of causing insertional mutagenesis, for example, because it does not substantially integrate into the host genome.

In some embodiments, AAV has a packaging limit of about 4.4, 4.5, 4.6, 4.7, or 4.75 kb. In some embodiments, a Gene Writer, promoter, and transcription terminator can fit into a single viral vector. SpCas9 (4.1 kb) may, in some instances, be difficult to package into AAV. Therefore, in some embodiments, a Gene Writer is used that is shorter in length than other Gene Writers or base editors. In some embodiments, the Gene Writers are less than about 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb.

An AAV can be AAV1, AAV2, AAV5 or any combination thereof. In some embodiments, the type of AAV is selected with respect to the cells to be targeted; e.g., AAV serotypes 1, 2, 5 or a hybrid capsid AAV1, AAV2, AAV5 or any combination thereof can be selected for targeting brain or neuronal cells; or AAV4 can be selected for targeting cardiac tissue. In some embodiments, AAV8 is selected for delivery to the liver. Exemplary AAV serotypes as to these cells are described, for example, in Grimm, D. et al, J. Virol. 82: 5887-5911 (2008) (incorporated herein by reference in its entirety). In some embodiments, AAV refers all serotypes, subtypes, and naturally-occurring AAV as well as recombinant AAV. AAV may be used to refer to the virus itself or a derivative thereof. In some embodiments, AAV includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV 12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. Additional exemplary AAV serotypes are listed in Table 36.

In some embodiment, the agent that promotes activity of the gene modifying system is fused to component of a delivery vehicle. In some embodiments, the component is fused to an AAV, e.g., an AAV capsid. In some embodiments the agent is a nucleic acid, e.g., an RNA, e.g., an inhibitory RNA, a small molecule, a large molecule, e.g., a biologic, e.g., a polypeptide, e.g., an antibody (including antibody-drug conjugates) or an enzyme, or a functional fragment thereof, e.g., a domain. In some embodiments, the agent modulates, e.g., inhibits or stimulates a host process. In some embodiments, the agent is an enzyme, e.g., an endopeptidase, e.g., Ig-cleaving endopeptidase, e.g., IdeS, that degrades host antibodies including anti-AAV neutralizing antibodies. In some embodiments, the agent is a molecule that promotes immunotolerance. In some embodiments, the agent is a complement inhibitor. In some embodiments, the agent is contained within a delivery vehicle with the gene modifying system. In some embodiments, the agent is embedded in a delivery system with the gene modifying system. In some embodiments, the agent is displayed on the outside of a delivery vehicle, e.g., fused to a capsid protein of an AAV or fused to a lipid of an LNP. In some embodiments, the agent is embedded in the capsid before creation of the delivery vehicle, e.g., expressed as a fusion protein for AAV. In some embodiments, the agent is embedded in the capsid after creation of the delivery vehicle, e.g., express a domain on a AAV capsid that could be used to subsequently attach, e.g., covalently attach or non-covalently attach, the agent (e.g., an enzyme) after formation of the particles, e.g., SpyTag-SpyCatcher or biotin-streptavidin system. In some embodiments, the agent may be covalently attached to a delivery vehicle, e.g., covalently attached to the capsid of an AAV. In some embodiments, the agent is co-formulated with the gene modifying system. In some embodiments, the agent is incorporated in the structure of a delivery vehicle, e.g., incorporated in the structure of an LNP. In some embodiments, the agent may be contained within a delivery vehicle.

TABLE 36
Exemplary AAV serotypes.

| Target | | |
|---|---|---|
| Tissue | Vehicle | Reference |
| Liver | AAV (AAV8¹, AAVrh.8¹, | 1. Wang et al., Mol. Ther. 18, |
| | AAVhu.37¹, AAV2/8, | 118-25 (2010) |
| | AAV2/rh10², AAV9, | 2. Ginn et al., JHEP Reports, |
| | AAV2, NP40³, NP59^{2, 3}, | 100065 (2019) |
| | AAV3B⁵, AAV-DJ⁴, | 3. Paulk et al., Mol. Ther. 26, |
| | AAV-LK01⁴, AAV-LK02⁴, | 289-303 (2018). |
| | AAV-LK03⁴, AAV-LK19⁴, | 4. L. Lisowski et al., Nature. |
| | AAV5⁷ Adenovirus | 506, 382-6 (2014). |
| | (Ad5, HC-AdV⁶) | 5. L. Wang et al., Mol. Ther. |
| | | 23, 1877-87 (2015). |
| | | 6. Hausl Mol Ther (2010) |
| | | 7. Davidoff et al., Mol. Ther. |
| | | 11, 875-88 (2005) |
| Lung | AAV (AAV4, AAV5, | 1. Duncan et al., Mol Ther |
| | AAV6¹, AAV9, H22²) | Methods Clin Dev (2018) |
| | Adenovirus (Ad5, Ad3, | 2. Cooney et al., Am J Respir |
| | Ad21, Ad14)³ | Cell Mol Biol (2019) |
| | | 3. Li et al., Mol Ther Methods |
| | | Clin Dev (2019) |
| Skin | AAV (AAV6¹, AAV-LK19²) | 1. Petek et al., Mol. Ther. |
| | | (2010) |
| | | 2. L. Lisowski et al., Nature. |
| | | 506, 382-6 (2014). |
| HSCs | Adenovirus (HDAd5/35⁺⁺) | Wang et al. Blood Adv (2019) |

In some embodiments, a pharmaceutical composition (e.g., comprising an AAV as described herein) has less than 10% empty capsids, less than 8% empty capsids, less than 7% empty capsids, less than 5% empty capsids, less than 3% empty capsids, or less than 1% empty capsids. In some embodiments, the pharmaceutical composition has less than about 5% empty capsids. In some embodiments, the number of empty capsids is below the limit of detection. In some embodiments, it is advantageous for the pharmaceutical composition to have low amounts of empty capsids, e.g., because empty capsids may generate an adverse response (e.g., immune response, inflammatory response, liver response, and/or cardiac response), e.g., with little or no substantial therapeutic benefit.

In some embodiments, the residual host cell protein (rHCP) in the pharmaceutical composition is less than or equal to 100 ng/ml rHCP per 1×10¹³ vg/ml, e.g., less than or equal to 40 ng/ml rHCP per 1×10¹³ vg/ml or 1-50 ng/ml rHCP per 1×10¹³ vg/ml. In some embodiments, the pharmaceutical composition comprises less than 10 ng rHCP per 1.0×10¹³ vg, or less than 5 ng rHCP per 1.0×10¹³ vg, less than 4 ng rHCP per 1.0×10¹³ vg, or less than 3 ng rHCP per 1.0×10¹³ vg, or any concentration in between. In some embodiments, the residual host cell DNA (hcDNA) in the pharmaceutical composition is less than or equal to 5×10⁶ pg/ml hcDNA per 1×10¹³ vg/ml, less than or equal to 1.2×10⁶ pg/ml hcDNA per 1×10¹³ vg/ml, or 1×10⁵ pg/ml hcDNA per 1×10¹³ vg/ml. In some embodiments, the residual host cell DNA in said pharmaceutical composition is less than 5.0×10⁵ pg per 1×10¹³ vg, less than 2.0×10⁵ pg per 1.0×10¹³ vg, less than 1.1×10⁵ pg per 1.0×10¹³ vg, less than 1.0×10⁵ pg hcDNA per 1.0×10¹³ vg, less than 0.9×10⁵ pg hcDNA per 1.0×10¹³ vg, less than 0.8×10⁵ pg hcDNA per 1.0×10¹³ vg, or any concentration in between.

In some embodiments, the residual plasmid DNA in the pharmaceutical composition is less than or equal to 1.7×10⁵ pg/ml per 1.0×10¹³ vg/ml, or 1×10⁵ pg/ml per 1×1.0×10¹³ vg/ml, or 1.7×10⁶ pg/ml per 1.0×10¹³ vg/ml. In some embodiments, the residual DNA plasmid in the pharmaceutical composition is less than 10.0×10⁵ pg by 1.0×10¹³ vg, less than 8.0×10⁵ pg by 1.0×10¹³ vg or less than 6.8×10⁵ pg by 1.0×10¹³ vg. In embodiments, the pharmaceutical composition comprises less than 0.5 ng per 1.0×10¹³ vg, less than 0.3 ng per 1.0×10¹³ vg, less than 0.22 ng per 1.0×10¹³ vg or less than 0.2 ng per 1.0×10¹³ vg or any intermediate concentration of bovine serum albumin (BSA). In embodiments, the benzonase in the pharmaceutical composition is less than 0.2 ng by 1.0×10¹³ vg, less than 0.1 ng by 1.0×10¹³ vg, less than 0.09 ng by 1.0×10¹³ vg, less than 0.08 ng by 1.0×10¹³ vg or any intermediate concentration. In embodiments, Poloxamer 188 in the pharmaceutical composition is about 10 to 150 ppm, about 15 to 100 ppm or about 20 to 80 ppm. In embodiments, the cesium in the pharmaceutical composition is less than 50 pg/g (ppm), less than 30 pg/g (ppm) or less than 20 pg/g (ppm) or any intermediate concentration.

In embodiments, the pharmaceutical composition comprises total impurities, e.g., as determined by SDS-PAGE, of less than 10%, less than 8%, less than 7%, less than 6%, less than 5%, less than 4%, less than 3%, less than 2%, or any percentage in between. In embodiments, the total purity, e.g., as determined by SDS-PAGE, is greater than 90%, greater than 92%, greater than 93%, greater than 94%, greater than 95%, greater than 96%, greater than 97%, greater than 98%, or any percentage in between. In embodiments, no single unnamed related impurity, e.g., as measured by SDS-PAGE, is greater than 5%, greater than 4%, greater than 3% or greater than 2%, or any percentage in between. In embodiments, the pharmaceutical composition comprises a percentage of filled capsids relative to total capsids (e.g., peak 1+peak 2 as measured by analytical ultracentrifugation) of greater than 85%, greater than 86%, greater than 87%, greater than 88%, greater than 89%, greater than 90%, greater than 91%, greater than 91.9%, greater than 92%, greater than 93%, or any percentage in between. In embodiments of the pharmaceutical composition, the percentage of filled capsids measured in peak 1 by analytical ultracentrifugation is 20-80%, 25-75%, 30-75%, 35-75%, or 37.4-70.3%. In embodiments of the pharmaceutical composition, the percentage of filled capsids measured in peak 2 by analytical ultracentrifugation is 20-80%, 20-70%, 22-65%, 24-62%, or 24.9-60.1%.

In one embodiment, the pharmaceutical composition comprises a genomic titer of 1.0 to 5.0×10¹³ vg/mL, 1.2 to 3.0×10¹³ vg/mL or 1.7 to 2.3×10¹³ vg/ml. In one embodiment, the pharmaceutical composition exhibits a biological load of less than 5 CFU/mL, less than 4 CFU/mL, less than 3 CFU/mL, less than 2 CFU/mL or less than 1 CFU/mL or any intermediate contraction. In embodiments, the amount of endotoxin according to USP, for example, USP <85> (incorporated by reference in its entirety) is less than 1.0 EU/mL, less than 0.8 EU/mL or less than 0.75 EU/mL. In embodiments, the osmolarity of a pharmaceutical composition according to USP, for example, USP <785> (incorporated by reference in its entirety) is 350 to 450 mOsm/kg, 370 to 440 mOsm/kg or 390 to 430 mOsm/kg. In embodiments, the pharmaceutical composition contains less than 1200 particles that are greater than 25 μm per container, less than 1000 particles that are greater than 25 μm per container, less than 500 particles that are greater than 25 μm per container or any intermediate value. In embodiments, the pharmaceutical composition contains less than 10,000 particles that are greater than 10 μm per container, less than 8000 particles that are greater than 10 μm per container or less than 600 particles that are greater than 10 pm per container.

In one embodiment, the pharmaceutical composition has a genomic titer of 0.5 to 5.0×10¹³ vg/mL, 1.0 to 4.0×10¹³ vg/mL, 1.5 to 3.0×10¹³ vg/ml or 1.7 to 2.3×10¹³ vg/ml. In one embodiment, the pharmaceutical composition described herein comprises one or more of the following: less than about 0.09 ng benzonase per 1.0×10¹³ vg, less than about 30 pg/g (ppm) of cesium, about 20 to 80 ppm Poloxamer 188, less than about 0.22 ng BSA per 1.0×10¹³ vg, less than about 6.8×10⁵ pg of residual DNA plasmid per 1.0×10¹³ vg, less than about 1.1×10⁵ pg of residual hcDNA per 1.0×10¹³ vg, less than about 4 ng of rHCP per 1.0×10¹³ vg, pH 7.7 to 8.3, about 390 to 430 mOsm/kg, less than about 600 particles that are >25 μm in size per container, less than about 6000 particles that are >10 μm in size per container, about 1.7×10¹³-2.3×10¹³ vg/mL genomic titer, infectious titer of about 3.9×10⁸ to 8.4×10¹⁰ IU per 1.0×10¹³ vg, total protein of about 100-300 μg per 1.0×10¹³ vg, mean survival of >24 days in A7SMA mice with about 7.5×10¹³ vg/kg dose of viral vector, about 70 to 130% relative potency based on an in vitro cell based assay and/or less than about 5% empty capsid. In various embodiments, the pharmaceutical compositions described herein comprise any of the viral particles discussed here, retain a potency of between ±20%, between ±15%, between ±10% or within ±5% of a reference standard. In some embodiments, potency is measured using a suitable in vitro cell assay or in vivo animal model.

Additional methods of preparation, characterization, and dosing AAV particles are taught in WO2019094253, which is incorporated herein by reference in its entirety.

Additional rAAV constructs that can be employed consonant with the invention include those described in Wang et al 2019, available at: //doi.org/10.1038/s41573-019-0012-9, including Table 1 thereof, which is incorporated by reference in its entirety.

### Lipid Nanoparticles

The methods and systems provided by the invention, may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference—e.g., a lipid-containing nanoparticle can comprise one or more of the lipids in table 4 of WO2019217941. Lipid nanoparticles can include additional elements, such as polymers, such as the polymers described in table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid (e.g., encoding the Gene Writer or template nucleic acid) can be varied as desired. For example, the total lipid to nucleic acid (mass or weight) ratio can be from about 10:1 to about 30:1.

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA) described herein (e.g., a template nucleic acid or a nucleic acid encoding a GeneWriter), encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., template RNA and/or a mRNA encoding the Gene Writer polypeptide.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-O13 or 503-O13 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 lZ)-heptatriaconta-6,9,28,3 l-tetraen-l9-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (l3Z,l6Z)-A,A-dimethyl-3-nonyldocosa-l3,l6-dien-l-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety). In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01) e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is; Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-lH-cyclopenta[a]phenanthren-3-yl 3-(1H-imidazol-4-yl)propanoate, e.g., Structure (I) from WO2020/106946 (incorporated by reference herein in its entirety).

Some non-limiting example of lipid compounds that may be used (e.g., in combination with other lipid components) to form lipid nanoparticles for the delivery of compositions described herein, e.g., nucleic acid (e.g., RNA) described herein (e.g., a template nucleic acid or a nucleic acid encoding a GeneWriter) includes,

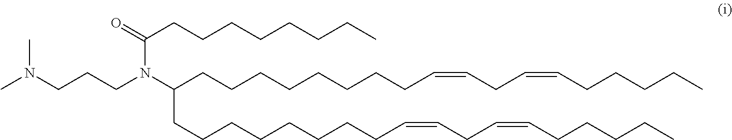 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a GeneWriter composition described herein to the liver and/or hepatocyte cells.

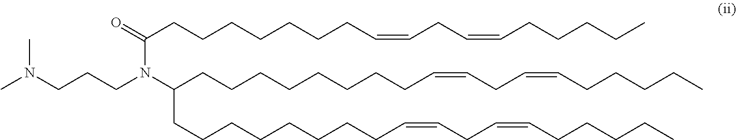 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a GeneWriter composition described herein to the liver and/or hepatocyte cells.

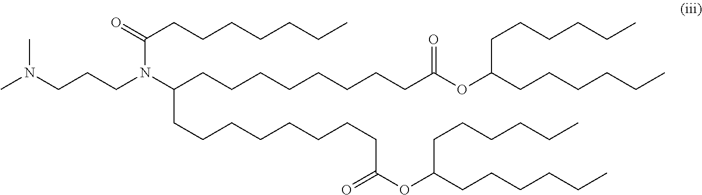 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a GeneWriter composition described herein to the liver and/or hepatocyte cells.

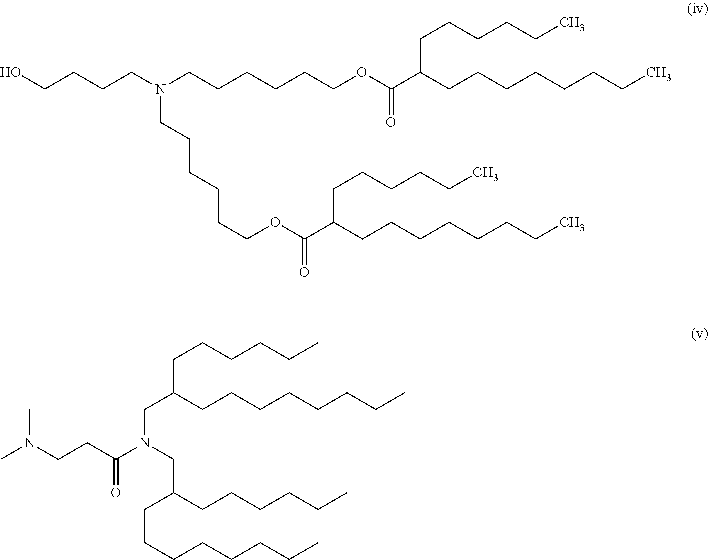

In some embodiments an LNP comprising Formula (v) is used to deliver a GeneWriter composition described herein to the liver and/or hepatocyte cells.

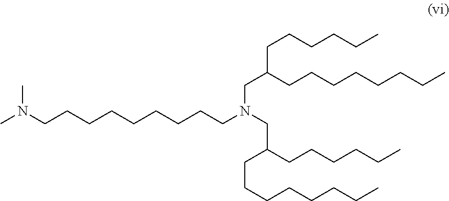 [SMILES: CCCCCCCCC(CCCCCC)CN(CCCCCCCCCN(C)C)CC(CCCCCC)CCCCCCCC]

In some embodiments an LNP comprising Formula (vi) is used to deliver a GeneWriter composition described herein to the liver and/or hepatocyte cells.

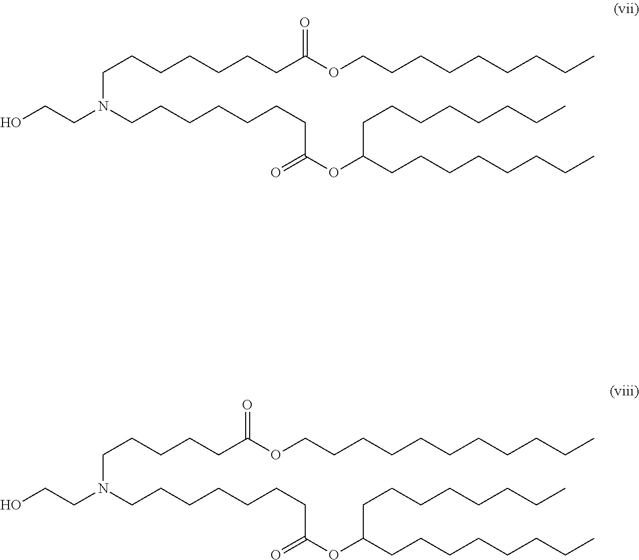 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a GeneWriter composition described herein to the liver and/or hepatocyte cells.

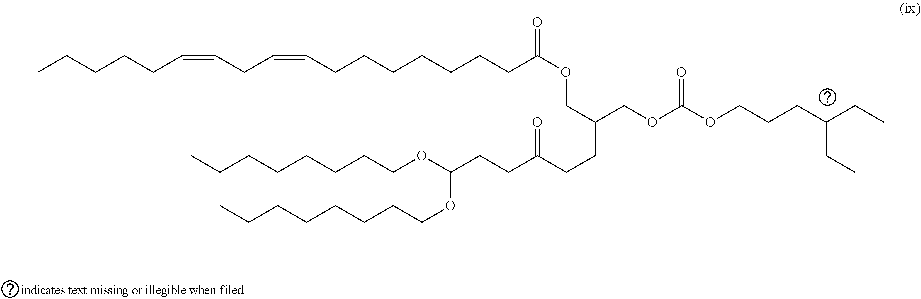 [SMILES: CCCCC/C=C\C/C=C\CCCCCCCC(=O)OCC(CCC(=O)CCC(OCCCCCCCC)OCCCCCCCC)COC(=O)OCCCC(CC)CC]

In some embodiments an LNP comprising Formula (ix) is used to deliver a GeneWriter composition described herein to the liver and/or hepatocyte cells.

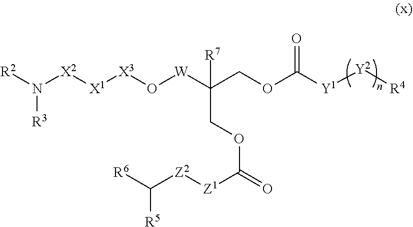

wherein
X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7 membered ring, Y¹ is C2-12 Y is selected from

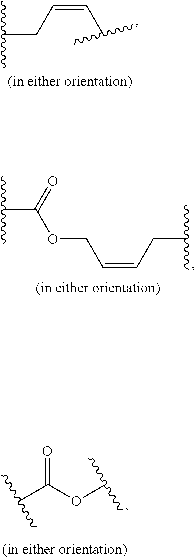 [SMILES: */C=C\C* ; *C/C=C\COC(*)=O ; *OC(*)=O]

n is 0 to 3, R⁴ is Ci-15 alkyl, is Ci-6 alkylene or a direct bond,

### Z² is

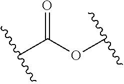 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

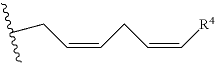

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a GeneWriter composition described herein to the liver and/or hepatocyte cells.

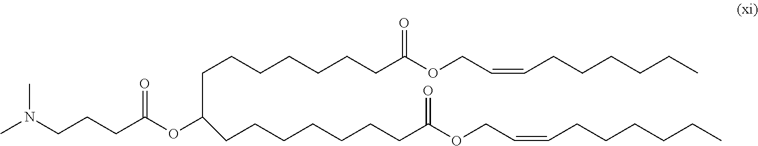 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a GeneWriter composition described herein to the liver and/or hepatocyte cells.

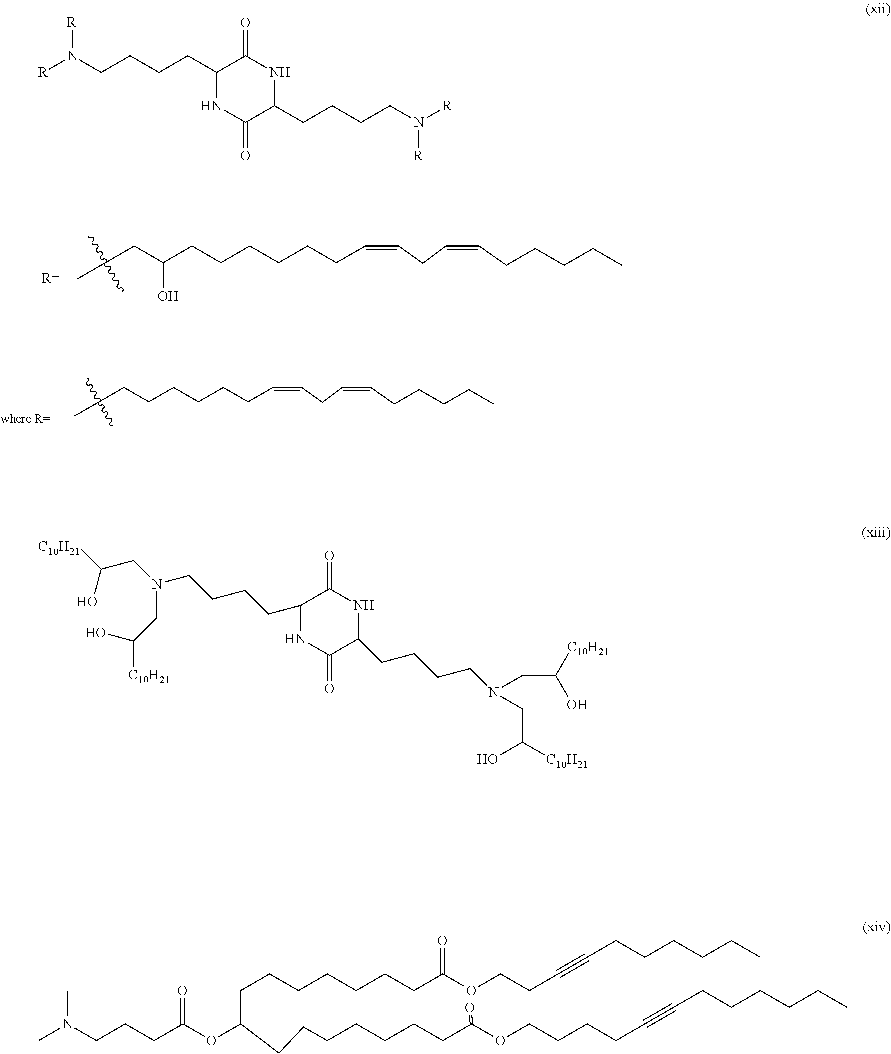

In some embodiments an LNP comprises a compound of Formula (xiii) and a compound of Formula (xiv).

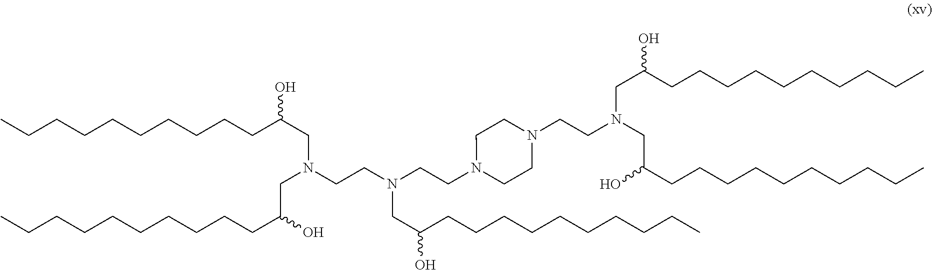 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver a GeneWriter composition described herein to the liver and/or hepatocyte cells.

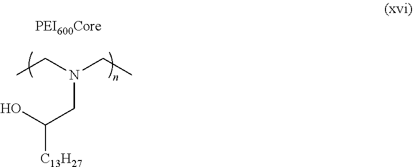

In some embodiments an LNP comprising a formulation of Formula (xvi) is used to deliver a GeneWriter composition described herein to the lung endothelial cells.

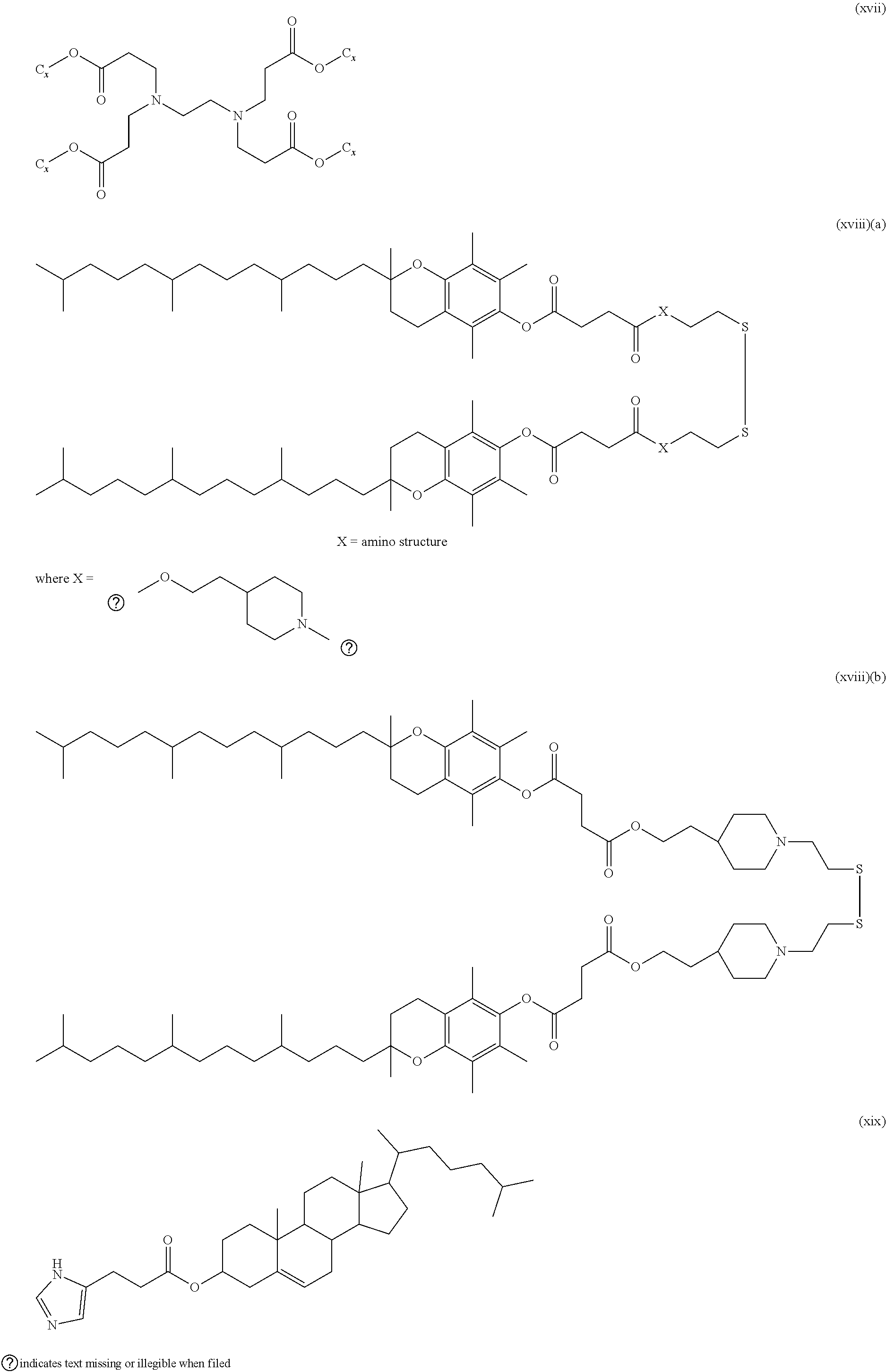

In some embodiments, a lipid compound used to form lipid nanoparticles for the delivery of compositions described herein, e.g., nucleic acid (e.g., RNA) described herein (e.g., a template nucleic acid or a nucleic acid encoding a GeneWriter) is made by one of the following reactions:

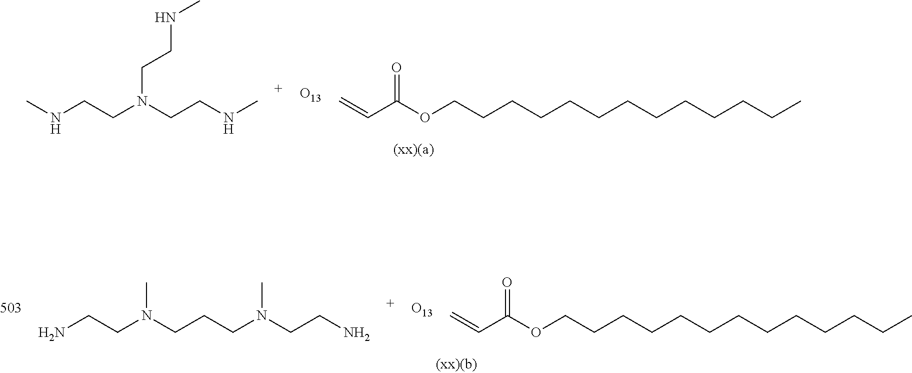

Exemplary non-cationic lipids include, but are not limited to, distearoyl-sn-glycero-phosphoethanolamine, di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), 1,2-dioleoyl-sn-glycero-3-phosphoethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS). In some embodiments, the non-cationic lipid may have the following structure,

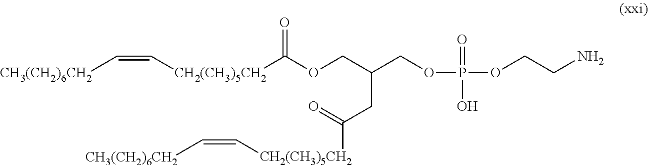

Other examples of non-cationic lipids suitable for use in the lipid nanopartieles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), 1,2-dimyristoyl-sn-glycerol, methoxypoly ethylene glycol (DMG-PEG-2K), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

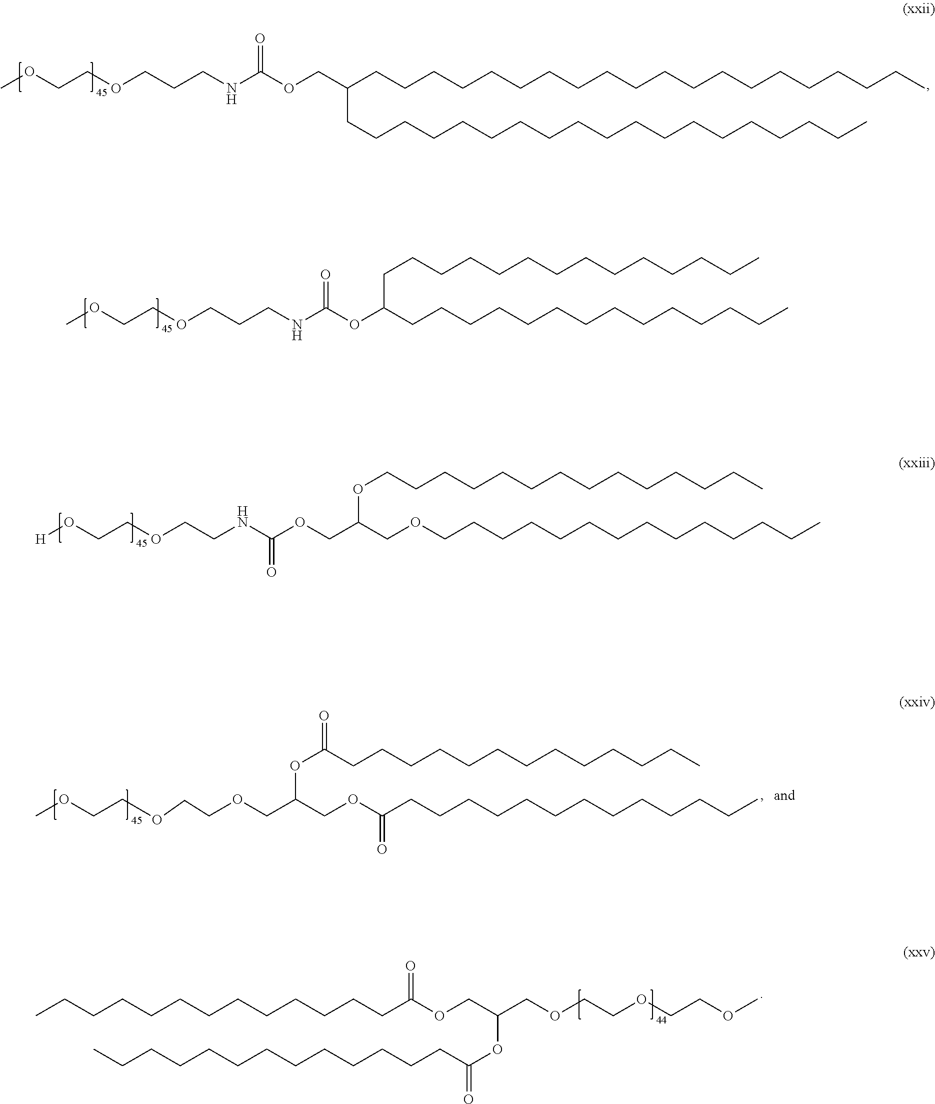

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9 and in WO2020106946A1, the contents of all of which are incorporated herein by reference in their entirety.

In some embodiments an LNP comprises a compound of Formula (xix), a compound of Formula (xxi) and a compound of Formula (xxv). In some embodiments a LNP comprising a formulation of Formula (xix), Formula (xxi) and Formula (xxv) is used to deliver a GeneWriter composition described herein to the lung or pulmonary cells.

In some embodiments, a lipid nanoparticle may comprise one or more cationic lipids selected from Formula (i), Formula (ii), Formula (iii), Formula (vii), and Formula (ix). In some embodiments, the LNP may further comprise one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, a lipid nanoparticle (or a formulation comprising lipid nanoparticles) lacks reactive impurities (e.g., aldehydes or ketones), or comprises less than a preselected level of reactive impurities (e.g., aldehydes or ketones). While not wishing to be bound by theory, in some embodiments, a lipid reagent is used to make a lipid nanoparticle formulation, and the lipid reagent may comprise a contaminating reactive impurity (e.g., an aldehyde or ketone). A lipid regent may be selected for manufacturing based on having less than a preselected level of reactive impurities (e.g., aldehydes or ketones). Without wishing to be bound by theory, in some embodiments, aldehydes can cause modification and damage of RNA, e.g., cross-linking between bases and/or covalently conjugating lipid to RNA (e.g., forming lipid-RNA adducts). This may, in some instances, lead to failure of a reverse transcriptase reaction and/or incorporation of inappropriate bases, e.g., at the site(s) of lesion(s), e.g., a mutation in a newly synthesized target DNA.

In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, a lipid nanoparticle formulation is produced using a lipid reagent comprising: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation is produced using a plurality of lipid reagents, and each lipid reagent of the plurality independently meets one or more criterion described in this paragraph. In some embodiments, each lipid reagent of the plurality meets the same criterion, e.g., a criterion of this paragraph.

In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation comprises: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, one or more, or optionally all, of the lipid reagents used for a lipid nanoparticle as described herein or a formulation thereof comprise: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, total aldehyde content and/or quantity of any single reactive impurity (e.g., aldehyde) species is determined by liquid chromatography (LC), e.g., coupled with tandem mass spectrometry (MS/MS), e.g., according to the method described in Example 5. In some embodiments, reactive impurity (e.g., aldehyde) content and/or quantity of reactive impurity (e.g., aldehyde) species is determined by detecting one or more chemical modifications of a nucleic acid molecule (e.g., an RNA molecule, e.g., as described herein) associated with the presence of reactive impurities (e.g., aldehydes), e.g., in the lipid reagents. In some embodiments, reactive impurity (e.g., aldehyde) content and/or quantity of reactive impurity (e.g., aldehyde) species is determined by detecting one or more chemical modifications of a nucleotide or nucleoside (e.g., a ribonucleotide or ribonucleoside, e.g., comprised in or isolated from a template nucleic acid, e.g., as described herein) associated with the presence of reactive impurities (e.g., aldehydes), e.g., in the lipid reagents, e.g., as described in Example 6. In embodiments, chemical modifications of a nucleic acid molecule, nucleotide, or nucleoside are detected by determining the presence of one or more modified nucleotides or nucleosides, e.g., using LC-MS/MS analysis, e.g., as described in Example 6.

In some embodiments, a nucleic acid (e.g., RNA) described herein (e.g., a template nucleic acid or a nucleic acid encoding a GeneWriter) does not comprise an aldehyde modification, or comprises less than a preselected amount of aldehyde modifications. In some embodiments, on average, a nucleic acid has less than 50, 20, 10, 5, 2, or 1 aldehyde modifications per 1000 nucleotides, e.g., wherein a single cross-linking of two nucleotides is a single aldehyde modification. In some embodiments, the aldehyde modification is an RNA adduct (e.g., a lipid-RNA adduct). In some embodiments, the aldehyde-modified nucleotide is cross-linking between bases. In some embodiments, a nucleic acid (e.g., RNA) described herein comprises less than 50, 20, 10, 5, 2, or 1 cross-links between nucleotide.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 therein). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,l2Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,l2Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, multiple components of a Gene Writer system may be prepared as a single LNP formulation, e.g., an LNP formulation comprises mRNA encoding for the Gene Writer polypeptide and an RNA template. Ratios of nucleic acid components may be varied in order to maximize the properties of a therapeutic. In some embodiments, the ratio of RNA template to mRNA encoding a Gene Writer polypeptide is about 1:1 to 100:1, e.g., about 1:1 to 20:1, about 20:1 to 40:1, about 40:1 to 60:1, about 60:1 to 80:1, or about 80:1 to 100:1, by molar ratio. In other embodiments, a system of multiple nucleic acids may be prepared by separate formulations, e.g., one LNP formulation comprising a template RNA and a second LNP formulation comprising an mRNA encoding a Gene Writer polypeptide. In some embodiments, the system may comprise more than two nucleic acid components formulated into LNPs. In some embodiments, the system may comprise a protein, e.g., a Gene Writer polypeptide, and a template RNA formulated into at least one LNP formulation.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, e.g., Gene Writer polypeptide or mRNA encoding the polypeptide, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TranslT-mRNA Transfection Reagent (Minis Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of Gene Writer LNP may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 10¹¹, 10¹², 10¹³, and 10¹⁴ vg/kg.

### Kits, Articles of Manufacture, and Pharmaceutical Compositions

In an aspect the disclosure provides a kit comprising a Gene Writer or a Gene Writing system, e.g., as described herein. In some embodiments, the kit comprises a Gene Writer polypeptide (or a nucleic acid encoding the polypeptide) and a template RNA (or DNA encoding the template RNA). In some embodiments, the kit further comprises a reagent for introducing the system into a cell, e.g., transfection reagent, LNP, and the like. In some embodiments, the kit is suitable for any of the methods described herein. In some embodiments, the kit comprises one or more elements, compositions (e.g., pharmaceutical compositions), Gene Writers, and/or Gene Writer systems, or a functional fragment or component thereof, e.g., disposed in an article of manufacture. In some embodiments, the kit comprises instructions for use thereof.

In an aspect, the disclosure provides an article of manufacture, e.g., in which a kit as described herein, or a component thereof, is disposed.

In an aspect, the disclosure provides a pharmaceutical composition comprising a Gene Writer or a Gene Writing system, e.g., as described herein. In some embodiments, the pharmaceutical composition further comprises a pharmaceutically acceptable carrier or excipient. In some embodiments, the pharmaceutical composition comprises a template RNA and/or an RNA encoding the polypeptide. In embodiments, the pharmaceutical composition has one or more (e.g., 1, 2, 3, or 4) of the following characteristics:

(a) less than 1% (e.g., less than 0.5%, 0.4%, 0.3%, 0.2%, or 0.1%) DNA template relative to the template RNA and/or the RNA encoding the polypeptide, e.g., on a molar basis;

(b) less than 1% (e.g., less than 0.5%, 0.4%, 0.3%, 0.2%, or 0.1%) uncapped RNA relative to the template RNA and/or the RNA encoding the polypeptide, e.g., on a molar basis;

(c) less than 1% (e.g., less than 0.5%, 0.4%, 0.3%, 0.2%, or 0.1%) partial length RNAs relative to the template RNA and/or the RNA encoding the polypeptide, e.g., on a molar basis;

(d) substantially lacks unreacted cap dinucleotides.

### EXAMPLES

### Example 1: Use of Dominant Negative mRNA for Transient Inhibition of P53

This example describes the use of an mRNA that expresses a dominant negative mutant form of a protein in a host response pathway, such that the effect is a transient inhibition of the pathway. Specifically, a P53 dominant negative mRNA, e.g., GSE56, is used to accomplish this inhibition as described in the literature (Schiroli et al Cell Stem Cell 24, 551-565 (2019)).

In this example, CD34+ hematopoietic stem cells (HSPCs) are acquired frozen from Lonza. Briefly, cells are seeded at a concentration of ˜5×10⁵ cells/mL in serum-free StemSpan medium (StemCell Technologies) supplemented with penicillin, streptomycin, glutamine, 1 mM SR-1 (Biovision), 50 nM UM171 (STEMCell Technologies), 10 mM PGE2 added only at the beginning of the culture (Cayman), and human early-acting cytokines (SCF 100 ng/mL, Flt3-L 100 ng/mL, TPO 20 ng/mL, and IL-6 20 ng/mL; all purchased from Peprotech). HSPCs are cultured in a 5% CO₂ humidified atmosphere at 37° C. After 3 days of stimulation, cells are washed with PBS and electroporated using P3 Primary Cell 4D-Nucleofector X Kit and program EO-100 (Lonza). Cells are electroporated with the following samples:
1. mRNA encoding Gene Writer polypeptide targeting AAVS1+Gene Writer RNA template carrying a GFP reporter gene
2. Condition 1 with genetically inactivated Gene Writer polypeptide
3. Condition 1+GSE56 mRNA (150 mg/mL)
4. Condition 1+control mRNA (RFP) (150 mg/mL)

Gene Writing efficiency is measured from cultured cells 3 days after electroporation by flow cytometry to assay the percentage of cells expressing GFP or by digital droplet PCR analysis with primers and probe on the junction between the template sequence and the targeted locus and on reference sequences as previously described (see PCT/US2019/048607). In some embodiments, Condition 3 will result in an increase in the percentage of cells expressing GFP as measured by flow cytometry and/or the integration efficiency as measured by ddPCR, as compared to Condition 4. In some embodiments, Condition 3 will result in a decrease in cytotoxicity (e.g., using PrestoBlue) at the three day timepoint post transfection, as compared to Condition 4.

### Example 2: Use of siRNA for Transient Inhibition of DNA Repair Pathway to Promote Integration

This example describes the use of a siRNA to modulate a host pathway. Specifically, siRNA targeting BRCA1 (and thus the BRCA1-dependent HR pathway) is used to transiently inhibit this pathway to enhance Gene Writer efficiency.

In this example, HeLa cells are cultured in DMEM with 10% FBS and 1 mM L-glutamine. After seeding, cells are transfected with the following samples:

1. mRNA encoding Gene Writer polypeptide targeting AAVS1+Gene Writer RNA template carrying a GFP reporter gene

2. Condition 1 with genetically inactivated Gene Writer polypeptide

3. Condition 1+siRNA targeting BRCA1 (siBRCA1)

4. Condition 1+control siRNA (SiScramble)

Gene Writing efficiency is measured from cultured cells 3 days after transfection by flow cytometry to assay the percentage of cells expressing GFP or by digital droplet PCR analysis with primers and probe on the junction between the template sequence and the targeted locus and on reference sequences as previously described (see PCT/US2019/048607). In some embodiments, Condition 3 will result in an increase in the percentage of cells expressing GFP as measured by flow cytometry and/or the integration efficiency as measured by ddPCR, as compared to Condition 4. In some embodiments, Condition 3 will result in a decrease in cytotoxicity (e.g., using PrestoBlue) at the three day timepoint post transfection, as compared to Condition 4.

### Example 3: Small Molecule-Mediated Repression of RNA Immune Response

This example describes the use of a small molecule to modulate a host pathway. Specifically, the compound BAY 11-7082 (CAS 19542-67-7) is used as an inhibitor of IKK complex activation, thus decoupling RNA sensing pathways from NFκB activation and an intracellular immune response that would lead to destabilization of RNA. BAY11 was shown previously to improve the expression of OCT4 from synthetic mRNA in human skin cells (Awe et al, Stem Cell Research & Therapy 4 (2013)).

In this example, primary human dermal fibroblasts (ATCC PCS-201-012) are cultured according to ATCC instructions. Cells are nucleofected (Lonza Nucleofector®) with the following samples:
1. mRNA encoding Gene Writer polypeptide targeting AAVS1+Gene Writer RNA template carrying a GFP reporter gene
2. Condition (1) with genetically inactivated Gene Writer polypeptide
3. Condition (1)+BAY 11-7082
4. Condition (1)+BAY 11-7082+IFN-beta

Gene Writing efficiency is measured from cultured cells 3 days after transfection by flow cytometry to assay the percentage of cells expressing GFP or by digital droplet PCR analysis with primers and probe on the junction between the template sequence and the targeted locus and on reference sequences as previously described (see PCT/US2019/048607). In some embodiments, Condition (3) will result in an increase in the percentage of cells expressing GFP as measured by flow cytometry and/or the integration efficiency as measured by ddPCR, as compared to Condition (1). In some embodiments, Condition (3) will result in an increase in the percentage of cells expressing GFP as measured by flow cytometry and/or the integration efficiency as measured by ddPCR, as compared to Condition (4). In some embodiments, Condition (3) will result in a decrease in cytotoxicity (e.g., using PrestoBlue) at the three day timepoint post transfection, as compared to Condition (1). In some embodiments, Condition (3) will result in a decrease in cytotoxicity (e.g., using PrestoBlue) at the three day timepoint post transfection, as compared to Condition (4). In some embodiments, the addition of BAY11 will increase one or both of the expression of the Gene Writer polypeptide, and the stability of the RNA template. In some embodiments, the addition of PAY11 will also reduce cytotoxicity, e.g., cytotoxicity that is due to intracellular immune pathways.

### Example 4: Application of a Virus-Derived Factor to Improve Gene Writer Function

This example describes the use of a virally derived protein, the lentivirus accessory protein viral protein X (Vpx), to modulate a host pathway. Specifically, the HIV-2 protein Vpx has been found to target the sterile alpha motif domain- and HD domain-containing protein 1 (SAMHD1) for proteasomal degradation (Hofmann et al J Virol 86, 12552-12560 (2012)). Without wishing to be bound by theory, SAMHD1 is thought to hydrolyze the cellular deoxynucleotide triphosphate pool to a level below that which is required for reverse transcription, thus inhibiting viruses and transposable elements requiring a reverse transcription step.

In this example, human myeloid U937 cells (ATCC CRL-1593.2) are cultured according to ATCC instructions. U937 cells are transfected with one or a combination of the following:
1. mRNA encoding Gene Writer polypeptide targeting AAVS1+Gene Writer RNA template carrying a GFP reporter gene
2. Condition (1) with genetically inactivated Gene Writer polypeptide
3. Condition (1)+Vpx mRNA
4. Condition (1)+RFP mRNA

Optionally, for Condition (4), cells are first transfected with Vpx mRNA one day prior to the experiment. Gene Writing efficiency is measured from cultured cells 3 days after transfection by flow cytometry to assay the percentage of cells expressing GFP or by digital droplet PCR analysis with primers and probe on the junction between the template sequence and the targeted locus and on reference sequences as previously described (see PCT/US2019/048607). In some embodiments, Condition (3) will result in an increase in the percentage of cells expressing GFP as measured by flow cytometry and/or the integration efficiency as measured by ddPCR, as compared to Condition (1). In some embodiments, Condition (3) will result in an increase in the percentage of cells expressing GFP as measured by flow cytometry and/or the integration efficiency as measured by ddPCR, as compared to Condition (4). In some embodiments, Condition (3) will result in a decrease in cytotoxicity (e.g., using PrestoBlue) at the three day timepoint post transfection, as compared to Condition (1) or Condition (4). In some embodiments, Condition (3) will result in a decrease in cytotoxicity (e.g., using PrestoBlue) at the three day timepoint post transfection, as compared to Condition (4).

### Example 5: Selection of Lipid Reagents with Reduced Aldehyde Content

In this example, lipids are selected for downstream use in lipid nanoparticle formulations containing Gene Writing component nucleic acid(s), and lipids are selected based at least in part on having an absence or low level of contaminating aldehydes. Reactive aldehyde groups in lipid reagents may cause chemical modifications to component nucleic acid(s), e.g., RNA, e.g., template RNA, during LNP formulation. Thus, in some embodiments, the aldehyde content of lipid reagents is minimized.

Liquid chromatography (LC) coupled with tandem mass spectrometry (MS/MS) can be used to separate, characterize, and quantify the aldehyde content of reagents, e.g., as described in Zurek et al. The Analyst 124(9):1291-1295 (1999), incorporated herein by reference. Here, each lipid reagent is subjected to LC-MS/MS analysis. The LC/MS-MS method first separates the lipid and one or more impurities with a C8 HPLC column and follows with the detection and structural determination of these molecules with the mass spectrometer. If an aldehyde is present in a lipid reagent, it is quantified using a staple-isotope labeled (SIL) standard that is structurally identical to the aldehyde, but is heavier due to C13 and N15 labeling. An appropriate amount of the SIL standard is spiked into the lipid reagent. The mixture is then subjected to LC-MS/MS analysis. The amount of contaminating aldehyde is determined by multiplying the amount of SIL standard and the peak ratio (unknown/SIL). Any identified aldehyde(s) in the lipid reagents is quantified as described. In some embodiments, lipid raw materials selected for LNP formulation are not found to contain any contaminating aldehyde content above a chosen level. In some embodiments, one or more, and optionally all, lipid reagents used for formulation comprise less than 3% total aldehyde content. In some embodiments, one or more, and optionally all, lipid reagents used for formulation comprise less than 0.3% of any single aldehyde species. In some embodiments, one or more, and optionally all, lipid reagents used in formulation comprise less than 0.3% of any single aldehyde species and less than 3% total aldehyde content.

### Example 6: Quantification of RNA Modification Caused by Aldehydes During Formulation

In this example, the RNA molecules are analyzed post-formulation to determine the extent of any modifications that may have happened during the formulation process, e.g., to detect chemical modifications caused by aldehyde contamination of the lipid reagents (see, e.g., Example 5).

RNA modifications can be detected by analysis of ribonucleosides, e.g., as according to the methods of Su et al. Nature Protocols 9:828-841 (2014), incorporated herein by reference in its entirety. In this process, RNA is digested to a mix of nucleosides, and then subjected to LC-MS/MS analysis. RNA post-formulation is contained in LNPs and must first be separated from lipids by coprecipitating with GlycoBlue in 80% isopropanol. After centrifugation, the pellets containing RNA are carefully transferred to a new Eppendorf tube, to which a cocktail of enzymes (benzonase, Phosphodiesterase type 1, phosphatase) is added to digest the RNA into nucleosides. The Eppendorf tube is placed on a preheated Thermomixer at 37□ C for 1 hour. The resulting nucleosides mix is directly analyzed by a LC-MS/MS method that first separates nucleosides and modified nucleosides with a C18 column and then detects them with mass spectrometry.

If aldehyde(s) in lipid reagents have caused chemical modification, data analysis will associate the modified nucleoside(s) with the aldehyde(s). A modified nucleoside can be quantified using a SIL standard which is structurally identical to the native nucleoside except heavier due to C13 and N15 labeling. An appropriate amount of the SIL standard is spiked into the nucleoside digest, which is then subjected to LC-MS/MS analysis. The amount of the modified nucleoside is obtained by multiplying the amount of SIL standard and the peak ratio (unknown/SIL). LC-MS/MS is capable of quantifying all the targeted molecules simultaneously.

In some embodiments, the use of lipid reagents with higher contaminating aldehyde content results in higher levels of RNA modification as compared to the use of higher purity lipid reagents as materials during the lipid nanoparticle formulation process. Thus, in preferred embodiments, higher purity lipid reagents are used that result in RNA modification below an acceptable level.

### Example 7: Gene Writer™ Enabling Large Insertion into Genomic DNA

This example describes the use of a Gene Writer™ gene editing system to alter a genomic sequence by insertion of a large string of nucleotides.

In this example, the Gene Writer™ polypeptide, gRNA, and writing template are provided as DNA transfected into HEK293T cells. The Gene Writer™ polypeptide uses a Cas9 nickase for both DNA-binding and endonuclease functions. The reverse transcriptase function is derived from the highly processive RT domain of an R² retrotransposase. The writing template is designed to have homology to the target sequence, while incorporating the genetic payload at the desired position, such that reverse transcription of the template RNA results in the generation of a new DNA strand containing the desired insertion.

To create a large insertion in the human HEK293T cell DNA, the Gene Writer™ polypeptide is used in conjunction with a specific gRNA, which targets the Cas9-containing Gene Writer™ to the target locus, and a template RNA for reverse transcription, which contains an RT-binding motif (3′ UTR from an R² element) for associating with the reverse transcriptase, a region of homology to the target site for priming reverse transcription, and a genetic payload (GFP expression unit). This complex nicks the target site and then performs TPRT on the template, initiating the reaction by using priming regions on the template that are complementary to the sequence immediately adjacent to the site of the nick and copying the GFP payload into the genomic DNA.

After transfection, cells are incubated for three days to allow for expression of the Gene Writing™ system and conversion of the genomic DNA target. After the incubation period, genomic DNA is extracted from cells. Genomic DNA is then subjected to PCR-based amplification using site-specific primers and amplicons are sequenced on an Illumina MiSeq according to manufacturer's protocols. Sequence analysis is then performed to determine the frequency of reads containing the desired edit.

### Example 8: Gene Writers can Integrate Genetic Cargo Independently of the Single-Stranded Template Repair Pathway

This example describes the use of a Gene Writer system in a human cell wherein the single-stranded template repair (SSTR) pathway is inhibited.

In this example, the SSTR pathway will be inhibited using siRNAs against the core components of the pathway: FANCA, FANCD2, FANCE, USP1. Control siRNAs of a non-target control will also be included. 200k U2OS cells will be nucleofected with 30 pmols (1.5 μM) siRNAs, as well as R²Tg driver and transgene plasmids (trans configuration). Specifically, 250 ng of Plasmids expressing R²Tg, control R²Tg with a mutation in the RT domain, or control R²Tg with an endonuclease inactivating mutation) are used in conjunction with transgene at a 1:4 molar ratio (driver to transgene). Transfections of U2OS cells is performed in SE buffer using program DN100. After nucleofection, cells are grown in complete medium for 3 days. gDNA is harvested on day 3 and ddPCR is performed to assess integration at the rDNA site. Transgene integration at rDNA is detected in the absence of core SSTR pathway components.

### Example 9: Formulation of Lipid Nanoparticles Encapsulating Firefly Luciferase mRNA

In this example, a reporter mRNA encoding firefly luciferase was formulated into lipid nanoparticles comprising different ionizable lipids. Lipid nanoparticle (LNP) components (ionizable lipid, helper lipid, sterol, PEG) were dissolved in 100% ethanol with the lipid component. These were then prepared at molar ratios of 50:10:38.5:1.5 using ionizable lipid LIPIDV004 or LIPIDV005 (Table A1), DSPC, cholesterol, and DMG-PEG 2000, respectively. Firefly Luciferase mRNA-LNPs containing the ionizable lipid LIPIDV003 (Table A1) were prepared at a molar ratio of 45:9:44:2 using LIPIDV003, DSPC, cholesterol, and DMG-PEG 2000, respectively. Firefly luciferase mRNA used in these formulations was produced by in vitro transcription and encoded the Firefly Luciferase protein, further comprising a 5′ cap, 5′ and 3′ UTRs, and a polyA tail. The mRNA was synthesized under standard conditions for T7 RNA polymerase in vitro transcription with co-transcriptional capping, but with the nucleotide triphosphate UTP 100% substituted with N1-methyl-pseudouridine triphosphate in the reaction. Purified mRNA was dissolved in 25 mM sodium citrate, pH 4 to a concentration of 0.1 mg/mL.

Firefly Luciferase mRNA was formulated into LNPs with a lipid amine to RNA phosphate (N:P) molar ratio of 6. The LNPs were formed by microfluidic mixing of the lipid and RNA solutions using a Precision Nanosystems NanoAssemblr™ Benchtop Instrument, using the manufacturer's recommended settings. A 3:1 ratio of aqueous to organic solvent was maintained during mixing using differential flow rates. After mixing, the LNPs were collected and dialyzed in 15 mM Tris, 5% sucrose buffer at 4° C. overnight. The Firefly Luciferase mRNA-LNP formulation was concentrated by centrifugation with Amicon 10 kDa centrifugal filters (Millipore). The resulting mixture was then filtered using a 0.2 μm sterile filter. The final LNP was stored at −80° C. until further use.

TABLE A1
Ionizable Lipids used in Example 9

| | | Molec- | |
|---|---|---|---|
| | | ular | |
| LIPID ID | Chemical Name | Weight | Structure |
| LIPIDV003 | (9Z,12Z)-3-((4,4- bis(octyloxy)butanoyl)oxy)-2- ((((3-(diethylamino) propoxy)carbonyl) oxy)methyl)propyl octadeca- 9,12-dienoate | 852.29 | 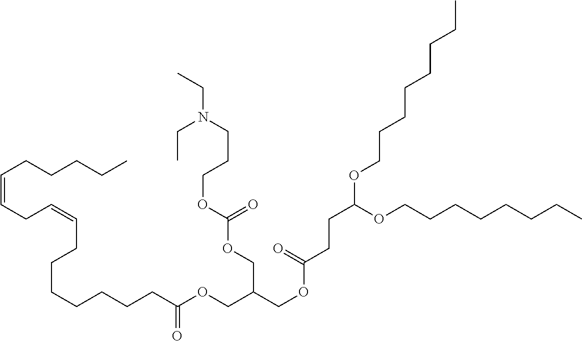 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| LIPIDV004 | Heptadecan-9-yl 8-((2- hydroxyethyl)(8-(nonyloxy)-8- oxooctyl)amino)octanoate | 710.18 | 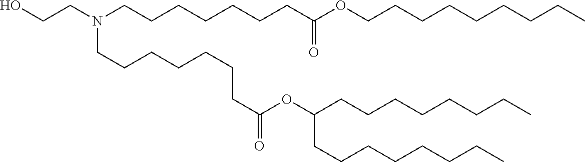 [SMILES: CCCCCCCCCOC(=O)CCCCCCCN(CCO)CCCCCCCC(=O)OC(CCCCCCCC)CCCCCCCC] |
| LIPIDV005 | | 919.56 | 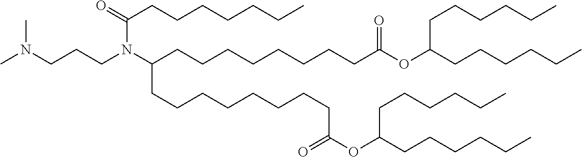 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |

Prepared LNPs were analyzed for size, uniformity, and % RNA encapsulation. The size and uniformity measurements were performed by dynamic light scattering using a Malvern Zetasizer DLS instrument (Malvern Panalytical). LNPs were diluted in PBS prior to being measured by DLS to determine the average particle size (nanometers, nm) and polydispersity index (pdi). The particle sizes of the Firefly Luciferase mRNA-LNPs are shown in Table A2.

TABLE A2
LNP particle size and uniformity

| LNP ID | Ionizable Lipid | Particle Size (nm) | pdi |
|---|---|---|---|
| LNPV019-002 | LIPIDV005 | 77 | 0.04 |
| LNPV006-006 | LIPIDV004 | 71 | 0.08 |
| LNPV011-003 | LIPIDV003 | 87 | 0.08 |

The percent encapsulation of luciferase mRNA was measured by the fluorescence-based RNA quantification assay Ribogreen (ThermoFisher Scientific). LNP samples were diluted in 1×TE buffer and mixed with the Ribogreen reagent per manufacturer's recommendations and measured on a i3 SpectraMax spectrophotomer (Molecular Devices) using 644 nm excitation and 673 nm emission wavelengths. To determine the percent encapsulation, LNPs were measured using the Ribogreen assay with intact LNPs and disrupted LNPs, where the particles were incubated with 1×TE buffer containing 0.2% (w/w) Triton-X100 to disrupt particles to allow encapsulated RNA to interact with the Ribogreen reagent. The samples were again measured on the i3 SpectraMax spectrophotometer to determine the total amount of RNA present. Total RNA was subtracted from the amount of RNA detected when the LNPs were intact to determine the fraction encapsulated. Values were multiplied by 100 to determine the percent encapsulation. The Firefly Luciferase mRNA-LNPs that were measured by Ribogreen and the percent RNA encapsulation is reported in Table A3.

TABLE A3
RNA encapsulation after LNP formulation

| LNP ID | Ionizable Lipid | % mRNA encapsulation |
|---|---|---|
| LNPV019-002 | LIPIDV005 | 98 |
| LNPV006-006 | LIPIDV004 | 92 |
| LNPV011-003 | LIPIDV003 | 97 |

### Example 10: In Vitro Activity Testing of mRNA-LNPs in Primary Hepatocytes

In this example, LNPs comprising the luciferase reporter mRNA were used to deliver the RNA cargo into cells in culture. Primary mouse or primary human hepatocytes were thawed and plated in collagen-coated 96-well tissue culture plates at a density of 30,000 or 50,000 cells per well, respectively. The cells were plated in 1×William's Media E with no phenol red and incubated at 37° C. with 5% CO₂. After 4 hours, the medium was replaced with maintenance medium (1×William's Media E with no phenol containing Hepatocyte Maintenance Supplement Pack (ThermoFisher Scientific)) and cells were grown overnight at 37° C. with 5% CO₂. Firefly Luciferase mRNA-LNPs were thawed at 4° C. and gently mixed. The LNPs were diluted to the appropriate concentration in maintenance media containing 7.5% fetal bovine serum. The LNPs were incubated at 37° C. for 5 minutes prior to being added to the plated primary hepatocytes. To assess delivery of RNA cargo to cells, LNPs were incubated with primary hepatocytes for 24 hours and cells were then harvested and lysed for a Luciferase activity assay. Briefly, medium was aspirated from each well followed by a wash with 1×PBS. The PBS was aspirated from each well and 200 μL passive lysis buffer (PLB) (Promega) was added back to each well and then placed on a plate shaker for 10 minutes. The lysed cells in PLB were frozen and stored at −80° C. until luciferase activity assay was performed.

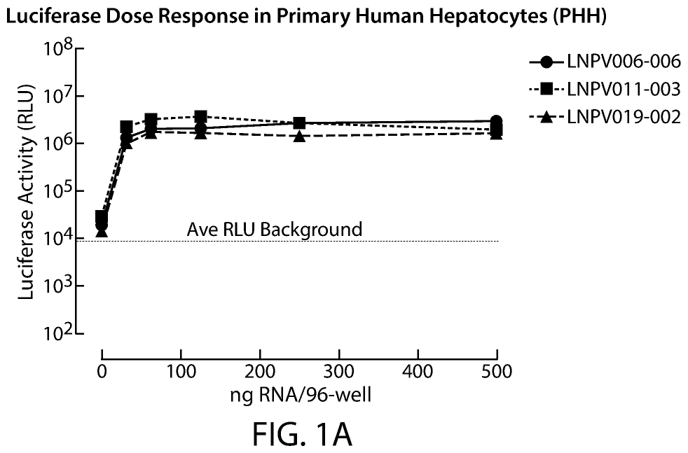

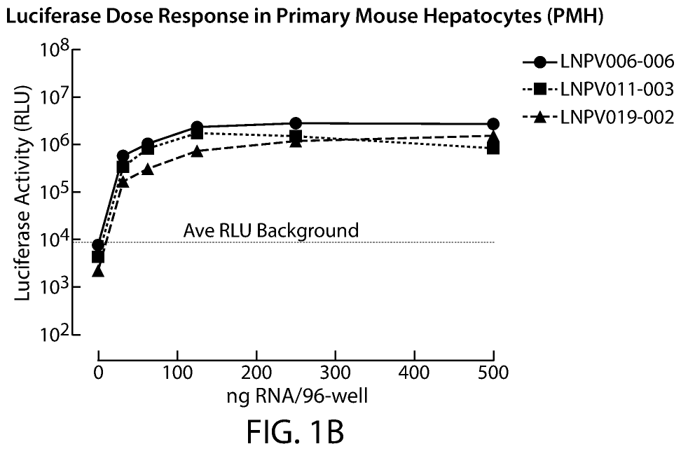

To perform the luciferase activity assay, cellular lysates in passive lysis buffer were thawed, transferred to a round bottom 96-well microtiter plate and spun down at 15,000 g at 4° C. for 3 min to remove cellular debris. The concentration of protein was measured for each sample using the Pierce™ BCA Protein Assay Kit (ThermoFisher Scientific) according to the manufacturer's instructions. Protein concentrations were used to normalize for cell numbers and determine appropriate dilutions of lysates for the luciferase assay. The luciferase activity assay was performed in white-walled 96-well microtiter plates using the luciferase assay reagent (Promega) according to manufacturer's instructions and luminescence was measured using an i3×SpectraMax plate reader (Molecular Devices). The results of the dose-response of Firefly luciferase activity mediated by the Firefly mRNA-LNPs are shown in FIG. 1A and indicate successful LNP-mediated delivery of RNA into primary cells in culture. As shown in FIG. 1A, LNPs formulated as according to Example 9 were analyzed for delivery of cargo to primary human (FIG. 1A) and mouse (FIG. 1B) hepatocytes, as according to Example 10. The luciferase assay revealed dose-responsive luciferase activity from cell lysates, indicating successful delivery of RNA to the cells and expression of Firefly luciferase from the mRNA cargo.

### Example 11: LNP-Mediated Delivery of RNA to the Mouse Liver

To measure the effectiveness of LNP-mediated delivery of firefly luciferase containing particles to the liver, LNPs were formulated and characterized as described in Example 9 and tested in vitro prior (Example 10) to administration to mice. C57BL/6 male mice (Charles River Labs) at approximately 8 weeks of age were dosed with LNPs via intravenous (i.v.) route at 1 mg/kg. Vehicle control animals were dosed i.v. with 300 μL phosphate buffered saline. Mice were injected via intraperitoneal route with dexamethasone at 5 mg/kg 30 minutes prior to injection of LNPs. Tissues were collected at necropsy at or 6, 24, 48 hours after LNP administration with a group size of 5 mice per time point. Liver and other tissue samples were collected, snap-frozen in liquid nitrogen, and stored at −80° C. until analysis.

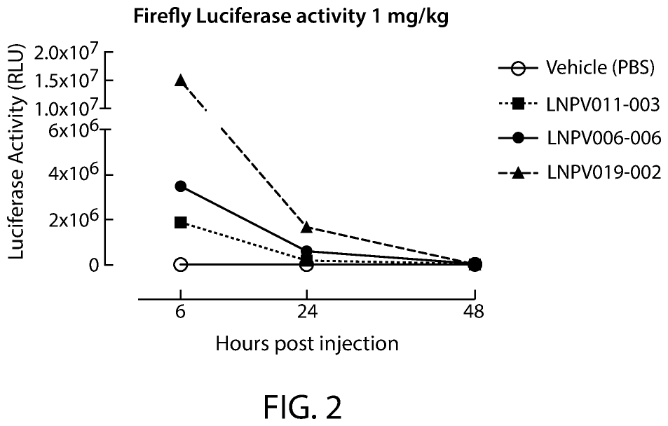

Frozen liver samples were pulverized on dry ice and transferred to homogenization tubes containing lysing matrix D beads (MP Biomedical). Ice-cold 1×luciferase cell culture lysis reagent (CCLR) (Promega) was added to each tube and the samples were homogenized in a Fast Prep-24 5G Homogenizer (MP Biomedical) at 6 m/s for 40 seconds. The samples were transferred to a clean microcentrifuge tube and clarified by centrifugation. Prior to luciferase activity assay, the protein concentration of liver homogenates was determined for each sample using the Pierce™ BCA Protein Assay Kit (ThermoFisher Scientific) according to the manufacturer's instructions. Luciferase activity was measured with 200 μg (total protein) of liver homogenate using the luciferase assay reagent (Promega) according to manufacturer's instructions using an i3X SpectraMax plate reader (Molecular Devices). Liver samples revealed successful delivery of mRNA by all lipid formulations, with reporter activity following the ranking LIPIDV005>LIPIDV004>LIPIDV003 (FIG. 2). As shown in FIG. 2, Firefly luciferase mRNA-containing LNPs were formulated and delivered to mice by iv, and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours. Post-administration. This assay validated the use of these ionizable lipids and their respective formulations for RNA systems for delivery to the liver.

Without wishing to be limited by example, the lipids and formulations described in this example are support the efficacy for the in vivo delivery of other RNA molecules beyond a reporter mRNA. All-RNA Gene Writing systems can be delivered by the formulations described herein. For example, all-RNA systems employing a Gene Writer polypeptide mRNA, Template RNA, and an optional second-nick gRNA are described for editing the genome in vitro by nucleofection, by using modified nucleotides, by lipofection, and for editing cells, e.g., primary T cells. As described in this application, these all-RNA systems have many unique advantages in cellular immunogenicity and toxicity, which is of importance when dealing with more sensitive primary cells, especially immune cells, e.g., T cells, as opposed to immortalized cell culture cell lines. Further, it is contemplated that these all RNA systems could be targeted to alternate tissues and cell types using novel lipid delivery systems as referenced herein, e.g., for delivery to the liver, the lungs, muscle, immune cells, and others, given the function of Gene Writing systems has been validated in multiple cell types in vitro here, and the function of other RNA systems delivered with targeted LNPs is known in the art. The in vivo delivery of Gene Writing systems has potential for great impact in many therapeutic areas, e.g., correcting pathogenic mutations), instilling protective variants, and enhancing cells endogenous to the body, e.g., T cells. Given an appropriate formulation, all-RNA Gene Writing is conceived to enable the manufacture of cell-based therapies in situ in the patient.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

1. A method of modifying a target DNA molecule in a mammalian host cell, the method comprising:
a) contacting the host cell with a gene modifying system; and
b) contacting the host cell with a host response modulator,
wherein the gene modifying system comprises a Gene Writer polypeptide, or a nucleic acid encoding the Gene Writer polypeptide, and a template nucleic acid, the template nucleic acid comprising i) a sequence that binds the Gene Writer polypeptide and ii) a heterologous object sequence;
wherein the Gene Writer polypeptide comprises a Cas9 nickase and the RT domain from a retrovirus.

2. A kit comprising:
a) a gene modifying system that comprises a Gene Writer polypeptide, or a nucleic acid encoding the Gene Writer polypeptide, and a template nucleic acid, the template nucleic acid comprising i) a sequence that binds the Gene Writer polypeptide and ii) a heterologous object sequence; and
b) a host response modulator;
wherein the Gene Writer polypeptide comprises a Cas9 nickase and the RT domain from a retrovirus.

3. A composition comprising:
a) a gene modifying system that comprises a Gene Writer polypeptide, or a nucleic acid encoding the Gene Writer polypeptide, and a template nucleic acid, the template nucleic acid comprising i) a sequence that binds the Gene Writer polypeptide and ii) a heterologous object sequence; and
b) a host response modulator;
wherein the Gene Writer polypeptide comprises a Cas9 nickase and the RT domain from a retrovirus.

4. The method of claim 1, wherein the host response modulator:
(i) comprises an antibody, a polypeptide (e.g., a dominant negative mutant of a polypeptide in a host response pathway), or a nucleic acid (e.g., an RNAi molecule);
(ii) is a host response inhibitor; or
(iii) is a host response stimulator.

5-6. (canceled)

7. The method of claim 1, wherein the contacting of the host cell with the Gene Writer polypeptide and the host response modulator results in increased levels of the heterologous object sequence in host cell genome compared to an otherwise similar cell not contacted with the host response modulator, e.g., wherein the number of copies of heterologous object sequence in the genome of a population of host cells is at least 5%, 10%, 20%, 30%, 40%, 50%, 60%, 70%, 80%, 90%, or 100% higher, or at least 2-fold, 5-fold, or 10-fold higher, than the number of copies of heterologous object sequence in the genome of otherwise similar cells that were contacted with the gene modifying system but not with the host response modulator.

8. The method of claim 1, wherein the host response modulator inhibits:
(i) activity of: one or more: DNA damage response pathway proteins, anti-viral response pathway proteins, protein inhibitors of mRNA therapy, DNA sensing proteins, mobile element restriction proteins, proinflammatory proteins, or a combination thereof, e.g., by at least: 5, 10, 15, 20, 25, 30, 35, 40, 45, 50, 55, 60, 65, 70, 75, 80, 85, 90, 95, 96, 97, 98, 99%, or more, e.g., by at least: 2, 4, 8, 10, 20, 50, 100, 200, 500, or 1000-fold, relative to control;
(ii) one or more proteins involved in Homology Directed Repair (HDR) (e.g., PARP1, PARP2, MRE11, RAD50, NBS1, BARD1, BRCA2, BRCA1, RTS, RECQ5, RPA3, PP4, PALB2, DSS1, RAD51, BACH1, FANCJ, Topbp1, TOPO III, FEN1, MUS81, EME1, SLX1, SLX4, RECQ1, WRN, CtIP, EXO1, DNA2, MRN complex), Fanconi Anaemia complementation group (FANC) (e.g., FANCA, FANCB, FANCC, FANCD1, FANCD2, FANCE, FANCF, FANCG, FANCI, FANCJ, FANCL, FANCM, FANCN, FANCO, FANCP, FANCQ, FANCR, FANCS, FANCT), Anti-HDR (e.g., FBH1, RECQ5, BLM, FANCJ, PARI, RECQ1, WRN, RTEL, RAP80, miR-155, miR-545, miR-107, miR-1255, miR-148, miR-193), Single Strand Annealing (SSA) (e.g., RPA, RPA1, RPA2, RPA3, RAD52, XPF, ERCC1), Canonical Non-Homologous End Joining (C-NHEJ) (e.g., DNA-PK, DNA-PKcs, 53BP1, XRCC4, LIG4, XLF, ARTEMIS, APLF, PNK, Rif1, PTIP, DNA polymerase, Ku70, Ku80), Alternative Non-Homologous End Joining (Alt-NHEJ) (PARP1, PARP2, CtIP, LIG3, MRE11, Rad50, Nbs1, XPF, ERCC1, LIG1, DNA Polymerase θ, MRN complex, XRCC1), Mismatch Repair (MMR) (e.g., EXO1, MSH2, MSH3, MSH6, MLH1, PMS2, MLH3, DNA polymerase delta, RPA, RFC, LIG1), Nucleotide Excision Repair (NER) (e.g., XPF, XPG, ERCC1, TTDA, UVSSA, USP7, CETN2, RAD23B, UV-DDB, CAK subcomplex, RPA, PCNA), Base Excision Repair (BER) (e.g., APE1, Pol β, Pol δ, Pol ε, XRCC1, LIG3, FEN-1, PCNA, RECQL4, WRN, MYH, PNKP, APTX), Single-Strand Break Repair (SSBR) (e.g., PARP1, PARP2, PARG, XRCC1, DNA pol β, DNA pol δ, DNA pol ε, PCNA, LIG1, PNK, PNKP, APE1, APTX, APLF, TDP1, LIG3, FEN1, CtIP, MRN, ERCC1), chromatin modification (e.g., Ezh2, HDAC-Class I, HDAC-Class IIKDM4A/JMJD2A, FACT), cell cycle (e.g., CDK1, CDC7, ATM, ATR), Translesion DNA Synthesis (TLS) (e.g., UBC13, or RAD18), cellular metabolism (e.g., mTOR), cell death (e.g., p53), or RNA:DNA resolution/R-Loop (e.g., SETX, RNH1, or RNH2), or Type I Interferon response (e.g., caspase-1, IFNα, IFNβ, NF-κB, TNF-α);
(iii) one or more proteins involved in anti-viral response, e.g., ZAP, TREX1, MOV10, hnRNPL, SAMHD1, RNase L, Melatonin receptor 1, APOBEC3 (A3) (e.g., A3 inhibitor Vif), SAMHD1 (e.g., SAMHD1 inhibitor Vpx), BST-2/tetherin (Vpu), or any combination thereof;
(iv) one or more proteins involved in inhibition of mRNA therapy;
(v) one or more proteins involved in RNA sensing and response, e.g., TLR3, TLR4, TLR7, TLR8, MyD88, TRIF, IKK, NF-κB, IRF3, IRF7, IFN-α, IFN-β, TNFα, IL-6, IL-12, JAK-1, TYK-2, STAT1, STAT2, IRF-9, PKR, OAS, ADAR, RIG-I, MDA5, LGP2, MAVS, NLRP3, NOD2, or caspase 1, or any combination thereof;
(vi) RIG-I, e.g., wherein the host response modulator comprises a HIV-1 protease, or a functional fragment or variant thereof;
(vii) IKK complex, e.g., the host response modulator inhibits IKK, e.g., wherein the host response modulator comprises BAY11;
(viii) TRIF, e.g., wherein the host response modulator comprises Pepinh-TRIF;
(ix) MyD88 complex, e.g., inhibits MyD88, wherein the host response modulator comprises Pepinh-MYD;
(x) IFN pathway, e.g., inhibits an IFN, wherein the host response modulator comprises an interferon-binding protein, e.g., Vaccinia B18R;
(xi) endosomal maturation, e.g., wherein the host response modulator comprises chloroquine or Bafilomycin A1, or a combination thereof;
(xii) one or more proteins involved in DNA sensing, e.g., cGAS, STING, TBK1, IRF3, DNA-PK, HSPA8/HSC70, or any combination thereof;
(xiii) cGAS, e.g., wherein the host response modulator comprises PF-06928215, RU.365, RU.521, RU.521, or G150, or any combination thereof; or
(xiv) STING, e.g., wherein the host response modulator comprises C-176, C-178, H151, the cyclopeptide astin C, Astin C, Screening Hit 1, Compound 13, E1A (hAd5), E7 (HPV18), or any combination thereof.

9-22. (canceled)

23. The method of claim 1, wherein the host response modulator inhibits:
(i) TBK1, e.g., wherein host response modulator comprises BX795, Tozasertib, Tozasertib-15a, 20b, azabenzimidazole hit 1a, CYT387, Domainex, Amgen Compound II, MRT67307, or AZ13102909 or any combination thereof;
(ii) IRF3, e.g., wherein host response modulator comprises BX795, Tozasertib, Tozasertib-15a, 20b, azabenzimidazole hit 1a, CYT387, Domainex, Amgen Compound II, MRT67307, AZ13102909) or any combination thereof; or
(iii) DNA-PK, e.g., wherein host response modulator comprises Nu-7441, hAd5 E1A, or HSV-1 ICP0 or any combination thereof.

24-25. (canceled)

26. The method of claim 1, wherein the host response modulator is an immunosuppressive agent, e.g., an immunosuppressive agent that reduces a host immune response to a viral polypeptide, e.g., a viral polypeptide involved in delivery of the gene modifying system, e.g., an AAV polypeptide, e.g., an AAV capsid protein;
optionally wherein the immunosuppressive agent is a steroid, an anti-inflammatory agent, cyclosporine (e.g., cyclosporine A), mycophenolate, Rituximab, or a derivative thereof.

27-29. (canceled)

30. The method of claim 1, wherein the host response modulator inhibits:
(i) one or more proteins involved in mobile element restriction, e.g., p53, BRCA1, or a combination thereof;
(ii) p53, e.g., wherein the host response modulator comprises a nucleic acid encoding GSE56, e.g., GSE56 mRNA (dominant negative); or
(iii) one or more proteins involved in the Type I interferon response, e.g., IFNα, IFNβ, NF-κB, TNF-α.

31-32. (canceled)

33. The method of claim 1, wherein the host response modulator comprises:
(i) an immune suppressant;
(ii) a protein that bends DNA (e.g., HMGB1), or nucleic acid encoding the protein, or an agent that upregulates expression of a gene encoding the protein (e.g., by CRISPRa);
(iii) a protein that stimulates cell cycle progression (e.g., PDGF), or a nucleic acid encoding the protein, or an agent that upregulates expression of a gene encoding the protein (e.g., by CRISPRa); or
(iv) a protein that increases biosynthesis of deoxynucleotides (e.g., increase biosynthesis of dNDPs from rNDPs) (e.g., Ribonucleotide reductase (RNR)), or a nucleic acid encoding the protein, or an agent that upregulates expression of a gene encoding the protein (e.g., by CRISPRa).

34-36. (canceled)

37. The method of claim 1, wherein the Gene Writer polypeptide comprises a reverse transcriptase domain and endonuclease domain, wherein optionally the reverse transcriptase domain and endonuclease domain are heterologous to each other.

38. The method of claim 1, wherein the template nucleic acid further comprises a gRNA region, e.g., a gRNA region that binds a target site.

39. The method of claim 1, wherein the mammalian host cell is a primate cell, such as a human cell.

40. The method of claim 1, wherein the contacting occurs:
(i) ex vivo, e.g., wherein the mammalian host cell's DNA is modified ex vivo; or
(ii) in vivo, e.g., wherein the mammalian host cell's DNA is modified in vivo.

41. (canceled)

42. The method of claim 1, wherein the gene modifying system and host response modulators are provided access to the host cell:
(i) substantially concurrently, e.g., by concurrent administration; or
(ii) sequentially, e.g., by sequential administration, e.g., wherein the host response modulator is provided before the gene modifying system or wherein the gene modifying system is provided before the host response modulator.

43. (canceled)

44. The method of claim 1, wherein the cell is contacted with the host response modulator a plurality of times, e.g., wherein a subject receives multiple administrations of the host response modulator.

45. The method of claim 1, wherein contacting the host cell with the gene modifying system comprises:
(i) allowing the gene modifying system to access the host cell;
(ii) administering the gene modifying system to a subject that has the host cell;
(iii) allowing the gene modifying system to access the host cell; and/or
(iv) administering the host response modulator to a subject that has the host cell.

46-48. (canceled)

49. The method of claim 1, which comprises contacting the host cell with a second host response modulator.

50. The method of claim 1, wherein contacting the host cell with the gene modifying system comprises contacting the host cell with a nucleic acid (e.g., DNA or RNA) encoding the Gene Writer polypeptide under conditions that allow for production of the Gene Writer polypeptide.

51. The method of claim 1, wherein relative to a similar method omitting step (b), the method results in reduced cytotoxicity to the mammalian host cell or a mammalian subject in which the mammalian host cell is disposed.

52. The method of claim 1, wherein the host response modulator inhibits:
(i) FANCA,
(ii) SAMHD1, or
(iii) BRCA2.

53. The method of claim 1, wherein the host response modulator stimulates:
(i) RNASEH2A, or
(ii) RAD18.

54. The method of claim 1, wherein the host response modulator is rucaparib.


SEQUENCE LISTING (466 sequences)

SEQ ID NO: 1 - protein, 1367 aa, synthetic construct
  REGION 1..1367: note = Description of Artificial Sequence: Syntheticpolypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 672 aa, Murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 3 - protein, 671 aa, synthetic construct
  REGION 1..671: note = Description of Artificial Sequence: Syntheticpolypeptide
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLL

SEQ ID NO: 4 - protein, 660 aa, Murine leukemia virus
TLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAA

SEQ ID NO: 5 - protein, 672 aa, synthetic construct
  REGION 1..672: note = Description of Artificial Sequence: Syntheticpolypeptide
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLI

SEQ ID NO: 6 - protein, 33 aa, synthetic construct
  REGION 1..33: note = Description of Artificial Sequence: Syntheticpolypeptide
SGGSSGGSSGSETPGTSESATPESSGGSSGGSS

SEQ ID NO: 7 - protein, 1369 aa, synthetic construct
  REGION 1..1369: note = Description of Artificial Sequence: Syntheticpolypeptide
SMDKKYSIGLAIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - DNA, 76 bp, synthetic construct
  misc_feature 1..76: note = Description of Artificial Sequence: Syntheticoligonucleotide
  source 1..76: mol_type = other DNA
gttttagagctagaaatagcaagttaaaataaggctagtccgttatcaacttgaaaaagtgggaccgagtcggtcc

SEQ ID NO: 9 - protein, 30 aa, synthetic construct
  REGION 1..30: note = Description of Artificial Sequence: Syntheticpolypeptide
MDSLLMNRRKFLYQFKNVRWAKGRRETYLC

SEQ ID NO: 10 - protein, 28 aa, synthetic construct
  REGION 1..28: note = Description of Artificial Sequence: Syntheticpeptide
PKKRKVEGADKRTADGSEFESPKKKRKV

SEQ ID NO: 11 - protein, 22 aa, synthetic construct
  REGION 1..22: note = Description of Artificial Sequence: Syntheticpeptide
RKSGKIAAIWKRPRKPKKKRKV

SEQ ID NO: 12 - protein, 18 aa, synthetic construct
  REGION 1..18: note = Description of Artificial Sequence: Syntheticpeptide
KRTADGSEFESPKKKRKV

SEQ ID NO: 13 - protein, 17 aa, synthetic construct
  REGION 1..17: note = Description of Artificial Sequence: Syntheticpeptide
KKTELQTTNAENKTKKL

SEQ ID NO: 14 - protein, 19 aa, Homo sapiens
KRGINDRNFWRGENGRKTR

SEQ ID NO: 15 - protein, 16 aa, unidentified
  REGION 1..16: note = Description of Unknown:Nucleoplasmin bipartite NLS sequence
KRPAATKKAGQAKKKK

SEQ ID NO: 16 - protein, 29 aa, unidentified
  REGION 1..29: note = Description of Unknown:Nucleoplasmin bipartite NLS sequence
PKKKRKVEGADKRTADGSEFESPKKKRKV

SEQ ID NO: 17 - protein, 25 aa, synthetic construct
  REGION 1..25: note = Description of Artificial Sequence: Syntheticpeptide
KRASSQALGTIPKRRSSSRFIKRKK

SEQ ID NO: 18 - protein, 7 aa, synthetic construct
  REGION 1..7: note = Description of Artificial Sequence: Syntheticpeptide
RKKRKKK

SEQ ID NO: 19 - protein, 4 aa, unidentified
  REGION 1..4: note = Description of Unknown:spCas9 binding domain sequence
VRQR

SEQ ID NO: 20 - protein, 4 aa, unidentified
  REGION 1..4: note = Description of Unknown:spCas9 binding domain sequence
VRER

SEQ ID NO: 21 - protein, 6 aa, unidentified
  REGION 1..6: note = Description of Unknown:spCas9 binding domain sequence
MQKSER

SEQ ID NO: 22 - protein, 6 aa, unidentified
  REGION 1..6: note = Description of Unknown:spCas9 binding domain sequence
LRKIQK

SEQ ID NO: 23 - protein, 6 aa, unidentified
  REGION 1..6: note = Description of Unknown:spCas9 binding domain sequence
LRVSQL

SEQ ID NO: 24 - protein, 16 aa, synthetic construct
  REGION 1..16: note = Description of Artificial Sequence: Syntheticpeptide
SGSETPGTSESATPES

SEQ ID NO: 25 - protein, 4 aa, synthetic construct
  REGION 1..4: note = Description of Artificial Sequence: Syntheticpeptide
SGGS

SEQ ID NO: 26 - protein, 4 aa, synthetic construct
  REGION 1..4: note = Description of Artificial Sequence: Syntheticpeptide
GGGS

SEQ ID NO: 27 - protein, 5 aa, synthetic construct
  REGION 1..5: note = Description of Artificial Sequence: Syntheticpeptide
GGGGS

SEQ ID NO: 28 - protein, 5 aa, synthetic construct
  REGION 1..5: note = Description of Artificial Sequence: Syntheticpeptide
EAAAK

SEQ ID NO: 29 - DNA, 306 bp, synthetic construct
  misc_feature 1..306: note = Description of Artificial Sequence: Syntheticpolynucleotide
  source 1..306: mol_type = other DNA
tgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaat

SEQ ID NO: 30 - protein, 102 aa, synthetic construct
  REGION 1..102: note = Description of Artificial Sequence: Syntheticpolypeptide
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 31 - DNA, 108 bp, synthetic construct
  misc_feature 1..108: note = Description of Artificial Sequence: Syntheticpolynucleotide
  source 1..108: mol_type = other DNA
atgatcaagatagctacaaggaagtatcttggcaaacaaaacgtttatgatattggagtcgaaagagatcacaactttgctctgaagaacggattcatagcttctaat

SEQ ID NO: 32 - protein, 36 aa, synthetic construct
  REGION 1..36: note = Description of Artificial Sequence: Syntheticpolypeptide
MIKIATRKYLGKQNVYDIGVERDHNFALKNGFIASN

SEQ ID NO: 33 - DNA, 303 bp, synthetic construct
  misc_feature 1..303: note = Description of Artificial Sequence: Syntheticpolynucleotide
  source 1..303: mol_type = other DNA
tgcctgtcttatgataccgagatacttaccgttgaatatggcttcttgcctattggaaagattgtcgaagagagaattgaatgcacagtatatactgtagacaagaatggtttcgtttacacacagcccattgctcaatggcacaatcgcggcgaacaagaagtatttgagtactgtctcgaggatggaagcatcatacgagcaactaaagatcataaattcatgaccactgacgggcagatgttgccaatagatgagatattcgagcggggcttggatctcaaacaagtggatggattgcca

SEQ ID NO: 34 - protein, 101 aa, synthetic construct
  REGION 1..101: note = Description of Artificial Sequence: Syntheticpolypeptide
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 35 - DNA, 159 bp, synthetic construct
  misc_feature 1..159: note = Description of Artificial Sequence: Syntheticpolynucleotide
  source 1..159: mol_type = other DNA
atgaagaggactgccgatggatcagagtttgaatctcccaagaagaagaggaaagtaaagataatatctcgaaaaagtcttggtacccaaaatgtctatgatattggagtggagaaagatcacaacttccttctcaagaacggtctcgtagccagcaac

SEQ ID NO: 36 - protein, 53 aa, synthetic construct
  REGION 1..53: note = Description of Artificial Sequence: Syntheticpolypeptide
MKRTADGSEFESPKKKRKVKIISRKSLGTQNVYDIGVEKDHNFLLKNGLVASN

SEQ ID NO: 37-43 - skipped (7 entries)

SEQ ID NO: 44 - DNA, 60 bp, synthetic construct
  misc_feature 1..60: note = Description of Artificial Sequence: Syntheticoligonucleotide
  source 1..60: mol_type = other DNA
tagcaagttaaaataaggctagtccgttatcaacttgaaaaagtggcaccgagtcggtgc

SEQ ID NO: 45-101 - skipped (57 entries)

SEQ ID NO: 102 - protein, 6 aa, synthetic construct
  REGION 1..6: note = Description of Artificial Sequence: Syntheticpeptide
GGSGGS

SEQ ID NO: 103 - protein, 9 aa, synthetic construct
  REGION 1..9: note = Description of Artificial Sequence: Syntheticpeptide
GGSGGSGGS

SEQ ID NO: 104 - protein, 12 aa, synthetic construct
  REGION 1..12: note = Description of Artificial Sequence: Syntheticpeptide
GGSGGSGGSGGS

SEQ ID NO: 105 - protein, 15 aa, synthetic construct
  REGION 1..15: note = Description of Artificial Sequence: Syntheticpeptide
GGSGGSGGSGGSGGS

SEQ ID NO: 106 - protein, 18 aa, synthetic construct
  REGION 1..18: note = Description of Artificial Sequence: Syntheticpeptide
GGSGGSGGSGGSGGSGGS

SEQ ID NO: 107 - protein, 5 aa, synthetic construct
  REGION 1..5: note = Description of Artificial Sequence: Syntheticpeptide
GGGGS

SEQ ID NO: 108 - protein, 10 aa, synthetic construct
  REGION 1..10: note = Description of Artificial Sequence: Syntheticpeptide
GGGGSGGGGS

SEQ ID NO: 109 - protein, 15 aa, synthetic construct
  REGION 1..15: note = Description of Artificial Sequence: Syntheticpeptide
GGGGSGGGGSGGGGS

SEQ ID NO: 110 - protein, 20 aa, synthetic construct
  REGION 1..20: note = Description of Artificial Sequence: Syntheticpeptide
GGGGSGGGGSGGGGSGGGGS

SEQ ID NO: 111 - protein, 25 aa, synthetic construct
  REGION 1..25: note = Description of Artificial Sequence: Syntheticpeptide
GGGGSGGGGSGGGGSGGGGSGGGGS

SEQ ID NO: 112 - protein, 30 aa, synthetic construct
  REGION 1..30: note = Description of Artificial Sequence: Syntheticpolypeptide
GGGGSGGGGSGGGGSGGGGSGGGGSGGGGS

SEQ ID NO: 113 - skipped

SEQ ID NO: 114 - protein, 4 aa, synthetic construct
  REGION 1..4: note = Description of Artificial Sequence: Syntheticpeptide
GGGG

SEQ ID NO: 115 - protein, 5 aa, synthetic construct
  REGION 1..5: note = Description of Artificial Sequence: Syntheticpeptide
GGGGG

SEQ ID NO: 116 - protein, 6 aa, synthetic construct
  REGION 1..6: note = Description of Artificial Sequence: Syntheticpeptide
GGGGGG

SEQ ID NO: 117 - protein, 7 aa, synthetic construct
  REGION 1..7: note = Description of Artificial Sequence: Syntheticpeptide
GGGGGGG

SEQ ID NO: 118 - protein, 8 aa, synthetic construct
  REGION 1..8: note = Description of Artificial Sequence: Syntheticpeptide
GGGGGGGG

SEQ ID NO: 119 - skipped

SEQ ID NO: 120 - protein, 6 aa, synthetic construct
  REGION 1..6: note = Description of Artificial Sequence: Syntheticpeptide
GSSGSS

SEQ ID NO: 121 - protein, 9 aa, synthetic construct
  REGION 1..9: note = Description of Artificial Sequence: Syntheticpeptide
GSSGSSGSS

SEQ ID NO: 122 - protein, 12 aa, synthetic construct
  REGION 1..12: note = Description of Artificial Sequence: Syntheticpeptide
GSSGSSGSSGSS

SEQ ID NO: 123 - protein, 15 aa, synthetic construct
  REGION 1..15: note = Description of Artificial Sequence: Syntheticpeptide
GSSGSSGSSGSSGSS

SEQ ID NO: 124 - protein, 18 aa, synthetic construct
  REGION 1..18: note = Description of Artificial Sequence: Syntheticpeptide
GSSGSSGSSGSSGSSGSS

SEQ ID NO: 125 - protein, 5 aa, synthetic construct
  REGION 1..5: note = Description of Artificial Sequence: Syntheticpeptide
EAAAK

SEQ ID NO: 126 - protein, 10 aa, synthetic construct
  REGION 1..10: note = Description of Artificial Sequence: Syntheticpeptide
EAAAKEAAAK

SEQ ID NO: 127 - protein, 15 aa, synthetic construct
  REGION 1..15: note = Description of Artificial Sequence: Syntheticpeptide
EAAAKEAAAKEAAAK

SEQ ID NO: 128 - protein, 20 aa, synthetic construct
  REGION 1..20: note = Description of Artificial Sequence: Syntheticpeptide
EAAAKEAAAKEAAAKEAAAK

SEQ ID NO: 129 - protein, 25 aa, synthetic construct
  REGION 1..25: note = Description of Artificial Sequence: Syntheticpeptide
EAAAKEAAAKEAAAKEAAAKEAAAK

SEQ ID NO: 130 - protein, 30 aa, synthetic construct
  REGION 1..30: note = Description of Artificial Sequence: Syntheticpolypeptide
EAAAKEAAAKEAAAKEAAAKEAAAKEAAAK

SEQ ID NO: 131 - skipped

SEQ ID NO: 132 - protein, 5 aa, synthetic construct
  REGION 1..5: note = Description of Artificial Sequence: Syntheticpeptide
PAPAP

SEQ ID NO: 133 - protein, 7 aa, synthetic construct
  REGION 1..7: note = Description of Artificial Sequence: Syntheticpeptide
PAPAPAP

SEQ ID NO: 134 - protein, 9 aa, synthetic construct
  REGION 1..9: note = Description of Artificial Sequence: Syntheticpeptide
PAPAPAPAP

SEQ ID NO: 135 - protein, 11 aa, synthetic construct
  REGION 1..11: note = Description of Artificial Sequence: Syntheticpeptide
PAPAPAPAPAP

SEQ ID NO: 136 - protein, 13 aa, synthetic construct
  REGION 1..13: note = Description of Artificial Sequence: Syntheticpeptide
PAPAPAPAPAPAP

SEQ ID NO: 137 - protein, 6 aa, synthetic construct
  REGION 1..6: note = Description of Artificial Sequence: Syntheticpeptide
GGSGGG

SEQ ID NO: 138 - protein, 6 aa, synthetic construct
  REGION 1..6: note = Description of Artificial Sequence: Syntheticpeptide
GGGGGS

SEQ ID NO: 139 - protein, 6 aa, synthetic construct
  REGION 1..6: note = Description of Artificial Sequence: Syntheticpeptide
GGSGSS

SEQ ID NO: 140 - protein, 6 aa, synthetic construct
  REGION 1..6: note = Description of Artificial Sequence: Syntheticpeptide
GSSGGS

SEQ ID NO: 141 - protein, 8 aa, synthetic construct
  REGION 1..8: note = Description of Artificial Sequence: Syntheticpeptide
GGSEAAAK

SEQ ID NO: 142 - protein, 8 aa, synthetic construct
  REGION 1..8: note = Description of Artificial Sequence: Syntheticpeptide
EAAAKGGS

SEQ ID NO: 143 - protein, 6 aa, synthetic construct
  REGION 1..6: note = Description of Artificial Sequence: Syntheticpeptide
GGSPAP

SEQ ID NO: 144 - protein, 6 aa, synthetic construct
  REGION 1..6: note = Description of Artificial Sequence: Syntheticpeptide
PAPGGS

SEQ ID NO: 145 - protein, 6 aa, synthetic construct
  REGION 1..6: note = Description of Artificial Sequence: Syntheticpeptide
GGGGSS

SEQ ID NO: 146 - protein, 6 aa, synthetic construct
  REGION 1..6: note = Description of Artificial Sequence: Syntheticpeptide
GSSGGG

SEQ ID NO: 147 - protein, 8 aa, synthetic construct
  REGION 1..8: note = Description of Artificial Sequence: Syntheticpeptide
GGGEAAAK

SEQ ID NO: 148 - protein, 8 aa, synthetic construct
  REGION 1..8: note = Description of Artificial Sequence: Syntheticpeptide
EAAAKGGG

SEQ ID NO: 149 - protein, 6 aa, synthetic construct
  REGION 1..6: note = Description of Artificial Sequence: Syntheticpeptide
GGGPAP

SEQ ID NO: 150 - protein, 6 aa, synthetic construct
  REGION 1..6: note = Description of Artificial Sequence: Syntheticpeptide
PAPGGG

SEQ ID NO: 151 - protein, 8 aa, synthetic construct
  REGION 1..8: note = Description of Artificial Sequence: Syntheticpeptide
GSSEAAAK

SEQ ID NO: 152 - protein, 8 aa, synthetic construct
  REGION 1..8: note = Description of Artificial Sequence: Syntheticpeptide
EAAAKGSS

SEQ ID NO: 153 - protein, 6 aa, synthetic construct
  REGION 1..6: note = Description of Artificial Sequence: Syntheticpeptide
GSSPAP

SEQ ID NO: 154 - protein, 6 aa, synthetic construct
  REGION 1..6: note = Description of Artificial Sequence: Syntheticpeptide
PAPGSS

SEQ ID NO: 155 - protein, 8 aa, synthetic construct
  REGION 1..8: note = Description of Artificial Sequence: Syntheticpeptide
EAAAKPAP

SEQ ID NO: 156 - protein, 8 aa, synthetic construct
  REGION 1..8: note = Description of Artificial Sequence: Syntheticpeptide
PAPEAAAK

SEQ ID NO: 157 - protein, 9 aa, synthetic construct
  REGION 1..9: note = Description of Artificial Sequence: Syntheticpeptide
GGSGGGGSS

SEQ ID NO: 158 - protein, 9 aa, synthetic construct
  REGION 1..9: note = Description of Artificial Sequence: Syntheticpeptide
GGSGSSGGG

SEQ ID NO: 159 - protein, 9 aa, synthetic construct
  REGION 1..9: note = Description of Artificial Sequence: Syntheticpeptide
GGGGGSGSS

SEQ ID NO: 160 - protein, 9 aa, synthetic construct
  REGION 1..9: note = Description of Artificial Sequence: Syntheticpeptide
GGGGSSGGS

SEQ ID NO: 161 - protein, 9 aa, synthetic construct
  REGION 1..9: note = Description of Artificial Sequence: Syntheticpeptide
GSSGGSGGG

SEQ ID NO: 162 - protein, 9 aa, synthetic construct
  REGION 1..9: note = Description of Artificial Sequence: Syntheticpeptide
GSSGGGGGS

SEQ ID NO: 163 - protein, 11 aa, synthetic construct
  REGION 1..11: note = Description of Artificial Sequence: Syntheticpeptide
GGSGGGEAAAK

SEQ ID NO: 164 - protein, 11 aa, synthetic construct
  REGION 1..11: note = Description of Artificial Sequence: Syntheticpeptide
GGSEAAAKGGG

SEQ ID NO: 165 - protein, 11 aa, synthetic construct
  REGION 1..11: note = Description of Artificial Sequence: Syntheticpeptide
GGGGGSEAAAK

SEQ ID NO: 166 - protein, 11 aa, synthetic construct
  REGION 1..11: note = Description of Artificial Sequence: Syntheticpeptide
GGGEAAAKGGS

SEQ ID NO: 167 - protein, 11 aa, synthetic construct
  REGION 1..11: note = Description of Artificial Sequence: Syntheticpeptide
EAAAKGGSGGG

SEQ ID NO: 168 - protein, 11 aa, synthetic construct
  REGION 1..11: note = Description of Artificial Sequence: Syntheticpeptide
EAAAKGGGGGS

SEQ ID NO: 169 - protein, 9 aa, synthetic construct
  REGION 1..9: note = Description of Artificial Sequence: Syntheticpeptide
GGSGGGPAP

SEQ ID NO: 170 - protein, 9 aa, synthetic construct
  REGION 1..9: note = Description of Artificial Sequence: Syntheticpeptide
GGSPAPGGG

SEQ ID NO: 171 - protein, 9 aa, synthetic construct
  REGION 1..9: note = Description of Artificial Sequence: Syntheticpeptide
GGGGGSPAP

SEQ ID NO: 172 - protein, 9 aa, synthetic construct
  REGION 1..9: note = Description of Artificial Sequence: Syntheticpeptide
GGGPAPGGS

SEQ ID NO: 173 - protein, 9 aa, synthetic construct
  REGION 1..9: note = Description of Artificial Sequence: Syntheticpeptide
PAPGGSGGG

SEQ ID NO: 174 - protein, 9 aa, synthetic construct
  REGION 1..9: note = Description of Artificial Sequence: Syntheticpeptide
PAPGGGGGS

SEQ ID NO: 175 - protein, 11 aa, synthetic construct
  REGION 1..11: note = Description of Artificial Sequence: Syntheticpeptide
GGSGSSEAAAK

SEQ ID NO: 176 - protein, 11 aa, synthetic construct
  REGION 1..11: note = Description of Artificial Sequence: Syntheticpeptide
GGSEAAAKGSS

SEQ ID NO: 177 - protein, 11 aa, synthetic construct
  REGION 1..11: note = Description of Artificial Sequence: Syntheticpeptide
GSSGGSEAAAK

SEQ ID NO: 178 - protein, 11 aa, synthetic construct
  REGION 1..11: note = Description of Artificial Sequence: Syntheticpeptide
GSSEAAAKGGS

SEQ ID NO: 179 - protein, 11 aa, synthetic construct
  REGION 1..11: note = Description of Artificial Sequence: Syntheticpeptide
EAAAKGGSGSS

SEQ ID NO: 180 - protein, 11 aa, synthetic construct
  REGION 1..11: note = Description of Artificial Sequence: Syntheticpeptide
EAAAKGSSGGS

SEQ ID NO: 181 - protein, 9 aa, synthetic construct
  REGION 1..9: note = Description of Artificial Sequence: Syntheticpeptide
GGSGSSPAP

SEQ ID NO: 182 - protein, 9 aa, synthetic construct
  REGION 1..9: note = Description of Artificial Sequence: Syntheticpeptide
GGSPAPGSS

SEQ ID NO: 183 - protein, 9 aa, synthetic construct
  REGION 1..9: note = Description of Artificial Sequence: Syntheticpeptide
GSSGGSPAP

SEQ ID NO: 184 - protein, 9 aa, synthetic construct
  REGION 1..9: note = Description of Artificial Sequence: Syntheticpeptide
GSSPAPGGS

SEQ ID NO: 185 - protein, 9 aa, synthetic construct
  REGION 1..9: note = Description of Artificial Sequence: Syntheticpeptide
PAPGGSGSS

SEQ ID NO: 186 - protein, 9 aa, synthetic construct
  REGION 1..9: note = Description of Artificial Sequence: Syntheticpeptide
PAPGSSGGS

SEQ ID NO: 187 - protein, 11 aa, synthetic construct
  REGION 1..11: note = Description of Artificial Sequence: Syntheticpeptide
GGSEAAAKPAP

SEQ ID NO: 188 - protein, 11 aa, synthetic construct
  REGION 1..11: note = Description of Artificial Sequence: Syntheticpeptide
GGSPAPEAAAK

SEQ ID NO: 189 - protein, 11 aa, synthetic construct
  REGION 1..11: note = Description of Artificial Sequence: Syntheticpeptide
EAAAKGGSPAP

SEQ ID NO: 190 - protein, 11 aa, synthetic construct
  REGION 1..11: note = Description of Artificial Sequence: Syntheticpeptide
EAAAKPAPGGS

SEQ ID NO: 191 - protein, 11 aa, synthetic construct
  REGION 1..11: note = Description of Artificial Sequence: Syntheticpeptide
PAPGGSEAAAK

SEQ ID NO: 192 - protein, 11 aa, synthetic construct
  REGION 1..11: note = Description of Artificial Sequence: Syntheticpeptide
PAPEAAAKGGS

SEQ ID NO: 193 - protein, 11 aa, synthetic construct
  REGION 1..11: note = Description of Artificial Sequence: Syntheticpeptide
GGGGSSEAAAK

SEQ ID NO: 194 - protein, 11 aa, synthetic construct
  REGION 1..11: note = Description of Artificial Sequence: Syntheticpeptide
GGGEAAAKGSS

SEQ ID NO: 195 - protein, 11 aa, synthetic construct
  REGION 1..11: note = Description of Artificial Sequence: Syntheticpeptide
GSSGGGEAAAK

SEQ ID NO: 196 - protein, 11 aa, synthetic construct
  REGION 1..11: note = Description of Artificial Sequence: Syntheticpeptide
GSSEAAAKGGG

SEQ ID NO: 197 - protein, 11 aa, synthetic construct
  REGION 1..11: note = Description of Artificial Sequence: Syntheticpeptide
EAAAKGGGGSS

SEQ ID NO: 198 - protein, 11 aa, synthetic construct
  REGION 1..11: note = Description of Artificial Sequence: Syntheticpeptide
EAAAKGSSGGG

SEQ ID NO: 199 - protein, 9 aa, synthetic construct
  REGION 1..9: note = Description of Artificial Sequence: Syntheticpeptide
GGGGSSPAP

SEQ ID NO: 200 - protein, 9 aa, synthetic construct
  REGION 1..9: note = Description of Artificial Sequence: Syntheticpeptide
GGGPAPGSS

SEQ ID NO: 201 - protein, 9 aa, synthetic construct
  REGION 1..9: note = Description of Artificial Sequence: Syntheticpeptide
GSSGGGPAP

SEQ ID NO: 202 - protein, 9 aa, synthetic construct
  REGION 1..9: note = Description of Artificial Sequence: Syntheticpeptide
GSSPAPGGG

SEQ ID NO: 203 - protein, 9 aa, synthetic construct
  REGION 1..9: note = Description of Artificial Sequence: Syntheticpeptide
PAPGGGGSS

SEQ ID NO: 204 - protein, 9 aa, synthetic construct
  REGION 1..9: note = Description of Artificial Sequence: Syntheticpeptide
PAPGSSGGG

SEQ ID NO: 205 - protein, 11 aa, synthetic construct
  REGION 1..11: note = Description of Artificial Sequence: Syntheticpeptide
GGGEAAAKPAP

SEQ ID NO: 206 - protein, 11 aa, synthetic construct
  REGION 1..11: note = Description of Artificial Sequence: Syntheticpeptide
GGGPAPEAAAK

SEQ ID NO: 207 - protein, 11 aa, synthetic construct
  REGION 1..11: note = Description of Artificial Sequence: Syntheticpeptide
EAAAKGGGPAP

SEQ ID NO: 208 - protein, 11 aa, synthetic construct
  REGION 1..11: note = Description of Artificial Sequence: Syntheticpeptide
EAAAKPAPGGG

SEQ ID NO: 209 - protein, 11 aa, synthetic construct
  REGION 1..11: note = Description of Artificial Sequence: Syntheticpeptide
PAPGGGEAAAK

SEQ ID NO: 210 - protein, 11 aa, synthetic construct
  REGION 1..11: note = Description of Artificial Sequence: Syntheticpeptide
PAPEAAAKGGG

SEQ ID NO: 211 - protein, 11 aa, synthetic construct
  REGION 1..11: note = Description of Artificial Sequence: Syntheticpeptide
GSSEAAAKPAP

SEQ ID NO: 212 - protein, 11 aa, synthetic construct
  REGION 1..11: note = Description of Artificial Sequence: Syntheticpeptide
GSSPAPEAAAK

SEQ ID NO: 213 - protein, 11 aa, synthetic construct
  REGION 1..11: note = Description of Artificial Sequence: Syntheticpeptide
EAAAKGSSPAP

SEQ ID NO: 214 - protein, 11 aa, synthetic construct
  REGION 1..11: note = Description of Artificial Sequence: Syntheticpeptide
EAAAKPAPGSS

SEQ ID NO: 215 - protein, 11 aa, synthetic construct
  REGION 1..11: note = Description of Artificial Sequence: Syntheticpeptide
PAPGSSEAAAK

SEQ ID NO: 216 - protein, 11 aa, synthetic construct
  REGION 1..11: note = Description of Artificial Sequence: Syntheticpeptide
PAPEAAAKGSS

SEQ ID NO: 217 - protein, 46 aa, synthetic construct
  REGION 1..46: note = Description of Artificial Sequence: Syntheticpolypeptide
AEAAAKEAAAKEAAAKEAAAKALEAEAAAKEAAAKEAAAKEAAAKA

SEQ ID NO: 218 - protein, 15 aa, synthetic construct
  REGION 1..15: note = Description of Artificial Sequence: Syntheticpeptide
GGGGSEAAAKGGGGS

SEQ ID NO: 219 - protein, 15 aa, synthetic construct
  REGION 1..15: note = Description of Artificial Sequence: Syntheticpeptide
EAAAKGGGGSEAAAK

SEQ ID NO: 220 - protein, 16 aa, synthetic construct
  REGION 1..16: note = Description of Artificial Sequence: Syntheticpeptide
SGSETPGTSESATPES

SEQ ID NO: 221 - protein, 12 aa, synthetic construct
  REGION 1..12: note = Description of Artificial Sequence: Syntheticpeptide
GSAGSAAGSGEF

SEQ ID NO: 222 - protein, 33 aa, synthetic construct; identical to SEQ ID NO: 6
  REGION 1..33: note = Description of Artificial Sequence: Syntheticpolypeptide

SEQ ID NO: 223 - protein, 30 aa, Rattus norvegicus
AHFKISGEKRPSTDPGKKAKNPKKKKKKDP

SEQ ID NO: 224 - protein, 12 aa, Saccharomyces cerevisiae
AHRAKKMSKTHA

SEQ ID NO: 225 - protein, 14 aa, unidentified
  REGION 1..14: note = Description of Unknown:NLS sequence
ASPEYVNLPINGNG

SEQ ID NO: 226 - protein, 7 aa, unidentified
  REGION 1..7: note = Description of Unknown:NLS sequence
CTKRPRW

SEQ ID NO: 227 - protein, 16 aa, unidentified
  REGION 1..16: note = Description of Unknown:NLS sequence
DKAKRVSRNKSEKKRR

SEQ ID NO: 228 - protein, 15 aa, unidentified
  REGION 1..15: note = Description of Unknown:NLS sequence
EELRLKEELLKGIYA

SEQ ID NO: 229 - protein, 16 aa, Caenorhabditis elegans
EEQLRRRKNSRLNNTG

SEQ ID NO: 230 - protein, 24 aa, unidentified
  REGION 1..24: note = Description of Unknown:NLS sequence
EVLKVIRTGKRKKKAWKRMVTKVC

SEQ ID NO: 231 - protein, 15 aa, unidentified
  REGION 1..15: note = Description of Unknown:NLS sequence
HHHHHHHHHHHHQPH

SEQ ID NO: 232 - protein, 17 aa, unidentified
  REGION 1..17: note = Description of Unknown:NLS sequence
HKKKHPDASVNFSEFSK

SEQ ID NO: 233 - protein, 6 aa, Oryza sativa
HKRTKK

SEQ ID NO: 234 - protein, 29 aa, unidentified
  REGION 1..29: note = Description of Unknown:NLS sequence
IINGRKLKLKKSRRRSSQTSNNSFTSRRS

SEQ ID NO: 235 - protein, 8 aa, Oryza sativa
KAEQERRK

SEQ ID NO: 236 - protein, 18 aa, unidentified
  REGION 1..18: note = Description of Unknown:NLS sequence
KEKRKRREELFIEQKKRK

SEQ ID NO: 237 - protein, 14 aa, Mus musculus
KKGKDEWFSRGKKP

SEQ ID NO: 238 - protein, 12 aa, Homo sapiens
KKGPSVQKRKKT

SEQ ID NO: 239 - protein, 16 aa, unidentified
  REGION 1..16: note = Description of Unknown:NLS sequence
KKKTVINDLLHYKKEK

SEQ ID NO: 240 - protein, 17 aa, unidentified
  REGION 1..17: note = Description of Unknown:NLS sequence
KKNGGKGKNKPSAKIKK

SEQ ID NO: 241 - protein, 13 aa, unidentified
  REGION 1..13: note = Description of Unknown:NLS sequence
KKPKWDDFKKKKK

SEQ ID NO: 242 - protein, 6 aa, unidentified
  REGION 1..6: note = Description of Unknown:NLS sequence
KKRKKD

SEQ ID NO: 243 - protein, 9 aa, unidentified
  REGION 1..9: note = Description of Unknown:NLS sequence
KKRRKRRRK

SEQ ID NO: 244 - protein, 9 aa, unidentified
  REGION 1..9: note = Description of Unknown:NLS sequence
KKRRRRARK

SEQ ID NO: 245 - protein, 7 aa, Homo sapiens
KKSKRGR

SEQ ID NO: 246 - protein, 8 aa, Zea mays
KKSRKRGS

SEQ ID NO: 247 - protein, 17 aa, unidentified
  REGION 1..17: note = Description of Unknown:NLS sequence
KKSTALSRELGKIMRRR

SEQ ID NO: 248 - protein, 17 aa, Dictyostelium discoideum
KKSYQDPEIIAHSRPRK

SEQ ID NO: 249 - protein, 17 aa, unidentified
  REGION 1..17: note = Description of Unknown:NLS sequence
KKTGKNRKLKSKRVKTR

SEQ ID NO: 250 - protein, 17 aa, Oryza sativa
KKVSIAGQSGKLWRWKR

SEQ ID NO: 251 - protein, 20 aa, unidentified
  REGION 1..20: note = Description of Unknown:NLS sequence
KKYENVVIKRSPRKRGRPRK

SEQ ID NO: 252 - protein, 6 aa, unidentified
  REGION 1..6: note = Description of Unknown:NLS sequence
KNKKRK

SEQ ID NO: 253 - protein, 6 aa, unidentified
  REGION 1..6: note = Description of Unknown:NLS sequence
KPKKKR

SEQ ID NO: 254 - protein, 19 aa, Danio rerio
KRAMKDDSHGNSTSPKRRK

SEQ ID NO: 255 - protein, 17 aa, Homo sapiens
KRANSNLVAAYEKAKKK

SEQ ID NO: 256 - protein, 22 aa, unidentified
  REGION 1..22: note = Description of Unknown:NLS sequence
KRASEDTTSGSPPKKSSAGPKR

SEQ ID NO: 257 - protein, 16 aa, unidentified
  REGION 1..16: note = Description of Unknown:NLS sequence
KRFKRRWMVRKMKTKK

SEQ ID NO: 258 - protein, 16 aa, Homo sapiens
KRGLNSSFETSPKKVK

SEQ ID NO: 259 - protein, 20 aa, unidentified
  REGION 1..20: note = Description of Unknown:NLS sequence
KRGNSSIGPNDLSKRKQRKK

SEQ ID NO: 260 - protein, 23 aa, unidentified
  REGION 1..23: note = Description of Unknown:NLS sequence
KRIHSVSLSQSQIDPSKKVKRAK

SEQ ID NO: 261 - protein, 15 aa, Homo sapiens
KRKGKLKNKGSKRKK

SEQ ID NO: 262 - protein, 13 aa, Homo sapiens
KRRRRRRREKRKR

SEQ ID NO: 263 - protein, 18 aa, Mus musculus
KRSNDRTYSPEEEKQRRA

SEQ ID NO: 264 - protein, 21 aa, unidentified
  REGION 1..21: note = Description of Unknown:NLS sequence
KRTVATNGDASGAHRAKKMSK

SEQ ID NO: 265 - protein, 20 aa, unidentified
  REGION 1..20: note = Description of Unknown:NLS sequence
KRVYNKGEDEQEHLPKGKKR

SEQ ID NO: 266 - protein, 18 aa, unidentified
  REGION 1..18: note = Description of Unknown:NLS sequence
KSGKAPRRRAVSMDNSNK

SEQ ID NO: 267 - protein, 19 aa, Homo sapiens
KVNFLDMSLDDIIIYKELE

SEQ ID NO: 268 - protein, 15 aa, Tomato yellow leaf curl China virus
KVQHRIAKKTTRRRR

SEQ ID NO: 269 - protein, 8 aa, unidentified
  REGION 1..8: note = Description of Unknown:NLS sequence
LSPSLSPL

SEQ ID NO: 270 - protein, 30 aa, Homo sapiens; identical to SEQ ID NO: 9

SEQ ID NO: 271 - protein, 52 aa, unidentified
  REGION 1..52: note = Description of Unknown:NLS sequence
MPQNEYIELHRKRYGYRLDYHEKKRKKESREAHERSKKAKKMIGLKAKLYHK

SEQ ID NO: 272 - protein, 10 aa, unidentified
  REGION 1..10: note = Description of Unknown:NLS sequence
MVQLRPRASR

SEQ ID NO: 273 - protein, 24 aa, Caenorhabditis elegans
NNKLLAKRRKGGASPKDDPMDDIK

SEQ ID NO: 274 - protein, 20 aa, unidentified
  REGION 1..20: note = Description of Unknown:NLS sequence
NYKRPMDGTYGPPAKRHEGE

SEQ ID NO: 275 - protein, 19 aa, unidentified
  REGION 1..19: note = Description of Unknown:NLS sequence
PDTKRAKLDSSETTMVKKK

SEQ ID NO: 276 - protein, 7 aa, unidentified
  REGION 1..7: note = Description of Unknown:NLS sequence
PEKRTKI

SEQ ID NO: 277 - protein, 8 aa, unidentified
  REGION 1..8: note = Description of Unknown:NLS sequence
PGGRGKKK

SEQ ID NO: 278 - protein, 18 aa, unidentified
  REGION 1..18: note = Description of Unknown:NLS sequence
PGKMDKGEHRQERRDRPY

SEQ ID NO: 279 - protein, 11 aa, Physarum polycephalum
PKKGDKYDKTD

SEQ ID NO: 280 - protein, 7 aa, unidentified
  REGION 1..7: note = Description of Unknown:NLS sequence
PKKKSRK

SEQ ID NO: 281 - protein, 7 aa, Caenorhabditis elegans
PKKNKPE

SEQ ID NO: 282 - protein, 7 aa, unidentified
  REGION 1..7: note = Description of Unknown:NLS sequence
PKKRAKV

SEQ ID NO: 283 - protein, 9 aa, unidentified
  REGION 1..9: note = Description of Unknown:NLS sequence
PKPKKLKVE

SEQ ID NO: 284 - protein, 7 aa, unidentified
  REGION 1..7: note = Description of Unknown:NLS sequence
PKRGRGR

SEQ ID NO: 285 - protein, 9 aa, Mammalian 1 orthobornavirus
PKRRLVDDA

SEQ ID NO: 286 - protein, 7 aa, unidentified
  REGION 1..7: note = Description of Unknown:NLS sequence
PKRRRTY

SEQ ID NO: 287 - protein, 6 aa, unidentified
  REGION 1..6: note = Description of Unknown:NLS sequence
PLFKRR

SEQ ID NO: 288 - protein, 7 aa, unidentified
  REGION 1..7: note = Description of Unknown:NLS sequence
PLRKAKR

SEQ ID NO: 289 - protein, 9 aa, unidentified
  REGION 1..9: note = Description of Unknown:NLS sequence
PPAKRKCIF

SEQ ID NO: 290 - protein, 8 aa, Homo sapiens
PPARRRRL

SEQ ID NO: 291 - protein, 8 aa, unidentified
  REGION 1..8: note = Description of Unknown:NLS sequence
PPKKKRKV

SEQ ID NO: 292 - protein, 10 aa, Mus musculus
PPNKRMKVKH

SEQ ID NO: 293 - protein, 13 aa, Mammalian 1 orthobornavirus
PPRIYPQLPSAPT

SEQ ID NO: 294 - protein, 13 aa, unidentified
  REGION 1..13: note = Description of Unknown:NLS sequence
PQRSPFPKSSVKR

SEQ ID NO: 295 - protein, 8 aa, Mammalian 1 orthobornavirus
PRPRKVPR

SEQ ID NO: 296 - protein, 9 aa, unidentified
  REGION 1..9: note = Description of Unknown:NLS sequence
PRRRVQRKR

SEQ ID NO: 297 - protein, 7 aa, unidentified
  REGION 1..7: note = Description of Unknown:NLS sequence
PRRVRLK

SEQ ID NO: 298 - protein, 7 aa, unidentified
  REGION 1..7: note = Description of Unknown:NLS sequence
PSRKRPR

SEQ ID NO: 299 - protein, 8 aa, unidentified
  REGION 1..8: note = Description of Unknown:NLS sequence
PSSKKRKV

SEQ ID NO: 300 - protein, 7 aa, Drosophila melanogaster
PTKKRVK

SEQ ID NO: 301 - protein, 9 aa, unidentified
  REGION 1..9: note = Description of Unknown:NLS sequence
QRPGPYDRP

SEQ ID NO: 302 - protein, 18 aa, unidentified
  REGION 1..18: note = Description of Unknown:NLS sequence
RGKGGKGLGKGGAKRHRK

SEQ ID NO: 303 - protein, 24 aa, Zea mays
RKAGKGGGGHKTTKKRSAKDEKVP

SEQ ID NO: 304 - protein, 9 aa, Xenopus laevis
RKIKLKRAK

SEQ ID NO: 305 - protein, 9 aa, Xenopus laevis
RKIKRKRAK

SEQ ID NO: 306 - protein, 17 aa, unidentified
  REGION 1..17: note = Description of Unknown:NLS sequence
RKKEAPGPREELRSRGR

SEQ ID NO: 307 - protein, 7 aa, unidentified
  REGION 1..7: note = Description of Unknown:NLS sequence
RKKRKGK

SEQ ID NO: 308 - protein, 9 aa, unidentified
  REGION 1..9: note = Description of Unknown:NLS sequence
RKKRRQRRR

SEQ ID NO: 309 - protein, 25 aa, Homo sapiens
RKKSIPLSIKNLKRKHKRKKNKITR

SEQ ID NO: 310 - protein, 20 aa, Homo sapiens
RKLVKPKNTKMKTKLRTNPY

SEQ ID NO: 311 - protein, 16 aa, unidentified
  REGION 1..16: note = Description of Unknown:NLS sequence
RKRLILSDKGQLDWKK

SEQ ID NO: 312 - protein, 7 aa, Homo sapiens
RKRLKSK

SEQ ID NO: 313 - protein, 9 aa, unidentified
  REGION 1..9: note = Description of Unknown:NLS sequence
RKRRVRDNM

SEQ ID NO: 314 - protein, 22 aa, Homo sapiens
RKRSPKDKKEKDLDGAGKRRKT

SEQ ID NO: 315 - protein, 22 aa, Homo sapiens
RKRTPRVDGQTGENDMNKRRRK

SEQ ID NO: 316 - protein, 10 aa, unidentified
  REGION 1..10: note = Description of Unknown:NLS sequence
RLPVRRRRRR

SEQ ID NO: 317 - protein, 10 aa, Feline immunodeficiency virus
RLRFRKPKSK

SEQ ID NO: 318 - protein, 6 aa, Homo sapiens
RQQRKR

SEQ ID NO: 319 - protein, 16 aa, Mus musculus
RRDLNSSFETSPKKVK

SEQ ID NO: 320 - protein, 8 aa, Arabidopsis thaliana
RRDRAKLR

SEQ ID NO: 321 - protein, 8 aa, unidentified
  REGION 1..8: note = Description of Unknown:NLS sequence
RRGDGRRR

SEQ ID NO: 322 - protein, 11 aa, unidentified
  REGION 1..11: note = Description of Unknown:NLS sequence
RRGRKRKAEKQ

SEQ ID NO: 323 - protein, 6 aa, unidentified
  REGION 1..6: note = Description of Unknown:NLS sequence
RRKKRR

SEQ ID NO: 324 - protein, 19 aa, Mus musculus
RRKRSKSEDMDSVESKRRR

SEQ ID NO: 325 - protein, 6 aa, unidentified
  REGION 1..6: note = Description of Unknown:NLS sequence
RRKRSR

SEQ ID NO: 326 - protein, 14 aa, Homo sapiens
RRPKGKTLQKRKPK

SEQ ID NO: 327 - protein, 18 aa, unidentified
  REGION 1..18: note = Description of Unknown:NLS sequence
RRRGFERFGPDNMGRKRK

SEQ ID NO: 328 - protein, 15 aa, unidentified
  REGION 1..15: note = Description of Unknown:NLS sequence
RRRGKNKVAAQNCRK

SEQ ID NO: 329 - protein, 6 aa, unidentified
  REGION 1..6: note = Description of Unknown:NLS sequence
RRRKRR

SEQ ID NO: 330 - protein, 14 aa, unidentified
  REGION 1..14: note = Description of Unknown:NLS sequence
RRRQKQKGGASRRR

SEQ ID NO: 331 - protein, 13 aa, unidentified
  REGION 1..13: note = Description of Unknown:NLS sequence
RRRREGPRARRRR

SEQ ID NO: 332 - protein, 61 aa, unidentified
  REGION 1..61: note = Description of Unknown:NLS sequence
RRTIRLKLVYDKCDRSCKIQKKNRNKCQYCRFHKCLSVGMSHNAIRFGRMPRSEKAKLKAE

SEQ ID NO: 333 - protein, 17 aa, unidentified
  REGION 1..17: note = Description of Unknown:NLS sequence
RRVPQRKEVSRCRKCRK

SEQ ID NO: 334 - protein, 32 aa, Human mastadenovirus C
RVGGRRQAVECIEDLLNEPGQPLDLSCKRPRP

SEQ ID NO: 335 - protein, 9 aa, unidentified
  REGION 1..9: note = Description of Unknown:NLS sequence
RVVKLRIAP

SEQ ID NO: 336 - protein, 6 aa, Mus musculus
RVVRRR

SEQ ID NO: 337 - protein, 12 aa, unidentified
  REGION 1..12: note = Description of Unknown:NLS sequence
SKRKTKISRKTR

SEQ ID NO: 338 - protein, 16 aa, Squash leaf curl virus
SYVKTVPNRTRTYIKL

SEQ ID NO: 339 - protein, 12 aa, unidentified
  REGION 1..12: note = Description of Unknown:NLS sequence
TGKNEAKKRKIA

SEQ ID NO: 340 - protein, 30 aa, unidentified
  REGION 1..30: note = Description of Unknown:NLS sequence
TLSPASSPSSVSCPVIPASTDESPGSALNI

SEQ ID NO: 341 - protein, 12 aa, unidentified
  REGION 1..12: note = Description of Unknown:NLS sequence
VSKKQRTGKKIH

SEQ ID NO: 342 - protein, 9 aa, unidentified
  REGION 1..9: note = Description of Unknown:NLS sequence
SPKKKRKVE

SEQ ID NO: 343 - protein, 19 aa, unidentified
  REGION 1..19: note = Description of Unknown:NLS sequence
KRTADGSEFESPKKKRKVE

SEQ ID NO: 344 - protein, 9 aa, unidentified
  REGION 1..9: note = Description of Unknown:NLS sequence
PAAKRVKLD

SEQ ID NO: 345 - protein, 7 aa, unidentified
  REGION 1..7: note = Description of Unknown:NLS sequence
PKKKRKV

SEQ ID NO: 346 - protein, 30 aa, unidentified; identical to SEQ ID NO: 9
  REGION 1..30: note = Description of Unknown:NLS sequence

SEQ ID NO: 347 - protein, 11 aa, unidentified
  REGION 1..11: note = Description of Unknown:NLS sequence
SPKKKRKVEAS

SEQ ID NO: 348 - protein, 16 aa, unidentified
  REGION 1..16: note = Description of Unknown:NLS sequence
MAPKKKRKVGIHRGVP

SEQ ID NO: 349 - protein, 1082 aa, synthetic construct
  REGION 1..1082: note = Description of Artificial Sequence: Syntheticpolypeptide
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 350 - protein, 1055 aa, synthetic construct
  REGION 1..1055: note = Description of Artificial Sequence: Syntheticpolypeptide
MQNNPLNYILGLDLGIASIGWAVVEIDEESSPIRLIDVGVRTFERAEVAKTGESLALSRRLARSSRRLIKRRAERLKKAKRLLKAEKILHSIDEKLPINVWQLRVKGLKEKLERQEWAAVLLHLSKHRGYLSQRKNEGKSDNKELGALLSGIASNHQMLQSSEYRTPAEIAVKKFQVEEGHIRNQRGSYTHTFSRLDLLAEMELLFQRQAELGNSYTSTTLLENLTALLMWQKPALAGDAILKMLGKCTFEPSEYKAAKNSYSAERFVWLTKLNNLRILENGTERALNDNERFALLEQPYEKSKLTYAQVRAMLALSDNAIFKGVRYLGEDKKTVESKTTLIEMKFYHQIRKTLGSAELKKEWNELKGNSDLLDEIGTAFSLYKTDDDICRYLEGKLPERVLNALLENLNFDKFIQLSLKALHQILPLMLQGQRYDEAVSAIYGDHYGKKSTETTRLLPTIPADEIRNPVVLRTLTQARKVINAVVRLYGSPARIHIETAREVGKSYQDRKKLEKQQEDNRKQRESAVKKFKEMFPHFVGEPKGKDILKMRLYELQQAKCLYSGKSLELHRLLEKGYVEVDHALPFSRTWDDSFNNKVLVLANENQNKGNLTPYEWLDGKNNSERWQHFVVRVQTSGFSYAKKQRILNHKLDEKGFIERNLNDTRYVARFLCNFIADNMLLVGKGKRNVFASNGQITALLRHRWGLQKVREQNDRHHALDAVVVACSTVAMQQKITRFVRYNEGNVFSGERIDRETGEIIPLHFPSPWAFFKENVEIRIFSENPKLELENRLPDYPQYNHEWVQPLFVSRMPTRKMTGQGHMETVKSAKRLNEGLSVLKVPLTQLKLSDLERMVNRDREIALYESLKARLEQFGNDPAKAFAEPFYKKGGALVKAVRLEQTQKSGVLVRDGNGVADNASMVRVDVFTKGGKYFLVPIYTWQVAKGILPNRAATQGKDENDWDIMDEMATFQFSLCQNDLIKLVTKKKTIFGYFNGLNRATSNINIKEHDLDKSKGKLGIYLEVGVKLAISLEKYQVDELGKNIRPCRPTKRQHVR

SEQ ID NO: 351 - protein, 1053 aa, synthetic construct
  REGION 1..1053: note = Description of Artificial Sequence: Syntheticpolypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 352 - protein, 1053 aa, synthetic construct
  REGION 1..1053: note = Description of Artificial Sequence: Syntheticpolypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 353 - protein, 1061 aa, synthetic construct
  REGION 1..1061: note = Description of Artificial Sequence: Syntheticpolypeptide
MQENQQKQNYILGLDIGITSVGYGLIDSKTREVIDAGVRLFPEADSENNSNRRSKRGARRLKRRRIHRLNRVKDLLADYQMIDLNNVPKSTDPYTIRVKGLREPLTKEEFAIALLHIAKRRGLHNISVSMGDEEQDNELSTKQQLQKNAQQLQDKYVCELQLERLTNINKVRGEKNRFKTEDFVKEVKQLCETQRQYHNIDDQFIQQYIDLVSTRREYFEGPGNGSPYGWDGDLLKWYEKLMGRCTYFPEELRSVKYAYSADLFNALNDLNNLVVTRDDNPKLEYYEKYHIIENVFKQKKNPTLKQIAKEIGVQDYDIRGYRITKSGKPQFTSFKLYHDLKNIFEQAKYLEDVEMLDEIAKILTIYQDEISIKKALDQLPELLTESEKSQIAQLTGYTGTHRLSLKCIHIVIDELWESPENQMEIFTRLNLKPKKVEMSEIDSIPTTLVDEFILSPVVKRAFIQSIKVINAVINRFGLPEDIIIELAREKNSKDRRKFINKLQKQNEATRKKIEQLLAKYGNTNAKYMIEKIKLHDMQEGKCLYSLEAIPLEDLLSNPTHYEVDHIIPRSVSFDNSLNNKVLVKQSENSKKGNRTPYQYLSSNESKISYNQFKQHILNLSKAKDRISKKKRDMLLEERDINKFEVQKEFINRNLVDTRYATRELSNLLKTYFSTHDYAVKVKTINGGFTNHLRKVWDFKKHRNHGYKHHAEDALVIANADFLFKTHKALRRTDKILEQPGLEVNDTTVKVDTEEKYQELFETPKQVKNIKQFRDFKYSHRVDKKPNRQLINDTLYSTREIDGETYVVQTLKDLYAKDNEKVKKLFTERPQKILMYQHDPKTFEKLMTILNQYAEAKNPLAAYYEDKGEYVTKYAKKGNGPAIHKIKYIDKKLGSYLDVSNKYPETQNKLVKLSLKSFRFDIYKCEQGYKMVSIGYLDVLKKDNYYYIPKDKYEAEKQKKKIKESDLFVGSFYYNDLIMYEDELFRVIGVNSDINNLVELNMVDITYKDFCEVNNVTGEKRIKKTIGKRVVLIEKYTTDILGNLYKTPLPKKPQLIFKRGEL

SEQ ID NO: 354 - protein, 1061 aa, synthetic construct
  REGION 1..1061: note = Description of Artificial Sequence: Syntheticpolypeptide
MQENQQKQNYILGLDIGITSVGYGLIDSKTREVIDAGVRLFPEADSENNSNRRSKRGARRLKRRRIHRLNRVKDLLADYQMIDLNNVPKSTDPYTIRVKGLREPLTKEEFAIALLHIAKRRGLHNISVSMGDEEQDNELSTKQQLQKNAQQLQDKYVCELQLERLTNINKVRGEKNRFKTEDFVKEVKQLCETQRQYHNIDDQFIQQYIDLVSTRREYFEGPGNGSPYGWDGDLLKWYEKLMGRCTYFPEELRSVKYAYSADLFNALNDLNNLVVTRDDNPKLEYYEKYHIIENVFKQKKNPTLKQIAKEIGVQDYDIRGYRITKSGKPQFTSFKLYHDLKNIFEQAKYLEDVEMLDEIAKILTIYQDEISIKKALDQLPELLTESEKSQIAQLTGYTGTHRLSLKCIHIVIDELWESPENQMEIFTRLNLKPKKVEMSEIDSIPTTLVDEFILSPVVKRAFIQSIKVINAVINRFGLPEDIIIELAREKNSKDRRKFINKLQKQNEATRKKIEQLLAKYGNTNAKYMIEKIKLHDMQEGKCLYSLEAIPLEDLLSNPTHYEVDHIIPRSVSFDNSLNNKVLVKQSENSKKGNRTPYQYLSSNESKISYNQFKQHILNLSKAKDRISKKKRDMLLEERDINKFEVQKEFINRNLVDTRYATRELSNLLKTYFSTHDYAVKVKTINGGFTNHLRKVWDFKKHRNHGYKHHAEDALVIANADFLFKTHKALRRTDKILEQPGLEVNDTTVKVDTEEKYQELFETPKQVKNIKQFRDFKYSHRVDKKPNRKLINDTLYSTREIDGETYVVQTLKDLYAKDNEKVKKLFTERPQKILMYQHDPKTFEKLMTILNQYAEAKNPLAAYYEDKGEYVTKYAKKGNGPAIHKIKYIDKKLGSYLDVSNKYPETQNKLVKLSLKSFRFDIYKCEQGYKMVSIGYLDVLKKDNYYYIPKDKYEAEKQKKKIKESDLFVGSFYKNDLIMYEDELFRVIGVNSDINNLVELNMVDITYKDFCEVNNVTGEKHIKKTIGKRVVLIEKYTTDILGNLYKTPLPKKPQLIFKRGEL

SEQ ID NO: 355 - protein, 1375 aa, synthetic construct
  REGION 1..1375: note = Description of Artificial Sequence: Syntheticpolypeptide
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGADKKLRKRSGKLATEEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASAKELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 356 - protein, 1368 aa, synthetic construct [sequence omitted]

SEQ ID NO: 357 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 358 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 359 - protein, 1121 aa, synthetic construct
  REGION 1..1121: note = Description of Artificial Sequence: Syntheticpolypeptide
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 360 - protein, 1092 aa, synthetic construct
  REGION 1..1092: note = Description of Artificial Sequence: Syntheticpolypeptide
MAYTMGIDVGIASCGWAIVDLERQRIIDIGVRTFEKAENPKNGEALAVPRREARSSRRRLRRKKHRIERLKHMFVRNGLAVDIQHLEQTLRSQNEIDVWQLRVDGLDRMLTQKEWLRVLIHLAQRRGFQSNRKTDGSSEDGQVLVNVTENDRLMEEKDYRTVAEMMVKDEKFSDHKRNKNGNYHGVVSRSSLLVEIHTLFETQRQHHNSLASKDFELEYVNIWSAQRPVATKDQIEKMIGTCTFLPKEKRAPKASWHFQYFMLLQTINHIRITNVQGTRSLNKEEIEQVVNMALTKSKVSYHDTRKILDLSEEYQFVGLDYGKEDEKKKVESKETIIKLDDYHKLNKIFNEVELAKGETWEADDYDTVAYALTFFKDDEDIRDYLQNKYKDSKNRLVKNLANKEYTNELIGKVSTLSFRKVGHLSLKALRKIIPFLEQGMTYDKACQAAGFDFQGISKKKRSVVLPVIDQISNPVVNRALTQTRKVINALIKKYGSPETIHIETARELSKTFDERKNITKDYKENRDKNEHAKKHLSELGIINPTGLDIVKYKLWCEQQGRCMYSNQPISFERLKESGYTEVDHIIPYSRSMNDSYNNRVLVMTRENREKGNQTPFEYMGNDTQRWYEFEQRVTTNPQIKKEKRQNLLLKGFTNRRELEMLERNLNDTRYITKYLSHFISTNLEFSPSDKKKKVVNTSGRITSHLRSRWGLEKNRGQNDLHHAMDAIVIAVTSDSFIQQVTNYYKRKERRELNGDDKFPLPWKFFREEVIARLSPNPKEQIEALPNHFYSEDELADLQPIFVSRMPKRSITGEAHQAQFRRVVGKTKEGKNITAKKTALVDISYDKNGDFNMYGRETDPATYEAIKERYLEFGGNVKKAFSTDLHKPKKDGTKGPLIKSVRIMENKTLVHPVNKGKGVVYNSSIVRTDVFQRKEKYYLLPVYVTDVTKGKLPNKVIVAKKGYHDWIEVDDSFTFLFSLYPNDLIFIRQNPKKKISLKKRIESHSISDSKEVQEIHAYYKGVDSSTAAIEFIIHDGSYYAKGVGVQNLDCFEKYQVDILGNYFKVKGEKRLELETSDSNHKGKDVNSIKSTSR

SEQ ID NO: 361 - protein, 1053 aa, synthetic construct
  REGION 1..1053: note = Description of Artificial Sequence: Syntheticpolypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNSDKNNLIEVNMIDITYREYLENMNDKRPPHIIKTIASKTQSIKKYSTDILGNLYEVKSKKHPQIIKKG

SEQ ID NO: 362 - protein, 1053 aa, synthetic construct
  REGION 1..1053: note = Description of Artificial Sequence: Syntheticpolypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNSTRNIVELNMIDITYREYLENMNDKRPPHIIKTIASKTQSIKKYSTDILGNLYEVKSKKHPQIIKKG

SEQ ID NO: 363 - protein, 1053 aa, synthetic construct
  REGION 1..1053: note = Description of Artificial Sequence: Syntheticpolypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNSDDRNIIELNMIDITYREYLENMNDKRPPHIIKTIASKTQSIKKYSTDILGNLYEVKSKKHPQIIKKG

SEQ ID NO: 364 - protein, 1053 aa, synthetic construct
  REGION 1..1053: note = Description of Artificial Sequence: Syntheticpolypeptide
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNNRLNKIELNMIDITYREYLENMNDKRPPHIIKTIASKTQSIKKYSTDILGNLYEVKSKKHPQIIKKG

SEQ ID NO: 365 - protein, 1084 aa, synthetic construct
  REGION 1..1084: note = Description of Artificial Sequence: Syntheticpolypeptide
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 366 - protein, 984 aa, synthetic construct
  REGION 1..984: note = Description of Artificial Sequence: Syntheticpolypeptide
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 367 - protein, 1087 aa, synthetic construct
  REGION 1..1087: note = Description of Artificial Sequence: Syntheticpolypeptide
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 368 - protein, 1378 aa, synthetic construct
  REGION 1..1378: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEIQTVGQNGGLFDDNPKSPLEVTPSKLVPLKKELNPKKYGGYQKPTTAYPVLLITDTKQLIPISVMNKKQFEQNPVKFLRDRGYQQVGKNDFIKLPKYTLVDIGDGIKRLWASSKEIHKGNQLVVSKKSQILLYHAHHLDSDLSNDYLQNHNQQFDVLFNEIISFSKKCKLGKEHIQKIENVYSNKKNSASIEELAESFIKLLGFTQLGATSPFNFLGVKLNQKQYKGKKDYILPCTEGTLIRQSITGLYETRVDLSKIGEDSGGSGGSKRTADGSEFES

SEQ ID NO: 369 - protein, 1082 aa, synthetic construct
  REGION 1..1082: note = Description of Artificial Sequence: Syntheticpolypeptide
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 370 - protein, 1375 aa, synthetic construct
  REGION 1..1375: note = Description of Artificial Sequence: Syntheticpolypeptide
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 371 - protein, 1375 aa, synthetic construct
  REGION 1..1375: note = Description of Artificial Sequence: Syntheticpolypeptide
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGADKKLRKRSGKLATEEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNANFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASAKELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 372 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTAAYSVLVVAKVEKGKSKKLKSVKELLGITIMERSSFEKNPIGFLEAKGYKEVKKDLIIKLPKYSLFELENGRKRMLASAGVLHKGNELALPSKYVNFLYLASHYEKLKGSPEDNEQKQLFVEQHKHYLDEIIEQISEFSKRVILADANLDKVLSAYNKHRDKPIREQAENIIHLFTLTNLGVPAAFKYFDTTIDKKRYTSTKEVLDATLIHQSITGLYETRIDLSQLGGD

SEQ ID NO: 373 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIGFLEAKGYKEVKKDLIIKLPKYSLFELENGRKRMLASASVLHKGNELALPSKYVNFLYLASHYEKLKGSSEDNKQKQLFVEQHKHYLDEIIEQISEFSKRVILADANLDKVLSAYNKHRDKPIREQAENIIHLFTLTNLGASAAFKYFDTTIGRKLYTSTKEVLDATLIHQSITGLYETRIDLSQLGGD

SEQ ID NO: 374 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGD

SEQ ID NO: 375 - protein, 1368 aa, synthetic construct [sequence omitted]

SEQ ID NO: 376 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADAQLDKVLSAYNKHRDKPIREQAENIIHLFTLTNLGAPAAFKYFDTTFKQKQYRSTKEVLDATLIHQSITGLYETRIDLSQLGGD

SEQ ID NO: 377 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 378 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 379 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGD

SEQ ID NO: 380 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 381 - protein, 1368 aa, synthetic construct
  REGION 1..1368: note = Description of Artificial Sequence: Syntheticpolypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 382 - protein, 1122 aa, synthetic construct
  REGION 1..1122: note = Description of Artificial Sequence: Syntheticpolypeptide
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEEQLLDIETGELISDDEYKESVFKAPYQHFVDTLKSKEFEDSILFSYQVDSKFNRKISDATIYATRQAKVGKDKKDETYVLGKIKDIYTQDGYDAFMKIYKKDKSKFLMYRHDPQTFEKVIEPILENYPNKQMNEKGKEVPCNPFLKYKEEHGYIRKYSKKGNGPEIKSLKYYDSKLLGNPIDITPENSKNKVVLQSLKPWRTDVYFNKATGKYEILGLKYADLQFEKGTGTYKISQEKYNDIKKKEGVDSDSEFKFTLYKNDLLLVKDTETKEQQLFRFLSRTLPKQKHYVELKPYDKQKFEGGEALIKVLGNVANGGQCIKGLAKSNISIYKVRTDVLGNQHIIKNEGDKPKLDF

SEQ ID NO: 383 - protein, 1122 aa, synthetic construct
  REGION 1..1122: note = Description of Artificial Sequence: Syntheticpolypeptide
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEEQLLDIETGELISDDEYKESVFKAPYQHFVDTLKSKEFEDSILFSYQVDSKFNRKISDATIYATRQAKVGKDKKDETYVLGKIKDIYTQDGYDAFMKIYKKDKSKFLMYRHDPQTFEKVIEPILENYPNKQMNEKGKEVPCNPFLKYKEEHGYIRKYSKKGNGPEIKSLKYYDSKLLGNPIDITPENSKNKVVLQSLKPWRTDVYFNKNTGKYEILGLKYADLQFEKKTGTYKISQEKYNGIMKEEGVDSDSEFKFTLYKNDLLLVKDTETKEQQLFRFLSRTMPNVKYYVELKPYSKDKFEKNESLIEILGSADKSGRCIKGLGKSNISIYKVRTDVLGNQHIIKNEGDKPKLDF

SEQ ID NO: 384 - protein, 1120 aa, synthetic construct
  REGION 1..1120: note = Description of Artificial Sequence: Syntheticpolypeptide
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSLKPWRTDVYFNKNTGKYEILGLKYSDMQFEKGTGKYSISKEQYENIKVREGVDENSEFKFTLYKNDLLLLKDSENGEQILLRFTSRNDTSKHYVELKPYNRQKFEGSEYLIKSLGTVAKGGQCIKGLGKSNISIYKVRTDVLGNQHIIKNEGDKPKLDF

SEQ ID NO: 385 - protein, 1120 aa, synthetic construct
  REGION 1..1120: note = Description of Artificial Sequence: Syntheticpolypeptide
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSLKPWRTDVYFNKNTGKYEILGLKYSDMQFEKGTGKYSISKEQYENIKVREGVDENSEFKFTLYKNDLLLLKDSENGEQILLRFTSRNDTSKHYVELKPYNRQKFEGSEYLIKSLGTVVKGGRCIKGLGKSNISIYKVRTDVLGNQHIIKNEGDKPKLDF

SEQ ID NO: 386 - DNA, 24 bp, synthetic construct
  misc_feature 1..24: note = Description of Artificial Sequence: Syntheticoligonucleotide
  source 1..24: mol_type = other DNA
gttgtagctccctttctcatttcg

SEQ ID NO: 387 - DNA, 93 bp, synthetic construct
  misc_feature 1..93: note = Description of Artificial Sequence: Syntheticoligonucleotide
  source 1..93: mol_type = other DNA
cgaaatgagaaccgttgctacaataaggccgtctgaaaagatgtgccgcaacgctctgccccttaaagcttctgctttaaggggcatcgttta

SEQ ID NO: 388 - DNA, 25 bp, synthetic construct
  misc_feature 1..25: note = Description of Artificial Sequence: Syntheticoligonucleotide
  source 1..25: mol_type = other DNA
gttgtagctccctttttcatttcgc

SEQ ID NO: 389 - DNA, 87 bp, synthetic construct
  misc_feature 1..87: note = Description of Artificial Sequence: Syntheticoligonucleotide
  source 1..87: mol_type = other DNA
gcgaaatgaaaaacgttgttacaataagagatgaatttctcgcaaagctctgcctcttgaaatttcggtttcaagaggcatcttttt

SEQ ID NO: 390 - DNA, 14 bp, synthetic construct
  misc_feature 1..14: note = Description of Artificial Sequence: Syntheticoligonucleotide
  source 1..14: mol_type = other DNA
gttttagtactctg

SEQ ID NO: 391 - DNA, 58 bp, synthetic construct
  misc_feature 1..58: note = Description of Artificial Sequence: Syntheticoligonucleotide
  source 1..58: mol_type = other DNA
cagaatctactaaaacaaggcaaaatgccgtgtttatctcgtcaacttgttggcgaga

SEQ ID NO: 392 - DNA, 18 bp, synthetic construct
  misc_feature 1..18: note = Description of Artificial Sequence: Syntheticoligonucleotide
  source 1..18: mol_type = other DNA
gttttagtactctgtaat

SEQ ID NO: 393 - DNA, 62 bp, synthetic construct
  misc_feature 1..62: note = Description of Artificial Sequence: Syntheticoligonucleotide
  source 1..62: mol_type = other DNA
attacagaatctactaaaacaaggcaaaatgccgtgtttatctcgtcaacttgttggcgaga

SEQ ID NO: 394 - DNA, 14 bp, synthetic construct
  misc_feature 1..14: note = Description of Artificial Sequence: Syntheticoligonucleotide
  source 1..14: mol_type = other DNA
gttttagtactctg

SEQ ID NO: 395 - DNA, 63 bp, synthetic construct
  misc_feature 1..63: note = Description of Artificial Sequence: Syntheticoligonucleotide
  source 1..63: mol_type = other DNA
cagaatctactaaaacaaggcaaaatgccgtgtttatctcgtcaacttgttggcgagattttt

SEQ ID NO: 396 - DNA, 14 bp, synthetic construct
  misc_feature 1..14: note = Description of Artificial Sequence: Syntheticoligonucleotide
  source 1..14: mol_type = other DNA
gttttagtactctg

SEQ ID NO: 397 - DNA, 63 bp, synthetic construct; identical to SEQ ID NO: 395
  misc_feature 1..63: note = Description of Artificial Sequence: Syntheticoligonucleotide
  source 1..63: mol_type = other DNA

SEQ ID NO: 398 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 399 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 400 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 401 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 402 - DNA, 17 bp, synthetic construct
  misc_feature 1..17: note = Description of Artificial Sequence: Syntheticoligonucleotide
  source 1..17: mol_type = other DNA
gtttaagagctatgctg

SEQ ID NO: 403 - DNA, 65 bp, synthetic construct
  misc_feature 1..65: note = Description of Artificial Sequence: Syntheticoligonucleotide
  source 1..65: mol_type = other DNA
cagcatagcaagtttaaataaggctagtccgttatcaacttgaaaaagtggcaccgagtcggtgc

SEQ ID NO: 404 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 405 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 406 - DNA, 66 bp, synthetic construct
  misc_feature 1..66: note = Description of Artificial Sequence: Syntheticoligonucleotide
  source 1..66: mol_type = other DNA
cagaagctacaaagataaggcttcatgccgaaatcaacaccctgtcattttatggcagggtgtttt

SEQ ID NO: 407 - DNA, 14 bp, synthetic construct
  misc_feature 1..14: note = Description of Artificial Sequence: Syntheticoligonucleotide
  source 1..14: mol_type = other DNA
gtctttgtactctg

SEQ ID NO: 408 - DNA, 16 bp, synthetic construct
  misc_feature 1..16: note = Description of Artificial Sequence: Syntheticoligonucleotide
  source 1..16: mol_type = other DNA
gctatagttccttact

SEQ ID NO: 409 - DNA, 127 bp, synthetic construct
  misc_feature 1..127: note = Description of Artificial Sequence: Syntheticpolynucleotide
  source 1..127: mol_type = other DNA
ggtaagttgctatagtaagggcaacagacccgaggcgttggggatcgcctagcccgtgtttacgggctctccccatattcaaaataatgacagacgagcaccttggagcatttatctccgaggtgct

SEQ ID NO: 410 - RNA, 14 nt, synthetic construct
  misc_feature 1..14: note = Description of Artificial Sequence: Syntheticoligonucleotide
  source 1..14: mol_type = other RNA
gtcttagtactctg

SEQ ID NO: 411 - RNA, 65 nt, synthetic construct
  misc_feature 1..65: note = Description of Artificial Sequence: Syntheticoligonucleotide
  source 1..65: mol_type = other RNA
cagaatctactaagacaaggcaaaatgccgtgtttatctcgtcaacttgttggcgagattttttt

SEQ ID NO: 412 - RNA, 14 nt, synthetic construct
  misc_feature 1..14: note = Description of Artificial Sequence: Syntheticoligonucleotide
  source 1..14: mol_type = other RNA
gtcttagtactctg

SEQ ID NO: 413 - RNA, 65 nt, synthetic construct; identical to SEQ ID NO: 411
  misc_feature 1..65: note = Description of Artificial Sequence: Syntheticoligonucleotide
  source 1..65: mol_type = other RNA

SEQ ID NO: 414 - RNA, 14 nt, synthetic construct
  misc_feature 1..14: note = Description of Artificial Sequence: Syntheticoligonucleotide
  source 1..14: mol_type = other RNA
gtcttagtactctg

SEQ ID NO: 415 - RNA, 65 nt, synthetic construct; identical to SEQ ID NO: 411
  misc_feature 1..65: note = Description of Artificial Sequence: Syntheticoligonucleotide
  source 1..65: mol_type = other RNA

SEQ ID NO: 416 - RNA, 14 nt, synthetic construct
  misc_feature 1..14: note = Description of Artificial Sequence: Syntheticoligonucleotide
  source 1..14: mol_type = other RNA
gtcttagtactctg

SEQ ID NO: 417 - RNA, 65 nt, synthetic construct; identical to SEQ ID NO: 411
  misc_feature 1..65: note = Description of Artificial Sequence: Syntheticoligonucleotide
  source 1..65: mol_type = other RNA

SEQ ID NO: 418 - RNA, 12 nt, synthetic construct
  misc_feature 1..12: note = Description of Artificial Sequence: Syntheticoligonucleotide
  source 1..12: mol_type = other RNA
actggggttcag

SEQ ID NO: 419 - RNA, 76 nt, synthetic construct
  misc_feature 1..76: note = Description of Artificial Sequence: Syntheticoligonucleotide
  source 1..76: mol_type = other RNA
ctgaacctcagtaagcattggctcgtttccaatgttgattgctccgccggtgctccttatttttaagggcgccggc

SEQ ID NO: 420 - DNA, 12 bp, synthetic construct
  misc_feature 1..12: note = Description of Artificial Sequence: Syntheticoligonucleotide
  source 1..12: mol_type = other DNA
gttttagtccct

SEQ ID NO: 421 - RNA, 16 nt, synthetic construct
  misc_feature 1..16: note = Description of Artificial Sequence: Syntheticoligonucleotide
  source 1..16: mol_type = other RNA
gtcatagttcccctga

SEQ ID NO: 422 - RNA, 98 nt, synthetic construct
  misc_feature 1..98: note = Description of Artificial Sequence: Syntheticoligonucleotide
  source 1..98: mol_type = other RNA
tcagggttactatgataagggctttctgcctaaggcagactgacccgcggcgttggggatcgcctgtcgcccgcttttggcgggcattccccatcctt

SEQ ID NO: 423 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 424 - DNA, 63 bp, synthetic construct
  misc_feature 1..63: note = Description of Artificial Sequence: Syntheticoligonucleotide
  source 1..63: mol_type = other DNA
agggactaaaataaagagtttgcgggactctgcggggttacaatcccctaaaaccgctttttt

SEQ ID NO: 425 - DNA, 24 bp, synthetic construct
  misc_feature 1..24: note = Description of Artificial Sequence: Syntheticoligonucleotide
  source 1..24: mol_type = other DNA
gttgtagctccctttctcatttcg

SEQ ID NO: 426 - DNA, 93 bp, synthetic construct; identical to SEQ ID NO: 387
  misc_feature 1..93: note = Description of Artificial Sequence: Syntheticoligonucleotide
  source 1..93: mol_type = other DNA

SEQ ID NO: 427 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 428 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 429 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 430 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 431 - DNA, 17 bp, synthetic construct
  misc_feature 1..17: note = Description of Artificial Sequence: Syntheticoligonucleotide
  source 1..17: mol_type = other DNA
gtttaagagctatgctg

SEQ ID NO: 432 - DNA, 65 bp, synthetic construct; identical to SEQ ID NO: 403
  misc_feature 1..65: note = Description of Artificial Sequence: Syntheticoligonucleotide
  source 1..65: mol_type = other DNA

SEQ ID NO: 433 - DNA, 17 bp, synthetic construct
  misc_feature 1..17: note = Description of Artificial Sequence: Syntheticoligonucleotide
  source 1..17: mol_type = other DNA
gtttaagagctatgctg

SEQ ID NO: 434 - DNA, 65 bp, synthetic construct; identical to SEQ ID NO: 403
  misc_feature 1..65: note = Description of Artificial Sequence: Syntheticoligonucleotide
  source 1..65: mol_type = other DNA

SEQ ID NO: 435 - DNA, 17 bp, synthetic construct
  misc_feature 1..17: note = Description of Artificial Sequence: Syntheticoligonucleotide
  source 1..17: mol_type = other DNA
gtttaagagctatgctg

SEQ ID NO: 436 - DNA, 65 bp, synthetic construct; identical to SEQ ID NO: 403
  misc_feature 1..65: note = Description of Artificial Sequence: Syntheticoligonucleotide
  source 1..65: mol_type = other DNA

SEQ ID NO: 437 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 438 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 439 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 440 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 441 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 442 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 443 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 444 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 445 - DNA, 12 bp, synthetic construct
  misc_feature 1..12: note = Description of Artificial Sequence: Syntheticoligonucleotide
  source 1..12: mol_type = other DNA
gttttagagcta

SEQ ID NO: 446 - DNA, 60 bp, synthetic construct; identical to SEQ ID NO: 44
  misc_feature 1..60: note = Description of Artificial Sequence: Syntheticoligonucleotide
  source 1..60: mol_type = other DNA

SEQ ID NO: 447 - DNA, 17 bp, synthetic construct
  misc_feature 1..17: note = Description of Artificial Sequence: Syntheticoligonucleotide
  source 1..17: mol_type = other DNA
gtttaagagctatgctg

SEQ ID NO: 448 - DNA, 65 bp, synthetic construct; identical to SEQ ID NO: 403
  misc_feature 1..65: note = Description of Artificial Sequence: Syntheticoligonucleotide
  source 1..65: mol_type = other DNA

SEQ ID NO: 449 - DNA, 17 bp, synthetic construct
  misc_feature 1..17: note = Description of Artificial Sequence: Syntheticoligonucleotide
  source 1..17: mol_type = other DNA
gtttaagagctatgctg

SEQ ID NO: 450 - DNA, 65 bp, synthetic construct; identical to SEQ ID NO: 403
  misc_feature 1..65: note = Description of Artificial Sequence: Syntheticoligonucleotide
  source 1..65: mol_type = other DNA

SEQ ID NO: 451 - DNA, 14 bp, synthetic construct
  misc_feature 1..14: note = Description of Artificial Sequence: Syntheticoligonucleotide
  source 1..14: mol_type = other DNA
gtctttgtactctg

SEQ ID NO: 452 - DNA, 66 bp, synthetic construct; identical to SEQ ID NO: 406
  misc_feature 1..66: note = Description of Artificial Sequence: Syntheticoligonucleotide
  source 1..66: mol_type = other DNA

SEQ ID NO: 453 - DNA, 14 bp, synthetic construct
  misc_feature 1..14: note = Description of Artificial Sequence: Syntheticoligonucleotide
  source 1..14: mol_type = other DNA
gtctttgtactctg

SEQ ID NO: 454 - DNA, 66 bp, synthetic construct; identical to SEQ ID NO: 406
  misc_feature 1..66: note = Description of Artificial Sequence: Syntheticoligonucleotide
  source 1..66: mol_type = other DNA

SEQ ID NO: 455 - DNA, 14 bp, synthetic construct
  misc_feature 1..14: note = Description of Artificial Sequence: Syntheticoligonucleotide
  source 1..14: mol_type = other DNA
gtctttgtactctg

SEQ ID NO: 456 - DNA, 66 bp, synthetic construct; identical to SEQ ID NO: 406
  misc_feature 1..66: note = Description of Artificial Sequence: Syntheticoligonucleotide
  source 1..66: mol_type = other DNA

SEQ ID NO: 457 - DNA, 14 bp, synthetic construct
  misc_feature 1..14: note = Description of Artificial Sequence: Syntheticoligonucleotide
  source 1..14: mol_type = other DNA
gtctttgtactctg

SEQ ID NO: 458 - DNA, 66 bp, synthetic construct; identical to SEQ ID NO: 406
  misc_feature 1..66: note = Description of Artificial Sequence: Syntheticoligonucleotide
  source 1..66: mol_type = other DNA

SEQ ID NO: 459 - RNA, 21 nt, Homo sapiens
  source 1..21: mol_type = other RNA
cataaagtagaaagcactact

SEQ ID NO: 460 - RNA, 23 nt, Homo sapiens
  source 1..23: mol_type = other RNA
tgtagtgtttcctactttatgga

SEQ ID NO: 461 - RNA, 24 nt, Homo sapiens
  source 1..24: mol_type = other RNA
tttggcaatggtagaactcacact

SEQ ID NO: 462 - RNA, 21 nt, Homo sapiens
  source 1..21: mol_type = other RNA
tggttctagacttgccaacta

SEQ ID NO: 463 - RNA, 22 nt, Homo sapiens
  source 1..22: mol_type = other RNA
tatggcactggtagaattcact

SEQ ID NO: 464 - RNA, 22 nt, Homo sapiens
  source 1..22: mol_type = other RNA
gtgaattaccgaagggccataa

SEQ ID NO: 465 - RNA, 22 nt, Homo sapiens
  source 1..22: mol_type = other RNA
tggagtgtgacaatggtgtttg

SEQ ID NO: 466 - RNA, 22 nt, Homo sapiens
  source 1..22: mol_type = other RNA
aacgccattatcacactaaata